(19) United States
(12) Patent Application Publication
(10) Pub. No.: US 2026/0144862 A1
(43) Pub. Date: May 28, 2026

(54) MATERIALS AND METHODS TO TREAT EPSTEIN-BARR VIRUS (EBV) AND EBV-INDUCED DISEASES

(71) Applicant: Medizinische Universität Wien, Wien (AT)

(72) Inventors: Hannes VIETZEN, Wien (AT); Elisabeth PUCHHAMMER STÖCKL, Wien (AT)

(73) Assignee: Medizinische Universität Wien, Wien (AT)

(21) Appl. No.: 19/121,909

(22) PCT Filed: Oct. 20, 2023
(86) PCT No.: PCT/EP2023/079379

§ 371 Date: Apr. 17, 2025

(30) Foreign Application Priority Data
Oct. 21, 2022 (EP) 22203139.5

(51) Int. Cl. A61K 39/245; A61K 39/00; A61P 37/04

(52) U.S. Cl. CPC A61K 39/245; A61P 37/04; A61K 2039/53; A61K 2039/54; A61K 2039/545

(57) ABSTRACT

The present invention relates to means and methods to prevent and/or treat Epstein-Barr virus (EBV) and EBV-induced diseases, such as EBV infection, infectious mononucleosis (IM), malignant or non-malignant post-transplant lymphoproliferative disorder (PTLD) and other EBV-associated diseases. In particular, the invention provides a SQAPLPCVL peptide that can be used in a treatment or a method of treatment to induce an EBV-specific immune response in a subject. The SQAPLPCVL can be used in a treatment or method of treatment as a vaccine against EBV and EBV-induced diseases. It is preferred herein that Epstein-Barr virus (EBV) and/or EBV-induced diseases are prevented.

The present invention relates to means and methods to prevent and/or treat Epstein-Barr virus (EBV) and EBV-induced diseases, such as EBV infection, infectious mononucleosis (IM), malignant or non-malignant post-transplant lymphoproliferative disorder (PTLD) and other EBV-associated diseases. In particular, the invention provides a SQAPLPCVL peptide that can be used in a treatment or a method of treatment to induce an EBV-specific immune response in a subject. The SQAPLPCVL can be used in a treatment or method of treatment as a vaccine against EBV and EBV-induced diseases. It is preferred herein that Epstein-Barr virus (EBV) and/or EBV-induced diseases are prevented.

Epstein-Barr virus (EBV) is a ubiquitous herpesvirus, which infects over 90% of the adult human population worldwide. Primary EBV infection may result in a self-limiting infectious mononucleosis (IM), which is hallmarked by fever, lymphadenopathy, and tonsillitis, often associated with splenomegaly and/or self-resolving hepatitis. In the absence of a licensed EBV vaccine, cases of IM result in a high economic burden for public health services¹. However, the cumulative risk to develop IM upon primary EBV infection is estimated only between 13.3%-22.4%², and it is so far an unresolved question, why during primary EBV infection some patients develop clinically evident IM, while others remain asymptomatic.

After primary infection, EBV establishes a life-long persistent infection in memory B cells, from which sporadic reactivations may occur. EBV is associated with the development of malignant EBV-associated diseases, resulting worldwide in >137,900 annual deaths³. In solid-organ (SOT) and hematopoietic stem cell transplant (HSCT) recipients, EBV may cause malignant post-transplant lymphoproliferative disorders (PTLD), which are associated with high morbidity and poor survival⁴.

The EBV-specific immune responses are hallmarked by potent cytotoxic CD8⁺ T cell and natural killer (NK) cell responses⁵⁻⁷. Among the broad EBV-specific CD8⁺ T cell responses, a small subset of CD8⁺ T cells bind with their ap T-cell receptor to the non-classical HLA molecule HLA-E⁸. HLA-E shows a strictly restricted expression pattern, including B cells⁹. HLA-E is highly conserved in European populations and only two allelic variants, the high-expressing HLA-E*0103 and the low-expressing HLA-E*0101 are prevalent⁹. The limited polymorphism results in a restricted set of distinct EBV-derived peptides, which can be presented via HLA-E on the surface of EBV-infected cells. It was shown that HLA-E is stabilized by the conserved EBV-encoded BZLF1-peptide or by highly polymorphic EBV LMP-1-derived peptides¹⁰. HLA-E further binds to the inhibitory NKG2A/CD94 receptor complex, which is expressed on distinct CD8⁺ T and NK cell subsets. By their peptides presented via HLA-E, EBV infections elicit the expansion of NKG2A⁺ NK cells; a NK cell subset, which respond to EBV-infected cells by the secretion of pro-inflammatory cytokines and cellular cytotoxicity^{6,11}. The EBV-encoded peptides result, however, in the inhibition of NKG2A⁺ NK cells and NKG2A⁺ CD8⁺ T cells, preventing a potent immune response against EBV-infected cells.

WO 2019/123169 discloses the EBV ZEBRA protein (Genbank No. P03206) and fragments thereof which comprise or partially comprise the SQAPLPCVL peptide. However, WO 2019/123169 does not suggest an immunization against EBV with the SQAPLPCVL peptide per se. Longer peptides comprising or partially comprising the SQAPLPCVL motif have been shown herein to not elicit the immune response which is elicited by the SQAPLPCVL peptide; see Example 4 and FIG. 15.

Ulbrecht et al. 1998³⁴ discloses the binding/stabilization of HLA-E by the SQAPLPCVL peptide and of related peptides (see Table I and II); however, the authors do not evaluate any HLA-E restricted T cell response and thus not any functional immune response as demonstrated herein. Ulbrecht et al. 1998³⁴ also does not disclose an EBV immunization via the SQAPLPCVL peptide. As shown herein, binding to HLA-E alone as done in Ulbrecht 34 does not allow any predictions about whether the binding peptides induce an immune response; see Examples 2 and 3 and FIGS. 13 and 14.

Sharpe et al. 2019³⁵ discloses binding/stabilization of HLA-E by the SQAPLPCVL peptide (see Table 2). The authors mention that an HLA-E mediated presentation of pathogen-derived peptides to T cells has been observed during infection with human Cytomegalovirus, Mycobacterium tuberculosis, Salmonella enterica and simian immunodeficiency virus (see page 171, right column, first para.). Sharpe et al.³⁵ does not suggest that an EBV specific HLA-E restricted T cell response or immunization via the SQAPLPCVL peptide might be elicited. From the binding to HLA-E alone no assumptions can be made whether the peptide could induce an immune response or could be used for immunization against EBV; see also Examples 2 and 3 as well as FIGS. 13 and 14.

Abels et al. 2018³⁶ discloses that the SQAPLPCVL can be recognized by CD8+CD94/NKG2C+ T cells. However, the document is not concerned with therapy or vaccination against EBV (see page 10, first para.). Moreover, the cell population shown herein to be elicited by the SQAPLPCVL peptide in context of immunization against EBV is not proposed in Abels et al.³⁶. This further illustrates the absence of any pointer in Abels et al.³⁶ that the SQAPLPCVL peptide might be useful in vaccination against EBV.

So far, there are no licensed EBV vaccines, and recent EBV vaccine candidates failed to induce protective immune responses, e.g. against IM or malignant, lymphoproliferative EBV-induced diseases³³. EBV-associated diseases result in more than 137,000 annual deaths and a high economic burden for public health services¹.

Thus, there is a need for a prophylactic and/or therapeutic EBV vaccine for the therapy of EBV and EBV-induced diseases. The technical problem underlying the present invention is the treatment and/or prevention of EBV and EBV-induced diseases.

The technical problem is solved by provision of the embodiments characterized in the claims and as provided herein below. Specifically, the technical problem is solved, and the above-mentioned difficulties are overcome by the provision of an EBV vaccine.

Accordingly, the invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease.

In another aspect, the invention relates to a method of treating EBV or an EBV-induced disease comprising administering an effective amount of an EBV vaccine to a subject, wherein the EBV vaccine comprises a SQAPLPCVL peptide or a nucleic acid encoding a SQAPLPCVL peptide and, optionally a pharmaceutically acceptable carrier.

In another aspect, the invention relates to a method of inducing an EBV-specific immune response in a subject, comprising administering an EBV vaccine to a subject, wherein the EBV vaccine comprises a SQAPLPCVL peptide or a nucleic acid encoding a SQAPLPCVL peptide and optionally a pharmaceutically acceptable carrier.

The treatment of EBV or of an EBV-induced disease preferably is a prevention of EBV or of an EBV-induced disease.

In another aspect, the invention relates to a method to elicit HLA-E-restricted CD8⁺ T cells optionally comprising HLA-E-restricted NKG2A⁺ CD8⁺ T cells and/or HLA-E-restricted NKG2A⁻ CD8⁺ T cells, or a HLA-E-restricted CD8⁺ T cell response optionally comprising a NKG2A⁺ CD8⁺ T cell response and/or a NKG2A⁻ CD8⁺ T cell response in a subject, comprising administering an EBV vaccine to the subject, wherein the EBV vaccine comprises a SQAPLPCVL peptide or a nucleic acid encoding a SQAPLPCVL peptide and, optionally, a pharmaceutically acceptable carrier.

In another aspect, the invention relates to an in vitro or ex vivo method to produce HLA-E-restricted CD8⁺ T cells optionally comprising HLA-E-restricted NKG2A⁺ CD8⁺ T cells and/or HLA-E-restricted NKG2A⁻ CD8⁺ T cells, or to elicit a HLA-E-restricted CD8⁺ T cell response optionally comprising a NKG2A⁺ CD8⁺ T cell response and/or a NKG2A⁻ CD8⁺ T cell response in a cell, comprising contacting a cell with an EBV vaccine, wherein the EBV vaccine comprises a SQAPLPCVL peptide or a nucleic acid encoding a SQAPLPCVL peptide and, optionally, a pharmaceutically acceptable carrier.

In another aspect, the invention relates to an Epstein-Barr-Virus (EBV) vaccine, comprising a SQAPLPCVL peptide or a nucleic acid encoding a SQAPLPCVL peptide and, optionally, a pharmaceutically acceptable carrier.

In another aspect, the invention relates to a SQAPLPCVL peptide or a nucleic acid encoding a SQAPLPCVL peptide for use as a vaccine.

In another aspect, the invention relates to a SQAPLPCVL peptide or a nucleic acid encoding a SQAPLPCVL peptide for use as a vaccine against EBV.

In another aspect, the invention relates to a SQAPLPCVL peptide or a nucleic acid encoding a SQAPLPCVL peptide for use as a medicament.

In another aspect, the invention relates to a SQAPLPCVL peptide or a nucleic acid encoding a SQAPLPCVL peptide for use in the treatment of EBV or an EBV-induced disease.

The invention provides, inter alia, the following advantages:
Prophylactic and therapeutic treatment of EBV infections and EBV-induced diseases
The SQAPLPCVL peptide induced EBV-specific immune response efficiently reduces and/or prevents the EBV spread and the increase of the virus load.
The EBV-specific HLA-E-restricted CD8⁺ T cell response confers protection from EBV and EBV-induced diseases by killing EBV infected host cells.
The EBV-specific HLA-E-restricted CD8⁺ T cell response can be activated in subjects that suffer from an acute symptomatic EBV infection or acute symptomatic EBV-induced diseases and can ameliorate the same by killing EBV infected host cells and reducing viral spread and/or replication.
The EBV vaccine is especially useful for early priming of the immune system to establish long-term protection from EBV spread thus preventing symptomatic EBV infections and EBV-induced diseases, a primary dose of the EBV vaccine can be administered e.g. to children.
Subjects undergoing immunosuppression or suffering from an immunodeficiency can be treated prophylactically to avoid an EBV reactivation, e.g. in anticipation of/prior to an organ- or stem cell transplantation.

In addition, the inventors provide evidence that a SQAPLPCVL-peptide vaccine elicits potent SQAPLPCVL-specific HLA-E-restricted CD8⁺ T cells, which can prevent the EBV-spread during primary infection, EBV-reinfections as well as EBV-reactivation and consequently reduce the risk for EBV-associated diseases in general and EBV-associated lymphoproliferative diseases in particular. Further, they demonstrate that SQAPLPCVL-specific, HLA-E-restricted CD8⁺ T cell responses are low in patients with symptomatic EBV-infections and are high in patients with asymptomatic EBV-infections; thus, it is shown that high vaccine-induced SQAPLPCVL-specific, HLA-E-restricted CD8⁺ T cell responses are protective against symptomatic EBV-infections, which T cell responses can be elicited with the EBV vaccine of the present invention.

Furthermore, the inventors could show that peptides similar to the SQAPLPCVL-peptide, such as alterations in sequence or length do not elicit an HLA-E-restricted CD8⁺ T cell response, i.e. cannot not confer immunity to EBV, EBV-associated disease and/or EBV-induced disease; see Examples 2-5.

The inventors also provide evidence that the SQAPLPCVL-specific HLA-E-restricted CD8⁺ T cell response, e.g. induced by a vaccine comprising the SQAPLPCVL-peptide, can be used to prevent or treat multiple sclerosis (Example 6), EBV-associated lymphoproliferative diseases (Example 7) and EBV-associated gastric carcinoma (Example 8). Thus, the vaccine comprising the SQAPLPCVL-peptide of the present invention can be used as a universal prophylactic/therapeutic treatment of EBV, EBV-associated disease and/or EBV-induced disease.

The above is illustrated in the appended examples.

In the following the invention is described in more detail.
1. An Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease.
2. The EBV vaccine for use of item 1, wherein the EBV-induced disease is selected from the group of infectious mononucleosis (IM), lymphoproliferative diseases and/or malignant diseases.
3. The EBV vaccine for use of item 1 or 2, wherein the EBV-induced disease is infectious mononucleosis (IM) caused by a primary infection with EBV (which may include postinfectious chronic fatigue syndrome), post-transplant lymphoproliferative disorder (PTLD), malignant post-transplant lymphoproliferative disorder (PTLD), Burkitt lymphoma, hemophagocytic lymphohistiocytosis, EBV-associated Hodgkin lymphoma, EBV-associated gastric carcinoma, nasopharyngeal carcinoma, B-cell lymphoma, lymphomatoid granulomatosis, multiple sclerosis, long COVID, myalgic encephalomyelitis or chronic fatigue syndrome.
4. The EBV vaccine for use of any one of items 1-3, wherein the treatment of EBV is the treatment of EBV infection, preferably wherein EBV infection is EBV primary infection or the treatment of EBV infection following EBV reactivation or EBV reinfections, preferably wherein the treatment is a prophylactic treatment (prevention).
5. The EBV vaccine for use of any one of items 1-4, wherein the treatment comprises administering the EBV vaccine to a subject in an effective amount.
6. The EBV vaccine for use of item 5, wherein an effective amount is a total dose of 30-3000 μg.
7. The EBV vaccine for use of item 5 or 6, wherein the EBV vaccine is administered preferably by intradermal injection, intramuscular injection or subcutaneous injection.
8. The EBV vaccine for use of any one of items 1-7, wherein the treatment is a prophylactic treatment (prevention) and/or a therapeutic/acute treatment.
9. The EBV vaccine for use of any one of items 1-8, wherein the subject has been exposed to EBV, is infected with EBV or is at risk of EBV infection or an EBV-induced disease.
10. The EBV vaccine for use of any one of items 1-9, wherein the subject has received or will receive a transplant, such as an organ or stem cell transplant and/or, wherein the subject has a congenital or acquired immunodeficiency.
11. The EBV vaccine for use of item 10, wherein a subject has received or will receive a solid-organ or hematopoietic stem cell transplant.
12. The EBV vaccine for use of any one of items 1-11, wherein the subject is not infected with EBV.
13. The EBV vaccine for use of any one of items 1-12, wherein the subject is a human.
14. The EBV vaccine for use of item 13, wherein the human has an HLA-E*0101/0101, HLA-E*0101/0103 or HLA-E*0103/0103 genotype.
15. The EBV vaccine for use of item 13 or 14, wherein the subject is a human in an EBV endemic region or population.
16. The EBV vaccine for use of any one of items 13-15, wherein the subject is a young child, an adolescent/teenager, an adult, or an elderly person.
17. The EBV vaccine for use of any one of items 1-16, wherein a single dose of the EBV vaccine is administered.
18. The EBV vaccine for use of item 17, wherein the treatment further comprises administering one or more booster doses of the EBV vaccine.
19. The EBV vaccine for use of any one of items 1-18, wherein the EBV vaccine produces or is capable of producing an EBV-specific immune response (in the subject to be treated/to whom the vaccine is to be administered).
20. The EBV vaccine for use of item 19, wherein the immune response is a T cell response.
21. The EBV vaccine for use of item 19 or 20, wherein the immune response is a HLA-E-restricted CD8⁺ T cell response.
22. The EBV vaccine for use of item 21, wherein the HLA-E-restricted CD8⁺ T cell response comprises a HLA-E-restricted NKG2A⁺ CD8⁺ T cell response and/or a NKG2A⁻ CD8⁺ T cell response.
23. The EBV vaccine for use of item 21 or 22, wherein the HLA-E-restricted CD8⁺ T cell response is a SQAPLPCVL-specific HLA-E-restricted CD8⁺ T cell response, optionally comprising a SQAPLPCVL-specific HLA-E-restricted NKG2A⁺ CD8⁺ T cell response and/or a SQAPLPCVL-specific HLA-E-restricted NKG2A⁻ CD8⁺ T cell response.
24. The EBV vaccine for use of any one of items 1 to 23, wherein the EBV vaccine and/or the immune response prevent EBV spread and/or EBV replication or is capable of preventing EBV spread and/or EBV replication (in the subject to be treated/to whom the vaccine is to be administered).
25. The EBV vaccine for use of any one of items 1-24, wherein the EBV vaccine results in long-term immunity to EBV or an EBV-induced disease.
26. The EBV vaccine for use of any one of items 1-25, wherein the EBV vaccine results in EBV-specific memory B cells or memory T cells.
27. The EBV vaccine for use of any one of items 1-26, wherein the nucleic acid encoding a SQAPLPCVL peptide is a non-self-replicating desoxyribonucleic acid (DNA), such as a vector.
28. The EBV vaccine for use of any one of items 1-27, wherein the nucleic acid encoding a SQAPLPCVL peptide is a non-self-replicating ribonucleic acid (RNA), such as a mRNA, optionally wherein the RNA is a modified RNA, such as a modified mRNA.
29. A method of treating EBV or an EBV-induced disease comprising administering an effective amount of an EBV vaccine to a subject, wherein the EBV vaccine comprises a SQAPLPCVL peptide or a nucleic acid encoding a SQAPLPCVL peptide and, optionally a pharmaceutically acceptable carrier.
30. The method of item 29, wherein the EBV-induced disease is selected from the group of infectious mononucleosis (IM), lymphoproliferative diseases or malignant diseases.
31. The method of item 29 or 30 wherein the EBV-induced disease is infectious mononucleosis (IM) caused by a primary infection with EBV (which may include postinfectious chronic fatigue syndrome), post-transplant lymphoproliferative disorder (PTLD), malignant post-transplant lymphoproliferative disorder (PTLD), Burkitt lymphoma, hemophagocytic lymphohistiocytosis, EBV-associated Hodgkin lymphoma, EBV-associated gastric carcinoma, nasopharyngeal carcinoma, B-cell lymphoma, lymphomatoid granulomatosis, multiple sclerosis, long COVID, myalgic encephalomyelitis or chronic fatigue syndrome.
32. The method of any one of items 29-31, wherein the treatment of EBV is the treatment of EBV infection, preferably wherein EBV infection is EBV primary infection or the treatment of EBV infection following EBV reactivation or EBV reinfections, preferably wherein the treatment is a prophylactic treatment (prevention).
33. The method of any one of items 29-32, wherein an effective amount is a total dose of 30-3000 μg.
34. The method of any one of items 29-33, wherein the EBV vaccine is administered by intradermal injection, intramuscular injection, or subcutaneous injection.
35. The method of any one of items 29-34, wherein the treatment is a prophylactic treatment (prevention) and/or a therapeutic/acute treatment.
36. The method of any one of items 29-35, wherein the subject has been exposed to EBV, is infected with EBV or is at risk of EBV infection.
37. The method of any one of items 29-36, wherein the subject has received or will receive a transplant such as an organ or stem cell transplant and/or, wherein the subject has a congenital or acquired immunodeficiency.
38. The method of item 37, wherein a subject has received or will receive a solid-organ or hematopoietic stem cell transplant.
39. The method of any one of items 29-38, wherein the subject is not infected with EBV.
40. The method of any one of items 29-39, wherein the subject is a human.
41. The method of item 40, wherein the human has an HLA-E*0101/0101, HLA-E*0101/0103 or HLA-E*0103/0103 genotype.
42. The method of item 40 or 41, wherein the subject is a human in an EBV endemic region or population.
43. The method of any one of items 40-42, wherein the subject is a young child, an adolescent/teenager, an adult or an elderly person.
44. The method of any one of items 29-43, wherein a single dose of the EBV vaccine is administered.
45. The method of item 44, wherein the method further comprises administering one or more booster doses of the EBV vaccine.
46. The method of any one of items 29-45, wherein the EBV vaccine produces an EBV-specific immune response.
47. The method of item 46, wherein the immune response is a T cell response.
48. The method of item 46 or 47, wherein the immune response is a HLA-E-restricted CD8⁺ T cell response.
49. The method of item 48, wherein the HLA-E-restricted CD8⁺ T cell response comprises a HLA-E-restricted NKG2A⁺ CD8⁺ T cell response and/or a NKG2A⁺ CD8⁺ T cell response.
50. The method of item 48 or 49, wherein the HLA-E-restricted CD8⁺ T cell response is a SQAPLPCVL-specific HLA-E-restricted CD8⁺ T cell response, optionally comprising a SQAPLPCVL-specific HLA-E-restricted NKG2A⁺ CD8⁺ T cell response and/or a SQAPLPCVL-specific HLA-E-restricted NKG2A⁻ CD8⁺ T cell response.
51. The method of any one of items 29-50, wherein the administration of the EBV vaccine and/or the immune response prevent EBV spread and/or EBV replication or is capable of preventing EBV spread and/or EBV replication (in the subject to be treated/to whom the vaccine is to be administered).
52. The method of any one of items 29-51, wherein the EBV vaccine results in long-term immunity to EBV or an EBV-induced disease.
53. The method of any one of items 29-52, wherein the EBV vaccine results in EBV-specific memory B cells or memory T cells.
54. The method of any one of items 29-53, wherein the nucleic acid encoding a SQAPLPCVL peptide is a non-self-replicating desoxyribonucleic acid (DNA), such as a vector.
55. The method of any one of items 29-53, wherein the nucleic acid encoding a SQAPLPCVL peptide is a non-self-replicating ribonucleic acid (RNA), such as a mRNA, optionally wherein the RNA is a modified RNA, such as a modified mRNA.
56. A method of inducing an EBV-specific immune response in a subject, comprising administering an EBV vaccine to a subject, wherein the EBV vaccine comprises a SQAPLPCVL peptide or a nucleic acid encoding a SQAPLPCVL peptide and optionally a pharmaceutically acceptable carrier.
57. A method to produce HLA-E-restricted CD8⁺ T cells optionally comprising HLA-E-restricted NKG2A⁺ CD8⁺ T cells and/or HLA-E-restricted NKG2A⁺ CD8⁺ T cells in a subject,
   or to elicit a HLA-E-restricted CD8⁺ T cell response optionally comprising a NKG2A⁺ CD8⁺ T cell response and/or a NKG2A⁻ CD8⁺ T cell response in a subject, comprising administering an EBV vaccine to the subject, wherein the EBV vaccine comprises a SQAPLPCVL peptide or a nucleic acid encoding a SQAPLPCVL peptide and, optionally, a pharmaceutically acceptable carrier.
58. An in vitro or ex vivo method to produce HLA-E-restricted CD8⁺ T cells optionally comprising HLA-E-restricted NKG2A⁺ CD8⁺ T cells and/or HLA-E-restricted NKG2A⁻ CD8⁺ T cells, or to elicit a HLA-E-restricted CD8⁺ T cell response optionally comprising a NKG2A⁺ CD8⁺ T cell response and/or a NKG2A⁻ CD8⁺ T cell response in a cell, comprising contacting a cell with an EBV vaccine, wherein the EBV vaccine comprises a SQAPLPCVL peptide or a nucleic acid encoding a SQAPLPCVL peptide and, optionally, a pharmaceutically acceptable carrier.
59. An Epstein-Barr-Virus (EBV) vaccine, comprising a SQAPLPCVL peptide or a nucleic acid encoding a SQAPLPCVL peptide and, optionally, a pharmaceutically acceptable carrier.
60. A SQAPLPCVL peptide or a nucleic acid encoding a SQAPLPCVL peptide for use as a vaccine.
61. A SQAPLPCVL peptide or a nucleic acid encoding a SQAPLPCVL peptide for use as a vaccine against EBV.
62. A SQAPLPCVL peptide or a nucleic acid encoding a SQAPLPCVL peptide for use as a medicament.
63. A SQAPLPCVL peptide or a nucleic acid encoding a SQAPLPCVL peptide for use in the treatment of EBV or an EBV-induced disease.
64. The SQAPLPCVL peptide or a nucleic acid encoding a SQAPLPCVL peptide for use of item 63, wherein the treatment of EBV is the treatment of EBV infection, preferably wherein EBV infection is EBV primary infection or the treatment of EBV infection following EBV reactivation or EBV reinfections, preferably wherein the treatment is a prophylactic treatment (prevention).

The invention provides an EBV vaccine for use in the treatment of EBV or an EBV-induced disease. As used herein, “treatment of EBV” preferably relates to the treatment of an EBV infection. The term “EBV infection” includes primary, secondary, or any further infection of EBV. In addition, the term “EBV infection” includes an EBV infection following reactivation of EBV. An EBV infection following reactivation may also be referred herein as “reinfection”. EBV is one of the eight known human herpesvirus types in the herpes family and is one of the most common viruses in humans. EBV is formally classified as Human gammaherpesvirus 4.

Accordingly, the terms “Epstein-Barr virus”, “EBV”, “Human gammaherpesvirus 4” and “HHV-4” may be used interchangeably herein and all relate to the same virus.

A vaccine is a composition that is capable of providing active acquired immunity to a disease. It is understood that the term “vaccine” or “EBV vaccine” refers to a composition comprising the SQAPLPCVL peptide as active agent either in peptide form or encoded by a nucleic acid and optionally a pharmaceutically acceptable carrier/excipient and the like. In general, a vaccine is a composition that is capable of evoking an immune response against a pathogen in a host.

It is envisioned herein that an EBV vaccine can be used therapeutically or prophylactically (i.e. in the prevention). When used therapeutically the subject is already suffering from an EBV infection or an EBV-induced disease, and particularly shows symptoms of an EBV infection or an EBV-induced disease. The therapeutical vaccine then triggers an immune response to treat the infection and/or disease. The EBV vaccine of the present invention may be used in the therapeutic treatment of subjects suffering from an EBV reinfection, such as a secondary or further EBV infection and/or EBV reactivation. As described herein, EBV reinfection and/or EBV reactivation may be accompanied by immunosuppressive events or an immunodeficiency in a subject.

When used prophylactically, the subject is protected from a future EBV infection or the pathological effects of a future EBV infection are ameliorated. In this context a future EBV infection can be EBV primary infection, EBV infection following EBV reactivation or EBV reinfections. A prophylactic treatment with the EBV vaccine is preferably performed on a subject which has yet not suffered from/experienced an EBV infection, e.g. a subject at a very young age. Prophylactically treating young subjects, such as human children, is preferred since they can be protected from an EBV infection in the future and can be immunized against EBV with as little as a single treatment (e.g. a single dose of the vaccine); however, maintenance of long-term immunity can require one or more booster doses. For example, a first booster dose may be given 2-6 weeks after an initial dose and a second booster dose may be given after one year.

The EBV vaccine of the present invention preferably comprises a peptide with the sequence SQAPLPCVL (SEQ ID NO: 1). SEQ ID NO: 1 encodes a BZLF1-derived EBV peptide that is highly conserved in all EBV strains (see FIG. 2). The present invention demonstrates that the SQAPLPCVL peptide successfully induces an immune response to EBV infection. In the sense of the present invention a peptide is a short chain of amino acids typically linked by peptide bonds between the alpha-amino and carbonyl groups of adjacent amino acids. The peptide of the present invention can comprise 9 amino acids. Preferably, the peptide of the present invention consists of 9 amino acids, i.e. consists of the sequence SQAPLPCVL (SEQ ID NO: 1). The terms “peptide”, “oligopeptide” and “protein” may be used interchangeably herein.

Accordingly, the present invention provides an Epstein-Barr-Virus (EBV) vaccine, comprising a SQAPLPCVL peptide, for use in the treatment of EBV or an EBV-induced disorder.

The peptide set forth in SEQ ID NO: 1 is highly conserved in all EBV strains. Thus, an exchange in the amino acid sequence of SEQ ID NO: 1 may result in no or a weaker immune response. The peptide is presented by HLA-E, which then induces an immune response towards EBV. Thus, deletion or addition of amino acids to the sequence set forth in SEQ ID NO: 1 may not be desired since said modification may interfere with HLA-E presentation. Preferably, the peptide SQAPLPCVL is not modified.

Accordingly, in a preferred aspect the present invention provides an Epstein-Barr-Virus (EBV) vaccine, comprising a peptide consisting of the amino acid sequence SQAPLPCVL, for use in the treatment of EBV or an EBV-induced disorder.

The invention provides in one aspect an Epstein-Barr-Virus (EBV) vaccine, comprising a peptide consisting of the sequence SQAPLPCVL (SEQ ID NO: 1) or a nucleic acid encoding a peptide consisting of the sequence SQAPLPCVL (SEQ ID NO: 1) and, optionally, a pharmaceutically acceptable carrier, for use in the treatment of EBV or an EBV-induced disease.

While the peptide SQAPLPCVL is necessary to induce an immune response against EBV, the peptide can still comprise one or more peptide modifications. A peptide modification may be a post-translational modification such as phosphorylation, hydroxylation, sulfonation, palmitoylation, glycosylation, and disulfide formation. A peptide modification may also be a modification of one or more amino acid side chains, for example, one or more of the amino acid side chains can be replaced with the side chain of another naturally occurring amino acid or any other side chain. Accordingly, 1, 2, 3, 4, 5, 6, 7, 8, or 9 amino acids of SEQ ID NO: 1 can be modified. 1, 2, 3, 4, 5, 6, 7, 8, or 9 amino acids of SEQ ID NO: 1 can comprise a post-translational modification such as phosphorylation, hydroxylation, sulfonation, palmitoylation, glycosylation, and disulfide formation. 1, 2, 3, 4, 5, 6, 7, 8, or 9 amino acid side chains of SEQ ID NO: 1 can be replaced with the side chain of another naturally occurring amino acid or any other side chain. In the sense of the present invention the peptide SQAPLPCVL can be modified in that one or more amino acids are added or deleted, e.g. 1, 2, 3, 4, 5, 6, 7, 8, 9 or more amino acids can be added to the peptide of SEQ ID NO: 1 or 1, 2, 3, 4, 5, or more amino acids can be deleted from the peptide of SEQ ID NO: 1. If more than 1 amino acid (e.g. 2 or more amino acids) are to be added or deleted, preferably the to be added or to be deleted amino acids are contiguous amino acids and/or preferably are added to or deleted from the N-terminus or the C-terminus of the peptide SQAPLPCVL. In one aspect, it is also envisaged that one or more of the amino acids are added to the peptide SQAPLPCVL and one or more (other) of the amino acids of the peptide SQAPLPCVL are deleted. Also, the use of a nucleic acid encoding such a modified peptide is envisaged.

In the sense of the present invention a peptide can be modified if it is still capable of triggering an immune response against EBV or EBV-induced diseases.

Accordingly, in one aspect the invention provides an Epstein-Barr-Virus (EBV) vaccine, comprising a modified SQAPLPCVL peptide, for use in the treatment of EBV or an EBV-induced disorder.

In one aspect, the invention provides an Epstein-Barr-Virus (EBV) vaccine, comprising a modified SQAPLPCVL peptide, for use in the treatment of EBV or an EBV-induced disorder, wherein the modification is a post-translational modification such as phosphorylation, hydroxylation, sulfonation, palmitoylation, glycosylation, and disulfide formation and/or, wherein one or more of the amino acid side chains is replaced with the side chain of another naturally occurring amino acid or any other side chain.

The EBV vaccine of the present invention can also comprise a nucleic acid encoding a SQAPLPCVL peptide. A “nucleic acid” in the sense of the present invention is a polymer of nucleotides, such as a polynucleotide. A nucleic acid can be any nucleic acid, preferably a deoxyribonucleic acid (DNA) or a ribonucleic acid (RNA).

In one aspect the invention provides an Epstein-Barr-Virus (EBV) vaccine, comprising a DNA encoding a SQAPLPCVL peptide, for use in the treatment of EBV or an EBV-induced disorder.

An EBV vaccine comprising a DNA encoding a SQAPLPCVL peptide may also be referred herein as DNA vaccine. In general, a DNA vaccine is a vaccine that transfects a DNA encoding a SQAPLPCVL peptide into cells of an organism, such as a human. The SQAPLPCVL peptide is then transcribed and translated in the cells which triggers an EBV specific immune response. A DNA vaccine can comprise a DNA plasmid or vector encoding a SQAPLPCVL peptide. The DNA plasmid or vector can comprise additional elements that aid the transcription and translation of the SQAPLPCVL peptide in the host cell, such as artificial or viral promoters. For example, a DNA plasmid or vector can further comprise a strong viral promoter, such as the simian vacuolating virus 40 (SV40) promoter, the Rous Sarcoma Virus (RSV) promoter, the cytomegalovirus (CMV) immediate early promoter or a retroviral cis-acting transcriptional element. A DNA plasmid or vector can further comprise strong polyadenylation/transcriptional termination signals, such as bovine growth hormone or rabbit beta-globulin polyadenylation sequences. A DNA plasmid or vector can further comprise modifications, such as insertion of enhancer sequences, synthetic introns, adenovirus tripartite leader (TPL) sequences and modifications to the polyadenylation and transcriptional termination sequences. The DNA plasmid or vector of the present invention can be codon optimized for expression in a host cell, such as a eukaryotic cell. Preferably, the DNA plasmid or vector is codon optimized for expression in a human cell. The DNA of the present invention can be single-stranded DNA or double-stranded DNA. The DNA of the present invention is preferably double stranded.

In one aspect the invention provides an Epstein-Barr-Virus (EBV) vaccine, comprising a DNA encoding a SQAPLPCVL peptide, for use in the treatment of EBV or an EBV-induced disorder, wherein the DNA is a DNA plasmid or DNA vector.

In another aspect the invention provides an Epstein-Barr-Virus (EBV) vaccine, comprising a DNA encoding a SQAPLPCVL peptide, for use in the treatment of EBV or an EBV-induced disorder, wherein the DNA is a DNA plasmid or DNA vector and wherein the plasmid or vector comprises a strong viral promoter, a coding sequence encoding a SQAPLPCVL peptide and a polyadenylation sequence.

In another aspect the invention provides an Epstein-Barr-Virus (EBV) vaccine, comprising an RNA encoding a SQAPLPCVL peptide, for use in the treatment of EBV or an EBV-induced disorder. Peptide can be encoded by a messenger ribonucleic acid (mRNA) which is typically a single-stranded molecule of RNA that can be used by a ribosome for peptide synthesis. An EBV vaccine comprising a RNA or mRNA encoding a SQAPLPCVL peptide may also be referred to herein as “RNA vaccine” or “mRNA vaccine”. In the sense of the present invention an RNA encoding a SQAPLPCVL peptide is preferably a mRNA. The mRNA encoding a SQAPLPCVL peptide is delivered to a host cell where it is translated into a SQAPLPCVL peptide which then triggers an EBV specific immune response. In general, any mRNA construct encoding a SQAPLPCVL peptide that is capable of triggering an EBV specific immune response can be used in the sense of the present invention.

Accordingly, the invention provides an Epstein-Barr-Virus (EBV) vaccine, comprising an mRNA encoding a SQAPLPCVL peptide, for use in the treatment of EBV or an EBV-induced disorder.

The skilled person is aware that, except where otherwise noted, polynucleotide sequences set forth in the present application will recite “T”s in a representative DNA sequence but where the sequence represents RNA (e.g., mRNA), the “T”s would be substituted for “U”s. Thus, any of the RNA polynucleotides encoded by a DNA identified by a particular sequence identification number may also comprise the corresponding RNA (e.g., mRNA) sequence encoded by the DNA, where each “T” of the DNA sequence is substituted with “U”.

The mRNA of the present invention comprises at least an open reading frame encoding a SQAPLPCVL peptide. The basic components of an mRNA molecule typically include at least one coding regions, a 5′ untranslated region (UTR), a 3′ UTR, a 5′ cap and a poly-A tail. Polynucleotides of the present invention may function as mRNA but can be distinguished from wild-type mRNA in their functional and/or structural design features which serve to overcome existing problems of effective polypeptide expression using nucleic-acid based therapeutics. The mRNAs of the present invention may be codon optimized for expression in the host cell, such as a human.

Accordingly, the invention provides an Epstein-Barr-Virus (EBV) vaccine, comprising an mRNA encoding a SQAPLPCVL peptide, further comprising a 5′ untranslated region (UTR), a 3′ UTR, a 5′ cap and a poly-A tail, for use in the treatment of EBV or an EBV-induced disorder.

An EBV vaccine of the present invention can additionally comprise a pharmaceutically acceptable carrier. In general, the pharmaceutically acceptable carrier can be optional in the EBV vaccine of the present invention. A “pharmaceutically acceptable carrier” or “pharmaceutically acceptable excipient”, after administration to or upon a subject, does not cause undesirable physiological effects. The carrier in the pharmaceutical composition must be “acceptable” also in the sense that it is compatible with the active ingredient and can be capable of stabilizing it. Thus, a pharmaceutically acceptable carrier should not interfere with an EBV specific immune response triggered by a SQAPLPCVL peptide (or nucleic acid). One or more solubilizing agents can be utilized as pharmaceutical carriers for delivery of an active agent. Examples of a pharmaceutically acceptable carrier include, but are not limited to, biocompatible vehicles, adjuvants, additives, diluents, solvents, dispersion media, coatings, antibacterial agents, antifungal agents, isotonic absorption delaying agents, to achieve a composition usable as a dosage form. A pharmaceutically acceptable carrier can also comprise a pharmaceutically acceptable salt. Non-limiting examples of pharmaceutically acceptable salts include acid addition salts (formed from a free amino group of a polypeptide with an inorganic acid (e.g., hydrochloric or phosphoric acids), or an organic acid such as acetic, tartaric, mandelic, or the like), the salt formed with the free carboxyl groups is derived from an inorganic base (e.g., sodium, potassium, ammonium, calcium or ferric hydroxides), or an organic base such as isopropylamine, trimethylamine, 2-ethylamino ethanol, histidine, procaine, or the like).

For achieving an optimal T cell-mediated antitumor effect, the T cells may be antigen-specifically activated and expanded, which may be achieved by the three well-known signals of T cell receptor stimulation, appropriate co-stimulation, and specific cytokines.

Therefore, adjuvants can be used to deliver the peptide to dendritic cells and activate and mature these antigen-presenting cells (APCs) to accomplish a solid T cell response. The purpose of such adjuvants thus comprises the protection of the peptide and prevention of immediate degradation, the efficient uptake by APCs, as well as the appropriate and full activation of APCs. Delivery vehicles, which can have a depot effect, can be composed, for example, of oil depots such as Montanide ISA 51 (incomplete Freund's adjuvant analog). Montanide therefore can be mixed with the peptides, e.g. the peptide of the present invention, prior to vaccination to generate a water-in-oil emulsion. For efficient uptake, the peptides can also be encapsulated in structures such as liposomes and nanoparticles and/or can be covalently conjugated to adjuvants.

The cytokine granulocyte-macrophage colony stimulating factor (GM-CSF) that initiates the recruitment, maturation, and activation of dendritic cells is one of the most common adjuvants applied in peptide vaccination. For activation and maturation of APCs, signaling through Toll-like receptors (TLRs) and their ligands is known to induce optimal and strong activation. Therefore, potent adjuvants may mimic TLR ligands. Especially TLR4 ligands may be used as adjuvants to enable a potent activation of APCs. Chemically detoxified form MPL (3-O-desacyl-4′-monophosphoryl lipid A) is an approved adjuvant, for example, in human papillomavirus vaccines and furthermore investigated in different vaccination approaches. The most commonly used TLR agonist poly-ICLC (Hiltonol) is a polyinosinicpolycytidylic acid (poly-IC) stabilized by lysine and carboxymethylcellulose, which enhances vaccine-induced T cell responses by TLR3 signaling. Recently, the novel, water-soluble adjuvant XS15, a synthetic TLR1/2-binding Pam3-Cys-derivate covalently linked to a single synthetic-nonvaccine-peptide (GDPKHPKSF), was described as an effective vaccine adjuvant inducing unpreceded strong and long-lasting CD8⁺ and CD4⁺ T cell responses in first-in-man proof-of-concept experiments. The above is described in detail in Nelde, Annika, Hans-Georg Rammensee, and Juliane S. Walz. “The peptide vaccine of the future.” Molecular & Cellular Proteomics 20 (2021) and World Health Organization: “Guidelines for the production and quality control of synthetic peptide vaccines.” WHO technical report series 889 (1999).

An “adjuvant” as used herein refers to a compound or composition that enhances the immune response to an immunogen, such as the SQAPLPCVL peptide provided herein. A pharmaceutical composition or vaccine (composition) according to the invention that comprises an adjuvant can be used for immunization, e.g. of human subjects. An adjuvant can, prolong and/or enhance the quality and/or strength of an immune response to an antigen/immunogen, such as the SQAPLPCVL peptide provided herein, in comparison to the administration of the SQAPLPCVL peptide alone, thus, may reduce the quantity of SQAPLPCVL peptide necessary in a vaccine, and/or the frequency of injection necessary in order to generate an adequate immune response to the SQAPLPCVL peptide.

Examples of adjuvants that may be used in the context of the compositions and vaccine of the invention include inorganic adjuvants (e.g. inorganic metal salts such as aluminum phosphate or aluminum hydroxide), gel-like precipitates of aluminum hydroxide (alum); AlPO₄; alhydrogel; bacterial products from the outer membrane of Gram negative bacteria, in particular monophosphoryl lipid A (MPLA), lipopolysaccharides (LPS), muramyl dipeptides and derivatives thereof; Freund's incomplete adjuvant; liposomes, in particular neutral liposomes, liposomes containing the composition and optionally cytokines; AS01 B, AS01 E, AS02; non-ionic block copolymers; ISCOMATRIX adjuvant; unmethylated DNA comprising CpG dinucleotides (CpG motif), in particular CpG ODN with a phosphorothioate (PTO) backbone (CpG PTO ODN) or phosphodiester (PO) backbone (CpG PO ODN); synthetic lipopeptide derivatives, in particular Pam₃Cys; lipoarabinomannan; peptidoglycan; zymosan; heat shock proteins (HSP), in particular HSP 70; dsRNA and synthetic derivatives thereof, in particular Poly I:poly C; polycationic peptides, in particular poly-L-arginine; taxol; fibronectin; flagellin; imidazoquinoline; cytokines with adjuvant activity, in particular GM-CSF, interleukin- (IL-2, IL-6, IL-7, IL-18, type I and II interferons, in particular interferon-gamma, TNF-alpha; 25-dihydroxyvitamin D3 (calcitriol); and synthetic oligopeptides, in particular MHCII-presented peptides. Non-ionic block polymers containing polyoxyethylene (POE) and polyoxypropylene (POP), such as POE-POP-POE block copolymers may be used as an adjuvant.

Additional examples of adjuvants include inorganic adjuvants (e.g. inorganic metal salts such as aluminium phosphate or aluminium hydroxide), organic adjuvants (e.g. saponins, such as QS21, or squalene), oil-based adjuvants (e.g. Freund's complete adjuvant and Freund's incomplete adjuvant), cytokines (e.g. IL-1β, IL-2, IL-7, IL-12, IL-18, GM-CFS, and INF-γ) particulate adjuvants (e.g. immuno-stimulatory complexes (ISCOMS), liposomes, biodegradable microspheres, virosomes, bacterial adjuvants (e.g. monophosphoryl lipid A, such as 3-de-O-acylated monophosphoryl lipid A (3D-MPL), or muramyl peptides), synthetic adjuvants e.g. monophosphoryl lipid A (MPL), in particular 3-de-O-acylated monophosphoryl lipid A (3D-MPL) and muramyl peptide analogues, or synthetic lipid A, and synthetic polynucleotides adjuvants, e.g., polyarginine or polylysine.

Saponins are also suitable adjuvants, for example, the saponin Quil A, derived from the bark of the South American tree Quillaja Saponaria molina, and fractions thereof. Purified fractions of Quil A are also known as immunostimulants, such as squalene, QS21, QS17 and QS7, a non-haemolytic fraction of Quil-A.

Combinations of QS21 and polysorbate or cyclodextrin are also suitable. Another example of an adjuvant is an immunostimulatory oligonucleotide containing unmethylated cytosine-guanosine dinucleotide motifs present in DNA (CpG).

CpG is known as an adjuvant when administered by both systemic and mucosal routes. When formulated into vaccines, it may be administered in free solution together with free antigen, such as the SQAPLPCVL peptide provided herein, or covalently conjugated to an antigen, such as the SQAPLPCVL peptide provided herein, or formulated with a carrier such as aluminium hydroxide.

Activation of specific receptors can stimulate an immune response. Such receptors are known to the skilled artisan and comprise, for example, cytokine receptors, in particular type I cytokine receptors, type II cytokine receptors, TNF receptors; and a vitamin D receptor acting as transcription factor; and the Toll-like receptors 1 (TLR1), TLR2, TLR3, TLR4, TLR5, TLR6, TLR7, and TLR9. Agonists to such receptors have adjuvant activity, i.e., are immunostimulatory. Other suitable adjuvants include alkyl glucosaminide phosphates (AGPs) or pharmaceutically acceptable salts of AGPs. Some AGPs are TLR4 agonists, and some are TLR4 antagonists. An adjuvant of the pharmaceutical composition and/or EBV vaccine of the present invention may be one or more Toll-like receptor agonists.

Adjuvants such as those described above may be formulated together with carriers, such as liposomes, oil in water emulsions, and/or metallic salts (including aluminium salts such as aluminium hydroxide). For example, 3D-MPL may be formulated with aluminium hydroxide or oil in water emulsions; QS21 may be formulated with cholesterol containing liposomes, oil in water emulsions or alum; CpG may be formulated with alum or with other cationic carriers. Combinations of adjuvants may be utilized in the present invention, in particular a combination of a monophosphoryl lipid A and a saponin derivative, more particularly the combination of QS21 and 3D-MPL or a composition where the QS21 is quenched in cholesterol-containing liposomes (DQ). A combination of CpG plus a saponin such as QS21 can be an adjuvant suitable for use in the present invention, as a potent adjuvant formulation involving QS21, 3D-MPL and tocopherol in an oil in water emulsion. Saponin adjuvants may be formulated in a liposome and combined with an immunostimulatory oligonucleotide. Thus, suitable adjuvant systems include, for example, a combination of monophosphoryl lipid A, preferably 3D-MPL, together with an aluminium salt. A further exemplary adjuvant comprises QS21 and/or MPL and/or CpG. QS21 may be quenched in cholesterol-containing liposomes.

The fusion of the MHC class II invariant chain (also known as CD74) to an antigen, such as the SQAPLPCVL peptide provided herein, which is comprised by an expression system used for vaccination may increase the immune response against the SQAPLPCVL peptide provided herein, if it is e.g. administered with a viral vector, e.g. an adenovirus.

In some embodiments, a pharmaceutically acceptable carrier may comprise mineral gels, aluminium and/or calcium compounds, such as aluminium hydroxide, aluminium or calcium phosphate.

Accordingly, the invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease.

In one aspect, the invention provides an Epstein-Barr-Virus (EBV) vaccine, comprising a peptide consisting of the sequence SQAPLPCVL (SEQ ID NO: 1) or a nucleic acid encoding a peptide consisting of the sequence SQAPLPCVL (SEQ ID NO: 1) and, optionally, a pharmaceutically acceptable carrier.

The invention also provides an Epstein-Barr-Virus (EBV) vaccine, comprising a SQAPLPCVL peptide or a nucleic acid encoding a SQAPLPCVL peptide and, a pharmaceutically acceptable carrier, for use in the treatment of EBV or an EBV-induced disease, wherein the pharmaceutically acceptable carrier comprises or is an adjuvant as provided herein.

In one aspect, the invention provides an Epstein-Barr-Virus (EBV) vaccine, comprising a peptide consisting of the sequence SQAPLPCVL (SEQ ID NO: 1) or a nucleic acid encoding a peptide consisting of the sequence SQAPLPCVL (SEQ ID NO: 1) and, a pharmaceutically acceptable carrier, wherein the pharmaceutically acceptable carrier comprises or is an adjuvant as provided herein.

In another aspect, the invention provides an Epstein-Barr-Virus (EBV) vaccine, comprising a SQAPLPCVL peptide or a nucleic acid encoding a SQAPLPCVL peptide and a pharmaceutically acceptable carrier, for use in the treatment of EBV or an EBV-induced disease, wherein the pharmaceutically acceptable carrier comprises one or more of Montanide ISA 51, GM-CSF, TLR ligands, such as MPL (3-O-desacyl-4′-monophosphoryl lipid A), poly-ICLC (Hiltonol), XS15, CRM 197 (Corynebacterium diphtheriae CRM197 protein).

In one aspect, the invention provides an Epstein-Barr-Virus (EBV) vaccine, comprising a peptide consisting of the sequence SQAPLPCVL (SEQ ID NO: 1) or a nucleic acid encoding a peptide consisting of the sequence SQAPLPCVL (SEQ ID NO: 1) and a pharmaceutically acceptable carrier, wherein the pharmaceutically acceptable carrier comprises one or more of Montanide ISA 51, GM-CSF, TLR ligands, such as MPL (3-O-desacyl-4′-monophosphoryl lipid A), poly-ICLC (Hiltonol), XS15, CRM 197 (Corynebacterium diphtheriae CRM197 protein).

The pharmaceutically acceptable carrier can also include means of delivery of a SQAPLPCVL peptide or a nucleic acid encoding the same. The EBV vaccine can comprise the SQAPLPCVL peptide either in free form or the form of a pharmaceutically acceptable salt. A SQAPLPCVL peptide in form of a pharmaceutically acceptable salts refers to acid or base salts of the peptide. For example, acid salts are prepared from the free base (typically wherein the neutral from of the drug has a neutral —NH2 group) involving reaction with a suitable acid. Suitable acids for preparing acid salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methane sulfonic acid, ethane sulfonic acid, p-toluenesulfonic acid, salicylic acid, and the like, as well as inorganic acids, e.g., hydrochloric acid, hydrobromic acid, sulfuric acid, nitric acid phosphoric acid and the like. Conversely, preparation of basic salts of acid moieties which may be present on a peptide of the invention are prepared using a pharmaceutically acceptable base such as sodium hydroxide, potassium hydroxide, ammonium hydroxide, calcium hydroxide, trimethylamine or the like.

An EBV vaccine of the present invention can further comprise a pharmaceutically acceptable carrier.

Accordingly, the present invention provides an Epstein-Barr-Virus (EBV) vaccine, comprising a SQAPLPCVL peptide or a nucleic acid encoding a SQAPLPCVL peptide and a pharmaceutically acceptable carrier, for use in the treatment of EBV or an EBV-induced disorder.

The EBV vaccine of the present invention can be used in the treatment of EBV or an EBV-induced disease or in a method of treating EBV. The invention is not particularly limited to a treatment of specific diseases, rather any EBV-induced or EBV-associated disease can be treated with the EBV vaccine of the invention. The terms EBV-induced disease and EBV-associated disease are used interchangeably herein.

The skilled physician is able, also in view of growing evidence of EBV association with different diseases to determine which EBV-induced or associated disease is to be treated with the EBV vaccine of the present invention. The ultimate decision if a disease is to be treated be it prophylactically or therapeutically is in the scope of sound medical judgment of the skilled physician. In general, it is desired to treat any individual with the EBV vaccine of the present invention to build up an immunity and therefore provide a prophylactic treatment of any EBV-induced or associated diseases and EBV infection per se. Exemplary diseases and pathologies that can be treated with the EBV vaccine of the present invention prophylactically as well as therapeutically are described herein.

In general, treatment of EBV-associated diseases means a treatment of the Epstein-Barr-Virus and EBV-infected cells per se. Thus, it is clear to the skilled person that a treatment of EBV includes the prophylactic treatment (prevention) of an infection with EBV, which may include a primary, secondary or any further infection with EBV. A primary infection can include a latent/persistent EBV infection (i.e. a salient state of the EBV following initial infection with EBV). A secondary or further infection with EBV is, for example, EBV infection following EBV reactivation or EBV reinfections. A secondary or further infection with EBV can also refer to any subsequent EBV infection following a primary EBV infection. It is preferred herein, that a treatment of EBV can relate to a prevention of EBV, such as a prevention of EBV infection, prevention of symptomatic acute EBV infection or prevention of a disease caused by EBV (EBV-linked disease or EBV associated disease).

It is understood that the vaccine might not completely inhibit infection with EBV in a vaccinated subject (e.g. that a vaccinated subject is infected with EBV subsequent to vaccination). For example, a vaccinated subject might show viral spread and replication, if the subject is infected with EBV after vaccination. However, in accordance with the invention, a vaccinated subject (or a group of vaccinated subjects) can be or is asymptomatic, e.g. if the subject is infected with EBV after vaccination. A vaccinated subject (or a group of vaccinated subjects) can show or shows reduced viral spread and/or reduced replication (e.g. if the subject is infected with EBV after vaccination), e.g. compared to viral spread and/or replication in a reference subject or in a group of reference subjects (like non-vaccinated subjects), e.g. 90% reduced viral spread and/or 90% reduced viral replication, as described elsewhere herein. A vaccinated subject (or a group of vaccinated subjects) can show or shows a milder course of EBV infection (e.g. symptoms of the EBV infection and/or (the symptoms of) EBV-linked or EBV-associated disease, reduced viral spread and/or reduced replication (e.g. if the subject is infected with EBV after vaccination), e.g. compared to the course of EBV infection in a reference subject or in a group of reference subjects (like non-vaccinated subjects). In line with this, the symptoms of the EBV infection and/or (the symptoms of) EBV-linked or EBV-associated disease can be or is reduced in a vaccinated subject, e.g. compared to a reference subject or a group of reference subjects (like non-vaccinated subjects), e.g. 90% reduced, as described elsewhere herein.

As described herein, the therapeutic effect (e.g. asymptomatic (course), reduced viral spread/replication, reduced symptoms, milder course) can be or is associated with the induced T cell response in a vaccinated subject, particularly the SQAPLPCVL-specific HLA-E-restricted CD8⁺ T cells (which are elicited by the vaccine). It is envisaged herein that the vaccination induces a T cell response in an amount sufficient to confer the intended therapeutic effect in the vaccinated subject (e.g. asymptomatic (course), reduced viral spread/replication, reduced symptoms, milder course as described above), e.g. if the subject is infected with EBV after vaccination. In line with the above, the herein provided vaccine provides protection against EBV, including EBV infection, symptomatic acute EBV infection or a disease caused by EBV. The term “infected with EBV” refers to any of the infections with EBV described herein (e.g. primary, secondary or any further infection with EBV). In this sense a subject “infected with EBV” is a subject suffering from or having suffered from any of the infections with EBV described herein.

Treatment of EBV also includes the treatment of an EBV infection that is acute symptomatic or an asymptomatic EBV infection (therapeutic treatment). Specifically, the term “treatment of EBV” also relates to a treatment of EBV reactivation of already existing latent/persistent EBV infection in a person. Treatment may be especially important in persons under immunosuppression which may be e.g. due to an immunosuppressive therapy (e.g. in the course of transplantation) or to a congenital (e.g. X-chromosomal lymphoproliferative disease) or acquired immunodeficiency (e.g. HIV infection). Thus, the “term treatment of EBV” also relates to treatment of EBV infections following EBV reactivation or EBV reinfections. An acute symptomatic EBV infection can cause infectious mononucleosis (IM).

Accordingly, the present invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV.

The present invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the prophylactic treatment of an EBV infection.

The present invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of a primary EBV infection.

The present invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of a primary, secondary, or further EBV infection.

The present invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of an acute symptomatic EBV infection/infectious mononucleosis (IM).

The present invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of an asymptomatic EBV infection.

The present invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of a reactivation of an EBV infection.

The present invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of a reactivation of an EBV infection, wherein the reactivation is caused or facilitated by another medical treatment resulting in an immunodeficiency or by a congenital or acquired immunodeficiency.

The present invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of a reactivation of an EBV infection, wherein the reactivation is caused or facilitated by an organ or stem cell transplant.

The EBV vaccine of the present invention can be used in the treatment of EBV or an EBV-induced disease or in a method of treating an EBV-induced disease. In general, an EBV-induced disease in the sense of the present invention may be any disease whose development, progression or symptoms are caused or facilitated by EBV. For example, a primary infection with EBV can cause infectious mononucleosis (IM). IM caused by EBV infection can also further cause postinfectious chronic fatigue syndrome.

Accordingly, the invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of infectious mononucleosis (IM).

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of infectious mononucleosis (IM) caused by primary infection with EBV.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of postinfectious syndromes as chronic fatigue syndrome following infectious mononucleosis (IM) and/or primary infection with EBV.

EBV infection is also associated with the development of lymphoproliferative diseases, such as post-transplant lymphoproliferative disorder (PTLD) and/or other EBV associated lymphoproliferative diseases (LPDs) in patients with and without immunosuppression. EBV may cause or facilitate PTLD and/or LPDs specifically in immunosuppressed subjects, such as after a transplantation, or in subjects that have an immunodeficiency, such as a congenital or acquired immunodeficiency.

Accordingly, the invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of lymphoproliferative diseases.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV-induced lymphoproliferative diseases.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of post-transplant lymphoproliferative disorder (PTLD) and/or other LPDs.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV-induced post-transplant lymphoproliferative disorder (PTLD) and/or other LPDs.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV associated lymphoproliferative diseases.

The invention provides in one aspect an Epstein-Barr-Virus (EBV) vaccine, comprising a peptide consisting of the sequence SQAPLPCVL (SEQ ID NO: 1) or a nucleic acid encoding a peptide consisting of the sequence SQAPLPCVL (SEQ ID NO: 1) and, optionally, a pharmaceutically acceptable carrier, for use in the treatment of EBV associated lymphoproliferative diseases.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV-induced post-transplant lymphoproliferative disorder (PTLD), and/or other LPDs, wherein PTLD and/or other LPDs are further facilitated by an immunosuppression or an immunodeficiency.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV-induced post-transplant lymphoproliferative disorder (PTLD), and/or other LPDs, wherein PTLD and/or other LPDs are further facilitated by a congenital or acquired immunodeficiency.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV-induced post-transplant lymphoproliferative disorder (PTLD) and/or other LPDs. wherein PTLD and/or other LPDs are further facilitated by an immunosuppression due to an organ or stem cell transplant.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV-induced post-transplant lymphoproliferative disorder (PTLD), and/or other LPDs wherein PTLD and/or other LPDs are further facilitated by an immunosuppression due to a solid-organ or hematopoietic stem cell transplant.

EBV infection is also associated with the development of malignancies and/or lymphoproliferative diseases (LPDs), such as malignant lymphoproliferative diseases (LPDs).

For example, EBV plays a role in the development or can facilitate the development of exemplary malignancies/lymphoproliferative diseases (LPDs), such as malignant post-transplant lymphoproliferative disorder (PTLD), Burkitt lymphoma, hemophagocytic lymphohistiocytosis, EBV-associated Hodgkin lymphoma, EBV-associated gastric carcinoma, nasopharyngeal carcinoma, B-cell lymphoma, lymphomatoid granulomatosis and the like.

Exemplary lymphoproliferative diseases (LPDs) are Burkitt lymphoma, EBV-associated Hodgkin lymphoma, B-cell lymphoma, hemophagocytic lymphohistiocytosis and lymphomatoid granulomatosis. Exemplary malignancies are EBV-associated gastric carcinoma, nasopharyngeal carcinoma.

Accordingly, the invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of malignancies.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV-induced malignancies.

The invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV-induced malignant post-transplant lymphoproliferative disorder (PTLD).

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV-induced malignant post-transplant lymphoproliferative disorder (PTLD), wherein malignant PTLD is further facilitated by an organ or stem cell transplant.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV-induced malignant post-transplant lymphoproliferative disorder (PTLD), wherein malignant PTLD is further facilitated by a solid-organ or hematopoietic stem cell transplant.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV-induced malignant post-transplant lymphoproliferative disorder (PTLD), wherein malignant PTLD is further facilitated by an immunosuppression or an immunodeficiency.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV-induced malignant post-transplant lymphoproliferative disorder (PTLD), wherein malignant PTLD is further facilitated by a congenital or acquired immunodeficiency.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV-induced malignant post-transplant lymphoproliferative disorder (PTLD), wherein malignant PTLD is further facilitated by an immunosuppression due to an organ or stem cell transplant.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV-induced malignant post-transplant lymphoproliferative disorder (PTLD), wherein malignant PTLD is further facilitated by an immunosuppression due to a solid-organ or hematopoietic stem cell transplant.

The invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV-induced Burkitt lymphoma.

The invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV-induced hemophagocytic lymphohistiocytosis.

The invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV-associated Hodgkin lymphoma.

The invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV-associated gastric carcinoma.

The invention provides in one aspect an Epstein-Barr-Virus (EBV) vaccine, comprising a peptide consisting of the sequence SQAPLPCVL (SEQ ID NO: 1) or a nucleic acid encoding a peptide consisting of the sequence SQAPLPCVL (SEQ ID NO: 1) and, optionally, a pharmaceutically acceptable carrier, for use in the treatment of EBV-associated gastric carcinoma.

The invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV-induced nasopharyngeal carcinoma.

The invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV-induced B-cell lymphoma.

The EBV vaccine of the present invention can also be useful in the treatment of diseases which are partially caused by or whose course of disease is adversely affected by an EBV infection. Examples for such diseases are multiple sclerosis, myalgic encephalomyelitis/chronic fatigue syndrome or eventually long COVID.

Accordingly, the invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of multiple sclerosis.

The invention provides in one aspect an Epstein-Barr-Virus (EBV) vaccine, comprising a peptide consisting of the sequence SQAPLPCVL (SEQ ID NO: 1) or a nucleic acid encoding a peptide consisting of the sequence SQAPLPCVL (SEQ ID NO: 1) and, optionally, a pharmaceutically acceptable carrier, for use in the treatment of multiple sclerosis.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of long COVID.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of myalgic encephalomyelitis.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of chronic fatigue syndrome.

The EBV vaccine can thus be used to treat the development or progression of these diseases prophylactically or therapeutically with treatment of the adverse effects caused by EBV.

Accordingly, the invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV-induced adverse effects in multiple sclerosis.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV-induced adverse effects in long COVID.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV-induced adverse effects in myalgic encephalomyelitis.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV-induced adverse effects in chronic fatigue syndrome.

The EBV vaccine can also be used to treat or prevent symptoms associated with EBV. Such symptoms are for example, fatigue, fever, inflamed throat, swollen lymph nodes in the neck, enlarged spleen, swollen liver and rash.

Accordingly, the invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of symptoms associated with EBV infection.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of symptoms associated with EBV infection, wherein the symptoms are one or more of fatigue, fever, inflamed throat, swollen lymph nodes in the neck, enlarged spleen, swollen liver and rash.

Any of the treatments described herein can be prophylactic, i.e. preventing primary EBV infection or preventing a EBV-induced disease during asymptomatic EBV infection, or therapeutic, i.e. treating an EBV infection or treating an acute symptomatic EBV infection or EBV-induced disease.

Accordingly, the invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the treatment is a prophylactic treatment.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of a primary EBV infection, wherein the treatment is a prophylactic treatment.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of an EBV-induced disease, wherein the treatment is a prophylactic treatment.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of an EBV-induced disease during asymptomatic EBV infection, wherein the treatment is a prophylactic treatment.

Accordingly, the invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the treatment is a therapeutic/acute treatment.

Accordingly, the invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of an EBV infection, wherein the treatment is a therapeutic/acute treatment.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of an acute symptomatic EBV infection, wherein the treatment is a therapeutic/acute treatment.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of an acute symptomatic EBV-induced disease, wherein the treatment is a therapeutic/acute treatment.

The EBV vaccine of the present invention can be delivered by any means that allows for efficient delivery of the peptide or the nucleic acid encoding into a host organism or a host cell, such as a human or human cell. For example, the SQAPLPCVL peptide can be delivered directly, i.e. in free form/peptide from comprised in the EBV vaccine. In another example, the SQAPLPCVL peptide can be delivered directly, i.e. in free form/peptide from, e.g. in an appropriate solution, e.g. comprised in the EBV vaccine. The EBV vaccine comprising a SQAPLPCVL peptide or a nucleic acid encoding a SQAPLPCVL peptide can also be formulated into a nanoparticle. Exemplary nanoparticles of the present invention are a lipid nanoparticle, a dendrimer nanoparticle, a micelle nanoparticle, a protein nanoparticle, a liposome, a non-porous silica nanoparticle, a mesoporous silica nanoparticle, a silicon nanoparticle, a gold nanoparticle, a gold nanowire, a silver nanoparticle, a platinum nanoparticle, a palladium nanoparticle, a titanium dioxide nanoparticle, a carbon nanotube, a carbon dot nanoparticle, a polymer nanoparticle, a zeolite nanoparticle, an aluminium oxide nanoparticle, a hydroxyapatite nanoparticle, a quantum dot nanoparticle, a zinc oxide nanoparticle, a zirconium oxide nanoparticle, graphene or a graphene oxide nanoparticle.

Accordingly, the present invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disorder, wherein the SQAPLPCVL peptide or a nucleic acid encoding a SQAPLPCVL peptide is formulated into a nanoparticle, such as a lipid nanoparticle.

The present invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disorder, wherein the SQAPLPCVL peptide or a nucleic acid encoding a SQAPLPCVL peptide is formulated into a lipid nanoparticle.

The present invention also provides an Epstein-Barr-Virus (EBV) vaccine, comprising a SQAPLPCVL peptide, for use in the treatment of EBV or an EBV-induced disorder, wherein the SQAPLPCVL peptide is formulated in free form/peptide from, optionally in an appropriate solution.

The treatment of the present invention comprises administering the EBV vaccine to a subject. As used herein, “administering” means that e.g. an EBV vaccine is delivered into a subject. The EBV vaccine of the present invention can be administered to a subject by any means which result in a therapeutically effective outcome. These include, but are not limited, to intradermal, intramuscular, subcutaneous, mucosal, ophthalmic and/or pulmonary administration (e.g. via inhalation). Preferably, the EBV vaccine is administered by intradermal injection, intramuscular injection or subcutaneous injection. The EBV vaccine of the present invention may be administered with a pharmaceutically acceptable carrier and in a nanoparticle formulation as described herein above. As used herein, “therapeutically effective outcome” means that a development, progression or burden of a disease and symptoms thereof is reduced, ameliorated by treatment with the EBV vaccine of the present invention in a subject compared to a subject that has not been treated.

In general, the EBV vaccine is administered to a subject in an effective amount. As used herein, “effective amount” relates to the amount of the EBV vaccine that is required to achieve a therapeutically effective outcome. The exact amount required will vary from subject to subject, depending on species, age, body weight, sex and diet of the subject, general condition of the subject, severity of EBV infection or the EBV-induced disease, the particular composition including a pharmaceutically acceptable carrier, the mode of delivery, the mode of administration and whether the EBV vaccine is administered in peptide from, as DNA vaccine or RNA vaccine, and the like. The exact amount required to achieve a therapeutically effective outcome may be decided by the attending physician within the scope of sound medical judgment. The effective amount of the EBV vaccine may be a total dose (of a single administration event) of about 30-3000 μg. The effective amount of the EBV vaccine may be a total dose (of a single administration event) of about 1, 2, 3, 4, 5, 6, 7, 8, 9, 10, 20, 30, 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, or 3000 μg. As described herein above, the effective amount can depend on the body weight of the subject. Accordingly, the effective amount of the EBV vaccine may be a total dose (of a single administration event) of about 0.5-12 μg/kg body weight. The effective amount of the EBV vaccine may be a total dose (of a single administration event) of about 0.01, 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, 19.5, 20, 20.5, 21, 21.5, 22, 22.5, 23, 23.5, 24, 24.5, 25, 25.5, 26, 26.5, 27, 27.5, 28, 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 49, 49.5, or 50 μg/kg body weight.

The effective amount as described above can apply to a single dose of an EBV vaccine and also to additional doses of an EBV vaccine, such as booster doses as described herein below. As used herein “dose” relates to a EBV vaccine comprising an effective amount of the SQAPLPCVL peptide or the nucleic acid encoding a SQAPLPCVL peptide. As used herein “booster dose” relates to a EBV vaccine comprising an effective amount of the SQAPLPCVL peptide or the nucleic acid encoding a SQAPLPCVL peptide that is administered to a subject having received at least a first dose of an EBV vaccine.

As described herein above, the EBV vaccine is capable of triggering an EBV specific immune response. This immune response can be achieved by a single treatment, e.g. a single administration of the EBV vaccine. For example, when the EBV vaccine of the present invention is used in a prophylactic treatment a single dose may suffice to achieve acquired EBV immunity.

Accordingly, the invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disorder, wherein a single dose of the EBV vaccine is administered.

However, over time the developed acquired EBV immunity from the single dose may decline. As a countermeasure, the EBV vaccine of the present invention can be administered repeatedly over a period of time to ensure maintenance of the acquired EBV immunity. These additional doses over time that aim to maintain EBV immunity may be referred herein as booster doses. In this case, the initial single dose of the EBV vaccine can be the first dose. A booster dose may be administered whenever the acquired EBV immunity declines. The skilled practitioner is capable of detecting a decline in the acquired EBV immunity, e.g. via an antibody titre. One or more booster doses may be administered 3 months, 6 months, 1 year, 2, years, 3 years, 4 years, 5 years, 6 years, 7 years, 8 years, 9 years, 10 years, 11 years, 12 years, 13 years, 14 years, 15 years, 16 years, 17 years, 18 years, 19 years, or 20 years after the initial dose. Following a first booster dose, a second or further booster doses may be administered 3 months, 6 months, 1 year, 2, years, 3 years, 4 years, 5 years, 6 years, 7 years, 8 years, 9 years, 10 years, 11 years, 12 years, 13 years, 14 years, 15 years, 16 years, 17 years, 18 years, 19 years, or 20 years after the previous booster dose. A booster dose can be administered regularly over the lifetime of a subject to ensure maintenance of the acquired EBV immunity. Such a treatment including one or more booster doses after an initial dose of the EBV vaccine may be a prophylactic treatment.

Accordingly, the invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disorder, wherein a single dose of the EBV vaccine is administered, further comprising administration of one or more booster doses of the EBV vaccine.

The EBV vaccine of the present invention can also be given throughout the course of a disease, such as during an acute or active EBV infection or EBV-induced disease (also referred herein as therapeutic treatment). When administered as a therapeutic treatment, additional doses of the EBV vaccine may be desired to induce a strong immune response.

Accordingly, the invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the treatment is a therapeutic/acute treatment.

In such a therapeutic or acute treatment, the doses can be increased. In general, the invention is not limited to any doses or administration schemes of the EBV vaccine during a therapeutic or acute treatment. The skilled physician is able to determine the same within the scope of sound medical judgement. Accordingly, the EBV vaccine can be administered three times a day, two times a day, once a day, every other day, every third day, every week, every two weeks, every three weeks, every four weeks, every 2 months, every 3 months, every 6 months in a therapeutic treatment.

An acute treatment (therapeutic treatment) with the EBV vaccine of the present invention may be necessary when a subject suffers of an EBV infection (specifically when the infection is not asymptomatic and/or (in case of reactivation) when the infection is no longer latent), such as a primary EBV infection, EBV infection following EBV reactivation or EBV reinfection, and/or when the subject suffers from one or more of the following diseases: infectious mononucleosis (IM), post-transplant lymphoproliferative disorder (PTLD), malignant post-transplant lymphoproliferative disorder (PTLD), Burkitt lymphoma, hemophagocytic lymphohistiocytosis, EBV-associated Hodgkin lymphoma, EBV-associated gastric carcinoma, nasopharyngeal carcinoma, B-cell lymphoma, lymphomatoid granulomatosis, multiple sclerosis, long COVID, myalgic encephalomyelitis or chronic fatigue syndrome and other EBV associated diseases.

A prophylactic treatment (prevention) with the EBV vaccine of the present invention may be necessary to prevent a primary EBV infection, EBV infection following EBV reactivation or EBV-reinfection, infectious mononucleosis (IM), post-transplant lymphoproliferative disorder (PTLD), malignant post-transplant lymphoproliferative disorder (PTLD), Burkitt lymphoma, hemophagocytic lymphohistiocytosis, EBV-associated Hodgkin lymphoma, EBV-associated gastric carcinoma, nasopharyngeal carcinoma, B-cell lymphoma, lymphomatoid granulomatosis, multiple sclerosis, long COVID, myalgic encephalomyelitis or chronic fatigue syndrome.

As used herein “prevent” relates to a prophylactic treatment of a disease to prevent the development or ameliorate the burden of the disease in a subject. For example, the vaccine can be used to prevent an infection to become symptomatic and/or to prevent a latent infection to become reactivated.

As used herein, the term “subject” means any individual. A subject can be for example an animal that is susceptible to EBV infection. Subjects where an immunization against EBV can be desired are for example subjects that have been exposed to EBV, subjects that are infected with EBV or subjects that are at risk of EBV infection. Similarly, an immunization against EBV can be desired may be in subjects that suffer from or are at risk of contracting a EBV induced disease. In the sense of the present invention, a subject that has been treated with the EBV vaccine is preferably immune to EBV and EBV-induced diseases or experiences asymptomatic EBV infection and is immune or less susceptible to EBV-induced diseases.

Accordingly, the invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of a subject that has been exposed to EBV.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of a subject that is infected with EBV.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of a subject that is at risk of EBV infection.

Immunosuppressed subjects and subjects having an immunodeficiency can be specifically susceptible to EBV infection, EBV reactivation and/or EBV-induced diseases. For example, a subject may be immunosuppressed due to immunosuppressive therapy. Such a therapy can be necessary before, during and after an organ or stem cell transplantation to avoid rejection and associated conditions such as graft versus host disease. Thus, it is especially useful to treat subjects that (will) undergo immunosuppressive therapy for example during the course of an organ or stem cell transplantation with the EBV vaccine of the present invention.

Accordingly, the invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the subject is immunosuppressed.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the subject will undergo, is undergoing or has undergone an immunosuppressive treatment.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the subject will undergo, is undergoing or has undergone an organ or stem cell transplantation.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the subject will undergo, is undergoing or has undergone a solid-organ or hematopoietic stem cell transplantation.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the subject will undergo, is undergoing or has undergone an immunosuppressive treatment during the course of an organ or stem cell transplantation.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the subject will undergo, is undergoing or has undergone an immunosuppressive treatment during the course of a solid-organ or hematopoietic stem cell transplantation.

As described, subjects having an immunodeficiency can also be specifically susceptible to EBV infection, EBV reactivation and/or EBV-induced diseases. For example, an immunodeficiency can be a congenital or acquired immunodeficiency. Non-limiting examples of congenital immunodeficiencies are primary immunodeficiencies such as ataxia-telangiectasia, cartilage-hair hypoplasia, DiGeorge syndrome, hyper-IgE syndrome, and Wiskott-Aldrich syndrome, Selective IgA deficiency, Chronic mucocutaneous candidiasis, DiGeorge syndrome, X-linked lymphoproliferative syndrome, Zeta-associated protein 70 (ZAP-70) deficiency, Severe combined immunodeficiency (SCID), Chediak-Higashi syndrome, Chronic granulomatous disease, Cyclic neutropenia, Leukocyte adhesion deficiency (types 1 and 2), and the like. Non-limiting examples of acquired immunodeficiency are secondary immunodeficiencies such as diabetes, undernutrition, HIV infection, prolonged serious illness, old age, nephrotic syndrome, severe burns or dermatitis, enteropathy, lymphopenia, autoimmune diseases, and the like as well as immunosuppression due to immunosuppressive treatment (eg, cytotoxic chemotherapy, bone marrow ablation before transplantation, radiation therapy, posttransplant immunosuppressive therapy, therapy of rheumatic and other autoimmune diseases and the like).

Accordingly, the invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the subject has a congenital or acquired immunodeficiency.

The invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the subject suffers from a primary or secondary immunodeficiency.

The EBV vaccine of the present invention is particularly useful for a prophylactic treatment of subjects. Such a prophylactic treatment can be used to treat a subject that is not infected with EBV. The EBV vaccine triggers an EBV specific immune response resulting in immunization of the subject. As used herein “immunity” means that a subject develops a specific immune response to EBV so that a severe course of an EBV infection or an EBV-induced disorder is unlikely to manifest in said subject. It is envisioned that the term “immunity” also includes subjects that still may be infected with EBV which however only lead to an asymptomatic course of disease of the EBV infection or EBV-induced disease. Further, the term “immunity” also includes that EBV replication and severe, symptomatic EBV infections or EBV-induced diseases are prevented. In the sense of the present invention a subject being immune to an EBV infection or EBV-induced diseases may still contract EBV or can still be infected with EBV. “immunity” also relates to long-term protection and long-term immunity against EBV infection or EBV-induced diseases. It is preferred herein, that an EBV infection or EBV-induced diseases cannot manifest in a subject that is immune to EBV following a treatment with the EBV vaccine of the invention. Thus, it is useful to treat both, subjects that are not infected with EBV and subjects that are infected with EBV, with the EBV vaccine of the present invention. It is particularly useful to treat subjects that are not infected with EBV with the EBV vaccine of the present invention, e.g. before they have been exposed to EBV.

Accordingly, the invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the subject is not infected with EBV.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the prophylactic treatment of EBV or an EBV-induced disease, wherein the subject is not infected with EBV.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the EBV vaccine confers immunity against EBV.

In the sense of the present invention a subject is preferably a mammal. A subject is most preferably a human. As used herein “human” includes humans of all ages and sex, for example female or male children, adults, or elderly people/person. In general, the EBV vaccine of the present invention can be used to treat any subject where an immunization against EBV or treatment of EBV infection or an EBV-induced disease is desired. As described herein above, a subject that is not infected with EBV can be treated prophylactically to protect against a future EBV infection or a future EBV-induced disease. EBV is one of the most common viruses in humans infecting more than 90% of the adult human population worldwide. Thus, a prophylactic treatment is especially useful when performed at an early stage of life before infection takes place. Accordingly, the EBV vaccine of the invention is especially useful in a prophylactic treatment of children, such as young human children. The EBV vaccine of the invention is also especially useful in a prophylactic treatment of adolescents/teenagers. Treatment of children can be preferred as an early immunization against EBV infection is desirable. As used herein, “child” is preferably a human child between birth and 18 years of age. As used herein, “young child” is preferably a human child between birth and 5 years of age. As used herein, “adolescent” or “teenager” is preferably a human between 10 and 19 years of age. Accordingly, a first or initial dose of the EBV vaccine of the present invention can be administered to a young child for example from birth or being 1 week, 2 weeks, 3 weeks, 1 month, 2 months, 3 months, 4 months, 5 months, 6 months, 7 months, 8 months, 9 months, 10 months, 11 months, 1 year, 2 years, 3 years, 4 years, or 5 years old. EBV spreads most commonly through bodily fluids, especially saliva, blood and semen during sexual contact, blood transfusions, and organ transplantations. A first or initial dose of the EBV vaccine of the present invention is preferably administered before puberty, e.g. before being sexually active, or before a sexual context. Accordingly, a first or initial dose of the EBV vaccine of the present invention is preferably administered to a subject being younger than 10, 11, 12, 13, 14, 15, 16, or 17 years. A first or initial dose of the EBV vaccine of the present invention may also be administered to an adolescent/teenager being 10, 11, 12, 13, 14, 15, 16, 17, 18, or 19 years of age. As used herein “adult” is preferably a human adult between 18 and 60 years of age. As used herein “elderly people” or “elderly person” is preferably a human adult of more than 60 years of age. It is preferred herein that a child is treated with the EBV vaccine of the present invention as early as possible since the possibility of EBV infection increases with age. The skilled physician is able to determine an appropriate age for a first treatment with the EBV vaccine of the invention.

Accordingly, the invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease in a human.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease in a human, wherein the human is a child an adult or an elderly person.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the prophylactic treatment of EBV or an EBV-induced disease in a human child.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the prophylactic treatment of EBV or an EBV-induced disease in a human child that is not infected with EBV.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the prophylactic treatment of EBV or an EBV-induced disease in a young human child that is not infected with EBV.

Also provided herein is the prophylactic treatment of a human, such as a human child, that is not infected with EBV, or has never been infected with EBV.

An initial immunization before a primary EBV infection is certainly desired, however, the EBV vaccine of the present invention is also capable of initiating an immune response in subjects that have been exposed to EBV or are infected with EBV. Accordingly, the subject can also be a young child that is infected with EBV or has been exposed to EBV. The same applies to adolescents/teenagers, adults or elderly people, however, as EBV is highly prevalent in the population, the chances are higher that an adult or elderly person are infected with EBV. Thus, when primary infection of EBV is to be prevented by e.g. an prophylactic treatment, the subject is preferably an adolescent/teenager, more preferably a child, and most preferably a young child.

As described herein above, the EBV vaccine of the present invention can be used to treat a EBV infection or EBV-induced disease prophylactically or therapeutically, i.e. irrespective of the infection or disease status of the subject. Accordingly, it is not decisive if the subject is infected with EBV or not as the EBV vaccine of the invention triggers an EBV-specific immune response in any case. Thus, the prophylactic or therapeutic treatments of the present invention can thus be generally applied to all subjects irrespective of their current EBV infection status. For example, the EBV vaccine of the present invention can be used to treat (acute) infectious mononucleosis (IM) in an adolescent/teenager. This treatment may be independent of the fact that the adolescent/teenager has received an initial dose of the EBV vaccine of the present invention before contracting IM or has not received an initial dose of the EBV vaccine of the present invention before contracting IM.

Subject may react differently to the EBV vaccine of the present invention. HLA-E is highly conserved. In European populations there are two allelic variants, the high expressing HLA-E*0103 and low expressing HLA-E*0101 are prevalent. For example, a subject having an HLA-E*0103/0103 genotype might develop a stronger immune response following administration of the vaccine than subject having an HLA-E*0101/0101 genotype. In general, a subject having an HLA-E*0103/0103 genotype has a lower risk of developing an infectious mononucleosis than a subject having an HLA-E*0101/0101. However, the treatment of both genotypes is desired in any case since the immune response is triggered by the EBV vaccine in both genotypes and merely variates in strength. In addition, while the HLA-E*0103/0103 genotype may have a lower risk of contracting infectious mononucleosis, it has a higher risk of developing EBV-induced malignancies. Protection is achieved by the HLA-E-restricted CD8⁺ T cell response in both genotypes. Thus, any subject irrespective of the HLA-E genotype will benefit from the EBV vaccine as its administration triggers the HLA-E-restricted CD8⁺ T cell response irrespective of the genotype. In addition, the determination of the HLA-E genotypes above can be tedious and resource intensive, accordingly a subject can be treated without testing for said genotypes since the immune response is independent thereof. In a non-limiting aspect of the present invention, treatment of a subject having an HLA-E*0103/0103 genotype may be preferred.

Accordingly, the invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the human subject has an HLA-E*0101/0101, HLA-E*0101/0103 or HLA-E*0103/0103 genotype.

The EBV vaccine of the present invention takes advantage of the BZLF1-derived EBV peptide SQAPLPCVL to initiate a host immune response. The peptide can be bound and presented by HLA class I histocompatibility antigen, alpha chain E (HLA-E), which is a non-classical major histocompatibility complex (MHC) class I molecule. The inventors have found that the EBV specific immune response is HLA-E-restricted which means that a T cell can interact with a MHC and the SQAPLPCVL peptide but will only respond when the antigen is bound by HLA-E. In the sense of the present invention, a cytotoxic CD8⁺ T is a cell that can interact with HLA-E bound SQAPLPCVL. Thus, the inventors have found that the EBV specific immune response initiated by the SQAPLPCVL peptide is an HLA-E-restricted CD8⁺ T cell response. When a mature CD8⁺ T cell binds the HLA-E bound SQAPLPCVL peptide, it initiates lysis of the target cell, which ultimately results in the death of EBV infected cells and successful immune response. In the sense of the present invention an immune response is preferably HLA-E-restricted CD8⁺ T cell response. Thus, the present invention provides an EBV vaccine that produces a HLA-E-restricted CD8⁺ T cell response when administered to a subject. The present invention also provides an EBV vaccine that produces a HLA-E-restricted CD8⁺ T cell response in a subject.

Accordingly, the invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the EBV vaccine produces or is capable of producing an EBV-specific immune response.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the EBV vaccine produces or is or is capable of producing an EBV-specific immune response in the subject to be treated or in the subject to whom the vaccine is to be administered.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the EBV vaccine produces or is or is capable of producing an EBV-specific T cell response.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the EBV vaccine produces or is or is capable of producing an EBV-specific HLA-E-restricted CD8⁺ T cell response.

The herein provided Epstein-Barr-Virus (EBV) vaccine, comprising a SQAPLPCVL peptide or a nucleic acid encoding a SQAPLPCVL peptide and, optionally, a pharmaceutically acceptable carrier, can (is capable of) produce/induce/elicit an immune response or a T cell population (in the subject to be treated/to whom the vaccine is to be administered). Preferably, the immune response or T cell population is produced/induced/elicited in an amount sufficient to confer protection against EBV/EBV infection. For example, in prophylactic treatment a vaccinated subject is asymptomatic following EBV infection and/or EBV spread is prevented/reduced and/or EBV replication is prevented/reduced in a). In terms of therapeutic treatment, a vaccinated subject shows reduced symptoms following EBV infection and/or reduced EBV spread and/or reduced EBV replication compared to prior the vaccination or compared to a subject/control group that is not vaccinated.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the EBV vaccine produces or is capable of producing an EBV-specific immune response, wherein the immune response is produced/induced/present in an amount sufficient to confer protection/immunity to an infected subject, preferably, resulting in an asymptomatic subject and/or prevention or reduction of EBV spread and/or replication.

The immune response or T cell population is described in more detail further below.

The HLA-E-restricted CD8⁺ T cell response elicited by the EBV vaccine of the invention may optionally comprise a HLA-E-restricted NKG2A⁺ CD8⁺ T cell response and/or a NKG2A⁻ CD8⁺ T cell response. NKG2A is a C-type lectin receptor expressed on distinct CD8⁺ T and NK cell subsets. The NKG2A receptor can inhibit the cytotoxic activity of NK cells. Thus, the HLA-E-restricted CD8⁺ T cell response of the present invention can comprise CD8⁺ T cells that are NKG2A⁺ or NKG2A⁻. The inventors have shown that HLA-E-restricted NKG2A⁺ CD8⁺ T cells and HLA-E-restricted NKG2A⁻ CD8⁺ T cells can inhibit viral spread and replication. In a non-limiting example, a HLA-E-restricted CD8⁺ T cell response comprising a NKG2A⁻ CD8⁺ T cell response may be preferred.

Accordingly, the invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the EBV vaccine produces or is or is capable of producing an EBV-specific HLA-E-restricted CD8⁺ T cell response comprising a NKG2A⁺ CD8⁺ T cell response.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the EBV vaccine produces or is or is capable of producing an EBV-specific HLA-E-restricted CD8⁺ T cell response comprising a NKG2A⁻ CD8⁺ T cell response.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the EBV vaccine produces or is or is capable of producing an EBV-specific HLA-E-restricted CD8⁺ T cell response comprising a NKG2A⁺ CD8⁺ T cell response and/or a NKG2A⁻ CD8⁺ T cell response.

In a preferred aspect, the invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the EBV vaccine produces or is or is capable of producing an EBV-specific HLA-E-restricted CD8⁺ T cell response.

The EBV-specific immune response is initiated by presentation of the SQAPLPCVL peptide.

Accordingly, an EBV-specific immune response can be a SQAPLPCVL-specific immune response in the sense of the present invention.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the EBV vaccine produces or is or is capable of producing a SQAPLPCVL-specific HLA-E-restricted CD8⁺ T cell response.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the EBV vaccine produces or is or is capable of producing a SQAPLPCVL-specific HLA-E-restricted CD8⁺ T cell response comprising a NKG2A⁺ CD8⁺ T cell response.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the EBV vaccine produces or is or is capable of producing a SQAPLPCVL-specific HLA-E-restricted CD8⁺ T cell response comprising a NKG2A⁻ CD8⁺ T cell response.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the EBV vaccine produces or is or is capable of producing a SQAPLPCVL-specific HLA-E-restricted CD8⁺ T cell response comprising a NKG2A⁺ CD8⁺ T cell response and/or a NKG2A⁻ CD8⁺ T cell response.

In a preferred aspect, the invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the EBV vaccine produces or is or is capable of producing a SQAPLPCVL-specific HLA-E-restricted CD8⁺ T cell response.

The EBV vaccine of the present invention efficiently prevents and inhibits or reduces the viral spread of EBV. In general, the viral spread can be prevented in a subject that is not infected with EBV or in a subject that is infected with EBV. The viral spread or the virus replication can be inhibited and/or reduced in a subject that has an acute symptomatic EBV infection or reactivation or an asymptomatic EBV infection or reactivation. The viral spread or the virus replication can also be inhibited and/or reduced in a subject that has an acute symptomatic EBV-induced disease or an asymptomatic EBV-induced disease. The prevention, inhibition or reduction of viral spread or virus replication is achieved by the EBV-specific or QAPLPCVL-specific HLA-E-restricted CD8⁺ T cell response which results in the killing of EBV infected cells. The viral spread of EBV and therefore the virus replication in an individual can be reduced by up to 100% when comparing a subject treated with the EBV vaccine of the present invention with an untreated subject.

In general, the efficiency of the vaccine can be shown in cell culture experiments, in which the viral spread is compared between cells, which had been treated with the EBV vaccine and cells, which had not been treated with the EBV vaccine. The administration of the EBV vaccine results in a 90% reduction of the viral spread and the viral replication. A reduction of 100% can indicate that a subject is asymptomatic for the EBV infection and/or that the subject developed an EBV specific immune response manages infection and confers immunity.

Treatment with the EBV vaccine of the present invention can reduce the viral spread or virus replication in a subject for example between about 10% and 100%. The EBV vaccine of the present invention can reduce the viral spread or virus replication in a subject about 10%, 15%, 20%, 25%, 30%, 35%, 40%, 45%, 50%, 55%, 60%, 65%, 70%, 75%, 80%, 85%, 90%, 95%, or 100%. The EBV vaccine can reduce the viral spread or virus replication in a subject about 60-90%.

Accordingly, the invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease wherein the EBV vaccine and/or the immune response prevent EBV spread and/or EBV replication or is capable of preventing EBV spread and/or EBV replication (in the subject to be treated/to whom the vaccine is to be administered).

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease wherein the EBV vaccine reduces the viral spread or virus replication in a subject about 10%, 15%, 20%, 25%, 30%, 35%, 40%, 45%, 50%, 55%, 60%, 65%, 70%, 75%, 80%, 85%, 90%, 95%, or 100%.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease wherein the EBV vaccine reduces the viral spread or virus replication in a subject about 60-90%.

A reduction of viral spread or virus replication of 100% can indicate that a subject is is asymptomatic for the EBV infection and/or that the subject developed an EBV specific immune response that manages infection and confers a high degree of long-term immunity. In this case, the EBV vaccine prevents the viral spread and reduces the virus replication in a subject. This is desired for long-term immunity to EBV or EBV-induced disease. As used herein, “immunity to EBV” or “immune to EBV” also includes that a subject may still contract a EBV infection or may have a detectable virus replication, however it also means that the respective subject developed and acquired immunity that is able to suppress and manage EBV infection and development of EBV-induced diseases resulting in at most asymptomatic EBV infection, asymptomatic EBV reactivation or asymptomatic EBV-induced diseases. Preferably, “immunity to EBV” or “immune to EBV” means that a subject developed and acquired immunity that is able to prevent acute symptomatic EBV infection, acute symptomatic EBV reactivation and/or EBV-induced diseases. This may result in long-term EBV immunity. Such an immunity may be achieved by memory T or B cells which can confer long-term protection e.g. by antibody production or through an augmented immune response after reactivation upon a reinfection event.

Accordingly, the invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the EBV vaccine prevents the viral spread and reduces the virus replication in a subject up to about 100%.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the EBV vaccine results in a long-term immunity to EBV or an EBV-induced disease.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the EBV- or SQAPLPCVL-specific HLA-E-restricted CD8⁺ T cell response results in long-term immunity to EBV or an EBV-induced disease.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the EBV vaccine results in EBV-specific memory B cells or memory T cells.

The invention also provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the EBV- or SQAPLPCVL-specific HLA-E-restricted CD8⁺ T cell response results in EBV-specific memory B cells or memory T cells which enable long-term immunity to EBV or an EBV-induced disease.

The inventors have found that the EBV- or SQAPLPCVL-specific HLA-E-restricted CD8⁺ T cell response is maintained throughout a long time in a subject over the course of years after primary infection. Thus, the EBV vaccine of the present invention is especially useful when long-term immunity to EBV or EBV-induced diseases is desired in a subject. As used herein, “EBV immunity” includes immunity to EBV infection, EBV reactivation and EBV-induced or associated diseases. The HLA-E-restricted CD8⁺ T cell acquired immunity to EBV can be maintained in a subject up to 25 years, such as up to 5, 10, 15, 20, or 25 years. The immunity may further be maintained as described herein by booster doses of the EBV vaccine.

Accordingly, a booster dose may be administered to a subject every 5, 10, 15, 20 or 25 years.

As described herein, the EBV vaccine of the present invention can trigger EBV-specific T cell responses. Such a response can comprise for example activation, expansion, and proliferation of T cells. Activation of T cells by the SQAPLPCVL peptide may lead to increased secretion of cytokines such as interferon gamma (IFN-γ). Expansion and proliferation of T cells elicited by the SQAPLPCVL peptide may leads to an increased number of T cells. The T cell may be a HLA-E-restricted CD8⁺ T cell.

Accordingly, the invention provides to an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the EBV vaccine activates T cells or is capable of T cell activation in a subject.

The invention also provides to an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the EBV vaccine activates HLA-E-restricted CD8⁺ T cells or is capable of HLA-E-restricted CD8⁺ T cell activation in a subject.

The invention also provides to an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the EBV vaccine initiates T cell expansion or is capable of initiating T cell expansion in a subject.

The invention also provides to an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the EBV vaccine initiates HLA-E-restricted CD8⁺ T cell expansion or is capable of initiating HLA-E-restricted CD8⁺ T cell expansion in a subject.

The invention also provides to an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the EBV vaccine initiates T cell proliferation or is capable of initiating T cell proliferation in a subject.

The invention also provides to an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the EBV vaccine initiates HLA-E-restricted CD8⁺ T cell proliferation or is capable of initiating HLA-E-restricted CD8⁺ T cell proliferation in a subject.

The invention also provides to an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the EBV vaccine produces or is capable of producing an EBV-specific immune response comprising activation, expansion, and proliferation of HLA-E-restricted CD8⁺ T cells.

Similarly, the above-described EBV specific HLA-E-restricted CD8⁺ T cell immune response can be initiated without infection. In this case, the initial HLA-E-restricted CD8⁺ T cell response may result in generation of memory T or B cells which can confer long-term protection e.g. by antibody production or through an augmented immune response after reactivation upon a reinfection event. This immune response then confers long time protection of EBV and EBV-induced diseases and is preferably initiated in adolescents/teenagers or children, e.g. by vaccination of a young child.

Accordingly, the invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the EBV vaccine results in EBV-specific memory B cells or memory T cells, and optionally EBV-specific antibody production.

In another aspect, the invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the EBV vaccine results in EBV-specific antibody production.

In another aspect, the invention provides an Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease, wherein the EBV vaccine or the treatment with the EBV vaccine produces EBV-specific antibodies in a subject.

The invention also provides a method of inducing an EBV-specific immune response in a subject. As described herein, the EBV vaccine is especially useful to induce an immune response that can confer immunity to EBV or EBV-induced diseases. Accordingly, the invention provides in one aspect a method of inducing an EBV-specific immune response in a subject, comprising administering an EBV vaccine to a subject, wherein the EBV vaccine comprises a SQAPLPCVL peptide or a nucleic acid encoding a SQAPLPCVL peptide and optionally a pharmaceutically acceptable carrier. The immune response is preferably a HLA-E restricted CD8⁺ T cell response which can comprise NKG2A⁺ and/or NKG2A⁻ CD8⁺ T cells.

The invention also provides a method to produce HLA-E-restricted CD8⁺ T cells in a subject in vivo, or to elicit a HLA-E-restricted CD8⁺ T cell response in a subject in vivo. The CD8⁺ T cells or the CD8⁺ T cell response can optionally comprise NKG2A⁺ and/or NKG2A⁻ CD8⁺ T cells. The above can be realized by administering the EBV vaccine of the present invention to a subject. This is especially useful since the HLA-E-restricted CD8⁺ T cells confer the immunity to EBV and EBV-induced diseases.

Accordingly, the invention provides a method to produce HLA-E-restricted CD8⁺ T cells optionally comprising HLA-E-restricted NKG2A⁺ CD8⁺ T cells and/or HLA-E-restricted NKG2A⁺ CD8⁺ T cells, or to elicit a HLA-E-restricted CD8⁺ T cell response optionally comprising a NKG2A⁺ CD8⁺ T cell response and/or a NKG2A⁻ CD8⁺ T cell response in a subject, comprising administering an EBV vaccine to the subject, wherein the EBV vaccine comprises a SQAPLPCVL peptide or a nucleic acid encoding a SQAPLPCVL peptide and, optionally, a pharmaceutically acceptable carrier.

The invention also relates to in vitro or ex vivo methods to produce HLA-E-restricted CD8⁺ T cells or elicit a HLA-E-restricted CD8⁺ T cell response. The CD8⁺ T cells or the CD8⁺ T cell response can optionally comprise NKG2A⁺ and/or NKG2A⁻ CD8⁺ T cells. Producing such cells is especially useful since they can be administered to subjects that are not able to produce or elicit an EBV-specific immune response themselves, such as the immunosuppressed or immunodeficient subject described herein. Such cells can be allogeneic. It is also envisioned herein that cells from a subject can be used to elicit a HLA-E-restricted CD8⁺ T cell response in said cells. Such cells can be autologous. Accordingly, the invention provides An in vitro or ex vivo method to produce HLA-E-restricted CD8⁺ T cells optionally comprising HLA-E-restricted NKG2A⁺ CD8⁺ T cells and/or HLA-E-restricted NKG2A⁻ CD8⁺ T cells, or to elicit a HLA-E-restricted CD8⁺ T cell response optionally comprising a NKG2A⁺ CD8⁺ T cell response and/or a NKG2A⁻ CD8⁺ T cell response in a cell, comprising contacting a cell with an EBV vaccine, wherein the EBV vaccine comprises a SQAPLPCVL peptide or a nucleic acid encoding a SQAPLPCVL peptide and, optionally, a pharmaceutically acceptable carrier.

As described herein above, the EBV vaccine of the present invention can comprise the SQAPLPCVL peptide as an active ingredient, e.g. in free form. Accordingly, the invention provides an Epstein-Barr-Virus (EBV) vaccine, comprising a SQAPLPCVL peptide or a nucleic acid encoding a SQAPLPCVL peptide and, optionally, a pharmaceutically acceptable carrier. The invention also provides the SQAPLPCVL peptide or a nucleic acid encoding a SQAPLPCVL peptide as such for use in therapy, or for use as a vaccine, or for use as a vaccine against EBV or for use as a medicament. Accordingly, the invention provides a SQAPLPCVL peptide or a nucleic acid encoding a SQAPLPCVL peptide for use as a vaccine. The invention also provides a SQAPLPCVL peptide or a nucleic acid encoding a SQAPLPCVL peptide for use as a vaccine against EBV. The invention also provides a SQAPLPCVL peptide or a nucleic acid encoding a SQAPLPCVL peptide for use as a medicament. In a preferred aspect, the invention provides a SQAPLPCVL peptide or a nucleic acid encoding a SQAPLPCVL peptide for use in the treatment of EBV or an EBV-induced disease.

The disclosures in context of the methods described herein are disclosed as corresponding use mutatis mutandis. The disclosures in context of the use described herein are disclosed as corresponding methods mutatis mutandis. In particular, the disclosures in context of the EBV vaccine for use in a treatment are disclosed as the corresponding methods of treating EBV mutatis mutandis. The disclosures in the context of methods of treating EBV are disclosed as the corresponding EBV vaccine for use in treatment mutatis mutandis.

The disclosures in the context of the methods of the invention described herein are applicable to the corresponding uses and vice versa. In particular, the disclosures in context of the EBV vaccine for use in a treatment described herein are applicable to the corresponding methods of treating EBV or EBV-induced diseases comprising administering the EBV vaccine and vice versa.

The methods and uses of the present invention can be in vivo, ex vivo or in vitro.

In one aspect, the methods of the present invention are not methods for treatment of the human or animal body by therapy. In a further aspect, the methods of the present invention are not processes for modifying the germ line genetic identity of human beings. In one aspect, the methods of the present invention are in vitro or ex vivo methods. In a further aspect, the methods of the present invention are not processes for modifying the genetic identity of animals which are likely to cause them suffering without any substantial medical benefit to man or animal.

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art. As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise. The terms “include,” “such as,” and the like are intended to convey inclusion without limitation, unless otherwise specifically indicated.

As used herein, the term “or” is generally employed in its usual sense including “and/or” unless the content clearly dictates otherwise. The term “and/or” means one or all of the listed elements or a combination of any two or more of the listed elements.

As used herein, the term “comprising” also specifically includes embodiments “consisting of” and “consisting essentially of” the recited elements, unless specifically indicated otherwise.

As used herein, the term “about” indicates and encompasses an indicated value and a range above and below that value. In certain embodiments, the term “about” indicates the designated value±10%, ±5%, or ±1%. In certain embodiments, where applicable, the term “about” indicates the designated value(s)±one standard deviation of that value(s).

As used herein, the terms “treatment” and “to treat” mean therapy. Therapy in the sense of the present invention includes therapeutic treatments, such as acute treatments of infections and diseases, as well as prophylactic/preventive treatments, such as an immunisation with a vaccine.

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a subject and includes: (a) preventing a disease related to an insufficient immune response from occurring in a subject which may be predisposed to the disease; (b) inhibiting the disease, i.e. arresting its development; or (c) relieving the disease, i.e. causing regression of the disease.

In the most preferred aspect, the term “treatment”, treating” or “to treat” (and the like) refer to “prevention”, “preventing” or “to prevent” and the like, e.g. a prophylactic treatment (a treatment having a prophylactic effect), like completely or partially preventing a disease or symptom thereof, preferably completely preventing a disease or symptom thereof. The terms “prevention” (and the like) and “prophylactic treatment” (and the like) are used interchangeably herein.

In a preferred aspect, the invention thus relates to a Epstein-Barr-Virus (EBV) vaccine, comprising a SQAPLPCVL peptide or a nucleic acid encoding a SQAPLPCVL peptide and, optionally, a pharmaceutically acceptable carrier, for use in the prevention of EBV (particularly of EBV infection) or of an EBV-induced disease. Likewise, the invention relates in a preferred aspect to a SQAPLPCVL peptide or a nucleic acid encoding a SQAPLPCVL peptide for use in the prevention of EBV (particularly of EBV infection) or of an EBV-induced disease.

As used herein, “producing an EBV-specific immune response” or “being capable of producing an EBV-specific immune response” relates to the EBV vaccine that results in a therapeutically effective outcome in form of an immune response, such as a HLA-E-restricted CD8⁺ T cell response. In particular, the terms relate to the EBV vaccine eliciting an immune response resulting in long-term immunity in a subject that may be infected with EBV but experiences an asymptomatic course of disease. The terms may also relate to the EBV vaccine eliciting an immune response resulting in the prevention or reduction of EBV spread or EBV replication in a subject.

The invention is illustrated in the following examples.

### BRIEF DESCRIPTION OF THE DRAWINGS

FIG. 1: HLA-E and EBV LMP-1 variants in patients with IM and asymptomatic EBV-seropositives. (A-D) Distribution of HLA-E variants between (A) control persons (N=412), (B) asymptomatic EBV-seropositive persons (N=206), (C) adolescent/adult IM (N=412) and (D) pediatric IM (N=166) patients. Fractions represent the relative frequency of HLA-E*0101/0101, HLA-E*0101/0103 and HLA-E*0103/0103 variants. (E,F) LMP-1 peptide variants in (E) adolescent/adult IM (N=412) and (F) pediatric IM (N=166) patients. Fractions represent the relative frequency of the LMP-1 peptide GGDPHLPTL, GSDPHLPTL, GGDPHLPPL, GGDPPLPTL, GCDPHLPTL, GIDPHLPTL, GAGPHLPTL, GGDTPLPTL, GDDPHLPTL, GGDPHVPTL and GTDPHLPTL variants. IM: infectious mononucleosis

FIG. 2: Sequence logo alignment of the relative frequency of (A, C) LMP-1 peptide variants and (B, D) BZLF1-derived SQAPLPCVL from N=786 sequenced BZLF1 and partially sequenced LMP-1 genes. (A, B) Sequence logo alignment of the relative frequency of (A) LMP-1 peptide variants and (B) BZLF1-derived SQAPLPCVL from N=578 sequenced BZLF1 and partially sequenced LMP-1 genes from IM patients. (C, D) Sequence logo alignment of the relative frequency of (C) LMP-1 peptide variants and (D) BZLF1-derived SQAPLPCVL from N=208 sequenced BZLF1 and partially sequenced LMP-1 genes from elderly, NON-PTLD and PTLD patients with EBV reactivations. Sequence logos were created with the web-tool of the University of California, (https://weblogo.berkeley.edu/).

FIG. 3: BZLF1-derived SQAPLPCVL and HLA-E restricted CD8⁺ T cell responses prevent the development of IM. (A-B) Analysis of SQAPLPCVL-specific and HLA-E restricted CD8⁺ T cell responses, evaluated between 12 healthy EBV-seropositive individuals and 6 healthy EBV-seronegative blood donors by flow-cytometry. Enriched CD8⁺ T cells were stimulated either with K562-HLA-E*0103/0103 cells, K562-HLA-E*0101/0101 cells, K562 cells or without cells and either 300 μM of the SQAPLPCVL peptide, 300 μM of the LMP-1 peptide pool (consisting of equal concentrations 11 LMP-1 peptide variants) or without peptides. (C-D) Evaluation of the viral spread and the activation of SQAPLPCVL-specific, HLA-E restricted CD8⁺ T cells against EBV-infected K562-CR2-HLA-E*0103/0103 cells or K562-CR2-HLA-E*0101/0101 cells. EBV-infected K562-CR2-HLA-E*0103/0103 cells or EBV-infected K562-CR2-HLA-E*0101/0101 cells were either cultured alone or together with SQAPLPCVL-specific, HLA-E restricted CD8⁺ T cells from 12 healthy EBV-seropositive individuals. (C) K562-CR2-HLA-E*0103/0103 or K562-CR2-HLA-E*0101/0101 cells were analyzed for the expression of EBV-BZLF1 after 3, 6, 9, 12, and 15 days post infections by flow-cytometry. (D) CD8⁺ T cells were then analyzed for the expression of IFNγ after 3, 6, 9, 12 and 15 days post infections by flow-cytometry. (A-D) RM one-way ANOVA (with the Geisser-Greenhouse correction) was used to compare differences between the respective groups. Plots represent the mean (±SD) of 12 independent biological replicates. (E) Analysis of proliferating SQAPLPCVL-specific and HLA-E restricted CD8⁺ T cells. Enriched and CFSE stained CD8⁺ T cells from 12 healthy EBV-seropositive individuals were co-cultured together with SQAPLPCVL-peptide pulsed K562-HLA-E*0103/0103 or K562-HLA-E*0101/0101 cells. The percentage of proliferating (CFSElow) CD8⁺ T cells was analyzed by flow-cytometry and compared by the Wilcoxon signed-rank test. (F-G) Percentage of SQAPLPCVL-specific and HLA-E restricted CD8⁺ T cells in patients with a recent IM (1-8 years; HLA-E*0101/0101: N=3, HLA-E*0101/0103: N=3, HLA-E*0103/0103: N=4) or past IM (10-23 years; HLA-E*0101/0101: N=3, HLA-E*0101/0103: N=3, HLA-E*0103/0103: N=4) or asymptomatic EBV-infected individuals (HLA-E*0101/0101: N=7, HLA-E*0101/0103: N=7, HLA-E*0103/0103: N=6). The percentage of SQAPLPCVL-specific and HLA-E restricted CD8⁺ T cells was compared between the groups by the Kruskal-Wallis Test and the Dunn's post-test. p<0.05 was considered significant. *, p<0.05; **, p<0.01; ***, p<0.001; ****, p<0.0001. IFNγ: Interferon γ, IM: infectious mononucleosis, neg.: negative, ns: not significant, pos.: positive, w/o: without.

FIG. 4: SQAPLPCVL-mediated HLA-E stabilization and NKG2A⁺ inhibition assay (A) HLA-E stabilization assay: K562-HLA-E*0103/0103 or K562-HLA-E*0101/0101 cells were incubated together with 300 μM of the positive control (VMAPRTLIL) or the BZLF1-derived SQAPLPCVL peptide. The surface expression of HLA-E was assessed after 16 h of co-culture by flow-cytometry. Box plot represent the mean (±SD) of three independent replicates. Each peptide was compared to the negative control (dashed black line), i.e. K562-HLA-E*0103/0103 or K562-HLA-E*0101/0101 cells without peptides using the Mann-Whitney test. (B) Analysis of SQAPLPCVL-specific and HLA-E restricted CD8⁺ T cell responses, evaluated between 12 healthy EBV-seropositive individuals by flow-cytometry. Enriched CD8⁺ T cells were stimulated either with K562-HLA-E*0103/0103 or K562-HLA-E*0101/0101 cells and 300 M of the SQAPLPCVL peptide. CD8⁺ T cells were then analyzed for the expression of IFNγ. Box bpot represent the mean (±SD) of 12 independent replicates. The percentage of IFNγ-expressing cells was compared between with K562-HLA-E*0103/0103 and K562-HLA-E*0101/0101 cells by the paired T-test. (C) Representative examples and median MFI of the negative control (without peptide), positive control (VMAPRTLIL) or SQAPLPCVL-induced HLA-E surface stabilisation on peptide pulsed K562-HLA-E*0103/0103 cells. MFI: mean fluorescence intensities

FIG. 5: Representative example of established K562-CR2-HLA-E*0103/0103 cell line using CRISPR gene editing. Overlay of CR2-APC stained K562-CR2-HLA-E*0103/0103 cells (orange), CR2-APC stained K562-HLA-E*0103/0103 cells (blue) and unstained K562-CR2-HLA-E*0103/0103 cells.

FIG. 6: Representative example of BZLF1 stained (red) and unstained (blue) EBV-infected K562-CR2-HLA-E*0103/0103 cells, obtained after 15 days of infection in the absence of SQAPLPCVL-specific and HLA-E restricted CD8⁺ T cells.

FIG. 7: BZLF1-derived SQAPLPCVL peptides are a potent inhibitor of NKG2A⁺ cells. (A-D) NKG2A⁺ inhibition assays: K562-HLA-E*0103/0103 or K562-HLA-E*0101/0101 cells were first incubated together with 300 μM of the positive control (VMAPRTLIL) or the BZLF1-derived SQAPLPCVL peptide and then incubated together with (A-B) pre-activated enriched CD56⁺ NK or (C-D) pre-activated enriched CD8⁺ T cells. The percentage of (A) CD107 expressing NKG2A⁺ NK cells or (C) IFNγ expressing NKG2A⁺ CD8⁺ T cells was assessed by flow-cytometry. Each peptide was compared to the negative control (dashed black line), i.e. K562-HLA-E*0103/0103 or K562-HLA-E*0101/0101 cells without peptides using the Mann-Whitney test. (B, D) EBV dissemination assay: K562-CR2-HLA-E*0103/0103 or K562-CR2-HLA-E*0101/0101 cells were infected with the EBV B95-8 isolate and cultured together with (B) sorted NKG2A⁺ NK cells or (D) sorted NKG2A⁺ CD8⁺ T cells for 2, 4, 8, 12, or 14 days. The percentage (B, D) of BZLF1⁺ K562-CR2-HLA-E*0103/0103 or K562-CR2-HLA-E*0101/0101 cells was assessed by after 2, 4, 8, 12, or 14 days and (B) granzyme B-expressing NKG2A⁺ NK cells or (D) NKG2A⁺ CD8⁺ T cells was assessed by flow-cytometry after 14 days of co-culture. RM one-way ANOVA (with the Geisser-Greenhouse correction) was used to analyze differences between the peptides and the (B) granzyme expressing NKG2A⁺ NK or (D) NKG2A⁺ CD8⁺ T cells without peptides. (A, C) Plot represent the mean (±SD) of 12 independent biological replicates. (E-F) Analysis of (E) the percentage and (F) the expression level (MFI) of NKG2A⁺ expressing non-SQAPLPCVL-specific CD8⁺ T cells and NKG2A⁺ expressing SQAPLPCVL-specific T cells. The NKG2A MFI was assessed in both NKG2A⁺ CD8⁺ T cell subsets in 12 EBV-seropositive blood donors by flow cytometry and compared using the Wilcoxon matched T-test. (G) EBV dissemination assay: K562-CR2-HLA-E*0103/0103 or K562-CR2-HLA-E*0101/0101 cells were infected with the EBV B95-8 isolate and cultured together with sorted NKG2A⁺ expressing SQAPLPCVL-specific T cells or sorted NKG2A⁻SQAPLPCVL-specific T cells for 2, 4, 8, 12, or 14 days. The percentage of BZLF1⁺ K562-CR2-HLA-E*0103/0103 or K562-CR2-HLA-E*0101/0101 cells was assessed by flow-cytometry after 2, 4, 8, 12, or 14 days of co-culture. RM one-way ANOVA (with the Geisser-Greenhouse correction) was used to analyze differences between the cell subsets. p<0.05 was considered significant. *, p<0.05; **, p<0.01; ***, p<0.001; ****, p<0.0001. MFI: Mean Fluorescence Intensity. Pos. Ctrl.: positive control.

FIG. 8: Representative example of the NKG2A⁺ inhibition assay. K562-HLA-E*0103/0103 cells were first incubated together with 300 μM of the negative control (without peptide), positive control (VMAPRTLIL) or the BZLF1-derived SQAPLPCVL and then incubated together with pre-activated CD56⁺ NK cells from the same donor. The percentage of CD107 expressing NKG2A⁺ NK cells was assessed by flow-cytometry.

FIG. 9: Distribution of HLA-E and LMP-1 peptide variants in primary and reactivating EBV-infections. (A-B) Distribution of (A) HLA-E and (B) LMP-1 variants in immunocompetent elderly patients with EBV-reactivations (N=28). (C-F) Distribution of (C, D) HLA-E and (E, F) LMP-1 variants in (C, E) immunosuppressed non-PTLD (N=149) and (D, F) PTLD (N=36) transplant patients with EBV-reactivations. (A, C, D) Fractions represent the relative frequency of HLA-E*0101/0101, HLA-E*0101/0103 and HLA-E*0103/0103. (B, E, F) Fractions represent the relative frequency of the LMP-1 peptide GGDPHLPTL, GSDPHLPTL, GGDPHLPPL, GGDPPLPTL, GCDPHLPTL, GIDPHLPTL, GAGPHLPTL, GGDTPLPTL, GDDPHLPTL, GGDPHVPTL and GTDPHLPTL variants. PTLD: post-transplant lymphoproliferative disorders

FIG. 10: Distribution of HLA-E and LMP-1 peptide variants in transplant recipients with and without PTLD. (A-B) Distribution and comparison of HLA-E variants between (A) SOT Non-PTLD (N=129) and SOT PTLD (N=20) or (B) HSCT Non-PTLD (N=15) and HSCT PTLD (N=16). Fractions represent the relative frequency of HLA-E*0101/0101, HLA-E*0101/0103 and HLA-E*0103/0103. (C-D) Distribution and comparison of LMP-1 peptide variants between (A) SOT Non-PTLD (N=129) and SOT PTLD (N=20) or (B) HSCT Non-PTLD (N=15) and HSCT PTLD (N=16). Fractions represent the relative frequency of the LMP-1 peptide variants GGDPHLPTL, GSDPHLPTL, GGDPHLPPL, GGDPPLPTL, GCDPHLPTL, GIDPHLPTL, GAGPHLPTL, GGDTPLPTL, GDDPHLPTL, GGDPHVPTL and GTDPHLPTL. HSCT: hematopoietic stem cell transplantation, PTLD: post-transplant lymphoproliferative disorders, SOT: solid organ transplantation.

FIG. 11: LMP-1-derived peptides are a potent inhibitor of NKG2A⁺ NK cells. (A) HLA-E stabilization assay: K562-HLA-E*0103/0103 or K562-HLA-E*0101/0101 cells were incubated together with 300 μM of the positive control (VMAPRTLIL) or the LMP-1 derived GGDPHLPTL, GSDPHLPTL, GGDPHLPPL, GGDPPLPTL, GCDPHLPTL, GIDPHLPTL, GAGPHLPTL, GGDTPLPTL, GDDPHLPTL, GGDPHVPTL and GTDPHLPTL peptides. The surface expression of HLA-E was then assessed after 16 h of co-culture by flow-cytometry. Box plot represent the mean (±SD) of three independent replicates. Each peptide was compared to the negative control (dashed black line), i.e., K562-HLA-E*0103/0103 cells or K562-HLA-E*0101/0101 cells without peptides using the Mann-Whitney test. (B) NKG2A⁺ inhibition assay: K562-HLA-E*0103/0103 or K562-HLA-E*0101/0101 cells were first incubated together with 300 μM of the positive control (VMAPRTLTL) or the LMP-1 derived GGDPHLPTL, GSDPHLPTL, GGDPHLPPL, GGDPPLPTL, GCDPHLPTL, GIDPHLPTL, GAGPHLPTL, GGDTPLPTL, GDDPHLPTL, GGDPHVPTL and GTDPHLPTL peptides and then incubated together with pre-activated enriched CD56⁺ NK cells. The percentage of CD107 expressing NKG2A⁺ NK cells was assessed by flow-cytometry. Plots represent the mean (±SD) of 12 independent biological replicates. Each peptide was compared to the negative control (dashed black line), i.e. K562-HLA-E*0103/0103 cells or K562-HLA-E*0101/0101 cells without peptides using the Mann-Whitney test. (C-D) EBV dissemination assay: K562-CR2-HLA-E*0103/0103 or K562-CR2-HLA-E*0101/0101 cells were infected with the EBV B95-8 isolate and cultured together with sorted NKG2A⁺ NK cells for 2, 4, 8, 12, or 14 days. (C) The percentage of BZLF1⁺ K562-CR2-HLA-E*0103/0103 or K562-CR2-HLA-E*0101/0101 cells or (D) granzyme B-expressing NKG2A⁺ NK cells was assessed by flow-cytometry after 2, 4, 8, 12, or 14 days of co-culture. The dashed black line indicates the percentage of granzyme B-expressing NKG2A⁺ NK cells in the absence of any peptides. (C) Plots represent the mean (±SD) of 12 independent biological replicates. RM one-way ANOVA (with the Geisser-Greenhouse correction) was used to analyze differences between the peptides. (D) Box plot represent the mean (±SD) of 12 independent replicates. Each peptide was compared to the negative control (dashed black line), i.e. K562-HLA-E*0103/0103 cells or K562-HLA-E*0101/0101 cells without peptides using the Mann-Whitney test. p<0.05 was considered significant. *, p<0.05; **, p<0.01; ***, p<0.001; ****, p<0.0001. IM: infectious mononucleosis, MFI: Mean Fluorescence Intensity. Pos. Ctrl.: positive control, PTLD: post-transplant lymphoproliferative disorders.

FIG. 12: LMP-1-derived peptides are a potent Inhibitor of NKG2A⁺ CD8⁺ T cells. (A) NKG2A⁺ inhibition assay: K562-HLA-E*0103/0103 or K562-HLA-E*0101/0101 cells were first incubated together with 300 μM of the positive control (VMAPRTLIL) or the LMP-1 derived GGDPHLPTL, GSDPHLPTL, GGDPHLPPL, GGDPPLPTL, GCDPHLPTL, GIDPHLPTL, GAGPHLPTL, GGDTPLPTL, GDDPHLPTL, GGDPHVPTL and GTDPHLPTL peptides and then incubated together with pre-activated enriched NKG2A⁺ CD8⁺ T Cells. The percentage of IFNγ expressing NKG2A⁺ CD8⁺ T cells was assessed by flow-cytometry. Box plot represent the mean (±SD) of 12 independent biological replicates. Each peptide was compared to the negative control (dashed black line), i.e. K562-HLA-E*0103/0103 or K562-HLA-E*0101/0101 cells without peptides using the Mann-Whitney test. (B-C) EBV dissemination assay: K562-CR2-HLA-E*0103/0103 or K562-CR2-HLA-E*0101/0101 cells were infected with the EBV B95-8 isolate and cultured together with sorted NKG2A⁺ CD8⁺ T cells for 2, 4, 8, 12 or 14 days. The percentage of (B) BZLF1+K562-CR2-HLA-E*0103/0103 or K562-CR2-HLA-E*0101/0101 cells or (C) granzyme B-expressing NKG2A⁺ CD8⁺ T cells was assessed by flow-cytometry after 2, 4, 8, 12 or 14 days of co-culture. The dashed black line indicates the percentage of granzyme B-expressing CD8⁺ T cells in the absence of any peptides. (B) RM one-way ANOVA (with the Geisser-Greenhouse correction) was used to analyze differences between groups. (C) Each peptide was compared to the negative control (dashed black line), i.e. K562-HLA-E*0103/0103 cells or K562-HLA-E*0101/0101 cells without peptides using the Mann-Whitney test. (B-C) Plots represent the mean (±SD) of 12 independent biological replicates. p<0.05 was considered significant. *, p<0.05; **, p<0.01; ***, p<0.001; ****, p<0.0001. IFNγ: Interferon γ, Pos.Ctrl.: positive control.

FIG. 13: Sequence alterations of the SQAPLPCVL peptide do not elicit an EBV-specific, HLA-E restricted CD8+ T cell response (A-I) IFNγ (i.e. activated cells) expressing CD8+ T cells (±SD) are shown for each alteration of the SQAPLPCVL peptide and the SQAPLPCVL. Only cells pulsed with the SQAPLPCVL peptide show IFNγ (i.e. activated cells) expressing CD8+ T cells. All alterations led to an undetectable level of IFNγ (i.e. not activated cells) expressing CD8+ T cells. (J) For each amino acid, all peptides, i.e. all alterations (expect for the SQAPLPCVL) were combined into one graph. The mean of IFNγ (i.e. activated cells) expressing CD8+ T cells (±SD) is shown for the alterations of the SQAPLPCVL peptide (black dots) and the SQAPLPCVL (red/light grey dots). Only cells pulsed with the SQAPLPCVL peptide show IFNγ (i.e. activated cells) expressing CD8+ T cells. All alterations led to an undetectable level of IFNγ (i.e. not activated cells) expressing CD8+ T cells.

FIG. 14: Longer (+3AA, +2AA, +1AA) or shorter (−3AA, −2AA, −1AA) variants of the SQAPLPCVL peptide do not elicit any EBV-specific, HLA-E restricted CD8+ T cell responses The mean of the IFNγ (i.e. activated cells) expressing CD8+ T cells (±SD) is shown for the longer and shorter peptides. Only cells pulsed with the SQAPLPCVL peptide show IFNγ (i.e. activated cells) expressing CD8+ T cells. All longer and shorter peptides led to an undetectable level of IFNγ (i.e. not activated cells) expressing CD8+ T cells.

FIG. 15: Larger polypeptides comprising the SQAPLPCVL motif do not elicit any EBV-specific, HLA-E restricted CD8+ T cell response The mean of the IFNγ (i.e. activated cells) expressing CD8+ T cells (±SD) is shown for the larger polypeptides comprising the SQAPLPCVL motif and the SQAPLPCVL peptide per se. Only the 9 amino acid long peptide sequence (SQAPLPCVL) elicits an EBV-specific, HLA-E restricted CD8+ T cell response, as demonstrated by a detectable amount of IFNγ. (i.e. activated cells) expressing CD8+ T cells. None of the larger polypeptides comprising the SQAPLPCVL motif are capable of eliciting an EBV-specific, HLA-E restricted CD8+ T cell response, as demonstrated by an undetectable amount of IFNγ. (i.e. not activated cells) expressing CD8+ T cells.

FIG. 16: SQAPLPCVL-specific, HLA-E-restricted CD8+ T cells do not express NKG2C and CD94 The mean (±SD) of the NKG2C/CD94-expressing and non-expressing SQAPLPCVL-specific, HLA-E-restricted CD8+ T cells as well all remaining SQAPLPCVL-specific, HLA-E-restricted CD8+ T cells is shown. SQAPLPCVL-specific, HLA-E-restricted CD8+ T cells expressed no NKG2C/CD94 (green/top panel of bar, 100% and 94.8% respectively). Non-SQAPLPCVL-specific, HLA-E-restricted CD8+ T do express NKG2C/CD94 (red/bottom panel of right bar).

FIG. 17: Evaluation of EBNA₃₈₆₋₄₀₅ and GlialCAM₃₇₀₋₃₈₉-specific immune responses. (A-D) Evaluation of EBNA₃₈₆₋₄₀₅ IgG antibody responses. EBNA₃₈₆₋₄₀₅-specific IgG antibody titers were assessed in N=270 controls by ELISA. The ELISA titers were normalized to one control plasma. EBNA₃₈₆₋₄₀₅ IgG antibody titers were then compared according to the (A) sex, (B) age, (C) total EBNA-1-specific IgG antibody titers, and (D) HLA-DRB1 genotypes of the controls. The groups were compared using (A) the Mann-Whitney-Test, (B-C) simple linear regression and (D) Kruskal-Wallis and Dunn's multiple comparison test. Each dot represents one individual patient. The (red) line represents (A,D) the corresponding means or (B,C) the linear regression. (E-G) Evaluation of GlialCAM₃₇₀₋₃₈₉-specific B-cell, plasma cell and IgG antibody responses. GlialCAM₃₇₀₋₃₈₉-specific (E) CD19⁺ B-cell or (F) CD138⁺ plasma cells were assessed in N=20 MS patients, N=48 EBNA^{low}, and N=32 EBNA^{high} controls by flow cytometry. Data are shown as the percentage of CFSE^{low} GlialCAM₃₇₀₋₃₈₉-specific (E) CD19⁺ B-cell or (F) CD138⁺ plasma cells, in comparison to all (E) CD19⁺ B-cell or (F) CD138⁺ plasma cells, respectively. (G) GlialCAM₃₇₀₋₃₈₉ IgG antibody titers were assessed in N=270 MS patients, N=162 EBNA^{low}, and N=108 EBNA^{high} controls by ELISA. The ELISA titers were normalized to one control plasma. The dashed black line indicates the 46% normalized IgG titer (100% negative predictive value) to discriminate between EBNA^{low} and EBNA^{high} controls. (E-G) The groups were compared by Kruskal-Wallis and Dunn's multiple comparison test. Each dot represents one individual patient. The (red) line represents the corresponding means. (H) Gating strategy for the identification of EBNA₃₈₆₋₄₀₅-specific IgG antibody-mediated ADCP and ADCC responses. CD14⁺CD16⁺ monocytes from 12 healthy EBV-seropositive blood donors were differentiated into CD14⁺CD16⁺ M1 macrophages and then stimulated with AF647 NHS Ester-linked GlialCAM₃₇₀₋₃₈₉ peptides in the presence or absence of individual plasma of N=270 MS patients, N=162 EBNA^{low}, and N=108 EBNA^{high} controls. GlialCAM₃₇₀₋₃₈₉-specific ADCP responses were assessed as the median AF647 NHS Ester MFI of CD14+CD16+M1 macrophages. GlialCAM₃₇₀₋₃₈₉-specific ADCC responses were assessed in parallel as CD107a⁺CD14⁺CD16⁺ M1 macrophages. One representative example of two parallel experiments, in the presence or absence of plasma of one EBNA^{low} control individual is shown. (I) Gating strategy for the identification of EBNA₃₈₆₋₄₀₅-specific CD8⁺ T-cells (panels in first row), CD4⁺ T-cells (panels in second row) and CD138⁺ plasma cells (panels in third row). PBMCs from one control individual were cultured in the presence of the EBNA₃₈₆₋₄₀₅ peptide. EBNA₃₈₆₋₄₀₅-specific CD8⁺ T-cells, CD4⁺ T-cells and CD138⁺ plasma cells were identified as CFSE^{low} cells (blue). (J) CD4⁺ T-cells from one control individual were cultured in the presence or absence of the EBNA₃₈₆₋₄₀₅ peptide. EBNA₃₈₆₋₄₀₅-specific CD4⁺ T-cells were identified as CFSE^{low} cells (blue/light grey) in comparison to the non-peptide control (red/dark grey). One representative example is shown. ADCC: antibody-dependent cellular cytotoxicity, ADCP: antibody-dependent cellular phagocytosis, DU: DRG units, MFI: mean fluorescence intensity, MS: Multiple Sclerosis, ns: not significant.

FIG. 18: Evaluation of EBNA₃₈₆₋₄₀₅- and GlialCAM₃₇₀₋₃₈₉-specific immune responses. (A) EBNA₃₈₆₋₄₀₅-specific IgG antibodies titers were assessed in N=270 MS patients, N=162 EBNA^{low}, and N=108 EBNA^{high} controls by ELISA. The ELISA titers were normalized to one control plasma. The dashed black line indicates the 46% normalized IgG titer (100% negative predictive value) to discriminate between EBNA^{low} and EBNA^{high} controls. (B-C) M1 macrophages-mediated and GlialCAM₃₇₀₋₃₈₉-specific (B) ADCP and (C) ADCC responses from N=270 MS patients, N=162 EBNA^{low}, and N=108 EBNA^{high} controls were assessed using monocyte-derived M1 macrophages from 12 healthy EBV-seropositive blood donors. (B) GlialCAM₃₇₀₋₃₈₉-specific ADCP was assessed as AF647 MFI by flow cytometry. (C) GlialCAM₃₇₀₋₃₈₉-specific ADCC was assessed as CD107a⁺CD14⁺ cells by flow cytometry. (B-C) The data were normalized to one control plasma. (D-F) EBNA₃₈₆₋₄₀₅-specific (D) CD138⁺ plasma cells, (E) CD4⁺ T-cells, and (F) CD8⁺ T-cells were assessed in N=20 MS patients, N=48 EBNA^{low}, and N=32 EBNA^{high} controls by flow cytometry. Data are shown as the percentage of CFSE^{low} EBNA₃₈₆₋₄₀₅-specific (D) CD138⁺ plasma cells, (E) CD4⁺ T-cells, and (F) CD8⁺ T-cells in comparison to total (D) CD138⁺ plasma cells, (E) CD4⁺ T-cells and (F) CD8⁺ T-cell levels. (G-J) GlialCAM₃₇₀₋₃₈₉-specific (G, I) CD4⁺ T-cells and (H, J) CD8⁺ T-cells were assessed in N=20 MS patients, N=48 EBNA^{low}, and N=32 EBNA^{high} controls by flow cytometry. Data are shown as a percentage of CFSE^{low} GlialCAM₃₇₀₋₃₈₉-specific (G, I) CD4⁺ T-cells and (H, J) CD8⁺ T-cells in comparison to (G,I) total or (H,J) EBNA₃₈₆₋₄₀₅-specific CD4⁺ T-cells and CD8⁺ T-cell levels, respectively. (A-J). The groups were compared by Kruskal-Wallis and Dunn's multiple comparison test. Each dot represents one individual patient. The (red) line represents the corresponding means. ADCC: antibody-dependent cellular cytotoxicity, ADCP: antibody-dependent cellular phagocytosis, MFI: mean fluorescence intensity, MS: Multiple Sclerosis, ns: not significant.

FIG. 19: Control of GlialCAM₃₇₀₋₃₈₉-specific immune responses. (A-B) Phenotype of GlialCAM₃₇₀₋₃₈₉-specific cells after stimulation with the GlialCAM₃₇₀₋₃₈₉ peptide. Total PBMCs from 24 healthy control individuals were stimulated with the GlialCAM₃₇₀₋₃₈₉ peptide or a random 20 amino acid peptide pool (negative control, NC) or without any peptides (non-peptide control, NPC) for 14 days. GlialCAM₃₇₀₋₃₈₉-specific (GlialCAM₃₇₀₋₃₈₉) or total (NC, NPC) CD8⁺ T-cells, CD4⁺ T-cells or CD138⁺ plasma cells were then sorted, analyzed for the expression of PD-1 by cytometry and pooled for the cell killing experiments. (A) PD1 expression of GlialCAM₃₇₀₋₃₈₉-specific (GlialCAM₃₇₀₋₃₈₉) or total (NC, NPC) CD8⁺ T-cells, CD4⁺ T-cells or CD138⁺ plasma cells. Data are shown as the mean cellular fractions after pooling of 24 independent experiments±SEM. The individual panels in the bars relate from top to bottom to: PD1-CD8⁺ T cells, PD1+CD8⁺ T cells, PD1-CD4⁺ T cells, PD1+CD4⁺ T cells, PD1-CD138⁺ plasma cells and PD1+CD138⁺ T plasma cells. (B) Characterization of GlialCAM₃₇₀₋₃₈₉-specific CD8⁺ T-cells. GlialCAM₃₇₀₋₃₈₉-specific (GlialCAM₃₇₀₋₃₈₉) or total (NC, NPC) CD8⁺ T-cells were analyzed for the expression of LAG3, PD1, TGIT, TIM3, and NKG2A. Each fraction represent the mean frequency of the LAG3⁻PD1⁺/⁻TGIT⁺/⁻TIM3⁻NKG2A⁻, LAG3⁺PD1⁺TGIT⁺TIM3⁻NKG2A⁻ and LAG3⁺PD1⁺TGIT⁺TIM3⁺NKG2A⁺ CD8⁺ T cell subsets (±SEM). The individual panels in the bars relate from top to bottom to: LAG3⁺PD1⁺TGIT⁺TIM3⁺NKG2A⁺, LAG3+PD1⁺TGIT⁺TIM3⁻NKG2A⁻ and LAG3⁻PD1⁻TGIT⁻TIM3⁻NKG2A⁻. (A-B) Groups were compared using the Two-Way-ANOVA. (C-D) Control of GlialCAM₃₇₀₋₃₈₉-specific immune responses by CD8⁺ T-cells. PBMCs from 24 healthy control individuals were first stimulated with the GlialCAM₃₇₀₋₃₈₉-peptide or a random 20 amino acid peptide pool (negative control, NC) or without any peptides (non-peptide control, NPC) and then co-cultured with enriched CD8⁺ T-cells from the same donor. The CD8⁺ T-cell activation level was assessed either by (C) IL-2 ELISA or by (D) IFNγ ELISA. (C-D) The groups were compared by the Kruskal-Wallis and the Dunn's multiple comparison test. Each dot represents one individual sample from one healthy control. (E) Gating strategy for the identification of the NKG2D⁺ NK cell-mediated cytotoxicity against GlialCAM₃₇₀₋₃₈₉-specific cells. PBMCs from one healthy blood donor were first stimulated with the GlialCAM₃₇₀₋₃₈₉-peptide (GlialCAM₃₇₀₋₃₈₉) or a random 20 amino acid peptide pool (negative control, NC) or without any peptides (non-peptide control, NPC). GlialCAM₃₇₀₋₃₈₉-specific (GlialCAM₃₇₀₋₃₈₉) or total (NPC, NC) CD8⁺ T-cells, CD4⁺ T-cells or CD138⁺ plasma cells were then sorted and co-cultured with sorted NKG2D⁺ NK cell from the same donor. Cytotoxic NKG2D⁺ NK cell responses were assessed as CD107a⁺CD56⁺NKG2D⁺ NK cells in response to GlialCAM₃₇₀₋₃₈₉-specific B-cells and T-cells (blue, 12.4% CD107a⁺CD56⁺NKG2D⁺ NK cells), the NC (red 2.2% CD107a⁺CD56⁺NKG2D⁺ NK cells), or the NPC (orange 2.5% CD107a⁺CD56⁺NKG2D⁺ NK cells). One representative example is shown. (F-G) NKG2C⁺ NK cell responses: (F) Simple linear regression of EBNA₃₈₆₋₄₀₅-specific IgG antibody titers and NKG2C⁺ NK cell frequencies in N=20 MS patients, N=48 EBNA^{low}, and N=32 EBNA^{high} controls. Each dot represents one individual sample. (G) PBMCs from 24 healthy control individuals (KLRC2^{wt/wt}: N=12 or KLRC2^{wt/del}: N=12) were first stimulated with the GlialCAM₃₇₀₋₃₈₉-peptide or a random 20 amino acid peptide pool (negative control, NC) or without any peptides (non-peptide control, NPC). GlialCAM₃₇₀₋₃₈₉-specific (GlialCAM₃₇₀₋₃₈₉) or total (NPC, NC) CD8⁺ T-cells, CD4⁺ T-cells or CD138⁺ plasma cells were then sorted and co-cultured with sorted NKG2C⁺ NK cell from the same donor. The LDH release was then assessed in the supernatant. Results are shown as fold change of the GlialCAM₃₇₀₋₃₈₉-specific cells or the NC, normalized to the NPC. The dashed black line indicates an equal activation of NK cells against GlialCAM₃₇₀₋₃₈₉-specific and non-GlialCAM₃₇₀₋₃₈₉-specific cells. The groups were compared by the Wilcoxon matched T-test. (H-I) NKG2D⁺ NK cell responses: (H) Simple linear regression of EBNA₃₈₆₋₄₀₅-specific IgG antibody titers and NKG2D⁺ NK cell frequencies in N=20 MS patients, N=48 EBNA^{low}, and N=32 EBNA^{high} controls. Each dot represents one individual sample. (I) PBMCs from 24 healthy control individuals (NKG2D^{HNK/HNK}: N=8, NKG2D^{HNK/LNK}: N=8, or NKG2D^{LNK/LNK}: N=8) were first stimulated with the GlialCAM₃₇₀₋₃₈₉-peptide or a random 20 amino acid peptide pool (negative control, NC) or without any peptides (non-peptide control, NPC). GlialCAM₃₇₀₋₃₈₉-specific (GlialCAM₃₇₀₋₃₈₉) or total (NPC, NC) CD8⁺ T-cells, CD4⁺ T-cells or CD138⁺ plasma cells were then sorted and co-cultured with sorted NKG2D⁺ NK cell from the same donor. The LDH release was then assessed in the supernatant. Results are shown as fold change of the GlialCAM₃₇₀₋₃₈₉-specific cells or the NC, normalized to the NPC. The dashed black line indicated an equal activation of NK cells against GlialCAM₃₇₀₋₃₈₉-specific and non-GlialCAM₃₇₀₋₃₈₉-specific cells. The groups were compared by the Wilcoxon matched T-test. LDH: lactate dehydrogenase, MFI: mean fluorescence intensity, MS: Multiple Sclerosis, NC: negative control, NPC: non-peptide control, ns: not significant, wt: wild type.

FIG. 20: Control of GlialCAM₃₇₀₋₃₈₉-specific immune responses. (A-D) PBMCs from 24 healthy control individuals were first stimulated with the GlialCAM₃₇₀₋₃₈₉-peptide (GlialCAM₃₇₀₋₃₈₉) or a random 20 amino acid peptide pool (negative control, NC) or without any peptides (none-peptide control, NPC). Sorted GlialCAM₃₇₀₋₃₈₉-specific (GlialCAM₃₇₀₋₃₈₉) or total (NC, NPC) CD4⁺ T-cells, CD8⁺ T-cells, and CD138⁺ plasma cells were then pooled and co-cultured with (A) Jurkat T-cells, (B) THP-1 monocytes, or (C) NK92 NK cells. The cell activation level was either assessed by (A) IL-2 ELISA, (B) flow cytometry (AF647 MFI), or (C) IFNγ ELISA. (A-C) The groups were compared by the Kruskal-Wallis and the Dunn's multiple comparison test. (D) GlialCAM₃₇₀₋₃₈₉-specific CD4⁺ T-cells, CD8⁺ T-cells, and CD138⁺ plasma cells (GlialCAM₃₇₀₋₃₈₉) or total CD4⁺ T-cells, CD8⁺ T-cells and CD138⁺plasma cells (NC) from 24 healthy blood donors were co-cultured with sorted KIR2DS1⁺, KIR3DS1⁺, NKp46⁺ (NCR1), NKp44⁺ (NCR2), NKp30⁺ (NCR3), NKG2D⁺, or NKG2C⁺ CD56⁺ NK cell subsets from the same donor. The percentage of activated and cytotoxic (CD107a⁺) KIR2DS1⁺, KIR3DS1⁺, NKp46⁺ (NCR1), NKp44⁺ (NCR2), NKp30⁺ (NCR3), NKG2D⁺ or NKG2C⁺ CD56⁺ NK cell subsets was assessed by flow cytometry. Results are shown as fold change of the GlialCAM₃₇₀₋₃₈₉-specific (GlialCAM₃₇₀₋₃₈₉) to total cells, stimulated without peptides (NC). The dashed black line indicated an equal activation of NK cells against GlialCAM₃₇₀₋₃₈₉-specific cell (GlialCAM₃₇₀₋₃₈₉) and cells, stimulated without peptides (NC). Each dot represents one individual sample from one healthy control. The groups were compared by Wilcoxon matched T-test. (E-G) NKG2C⁺ NK cell responses. (E) NKG2C⁺ NK cell frequencies were assessed in N=20 MS patients, N=48 EBNA^{low}, and N=32 EBNA^{high} controls by flow cytometry and compared by the Kruskal-Wallis and the Dunn's multiple comparison test. (F) GlialCAM₃₇₀₋₃₈₉-specific CD4⁺ T-cells, CD8⁺ T-cells and CD138⁺ plasma cells (GlialCAM₃₇₀₋₃₈₉) or total CD4⁺ T-cells, CD8⁺ T-cells and CD138⁺ plasma cells (NC) from 24 healthy blood donors were co-cultured with sorted NKG2C⁺CD56⁺ NK cells from the same donor. The controls expressed either the KLRC2^{wt/wt} (N=12), or KLRC2^{wt/del} (N=12) genotype. The percentage of activated and cytotoxic (CD107a⁺) NKG2C⁺CD56⁺ NK cells was assessed by flow cytometry. Results are shown as fold change of the GlialCAM₃₇₀₋₃₈₉-specific (GlialCAM₃₇₀₋₃₈₉) to non-specific cells (NC). The dashed black line indicated an equal activation of NK cells against GlialCAM₃₇₀₋₃₈₉-specific (GlialCAM₃₇₀₋₃₈₉) and non-specific cells (NC). The groups were compared by the Wilcoxon matched T-test. (G) Distribution of the HCMV-serostatus, KLRC2 variants, and HCMV-encoded UL40 variants of the infecting HCMV-variant. Fractions represent the relative frequency of each combination of the HCMV-serostatus, KLRC2 variants, and HCMV-encoded UL40 variants. UL40 variants were categorized into high (↑↑)- and low (↑)-binders as shown in Table 4. The frequency of the combination was compared between the cohorts by the Chi² Test. The individual panels in the bars relate from left to right to: HCMV neg.; HCMV pos.+KLRC2^{del/del}; HCMV pos.+KLRC2^{wt/del}+Ul40↑; HCMV pos.+KLRC2^{wt/wt}+Ul40↑; HCMV pos.+KLRC2^{wt/del}+Ul40↑↑; and HCMV pos.+KLRC2^{wt/wt}+Ul40↑↑. (H-J) NKG2D⁺ NK cell responses. (H) NKG2D⁺ NK cell frequencies were assessed in N=20 MS patients, N=48 EBNA^{low}, and N=32 EBNA^{high} controls by flow cytometry and compared by the Kruskal-Wallis and the Dunn's multiple comparison test. (I) GlialCAM₃₇₀₋₃₈₉-specific CD4⁺ T-cells, CD8⁺ T-cells and CD138⁺ plasma cells (GlialCAM₃₇₀₋₃₈₉) or non-GlialCAM₃₇₀₋₃₈₉-specific CD4⁺ T-cells, CD8⁺ T-cells and CD138⁺ plasma cells (NC) from 24 healthy blood donors were co-cultured with sorted NKG2D⁺CD56⁺ NK cells from the same donor. The healthy blood donors expressed either the NKG2D^{HNK/HNK} (N=8), NKG2D^{HNK/LNK} (N=8) or NKG2D^{LNK/LNK} (N=8) genotype. The percentage of activated and cytotoxic (CD107a⁺) NKG2D⁺CD56⁺ NK cells was assessed by flow cytometry. Results are shown as fold change of the GlialCAM₃₇₀₋₃₈₉-specific to non-specific cells. The dashed black line indicated an equal activation of NK cells against GlialCAM₃₇₀₋₃₈₉-specific (GlialCAM₃₇₀₋₃₈₉) and non-GlialCAM₃₇₀₋₃₈₉-specific (NC) cells. The groups were compared by the Wilcoxon matched T-test. (J) Distribution of NKG2D^{HNK/LNK} variants in N=270 MS patients, N=162 EBNA^{low}, and N=108 EBNA^{high} controls. Fractions represent the relative frequency of NKG2D^{LNK/LNK}, NKG2D^{HNK/LNK} and NKG2D^{HNK/HNK} variants. The distribution of the variants was compared between the cohorts by the Chi² Test. The individual panels in the bars relate from right to left to: NKG2D^{LNK/LNK}, NKG2D^{HNK/LNK} and NKG2D^{HNK/HNK}. Del: deletion, HCMV: Human Cytomegalovirus, HNK: high NK cytotoxicity, LNK: low NK cytotoxicity, MS: Multiple Sclerosis, NC: negative control, NPC: non-peptide control, ns: not significant, wt: wild type.

FIG. 21: GlialCAM₃₇₀₋₃₈₉-specific immune cells from MS patients upregulate HLA-E via the EBV-mediated secretion of IL-27. (A-C) HLA-E expression of GlialCAM₃₇₀₋₃₈₉-specific (A) CD4⁺ T-cells, (B) CD8⁺ T-cells, and (C) CD138⁺ plasma cells, derived from N=20 MS patients, N=48 EBNA^{low}, and N=32 EBNA^{high} controls. The HLA-E expression was assessed after PBMC stimulation with the GlialCAM₃₇₀₋₃₈₉ peptide via flow cytometry. (D-F) NKG2A inhibition assay. Sorted GlialCAM₃₇₀₋₃₈₉-specific (D) CD4⁺ T-cells, (E) CD8⁺ T-cells and (F) CD138⁺ plasma cells, derived from N=20 MS patients, N=48 EBNA^{OW} and N=32 EBNA^{high} controls were co-cultured with sorted NKG2A⁺NKG2C⁻NKG2D⁻ NK cells from 24 healthy blood donors. The NKG2A⁺ NKG2C⁻NKG2D⁻ NK cell activation level was assessed by IFNγ ELISA. Data are shown as the mean from all 24 independent experiments, reflecting 24 healthy blood donors. (G-I) EBV reactivation in GlialCAM₃₇₀₋₃₈₉-specific immune cells. PBMCs from N=20 MS patients, N=48 EBNA^{OW}, and N=32 EBNA^{high} controls were stimulated with the GlialCAM₃₇₀₋₃₈₉ peptide. (G) The EBV reactivation was then assessed by EBV qPCR of the supernatant. The dashed black line indicates the detection limit of the EBV qPCR. (H-I) The number of EBV-infected (EBV EBER⁺) (H) total or (I) GlialCAM₃₇₀₋₃₈₉-specific B-cells was afterward assessed by flow cytometry. Data are shown as EBER⁺ per 1×10⁷ B-cells. (J) IL-27 secretion of EBV-infected GlialCAM₃₇₀₋₃₈₉-specific cells. PBMCs from N=20 MS patients, N=48 EBNA^{OW} and N=32 EBNA^{high} controls were stimulated with the GlialCAM₃₇₀₋₃₈₉ peptide and the IL-27 concentration in the supernatant was assessed by IL-27 ELISA. (K-L) Supernatant transfer experiments: PBMCs from N=20 MS patients, N=48 EBNA^{low}, and N=32 EBNA^{high} controls were cultured in the presence or absence of the GlialCAM₃₇₀₋₃₈₉ peptide. The supernatant was transferred to fresh PBMCs from the same donors in the (K) absence or (L) presence of a monoclonal IL27-blocking antibody. The HLA-E MFI was assessed from PBNCs by flow cytometry. The dashed black line indicates an equal HLA-E expression before and after the transfer of the supernatant. (M) PBMCs from N=20 MS patients, N=48 EBNA^{OW}, and N=32 EBNA^{high} controls were stimulated with the GlialCAM₃₇₀₋₃₈₉ peptide and the percentage of IL-27 expressing cells was assessed in EBV-infected (EBER⁺) B-cells, monocytes, NK cells, dendritic cells and non-EBV-infected (EBER⁻) B-cells by intracellular cytokine staining. (A-M). The groups were compared by the Kruskal-Wallis and the Dunn's multiple comparison test. Each dot represents one individual patient. EBER: Epstein-Barr virus (EBV)-encoded small RNAs, MFI: mean fluorescence intensity, MS: Multiple Sclerosis, ns: not significant.

FIG. 22: GlialCAM₃₇₀₋₃₈₉-specific immune cells from MS patients upregulate HLA-E via the EBV-mediated secretion of IL-27. (A-F) HLA-E upregulation: PBMCs from N=20 MS patients, N=48 EBNA^{low}, and N=32 EBNA^{high} controls were stimulated with the GlialCAM₃₇₀₋₃₈₉-peptide (GlialCAM₃₇₀₋₃₈₉) or a random 20 amino acid peptide pool (negative control, NC) or without any peptides (non-peptide control, NPC). The (A,C,E) percentage of HLA-E expressing cells or (B,D,F) HLA-E MFI of GlialCAM₃₇₀₋₃₈₉-specific (A-B) CD4⁺ T-cells, (C-D) CD8⁺ T-cells, and (E-F) CD138+ plasma cells (GlialCAM₃₇₀₋₃₈₉) or total (A-B) CD4⁺ T-cells, (C-D) CD8⁺ T-cells, and (E-F) CD138⁺ plasma cells (NC and NPC) was then assessed by flow-cytometry. (A-F) The groups were compared by the Kruskal-Wallis and the Dunn's multiple comparison test. Each dot represents one individual patient. (G-L) NKG2A inhibition: PBMCs from N=20 MS patients, N=48 EBNA^{low}, and N=32 EBNA^{high} controls were stimulated with the GlialCAM₃₇₀₋₃₈₉-peptide (GlialCAM₃₇₀₋₃₈₉) or a random 20 amino acid peptide pool (negative control, NC) or without any peptides (non-peptide control, NPC). Sorted GlialCAM₃₇₀₋₃₈₉-specific (GlialCAM₃₇₀₋₃₈₉) or total (NC and NPC) CD4⁺ T-cells, CD8⁺ T-cells, CD138⁺ plasma cells were co-cultured with NKG2A⁺ NK cells from 24 healthy blood donors in the presence or absence of monoclonal NKG2A-specific blocking antibodies (Monalizumab). The IFNγ concentration was then assessed by IFNγ ELISA in response to (G-H) CD4⁺ T-cells, (I-J) CD8⁺ T-cells, or (K-L) CD138+ plasma cells. (G-L) The groups were compared by the Kruskal-Wallis and the Dunn's multiple comparison test. Each dot represents one individual patient. (M-N) EBV reactivation: PBMCs from N=20 MS patients, N=48 EBNA^{OW}, and N=32 EBNA^{high} controls were stimulated with the GlialCAM₃₇₀₋₃₈₉-peptide (GlialCAM₃₇₀₋₃₈₉) or a random 20 amino acid peptide pool (negative control, NC) or without any peptides (non-peptide control, NPC). (M) The EBV viral load was assessed by EBV qPCR of the supernatant. The dashed black line indicates the detection limit of the EBV qPCR. (N) The frequency of EBV-infected (EBV EBER⁺) B-cells was assessed by flow cytometry. Data are shown as EBER⁺ B-cells per 1×10⁷ total B-cells. The groups were compared by the Kruskal-Wallis and the Dunn's multiple comparison test. (M-N) Each dot represents one individual patient. (O) IL-27: PBMCs from N=20 MS patients, N=48 EBNA^{OW}, and N=32 EBNA^{high} controls were stimulated with the GlialCAM₃₇₀₋₃₈₉-peptide (GlialCAM₃₇₀₋₃₈₉) or a random 20 amino acid peptide pool (negative control, NC) or without any peptides (non-peptide control, NPC). The IL-27 concentration in the supernatant was assessed by IL-27 ELISA. The groups were compared by the Kruskal-Wallis and the Dunn's multiple comparison test. Each dot represents one individual patient. EBER: Epstein-Barr virus (EBV)-encoded small RNAs, NC: negative control, NPC: non-peptide control, ns: not significant.

FIG. 23: EBV-specific immune responses in MS patients. (A) Distribution of LMP-1 peptide variants in EBV-isolates of N=270 MS patients, N=162 EBNA^{low}, and N=108 EBNA^{high} controls. Fractions represent the relative frequency of the LMP-1 peptide GGDPHLPTL, GSDPHLPTL, GGDPHLPPL, GGDPPLPTL, GCDPHLPTL, GIDPHLPTL, GAGPHLPTL, GGDTPLPTL, GDDPHLPTL, GGDPHVPTL, and GTDPHLPTL variants. The frequency of the LMP-1 variant was compared between the cohorts by the Chi² Test. The individual panels in the bars relate from left to right to: GTDPHLPTL, GGDPHLPTL, GGDPPLPTL, GIDPHLPTL, GCDPHLPTL, GDDPHLPTL, GAGPHLPTL, GGDPHVPTL, GGDTPLPTL, GSDPHLPTL and GGDPHLPPL. (B-C) HLA-E stabilization and NKG2A inhibition assays. GlialCAM₃₇₀₋₃₈₉-specific B-cells, derived from 24 healthy individuals, were individually transfected with the indicated concentrations of the positive control (VMAPRTLIL) or the LMP-1 derived GGDPHLPTL, GSDPHLPTL, GGDPHLPPL, GGDPPLPTL, GCDPHLPTL, GIDPHLPTL, GAGPHLPTL, GGDTPLPTL, GDDPHLPTL, GGDPHVPTL, and GTDPHLPTL variants. (B) The cell surface expression of HLA-E was then analyzed by flow cytometry. (C) The transfected cells were then co-cultured with sorted NKG2A⁺ NKG2C⁻NKG2D⁻ NK cells from the same donor. The inhibition of the NKG2A⁺ NKG2C⁻NKG2D⁻ NK cells was assessed by IFNγ ELISA. (B-C) Plots represent the mean (±SEM) of 24 independent experiments. Each peptide was compared to the positive control using the Friedman-Test. (D-H) Killing assays: (D-E) PBMCs from 60 healthy control individuals were first stimulated with the GlialCAM₃₇₀₋₃₈₉-peptide (GlialCAM₃₇₀₋₃₈₉) or a random 20 amino acid peptide pool (negative control, NC). Sorted GlialCAM₃₇₀₋₃₈₉-specific B-cells or non-GlialCAM₃₇₀₋₃₈₉-specific B-cells or RAJI cells were infected with the B95-8 derived EBV strain or left uninfected. (D) ADCP or (E) ADCC responses were assessed using monocyte-derived M1 macrophages and plasma from 60 healthy blood donors. (D) GlialCAM₃₇₀₋₃₈₉-specific ADCP was assessed as AF647 MFI by flow cytometry. (E) GlialCAM₃₇₀₋₃₈₉-specific ADCC was assessed as CD107a⁺CD14⁺ cells by flow cytometry. (F-H) EBV-specific CD8⁺ T-cell responses. PBMCs from 60 healthy control individuals were first stimulated with the GlialCAM₃₇₀₋₃₈₉-peptide (GlialCAM₃₇₀₋₃₈₉) or a random 20 amino acid peptide pool (negative control, NC). Sorted GlialCAM₃₇₀₋₃₈₉-specific B-cells or non-GlialCAM₃₇₀₋₃₈₉-specific B-cells or RAJI cells were infected with the B95-8 derived EBV strain or left uninfected. (F) B-cells or RAJI cells were then co-cultured with sorted total EBV-specific CD8⁺ T-cells from 60 healthy EBV-seropositive blood donors. The cell activation level was assessed by IFNγ ELISA. (G) The HLA-E-expression of EBV-infected or non-infected B-cells or RAJI cells was evaluated by flow-cytometry. (H) B-cells or RAJI cells were then co-cultured with sorted total EBV-specific and HLA-E restricted CD8⁺ T-cells from 60 healthy EBV-seropositive blood donors. The cell activation level was assessed by IFNγ ELISA. (D-H) The data were normalized to the corresponding non-infected control and the groups were compared by the Wilcoxon matched T-test. Each dot represents one individual sample from one healthy control. The dashed black line indicates an equal activation against infected and non-infected cells. (I) EBV-specific and HLA-E restricted CD8⁺ T-cell frequencies were assessed in N=20 MS patients, N=48 EBNA^{low} and N=32 EBNA^{high} controls by flow cytometry and compared by the Kruskal-Wallis and the Dunn's multiple comparison test. (J) Distribution of HLA-E variants in N=270 MS patients, N=162 EBNA^{low} and N=108 EBNA^{high} controls. Fractions represent the relative frequency of the HLA-E*0101/0101, HLA-E*0101/0103, and HLA-E*0103/0103. The frequency of the HLA-E variant was compared between the cohorts by the Chi² Test. (K-L) EBV-specific, HLA-E restricted CD8⁺ T-cells: EBV-infected GlialCAM₃₇₀₋₃₈₉-specific B-cells from 60 healthy controls, expressing either the HLA-E*0101/0101 (N=20), HLA-E*0101/0103 (N=20) or HLA-E*0103/0103 (N=20) genotype were cultured together with EBV-specific, HLA-E-restricted EBV-specific CD8⁺ T-cells. (J) The activation of the EBV-specific, HLA-E-restricted EBV-specific CD8⁺ T-cells were then assessed by IFNγ ELISA. The individual panels in the bars relate from left to right to: HLA-E*0101/0101, HLA-E*0101/0103 and HLA-E*0103/0103. (L) The proliferation of EBV-specific, HLA-E-restricted EBV-specific CD8⁺ T-cells were then assessed by flow cytometry. (K-L) The groups were compared by the Kruskal-Wallis and the Dunn's multiple comparison test. Each dot represents one individual patient. The dashed black line indicates an equal activation against infected and non-infected cells. ADCC: antibody dependent cellular cytotoxicity, ADCP: antibody dependent cellular phagocytosis, MFI: mean fluorescence intensity, MS: Multiple Sclerosis, ns: not significant.

FIG. 24: Forest plot for (A) individual and (B) combined risk factors in MS and healthy EBNA^{high} controls. The risk HLA-E*0101 allele (HLA-E*0101/0101 and HLA-E*0101/0103 genotypes) was assessed against the protective HLA-E*0103/0103 genotype. The risk NKG2D allele (NKG2D^{LNK/LNK} and NKG2D^{LNK/HNK} genotypes) was assessed against the protective NKG2D^{HNK/HNK} genotype. The risk EBV-encoded LMP-1 variants (GGDPHLPTL and GGDPPLPTL) were assessed against the protective GSDPHLPTL, GGDPHLPPL, GCDPHLPTL, GIDPHLPTL, GAGPHLPTL, GGDTPLPTL, GDDPHLPTL, GGDPHVPTL and GTDPHLPTL variants. The risk NKG2C phenotype (HCMV-seronegative, HCMV-seropositive and KLRC2^{del/del}, HCMV-seropositive and KLRC2^{wt/wt} or KLRC2^{wt/del} and low binding UL40 variants (Table 4) was assessed against the protective combination HCMV-seropositive and KLRC2^{wt/wt} or KLRC2^{wt/del} and high binding UL40 variants. The frequency of the combination was compared between the cohorts by the Chi² Test. CI: Confidence Interval, OR: Odds Ratio.

FIG. 25: EBV BZLF1-derived SQAPLPCVL HLA-E restricted CD8⁺ T cell responses may prevent the development of EBV⁺LDs. (A) Percentage of SQAPLPCVL-specific and HLA-E-restricted CD8⁺ T cells in non-transplanted, healthy blood donors (N=10, “healthy control”) as well as age and gender-matched EBV⁺DLBCL patients (N=10) or in transplanted SOT EBV⁺PTLD (lung transplant recipients: N=4, and kidney transplant recipients: N=6) as well as age, gender and type of transplantation-matched controls without EBV⁺PTLD (N=10, “healthy control”). EBV⁺DLBCLs and EBV⁺PTLDs were diagnosed and classified according to recently published WHO guidelines using histological and/or cytological findings^{11,14,15,114}. PBMCs from EBV⁺DLBCLs and EBV⁺PTLDs were available immediately (1-3 days) after diagnosis, respectively. Enriched CD8⁺ T cells were stimulated with SQAPLPCVL peptide-pulsed RMA/S-HLA-E*0103/0103 cells and SQAPLPCVL-specific HLA-E restricted CD8⁺ T cells were identified as IFNγ⁺ CD3⁺ CD8⁺ cells. Data are shown as frequency of SQAPLPCVL-specific HLA-E restricted CD8⁺ T cells in comparison to total CD8⁺ T cells. (B) CD8⁺ T cell phenotype of SQAPLPCVL-specific and HLA-E-restricted CD8⁺ T cells in non-transplanted, healthy blood donors (N=10), EBV⁺DLBCL patients (N=10), EBV⁺PTLD and transplantation-matched controls without EBV⁺PTLD (N=10), respectively. SQAPLPCV-specific HLA-E restricted CD8⁺ T cells were phenotyped by the expression of LAG3, PD1, TGIT, TIM3, and NKG2A by flow-cytometry. (C-D) RAJI cell proliferation assays: RAJI cells were either cultured alone or with total sorted CD8⁺ T cells from EBV-seronegative blood donors (N=5), sorted total EBV-specific CD8⁺ T cells from EBV-seropositive blood donors (N=5), or sorted SQAPLPCVL-specific HLA-E-restricted CD8⁺ T cells from EBV-seropositive blood donors (N=5) in a effector-to-target cell ratio of 1:1. Total EBV-specific as well as SQAPLPCVL-specific HLA-E restricted CD8⁺ T cells were identified as CD137⁺ CD3⁺ CD8⁺ cells after stimulation with an EBV peptide pool (PepTivator® EBV Consensus, Miltenyi Biotec) or SQAPLPCVL peptide-pulsed RMA/S-HLA-E*0103/0103 cells, respectively. After co-culture, all cells were then harvested, fixed with the FIX & PERM Cell Fixation & Cell Permeabilization Kit (Thermo-Scientific) and analysed by flow-cytometry at indicated time points. (C) RAJI cells were identified as CD19⁺ cells by flow cytometry. The individual lines in the graph relate from top to bottom to: RAJI, RAJI+total CD8+ T cells (EBV seronegative), RAJI+EBV-specific CD8+ T cells (EBV seropositive) and RAJI+SQAPLPCVL-specific HLA-E-restricted CD8+ T cells (EBV seropositive). (D) Cytotoxic CD8⁺ T were identified as Granzyme B⁺ CD3⁺CD8⁺ cells by flow cytometry. Data are shown as percentage of Granzyme B⁺ CD3⁺ CD8⁺ cells from total CD3⁺ CD8⁺ cells. The individual lines in the graph relate from top to bottom to: RAJI+total CD8+ T cells (EBV seronegative), RAJI+EBV-specific CD8+ T cells (EBV seropositive) and RAJI+SQAPLPCVL-specific HLA-E-restricted CD8+ T cells (EBV seropositive). (A,C,D) RM one-way ANOVA (with the Geisser-Greenhouse correction) or (B) Two-Way-ANOVA were used to compare the respective groups. p<0.05 was considered significant. *: p<0.05; **: p<0.01; ***: p<0.001; ****: p<0.0001. DLBCL: diffuse large B cell lymphoma, EBV: Epstein-Barr virus, GranB: Granzyme B, PTLD: post-transplant lymphoproliferative disorder.

FIG. 26: SQAPLPCVL-specific HLA-E-restricted CD8⁺ T cell immune responses contribute to the prevention of the development of EBV-associated gastric carcinoma. (A) SQAPLPCVL-specific HLA-E-restricted CD8⁺ T cells from healthy blood donors (N=100), EBV⁻GC (N=10), and EBV⁺GC (N=10) were identified as IFNγ⁺CD3⁺CD8⁺ cells in response to SQAPLPCVL-pulsed RMA/S-HLA-E cells by flow cytometry. (B) The cells were subsequently phenotyped and analyzed for the expression of LAG3, PD1, TGIT, TIM3, and NKG2A by flow cytometry. Data are shown as mean cell frequencies±SD. (C) Simple linear regression of SQAPLPCVL-specific HLA-E-restricted CD8⁺ T cell frequencies and SQAPLPCVL-specific IgG antibody titers. IgG antibody titers were evaluated with a peptide ELISA. (D) From EBV⁺GC (N=10) and EBV⁺GC (N=10) patients, 10 retrospective follow-up plasma samples were collected between 1 and 10 years before the GC diagnosis. Data are shown as simple linear regression or SQAPLPCVL-specific IgG antibody titers and the corresponding time points. ns: not significant.

### EXAMPLE 1

### HLA-E-Restricted Immune Responses are Crucial for the Control of Epstein-Barr-Virus 2 Infections and the Prevention of Post-Transplant Lymphoproliferative Disorders

### Introduction

Epstein-Barr virus (EBV) is a ubiquitous herpesvirus, which infects over 90% of the adult human population worldwide. Primary EBV infection may result in a self-limiting infectious mononucleosis (IM), which is hallmarked by fever, lymphadenopathy, and tonsillitis, often associated with splenomegaly and/or self-resolving hepatitis. In the absence of a licensed EBV vaccine, cases of IM result in a high economic burden for public health services¹. However, the cumulative risk to develop IM upon primary EBV infection is estimated only between 13.3%-22.4%², and it is so far an unresolved question, why during primary EBV infection some patients develop clinically evident IM, while others remain asymptomatic.

After primary infection, EBV establishes a life-long persistent infection in memory B cells, from which sporadic reactivations may occur. EBV is associated with the development of malignant EBV-associated diseases, resulting worldwide in >137,900 annual deaths³. In solid-organ (SOT) and hematopoietic stem cell transplant (HSCT) recipients, EBV may cause malignant post-transplant lymphoproliferative disorders (PTLD), which are associated with high morbidity and poor survival⁴. As these complications occur only in a part of the EBV seropositive patients, it was hypothesized that there are distinct, individually determined factors in the human immune response, that may control EBV replication and eliminate EBV infected and transformed cells.

The EBV-specific immune responses are hallmarked by potent cytotoxic CD8⁺ T cell and natural killer (NK) cell responses⁵⁻⁷. Among the broad EBV-specific CD8⁺ T cell responses, a small subset of CD8⁺ T cells bind with their ap T-cell receptor to the non-classical HLA molecule HLA-E⁸. HLA-E shows a strictly restricted expression pattern, including B cells⁹. HLA-E is highly conserved in European populations and only two allelic variants, the high-expressing HLA-E*0103 and the low-expressing HLA-E*0101 are prevalent⁹. The limited polymorphism results in a restricted set of distinct EBV-derived peptides, which can be presented via HLA-E on the surface of EBV-infected cells. It was shown that HLA-E is stabilized by the conserved EBV-encoded BZLF1-peptide or by highly polymorphic EBV LMP-1-derived peptides¹⁰. HLA-E further binds to the inhibitory NKG2A/CD94 receptor complex, which is expressed on distinct CD8⁺ T and NK cell subsets. By their peptides presented via HLA-E, EBV infections elicit the expansion of NKG2A⁺ NK cells; a NK cell subset, which respond to EBV-infected cells by the secretion of pro-inflammatory cytokines and cellular cytotoxicity^{6,11}.

In the present study, we hypothesized that EBV-specific, HLA-E-restricted CD8⁺ T cell and HLA-E mediated NKG2A⁺ cell responses play an important role in the control of EBV infections and in the prevention of EBV-associated diseases. By combining genetic association approaches in a large patient cohort with functional in vitro NK and CD8⁺ T cell activation and EBV-dissemination assays, we could demonstrate that HLA-E-restricted immune responses have a substantial impact in mediating protection from IM and EBV-associated PTLD.

### Results

The Low-Expressing HLA-E*0101/0101 Variant is Overrepresented in Individuals with IM

To analyze the impact of HLA-E-restricted immune responses on the prevention of clinically evident IM, we first compared the prevalence of the HLA-E variants between patients with IM and EBV seropositive persons who had, in spite of a past EBV infection, no history of IM. We recruited 578 patients with acute IM, 412 adolescent or adult (15-35 years, adolescent/adult IM) and 166 pediatric (1-14 years, pediatric IM) subjects. IM was diagnosed clinically by the triad of fever, lymphadenopathy and tonsillitis and was confirmed serologically by the detection of EBV-VCA-specific IgM, in the absence of EBV EBNA-specific IgG antibodies. In all IM patients, EBV-DNA was detected in plasma by PCR (Table 1). Furthermore, we recruited 206 EBV-IgG positive, VCA-IgM negative healthy persons, who had a past, but clinically inapparent EBV infection (asymptomatic EBV-seropositive). These individuals were matched to the adolescent/adult IM cohort in regard of age and gender (Table 1). In addition, we included a control cohort of 412 persons with unknown EBV serostatus, also matched to the adolescent/adult IM cohort, to reflect the overall distribution of HLA-E*0101/0103 variants in the population (control).

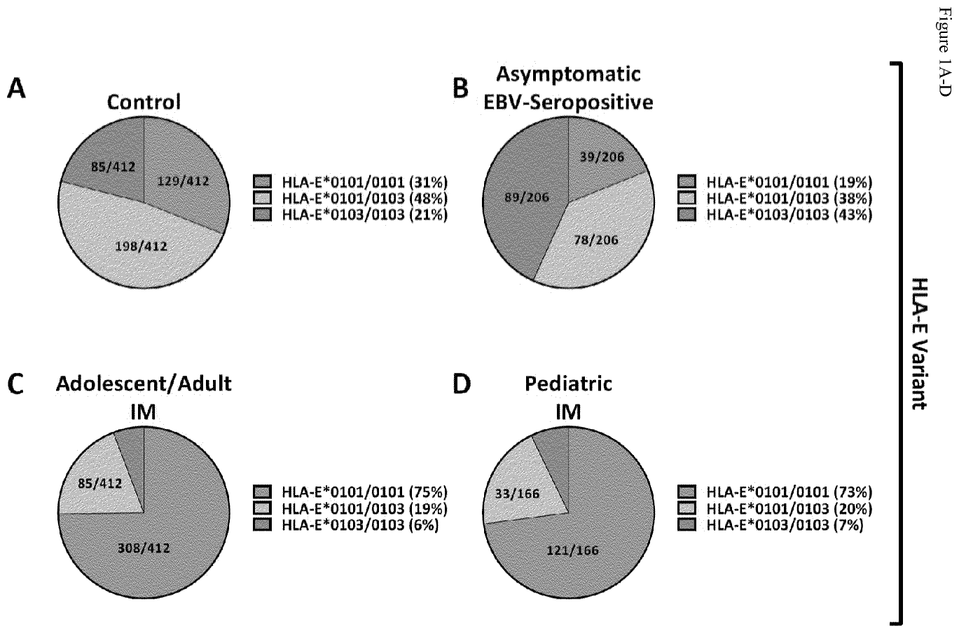

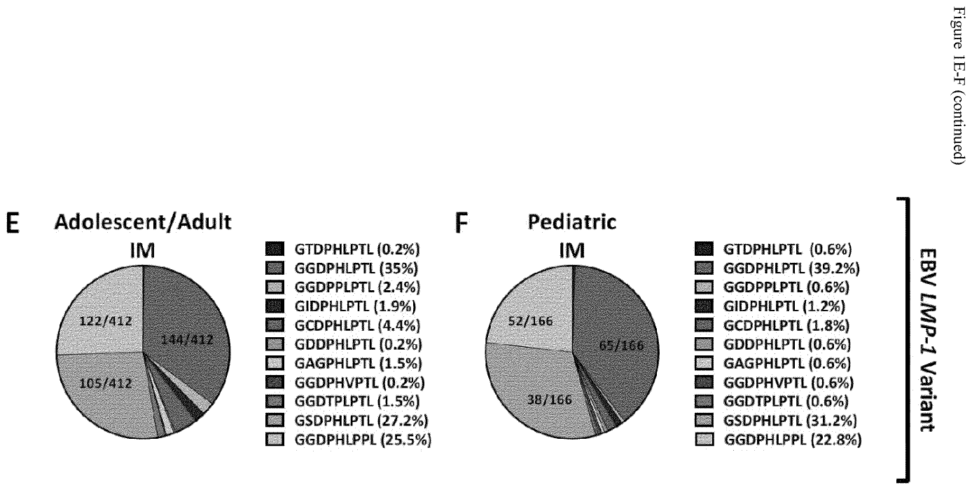

All groups were tested for their HLA-E*0101/0103 variants. As shown in FIG. 1A and FIG. 1B, the asymptomatic EBV-seropositive persons exhibited a significantly higher frequency of the HLA-E*0103/0103 genotype and a lower frequency of the HLA-E*0101 allele compared to the control (p<0.0001, OR:2.9). In contrast, the IM patients showed a significantly higher frequency of the HLA-E*0101/0101 genotype than the overall population (adolescent/adult IM: p<0.0001, OR:6.4; pediatric IM:p<0.0001, OR:5.9; FIGS. 1C,D), while the HLA-E*0103 allele occurred rarely in both IM groups. Thus, the high-expressing HLA-E*0103/0103 variant seems to be a protective factor against symptomatic IM.

### IM is Hallmarked by a High Diversity of the EBV LMP-1 Peptides

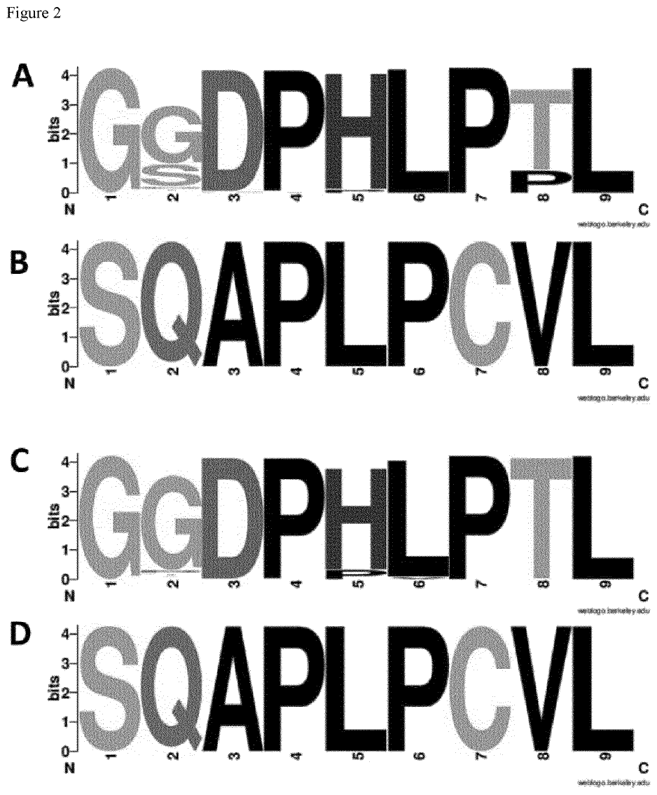

In EBV infections, the HLA-E upregulation is dependent on two EBV peptides; a highly polymorphic LMP-1 and a more conserved BZLF1-derived peptide. We then analyzed the LMP-1 and BZLF1-derived peptide sequences of the EBV strains infecting the individual IM patients by Sanger-Sequencing. In overall, eleven different LMP-1 peptide variants were identified (FIG. 2A). Among these, the GGDPHLPTL (N=242,41.9%), GSDPHLPTL (N=147,25.4%) and GGDPHLPPL (N=127,22%) variants were the most frequent ones, while the eight remaining variants only rarely occurred (FIG. 1E, F). The distribution of LMP-1 peptide variants was similar in adolescent/adult and pediatric IM patients (p=ns). In contrast, the BZLF1-derived SQAPLPCVL peptide was highly conserved in all EBV strains (FIG. 2B).

### EBV-Specific, HLA-E-Restricted CD8⁺ T Cell Responses are Highly Dependent on the HLA-E Variants

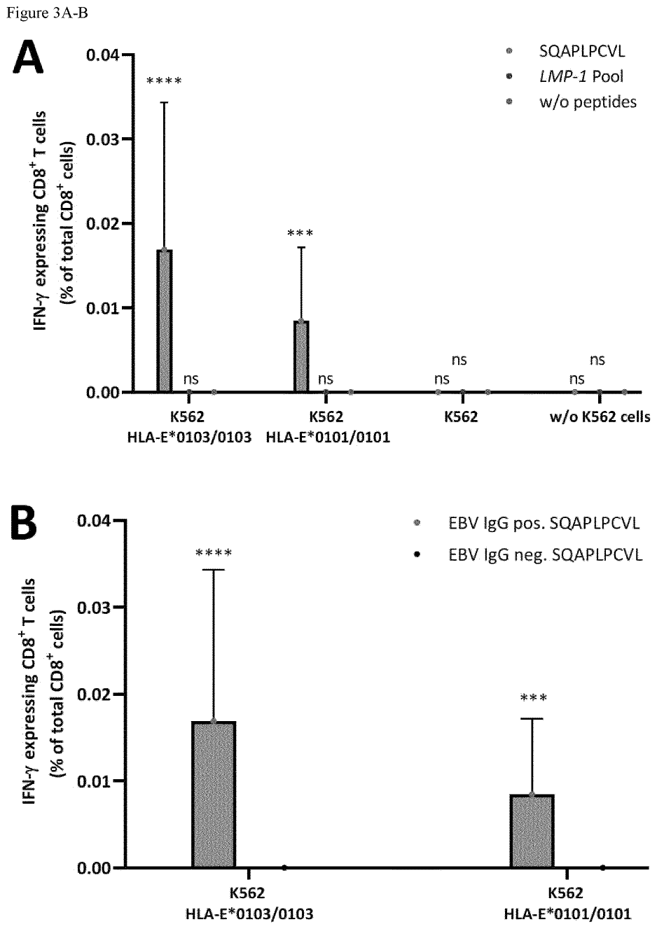

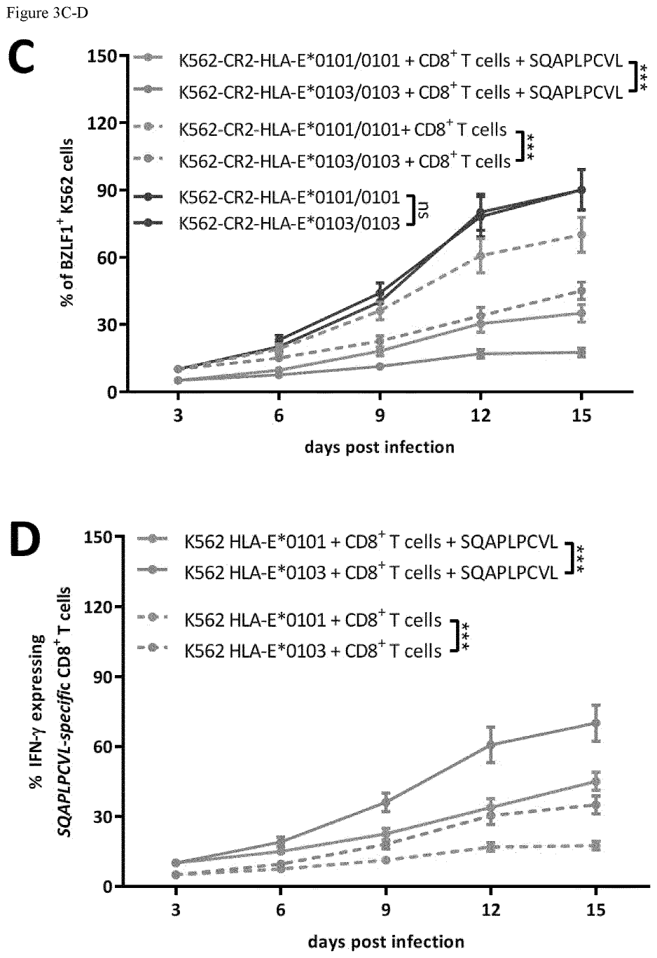

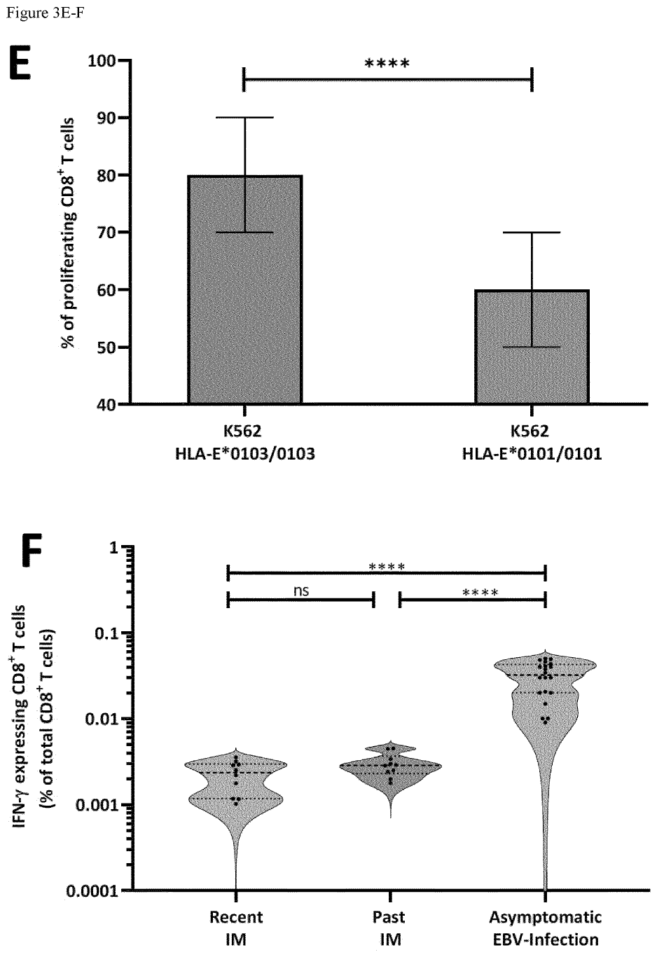

We then analysed the capacity of the BZLF1-derived SQAPLPCVL and of all LMP-1 peptide variants to elicit EBV-specific, HLA-E-restricted CD8⁺ T cell responses. We first isolated CD8⁺ T cells from 12 EBV-seropositive and 6 EBV-seronegative healthy individuals and co-cultured these cells with K562-HLA-E*0103/0103 or K562-HLA-E*0101/0101 cells, pulsed with SQAPLPCVL or with a peptide pool including all 11 LMP-1-peptide variants, respectively. As shown in FIG. 3A and FIG. 3B, an EBV-specific, HLA-E-restricted CD8⁺ T cell response was detected in EBV-seropositive persons, but only against SQAPLPCVL-pulsed K562-HLA-E-expressing cells, but neither in response to the LMP-1 pool, nor in the absence of HLA-E-expressing K562 cells, nor in EBV-seronegative individuals.

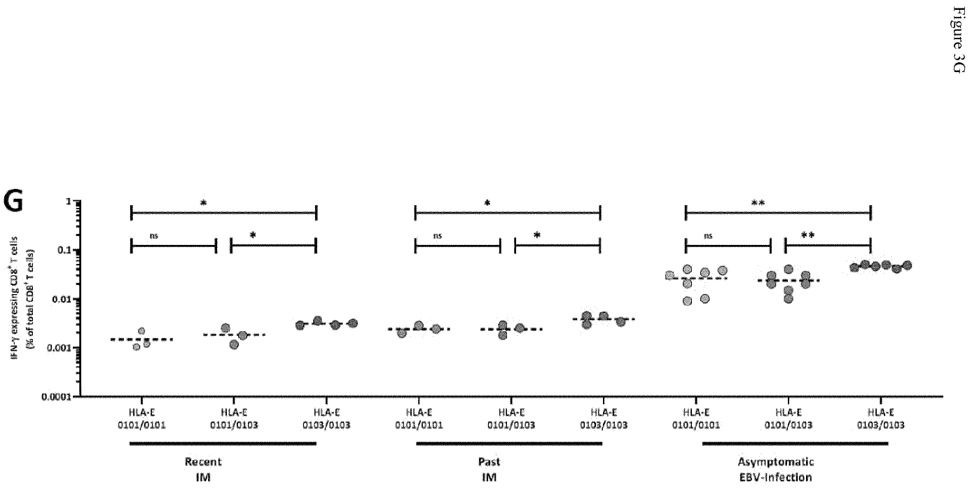

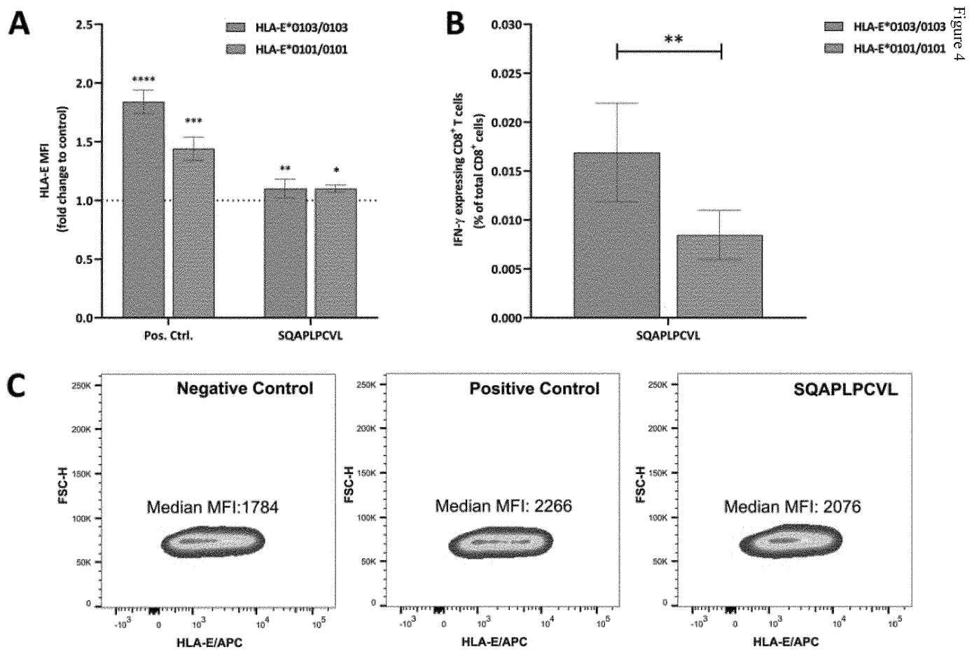

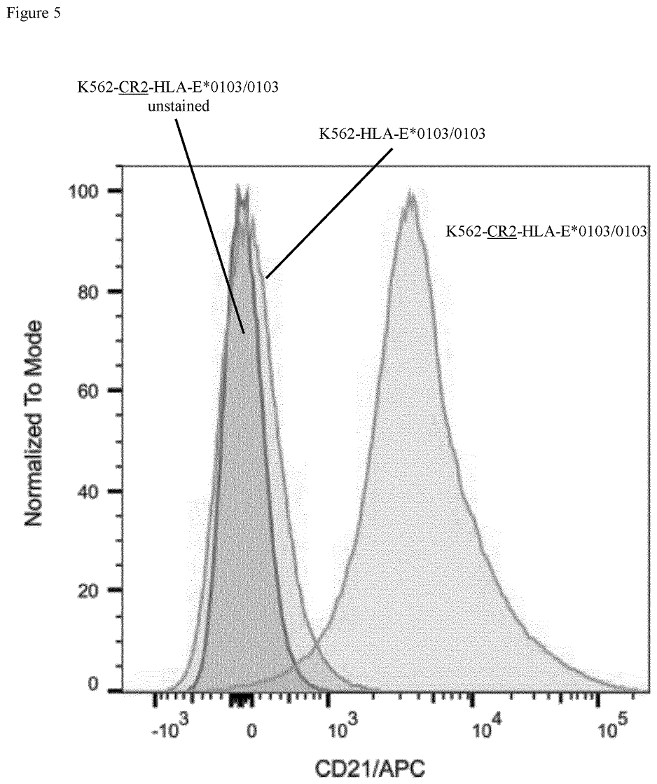

We further compared the SQAPLPCVL-specific, HLA-E-restricted CD8⁺ T cell response between K562-HLA-E*0103/0103- and K562-HLA-E*0101/0101-expressing cells. The SQAPLPCVL peptide led to a stable upregulation of HLA-E and to HLA-E-restricted CD8⁺ T cell responses, this effect was, however, significantly stronger on HLA-E*0103/0103-, compared to HLA-E*0101/0101-expressing K562 cells (FIG. 4).

We then analyzed to which extent the SQAPLPCVL-specific; HLA-E-restricted CD8⁺ T cell response is able to prevent the EBV-spread in vitro. As K562-HLA-E cells cannot be infected by EBV¹², we established K562-CR2-HLA-E*0103/0103 and K562-CR2-HLA-E*0101/0101 cell lines by CRISPR gene editing (Fig. S5).

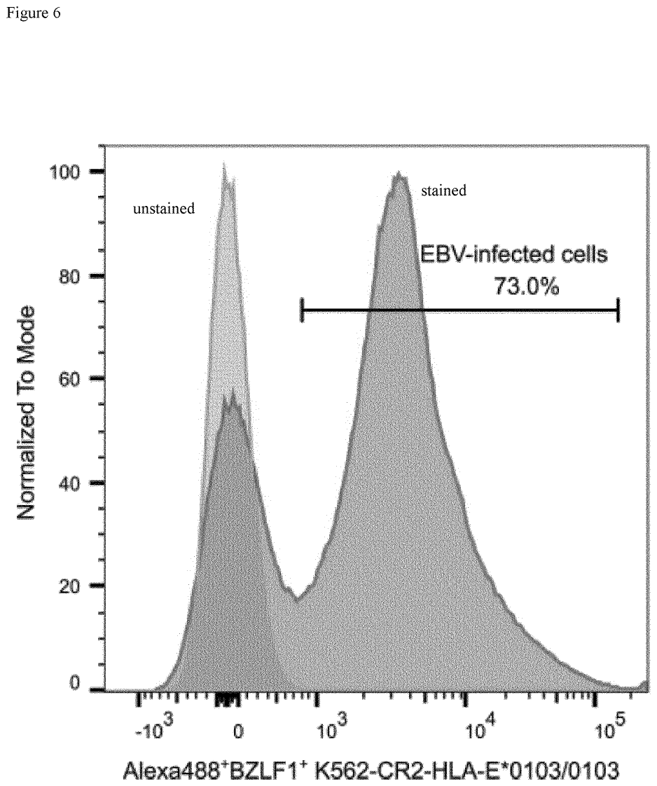

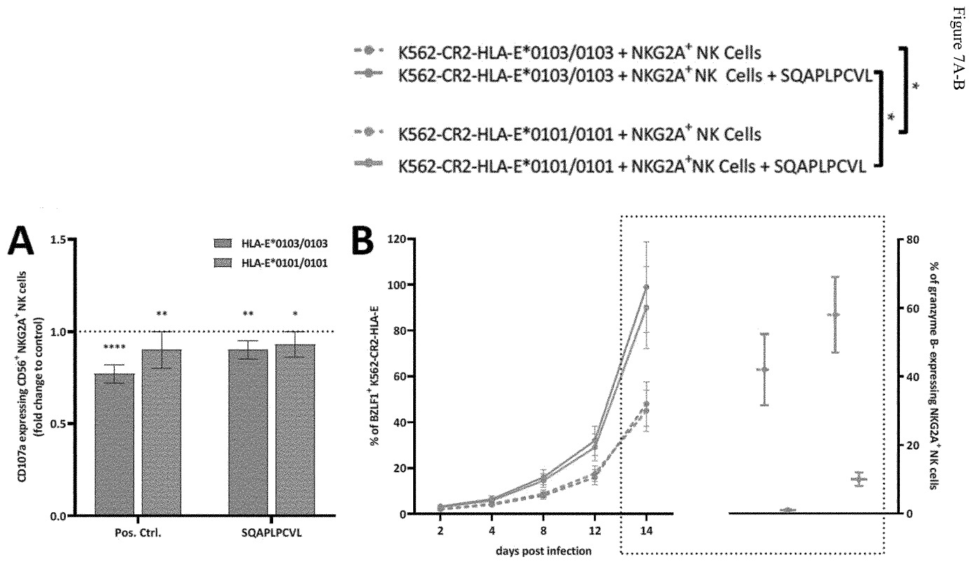

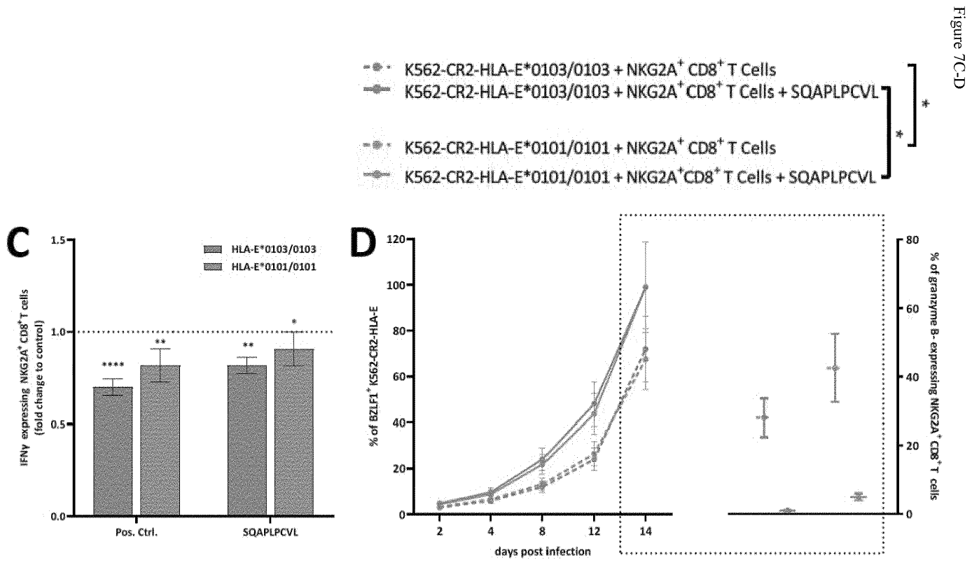

We infected both K562-CR2-HLA-E cell lines with the EBV B95-8 isolate and cultured the infected cells together with sorted SQAPLPCVL-specific, HLA-E-restricted CD8⁺ T cells of the healthy 12 EBV-seropositive donors, in the presence or absence of supplementary SQAPLPCVL peptides. The K562-CR2-HLA-E cells were then analysed for the expression of EBV-BZLF1 (FIG. 6) and the CD8⁺ T cells were tested for the activation marker IFNγ by flow-cytometry. As shown in FIG. 3C, SQAPLPCVL-specific, HLA-E-restricted CD8⁺ T cells efficiently inhibited the viral spread, as demonstrated by a reduction of BZLF1⁺K562-CR2-HLA-E cells in the presence of SQAPLPCVL-specific, HLA-E-restricted CD8⁺ T cells.

We then compared the viral spread and the CD8⁺ T cell activation levels between cells, expressing either the HLA-E*0103/0103 or the HLA-E*0101/0101 variant. SQAPLPCVL-specific, HLA-E-restricted CD8⁺ T cells prevented the viral spread to a significantly higher extent in K562-CR2-HLA-E*0103/0103, compared to K562-CR2-HLA-E*0101/0101 cells (FIG. 3C). Vice versa, K562-CR2-HLA-E*0103/0103 cells elicited a significantly higher level of CD8⁺ T cell activation, as reflected by more IFNγ-expressing cells, compared to K562-CR2-HLA-E*0101/0101 cells (FIG. 3D).

To analyse, whether the HLA-E variants also have an impact on the expansion of SQAPLPCVL-specific, HLA-E-restricted CD8⁺ T cells, we cultured K562-HLA-E*0103/0103 or K562-HLA-E*0101/0101 cells together with sorted CFSE-stained SQAPLPCVL-specific, HLA-E-restricted CD8⁺ T cells and subsequently analysed the proliferating CD8⁺ T cell subsets by flow-cytometry. As shown in FIG. 3E, stimulation with K562-HLA-E*0103/0103 cells led to a significantly higher proliferation of SQAPLPCVL-specific, HLA-E-restricted CD8⁺ T cells, compared to stimulation with K562-HLA-E*0101/0101 cells.

Overall, the data demonstrate that the HLA-E*0103/0103 variant is better stabilized by the BZLF1-derived SQAPLPCVL peptide, which further leads to a robust proliferation and activation of EBV-specific, HLA-E-restricted CD8⁺ T cells.

In conclusion, we provide evidence that a SQAPLPCVL-peptide vaccine elicit potent SQAPLPCVL-specific HLA-E-restricted CD8⁺ T cells, which can prevent the EBV-spread during primary infection, EBV-reinfections as well as EBV-reactivation and consequently reduce the risk for EBV-associated diseases in general and EBV-associated lymphoproliferative diseases in particular.

### High Levels of EBV-Specific, HLA-E-Restricted T Cells are Present in Asymptomatic EBV-Seropositive Persons

Based on these results, we hypothesized that SQAPLPCVL-specific, HLA-E-restricted CD8⁺ T cell responses are a key factor providing protection against symptomatic primary EBV infections and may differ between individuals with and without IM. We therefore analysed and compared the percentage of SQAPLPCVL-specific, HLA-E-restricted CD8⁺ T cells between the cohorts.

We first gained blood samples from 20 randomly selected patients in whom the primary EBV infection has occurred asymptomatically somewhere in the past. To enable a comparison between this patient group and patients with IM, we also gained blood samples from 20 randomly selected patients with past IM. From 10 IM patients, samples were obtained 1-8 years (recent IM), and from 10 patients 10-23 years (past IM) after the diagnosis of IM. We then compared the SQAPLPCVL-specific, HLA-E-restricted CD8⁺ T cell levels of those three groups. As shown in FIG. 3F, patients with asymptomatic EBV infections showed significantly higher SQAPLPCVL-specific, HLA-E-restricted CD8⁺ T cell levels, compared to patients with earlier IM. As shown in FIG. 3G, patients with IM and patients with asymptomatic EBV infection, encoding for the HLA-E*0103/0103 genotype had always significantly higher SQAPLPCVL-specific, HLA-E-restricted CD8⁺ T cell levels, compared to respective individuals of the same group, encoding for the HLA-E*0101 allele (FIG. 3G).

We provide evidence, that SQAPLPCVL-specific, HLA-E-restricted CD8⁺ T cell responses are low in patients with symptomatic EBV-infections. We thus show that high vaccine-induced SQAPLPCVL-specific, HLA-E-restricted CD8⁺ T cell responses are protective against symptomatic EBV-infections.

NKG2A⁺ SQAPLPCVL-Specific, HLA-E-Restricted CD8⁺ T Cells can Prevent EBV-Spread

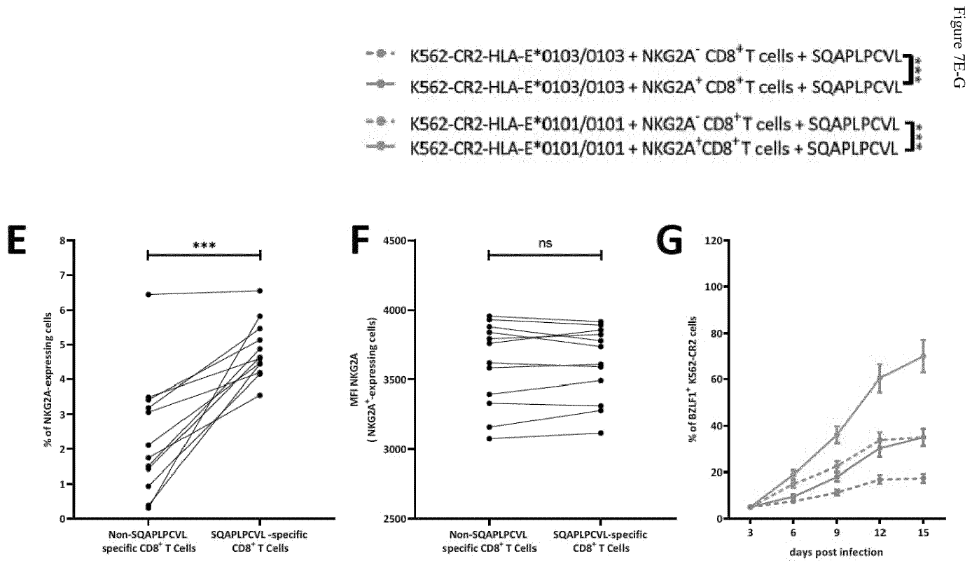

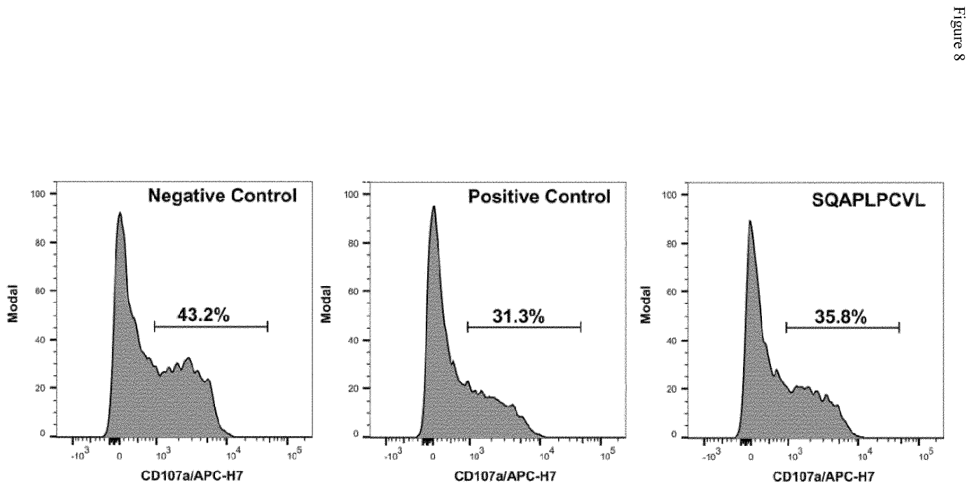

Since the SQAPLPCVL peptide has an important role in the stabilization of HLA-E, which in turn leads to stimulation of the inhibitory NKG2A⁺ NK and NKG2A⁺CD8⁺ T cells, we next analyzed the capacity of SQAPLPCVL to inhibit NKG2A⁺ NK and NKG2A⁺CD8⁺ T cells. Therefore, we isolated NK and CD8⁺ T cells from 12 healthy EBV-seropositive donors and co-cultured the pre-activated cells together with SQAPLPCVL-pulsed K562-HLA-E*0101/0101 or K562-HLA-E*0103/0103 cells. As shown in FIGS. 7A, B and FIG. 8, SQAPLPCVL-pulsed K562-HLA-E*0103/0103, and to a lower extent also SQAPLPCVL-pulsed K562-HLA-E*0101/0101 cells led to a significant inhibition of NKG2A⁺ NK cells, as reflected by a decrease of CD107a-expressing NKG2A⁺ NK cells in comparison to non-peptide pulsed cells. Similarly, we observed a significant inhibition of NKG2A⁺CD8⁺ T cells, that result in fewer IFNγ-expressing NKG2A⁺CD8⁺ T cells (FIG. 7C, D).

As our data demonstrate that the SQAPLPCVL peptide can activate HLA-E-restricted CD8⁺ T cells, but also inhibits the subpopulation of NKG2A⁺CD8⁺ T cells, we next analysed to which extent SQAPLPCVL-specific, HLA-E-restricted CD8⁺ T cells express NKG2A. Therefore, we analysed sorted SQAPLPCVL-specific and all non-SQAPLPCVL-specific, HLA-E-restricted CD8⁺ T cell subsets of the 12 seropositive persons for their NKG2A expression. As shown in FIG. 7E, only a small fraction of both, non-SQAPLPCVL-specific (>6.3%) and SQAPLPCVL-specific (>6.5%), HLA-E-restricted CD8⁺ T cells expressed NKG2A. A comparable level of NKG2A expression was observed between both CD8⁺ T cell subsets (FIG. 7F).

We then evaluated whether SQAPLPCVL-specific, HLA-E-restricted NKG2A⁺CD8⁺ T cells can prevent EBV spread in vitro. We subjected the sorted NKG2A⁺ and NKG2A⁻SQAPLPCVL-specific, HLA-E-restricted CD8⁺ T cells from 12 EBV-seropositive donors to EBV-dissemination assays. As shown in FIG. 7G, SQAPLPCVL-specific, HLA-E-restricted NKG2A⁺ CD8⁺ T cells were able to prevent viral spread in HLA-E*0101/0101- and HLA-E*0103/0103-expressing K562-CR2 cells, but to a significantly lesser extent than HLA-E-restricted NKG2A-CD8⁺ T cells, which had no inhibition via the HLA-E/NKG2A axis.

In summary, the data provide evidence that a minority of SQAPLPCVL-specific HLA-E-restricted CD8⁺ T cells express NKG2A and that these SQAPLPCVL-specific HLA-E-restricted NKG2A⁺CD8⁺ T cells can overcome to some extent the NKG2A mediated inhibition and prevent EBV viral dissemination.

HLA-E and LMP-1 Peptide Variants are Associated with Symptomatic EBV Reactivations in Elderly Persons

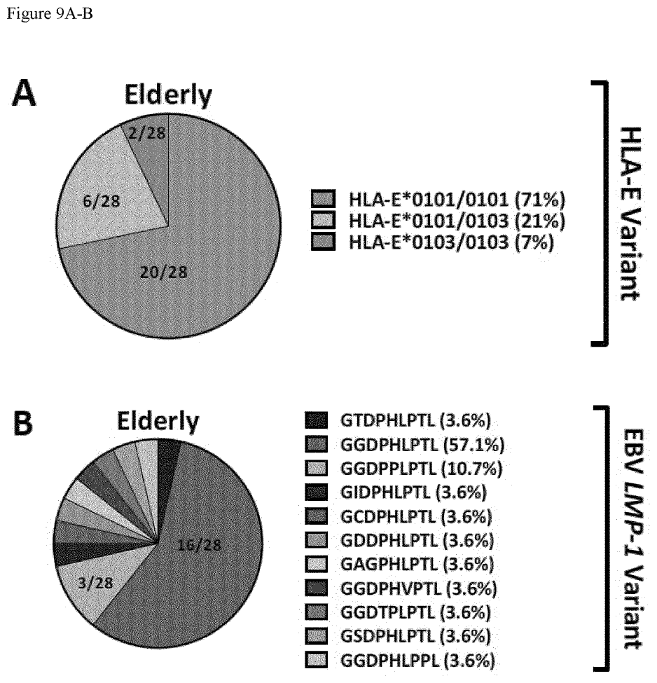

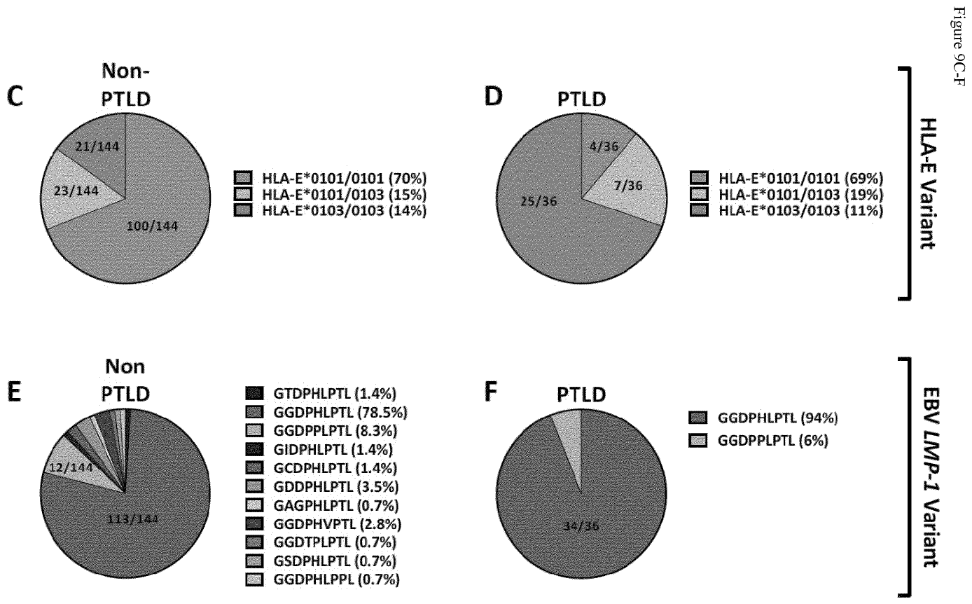

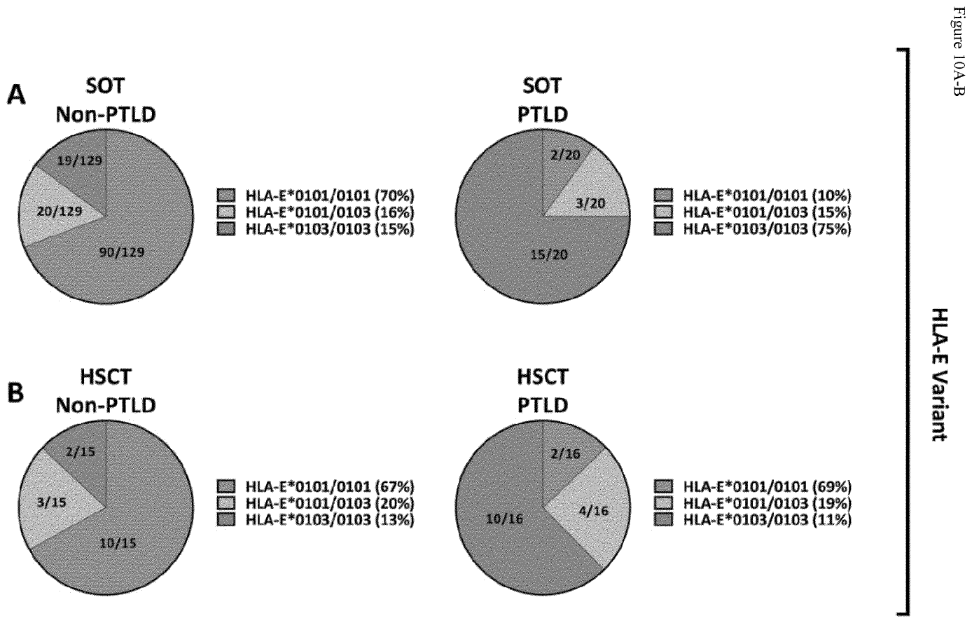

Based on the findings that HLA-E variants and HLA-E-restricted immune responses play an important role in primary EBV infections, we further analyzed the impact of host HLA-E*0101/0103 and EBV LMP-1 peptide variants in patients with symptomatic EBV reactivations. We first analyzed the HLA-E*0101/0103 variants in 28 elderly patients, >60 years old, with confirmed EBV reactivations. All patients had fever, detectable EBV-viremia (>1000 copies/mL plasma), EBV-VCA-specific IgM and EBV-VCA- and EBNA-specific IgG antibodies (Table 1). As shown in FIG. 9A, in these patients the HLA-E*0101/0101 genotype was significantly overrepresented, compared to the control cohort (FIG. 1A), while the HLA-E*0103 allele rarely occurred (p<0.0001, OR:5.5).

We then assessed the LMP-1 peptide variants of the patients' EBV strains identified during the reactivation episodes. The patients showed a significantly different LMP-1 peptide profile compared to IM patients, characterized by a higher frequency of the GGDPHLPTL (57.1%) and the GGDPPLPTL (10.7%) variant (p=0.02, OR:2.7, FIG. 9B, FIG. 2).

HLA-E Variants and LMP-1 Peptides in EBV Reactivations in Transplant Recipients with and without PTLD

EBV reactivations in transplant recipients may result in development of life threatening PTLD. We therefore assessed whether there is an association between host HLA-E and EBV LMP-1 peptide variants, EBV reactivation and PTLD in transplant recipients. We included 180 EBV-PCR positive patients after HSCT (N=31) or SOT (N=149), who were followed up for three years post-transplantation (Table 1). Patients were tested within this period for EBV-DNA, either because of unspecific symptoms, or in case of PTLD diagnosis according to international guidelines¹³⁻¹⁵ Of all transplant recipients 36 developed PTLD within 312 days, while in 144 recipients EBV viremia was detected within 302 days post-transplantation without progressing to PTLD in the follow-up (Table 1).

Similar to the elderly persons, PTLD-free transplant recipients with symptomatic EBV reactivations encoded significantly more frequently for the HLA-E*0101/0101 genotype, compared to the controls (p<0.0001, OR:5.2, FIG. 9C).

Compared to PTLD-free transplant recipients, patients with PTLD, however, carried significantly more often the HLA-E*0103/0103 genotype, while the HLA-E*0101 allele rarely occurred (p<0.0001, OR:31.25, FIG. 9D, Fig. S10). This implicates that a strong HLA-E-mediated immune response, as reflected by the high-expressing HLA-E*0103/0103 variant, was associated with the risk for PTLD.

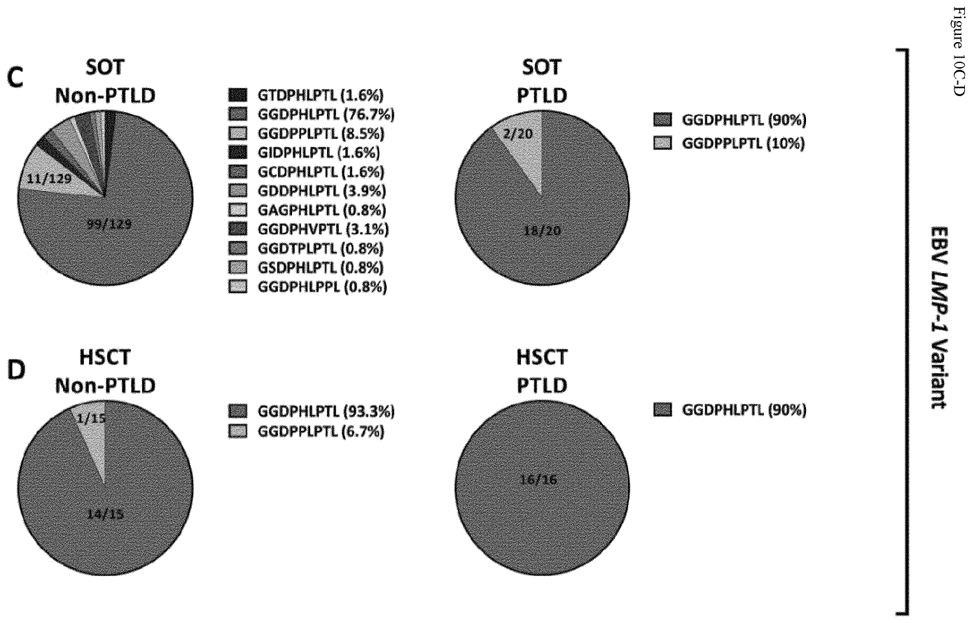

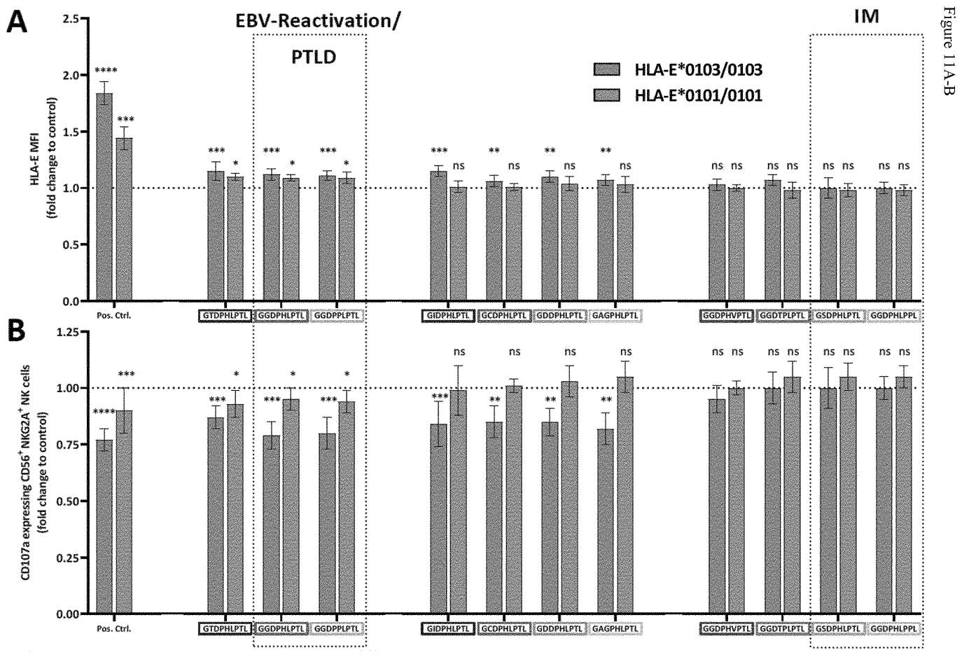

We then assessed the viral LMP-1 peptides in the EBV strains, infecting patients with and without PTLD. In comparison to IM patients (FIG. 1E, F), Non-PTLD (p<0.0001, OR:8.4, FIG. 9E) and PTLD patients (p<0.0001, OR:94.7, FIG. 9F) were infected with EBV strains, which encoded especially for the GGDPHLPTL and GGDPPLPTL variant. In patients with PTLD only the GGDPHLPTL or the GGDPPLPTL LMP-1 variants were found (PTLD vs. Non-PTLD: p=0.02, OR=11.3, FIG. 10).

Individual EBV LMP-1 Peptide Variants Elicit Altered NKG2A⁺ NK and CD8⁺ T Cell Responses

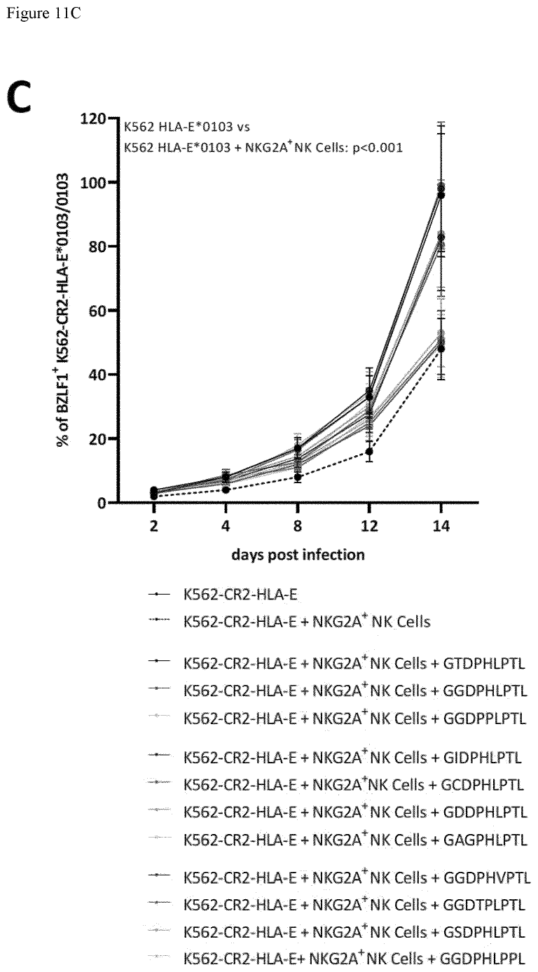

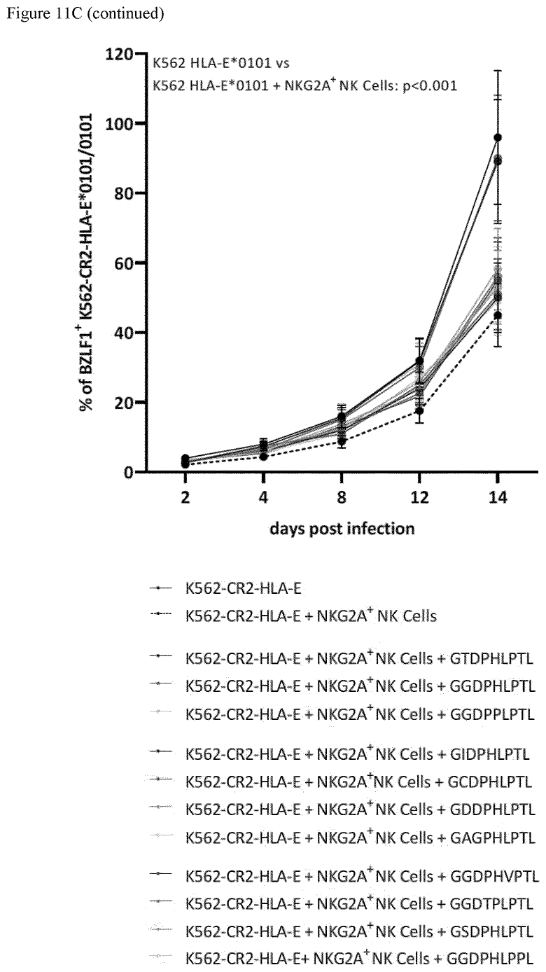

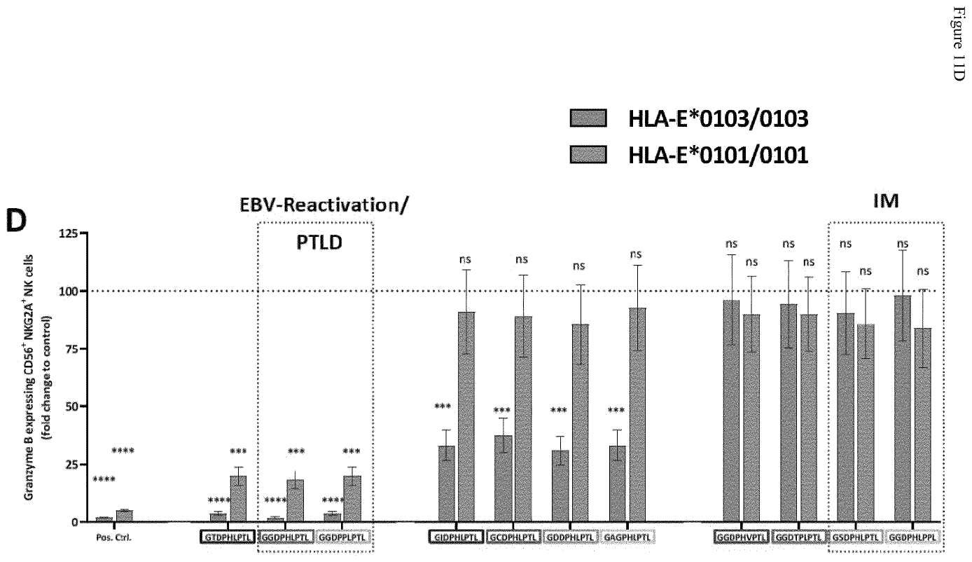

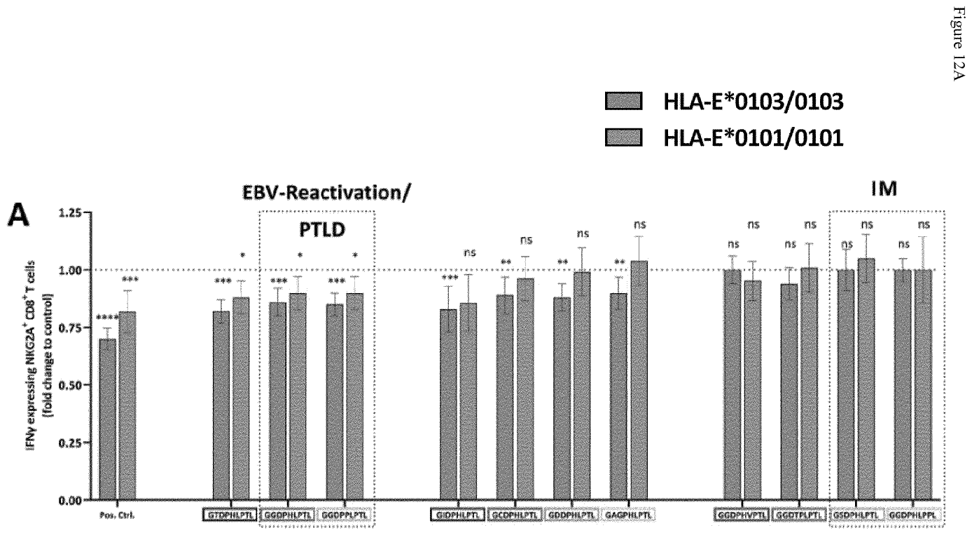

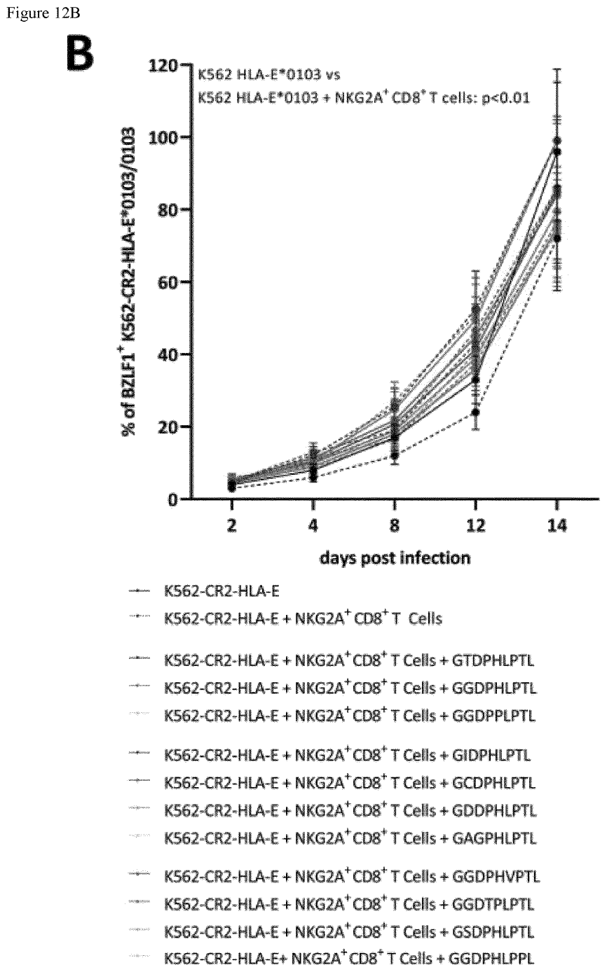

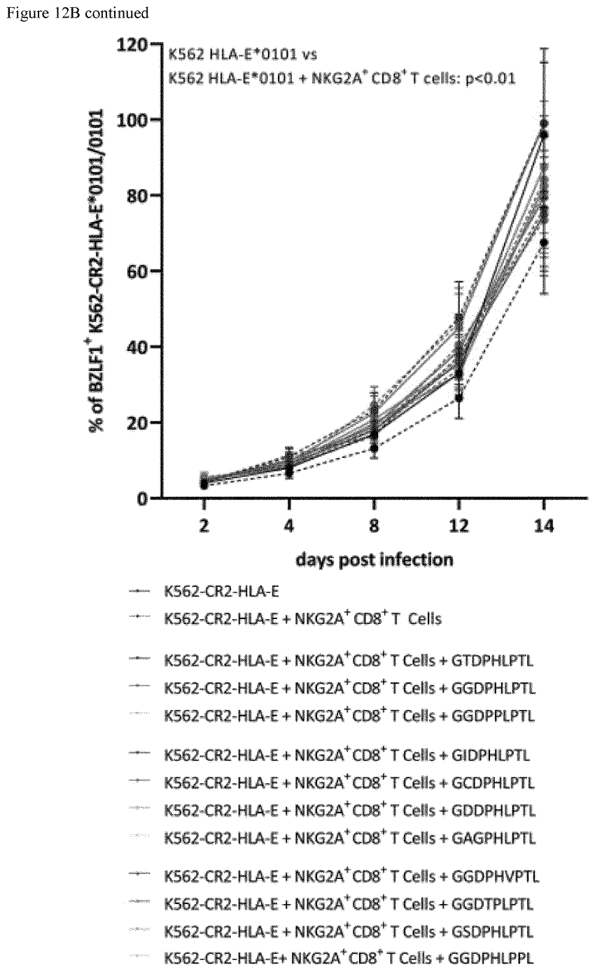

NKG2A⁺ NK cell mediated effector functions can prevent the B cell transformation of EBV-infected cells-^{6,11}, but NKG2A⁺ cells can also be inhibited by EBV-derived peptides, presented on HLA-E via the HLA-E/LMP-1/NKG2A axis. Thus, we further analyzed whether the individual LMP-1-derived peptides differ in their ability to stabilize HLA-E and to inhibit NKG2A⁺ NK as well as NKG2A⁺ CD8⁺ T cells. We therefore isolated CD56⁺ NK and CD8⁺ T cells from 12 healthy EBV-seropositive donors and co-cultured the cells together with K562-HLA-E cells, independently pulsed with each of the eleven different LMP-1 peptide variants. The LMP-1-derived peptides caused varying degrees of HLA-E stabilization (FIG. 11A), as well as of inhibition of NKG2A⁺ NK (FIG. 11B) and of NKG2A⁺ CD8⁺ T cells (FIG. 12). Especially the GGDPHLPTL and GGDPPLPTL peptides, which were highly prevalent in patients with EBV reactivation and PTLD (FIG. 9), led to potent inhibition of NKG2A⁺ NK and NKG2A⁺ CD8⁺ T cells, when stimulated with K562-HLA-E*0101/0101 or K562-HLA-E*0103/0103 target cells.

Other peptide variants, namely the GSDPHLPTL and GGDPHLPPL peptides, which were frequently found in EBV strains during IM, but rarely in EBV reactivations, elicited neither significant HLA-E upregulation nor inhibition of NKG2A⁺ NK or NKG2A⁺ CD8⁺ T cells against K562-HLA-E*0101/0101 and K562-HLA-E*0103/0103 target cells (FIG. 11B, FIG. 12).

This demonstrates that the two LMP-1 peptides GGDPHLPTL and GGDPPLPTL, which are found preferably in EBV strains causing reactivation and PTLD, elicit a strong NKG2A mediated inhibition of NK cell and CD8⁺ T cell mediated immune responses, and this may allow for an increased immune evasion of these EBV strains.

To investigate the different impact of the individual peptides on the prevention of EBV-spread, we further tested the LMP-1 peptides in EBV-dissemination assays, using sorted NKG2A⁺ NK or NKG2A⁺CD8⁺ T cells. NKG2A⁺ NK (FIG. 11C), and to a lower extent also NKG2A⁺CD8⁺ T cells (FIG. 12B) inhibited efficiently the spread of EBV in the absence of LMP-1-derived peptides, in both, K562-CR2-HLA-E*0103/0103 and K562-CR2-HLA-E*0101/0101 cells.

We then performed the EBV-dissemination assay including the K562-CR2-HLA-E*0103/0103 and K562-CR2-HLA-E*0101/0101 target cells pulsed by each of the eleven LMP-1 peptide variants. Only the LMP-1 GGDPHLPTL, GGDPPLPTL and the only rarely detected GTDPHLPTL peptide variants caused the inhibition of NKG2A⁺ NK (FIGS. 11C, D) and NKG2A⁺ CD8⁺ T cells (FIG. 12B, C), which further lead to increased viral spread in K562-CR2-HLA-E*0103/0103 and K562-CR2-HLA-E*0101/0101 cells.

As shown in FIG. 11C and FIG. 11D, four peptides inhibited the viral spread only in K562-CR2-HLA-E*0103/0103, but not in K562-CR2-HLA-E*0101/0101 cells. The remaining four peptides, including the GGDPHLPPL, GSDPHLPTL variants, which were observed frequently in IM but not in EBV reactivations, exhibited no inhibitory effect against the NKG2A⁺ NK or NKG2A⁺CD8⁺ T cell mediated prevention of the viral spread.

Thus, our data confirm that the LMP-1 GGDPHLPTL and GGDPPLPTL variants which are prevalent in patients developing PTLD are associated with a potent inhibition of NKG2A⁺ NK and T cells, especially in response to HLA*0103/0103-expressing cells. Thus, EBV strains, which encode for any of these both peptides, possess a specific NKG2A mediated immune evasion strategy.

### Discussion

In the present study, we reveal that the development of EBV-diseases is significantly associated with variations in the HLA-E-restricted immune responses of the human host. We provide evidence that the HLA-E*0101 allele is a risk factor for development of IM in children as well as in adolescents or adults. In contrast, the homozygous HLA-E*0103/0103 genotype was found to be significantly overrepresented in individuals who did not develop IM during primary EBV infections, suggesting that this genotype may contribute to a more efficient protection against EBV-associated diseases. Our finding is supported by earlier in vitro studies, which demonstrated that the HLA-E*0103/0103 provides a more efficient assembly with β2-microglobulin, a faster ER egress and a higher affinity to peptide ligands, compared to the HLA-E*0101/0101 genotype⁹. This high-level expression of HLA-E*0103/0103, may also prevent to some degree the development of IM during primary EBV infection. Previously published studies have already described familial aggregations of IM and concluded that a genetic susceptibility may exist for IM^{16,17}. While recent observational studies suggested that there might be a certain relation between classical HLA class I polymorphisms and the occurrence of IM 18,19 we now identified the HLA-E*0101 allele as a novel, host-encoded factor, which is highly associated with development of IM.

In the search for the functional background of these findings, we could show that it is the HLA-E-restricted immune response by CD8⁺ T cells, which is a key factor for the limitation of the viral replication in primary EBV infections. Using in vitro and ex vivo analyses we uncovered that a small subset of EBV-BZLF1 SQAPLPCVL-specific, HLA-E-restricted CD8⁺ T cells efficiently prevents the in vitro EBV spread. So far, most studies focused on the role of total EBV-specific CD8⁺ T cell responses in the defence against primary EBV infections²⁰ but could not find a correlation between large expansions of classical, highly activated EBV-specific CD8⁺ T cells and freedom from IM^{7,21-23}. In contrast, we found that the small subset of protective EBV-BZLF1 SQAPLPCVL-specific, HLA-E-restricted CD8⁺ T cells is in fact highly prevalent in EBV seropositive individuals, who had not undergone IM during primary infection.

Such SQAPLPCVL-specific, HLA-E-restricted T cells were earlier found in patients with multiple sclerosis⁵ and in in vitro studies²⁴, but their functional impact on the EBV spread was unknown. In addition, we could provide further evidence that HLA-E*0103/0103 expressing cells are associated with an especially efficient inhibition of EBV spread in vitro and with a pronounced activation and proliferation of these SQAPLPCVL-specific, HLA-E-restricted CD8⁺ T cells. Based on our results, it thus appears that a potent and early SQAPLPCVL-specific, HLA-E-restricted CD8⁺ T cell response limits the EBV spread especially when presented via the HLA-E*0103/0103 genotype.

Not only the well-conserved BZLF-1-derived SQAPLPCVL, but also a highly polymorphic LMP1-derived peptide are presented via HLA-E. We found eleven variants of this peptide in the EBV strains detected in patients with IM. Interestingly, the LMP-1 peptide profile of the infecting EBV strains was significantly different between the patients with IM and the elderly persons, who developed symptomatic EBV reactivation events. Upon reactivation, the LMP-1 peptide profile became more restricted towards two peptides, namely the GGDPHLPTL and GGDPPLPTL variants. Our functional analyses revealed that these peptides show a particularly strong inhibition of NKG2A⁺ NK cells and NKG2A⁺ CD8⁺ T cells. Our findings thus suggest that EBV strains carrying one of these two specific peptides may to some degree escape the human immune response by the efficient inhibition of NKG2A-expressing cells, thereby enabling the reactivation from latency. These data were further confirmed by our analyses of EBV reactivation events in transplant recipients, in whom an even higher frequency of EBV strains carrying either the LMP-1 derived GGDPHLPTL or GGDPPLPTL peptides was observed. EBV reactivations are a main concern in immunosuppressed transplant recipients and PTLD is a life-threatening malignant complication following EBV reactivations. While our data now suggest that the occurrence of reactivation events is highly associated with the LMP-1 peptide variant of the infecting EBV strain, the question remains why some of these reactivating patients progress towards PTLD, while in others, the reactivation episode is limited. So far the degree of immunosuppression, recipient age and ethnicity, allograft type, and distinct host genetic variations were considered as contributing to the risk for EBV associated PTLD²⁵, but cannot fully explain the progression towards this complication. We now uncovered, that it is the combination of the viral LMP-1 peptide variant of the infecting EBV strain, namely the GGDPHLPTL and GGDPPLPTL and the high expressing HLA-E*0103/0103 genotype of the host, which is associated with a particularly high-risk for PTLD in transplant recipients. We further revealed that the two LMP-1 peptides, GGDPHLPTL and GGDPPLPTL, if presented by the high-level expressing HLA-E*0103/0103 variant confer an especially potent inhibition of NKG2A⁺ NK cells. Thereby distinct EBV strains may efficiently escape the human immune responses, which otherwise would prevent the development of PTLD. Our data are supported by a recent ex vivo study of others, who identified a distinct NKG2A⁺ NK cell subset in the tonsils of EBV-carriers, which produced large amounts of IFNγ and prevented to some extent the B cell transformation in subsequent in vitro experiments²⁶. These data implicate that distinct NKG2A⁺ NK cell subsets can prevent the malignant transformation of B cells and may provide important immune responses preventing PTLD. Other authors showed that NKG2A⁺ NK cells provide potent cytotoxicity against latently EBV-infected B cells⁶. Our data now further reveal that a potent inhibition of NKG2A⁺ NK cells by specific viral peptides can also inhibit the NKG2A⁺ NK cell effector functions, thereby increasing the risk for PTLD. These data are of special interest as the identification of EBV LMP-1 peptides, combined with the analysis of the HLA-E variant of a transplant recipient may possibly allow the early identification of patients at high-risk for PTLD and may serve as prognostic biomarkers. Interestingly, a recently published study identified the HLA-E*0101 variant as a protective factor for EBV-related classical Hodgkin lymphoma²⁷. This not only supports our findings but also suggests that the LMP-1 peptide mediated upregulation of HLA-E may play a role in EBV-associated lymphoproliferative diseases in general. In combination with our data, this highlights the need for further assessment of the potential of LMP-1 variant and HLA-E analysis to identify patients generally at risk for EBV-associated lymphoproliferative diseases.

In summary, we have identified that HLA-E-restricted immune responses and the NKG2A/LMP-1/HLA-E axis are important factors, which contribute to clinically evident EBV associated diseases. Further extended studies are needed to evaluate whether the analysis of individual variations in the HLA-E-restricted immune response may serve as prognostic marker for malignant EBV-associated diseases. In addition, the presented data may substantially contribute to the development of efficient protective EBV vaccines.

### Patients and Methods

In our study 412 controls, 206 healthy individuals, who had never an IM in the past, 578 IM patients, 28 elderly and 144 transplant recipients with symptomatic EBV-reactivation and 36 PTLD patients were included. Details of the study cohort, HLA-E, LMP-1 and BZLF1 genotyping, the EBV-detection and serology, the functional assays and the statistical analysis are provided herein below.

### Study Cohort

In our study, a total of 1404 patients were included. Of these, 578 patients had confirmed infectious mononucleosis (IM), 412 adult or adolescent patients and 166 children. IM was clinically confirmed in all patients by the triad of fever, lymphadenopathy and tonsillitis and serologically by EBV-VCA-specific IgM, but non-detectable EBV-EBNA-specific IgG antibodies. In all IM patients, EBV-DNA was detected by EBV-PCR.

We further included 28 elderly patients with symptomatic EBV-reactivation. All elderly patients were >60 years old and were tested for EBV, due to a fever of unknown origin. All elderly patients had EBV-DNA detectable by PCR, as well as detectable EBV-VCA-specific IgM, as well as EBV-EBNA- and EBV-VCA-specific IgG antibodies.

Furthermore, 180 immunocompromised transplant recipients were included in the study. All had undergone either solid-organ- (SOT, N=149) or hematopoietic stem cell transplantation (HSCT, N=31) between 1999 and 2018, and were followed up for three years after transplantation.

SOT recipients underwent kidney (N=86), liver (N=29), lung (N=20), heart (N=11), or heart-lung transplantation (N=1). HSCT recipients underwent matched unrelated donor (N=11), matched related donor (N=10) or autologous (N=10) HSCT. All transplant patients were tested positive for EBV-DNA by EBV-PCR either due to unspecific symptoms, i.e. fever of unknown origin (N=144), or in case of PTLD diagnosis (N=36). EBV⁺ PTLD occurred between 35-302 days post-infection in 16 HSCT and 20 SOT patients, and was diagnosed using histological and/or cytological findings according to published international guidelines¹²⁻¹⁴.

In all non-PTLD patients an EBV-replication, exceeding >1000 copies/mL plasma, was detected by EBV-PCR. These patients had, however, no clinical evidence of PTLD (HSCT Non-PTLD, N=129 and SOT Non-PTLD, N=20).

We further included 206 healthy EBV-EBNA- and EBV-VCA-specific IgG positive individuals, who had never self-reported symptoms related to an IM in the past. At the time of sampling, none of the patients had positive EBV-PCR or VCA-specific IgM antibodies. Patients of the asymptomatic EBV-infection cohort were recruited using a questionnaire, sent to former patients of the Center for Virology, Medical University of Vienna. Details of the study cohort are shown in Table 1.

We then also included 412 control subjects (controls) in our study, who were selected independent of the EBV-status. Controls and asymptomatic EBV-infected individuals were matched to the adolescent/adult IM cohort in regard of the age and gender using case-control matching (SPSS 25).

From each patient, one plasma sample was available: From adolescent/adult IM and pediatric IM patients, the elderly and Non-PTLD patients during the EBV-viremic phase; from PTLD patients during the EBV-viremic phase, immediately (0-3 days) after the first diagnosis of PTLD. From asymptomatic EBV-infected individuals and controls, plasma samples were available, which was sent to the Center for Virology, Medical University of Vienna for routine vaccination titre controls. All plasma samples were part of the Biobank of the Center for Virology. From N=10 of all pediatric IM patients (6%) and N=10 of all adolescent/adult IM patients (2.4%), additional peripheral blood mononuclear cells (PBMC) were available after 10-23 and 1-8 years, respectively, after diagnosis of IM. From additional N=20 of all asymptomatic EBV-infected patients (9.7%), PBMC were also available.

For the functional assays, we also included 30 healthy, voluntary blood donors (EBNA EBV-IgG seropositive, N=24; EBNA EBV-IgG seronegative, N=6). From the healthy, voluntary blood donors, no information about the age, gender, or occurrence of an IM in the past were available.

### HLA-E, LMP-1 and BZLF1 Genotyping

Genomic and viral DNA was isolated from 200 μL plasma using the NucliSens EasyMag extractor (bioMerieux). Nucleic acids were eluted in 50 μl nuclease-free H2O. HLA-E genotyping was performed using a recently published TaqMan assay and HLA-E*0101- and HLA-E*0103-specific probes^{28,29}. LMP-1 variants were determined by nested PCR, followed by Sanger-Sequencing, as described before 10. BZLF1-peptides were analyzed by complete BZLF1 gene amplification by nested PCR and subsequent Sanger-sequencing as previously described in detail³⁰. DNA sequences were translated into protein sequences using the Expasy tool, developed by the Swiss-Prot group and supported by the SIB Swiss Institute of Bioinformatics (https://web.expasy.org/translate/).

### EBV-Detection and Serology

Viral DNA was isolated from plasma samples using NucliSens EasyMag extractor. Nucleic acids were eluted in 50 μl nuclease-free H2O. EBV-DNA was detected and quantified by TaqMan assay using recently published protocols³¹. EBV VCA IgM, EBNA IgG and VCA IgG antibodies were detected and quantified by ELISA (all: Euroimmune).

### Isolation of Primary Cells

Peripheral blood mononuclear cells (PBMCs) from 30 voluntary and healthy blood donors, from additional 20 former IM patients and from additional 20 asymptomatic EBV-infected individuals were isolated from buffy-coats by Ficoll-Paque PLUS density (Cytiva) gradient centrifugation according to the manufacture's instruction. CD56⁺ NK cells and CD8⁺ T cells were then enriched by magnetic labelling using the human CD56⁺ NK cell or the CD8⁺ T Cell Isolation Kit according to the manufacturer's instruction (both: Miltenyi Biotec). Cells were stored frozen at −80° C. in 4×106 viable cell per aliquots in 90% FCS+10% DMSO (Thermo-Fisher).

### HLA-E Stabilization Experiments

HLA-E surface stabilization was induced as previously described³². In brief, TAP-incompetent K562-HLA-E*0101/0101 and K562-HLA-E*0103/0103 (kindly provided by Thorbald van Hall, Leiden University Medical Center) were cultured in Iscove's Modified Dulbecco's Medium (IMDM)+10% FCS (both: Thermo-Scientific). 5×105 cells/ml were incubated together with 300 μM of VMAPRTLIL (positive control), BZLF1-derived SQAPLPCVL or LMP-1 peptides (Peptides&Elephants) in 1 ml Opti-MEM (Thermo-Fisher) for 16 h at 37° C. Peptide-pulsed cells were either stained for HLA-E surface expression by flow-cytometry analysis or washed with complete medium and used for in vitro stimulations.

### Identification of SQAPLPCVL-Specific, HLA-E-Restricted CD8⁺ T Cells

CD8⁺ MACS-enriched cells were quickly thawed at 37° C., washed once, and rested overnight in RPMI, 10% FCS, 1% L-glutamine at 37° C. CD8⁺ T cells were then harvested by centrifugation (400×g, 5 min) and then washed once with Opti-MEM I Reduced Serum Medium. CD8⁺ T cells were then cultured together with peptide pulsed K562-HLA-E*0101/0101, K562-HLA-E*0103/0103 or K562 cells (Effector:Target, E:T, 1:2), 1× monensin and 1× brefeldin A (both: Biolegend) for 6 h. CD8⁺ T cells were then harvested, fixed with the FIX & PERM Cell Fixation & Cell Permeabilization Kit (Thermo-Scientific) and analysed by flow-cytometry, as described below.

### Creation of CR2-Expressing Cell Lines

TAP-competent K562-HLA-E*0101/0101 and K562-HLA-E*0103/0103 (kindly provided by Thorbald van Hall, Leiden University Medical Center) were cultured in IMDM+10% FCS. The cells were transfected with the CR2/CD21-receptor using the CD21 CRISPR activation plasmid (h) according to the manufacture's instruction (Santa Cruz Biotechnology). Successful CR2/CD21 insertion was confirmed by CD21 surface expression by flow-cytometry. K562-CR2-HLA-E*0101/0101 and K562-CR2-HLA-E*0103/0103 were afterwards sorted for the high expression of CD21, as described below.

### Virus Dissemination Assays

For the virus dissemination assays, the marmoset B-lymphoblastoid cell line B95-8 was cultured in RPM1 1640 medium supplemented with 20 mM glutamine, 50 pg/ml gentamicin, 0.2 pg/ml amphotericin B and 10% FCS (Thermo-Scientific). Cells were then cultured in the presence of 12-O-tetradecanoyl-phorbol-12-acetate (Biomol) for two weeks. EBV was harvested from the supernatant and enriched by ultracentrifugation (30000 U/min, 90 min, 10° C.). The virus stocks were stored at −80° C. in RPMI 1640 medium+10% FCS+10% DMSO (all: Thermo-Scientific). The multiplicity of infection (MOI) of the viral stocks was determined on K562-CR2-HLA-E*0101/0101 and K562-CR2-HLA-E*0103/0103 cells by flow-cytometry.

RMA-S/HLA-E/LFA-3 cells (kindly provided by Chiara Romagnani, DRFZ, Berlin) were maintained in RPMI-1640+20 mM glutamine+10% FCS+20 μM β-mercaptoethanol+100 U/ml penicillin-streptomycin (all Thermo Fisher)+400 μg/ml hygromycin B+1 mg/ml G418 (both InvivoGen). 2×106 RMA-S/LFA3/HLA-E cells were incubated for 16 h at 37° C., 5% CO2, with 300 μM of respective peptides (Peptides&Elephants) in 1 ml serum-free OptiMEM (Thermo-Fisher). Cells were afterwards inactivated using 20 μg/mL Mitomycin C (Sigma Aldrich) at 37° C. for 30 minutes. Peptide pulsed RMA-S/LFA3/HLA-E cells were co-cultured with 2 mM CFSE (Thermo-Fisher) stained CD56⁺ or CD8⁺ MACS-enriched cells for 7 days in RPMI+10% FCS. Proliferating (CFSE^{low}) NKG2A⁺CD56⁺ NK cells, NKG2A⁺ CD8⁺ T cells or SQAPLPCVL-specific, HLA-E-restricted NKG2A⁺ or NKG2A⁻ T cells were then sorted by FACS as described below.

Fresh K562-CR2-HLA-E*0101/0101 and K562-CR2-HLA-E*0103/0103 cells were then inactivated using 20 μg/mL Mitomycin C at 37° C. for 30 minutes and then infected with the EBV B95-8 isolate (MOI=1) for 24 h. The infected cells were then co-cultured together with the sorted NKG2A⁺ CD56⁺ NK cells, NKG2A⁺ CD8⁺ T cells or SQAPLPCVL-specific, HLA-E-restricted NKG2A⁺ or NKG2A⁻ T cells (E:T, 1:2). In some experiments, 300 μM of respective peptides was also added. At indicated time points, the cells were harvested and analyzed by flow-cytometry, as described below.

### CD8⁺ Proliferation Assay

For the CD8⁺ T cell proliferation assays, SQAPLPCVL pulsed TAP-deficient K562-HLA-E*0101/0101 or K562-HLA-E*0103/0103 cells were first inactivated using 20 μg/mL Mitomycin C (Sigma Aldrich) at 37° C. for 30 minutes. The cells were then co-cultivated with CFSE stained MACS-enriched CD8⁺ T cells for 7 days (E:T, 2:1). After three days, fresh peptide-pulsed K562-HLA-E*0101/0101 or K562-HLA-E*0103/0103 cells were added to the culture. Cells were then analysed after 7 days by flow-cytometry, as described below.

NKG2A⁺ NK Cells and NKG2A⁺ CD8⁺ T Cell Inhibition Assay

For the NKG2A⁺ NK cell inhibition experiments, MACS-enriched CD56⁺ cells were quickly thawed at 37° C., washed, and pre-activated overnight in RPMI, 10% FCS, 1% L-glutamine (all: Thermo Fisher Scientific), 10 ng/ml IL-12 (PeproTec) and 100 ng/ml IL-18 (Biozym Scientific) at 37° C. CD56⁺ NK cells were then harvested by centrifugation at 400×g for 5 minutes and washed once with Opti-MVEM I Reduced Serum Medium (Gibco). The NK cells were then cultured together with peptide pulsed K562-HLA-E*0101/0101 or K562-HLA-E*0103/0103 cells (E:T, 1:2) and 5 μL mouse anti-human CD107-APC-H7 (BD) for 6 h. NK cells were then harvested, fixed with the FIX & PERM Cell Fixation & Cell Permeabilization Kit (Thermo-Scientific) and analysed by flow-cytometry, as described below.

For the CD8⁺ T cells experiments, CD8⁺ MACS-enriched cells were quickly thawed at 37° C., washed once, and pre-activated overnight in RPMI, 10% FCS, 1% L-glutamine and 20 ng/ml IL-2 (PeproTec) at 37° C. CD8⁺ T cells were then harvested by centrifugation (400×g, 5 min) and then washed once with Opti-MEM I Reduced Serum Medium. CD8⁺ T cells were then cultured together with peptide pulsed K562-HLA-E*0101/0101 or K562-HLA-E*0103/0103 cells (E:T, 1:2), 1× monensin and 1× brefeldin A for 6 h. NK cells were harvested, fixed with the FIX & PERM Cell Fixation & Cell Permeabilization Kit and analysed by flow-cytometry, as described below.

### Flow-Cytometry

The following conjugated mouse anti-human mAB were used for flow-cytometry: BV421-CD56 (NCAM 16.2), BV421-CD8 (RPA-T8), BV510-NKG2A (131411), APC/H7-CD107a (H4A3), APC/CD21 (all: BD), APC-CD3 (UCHT1), APC/Cy7-IFNγ (B27), APC-HLA/E (all: Biolegend), APC-Cy7-Granzyme B (Abcore), BZLF1-Alexa488 (Novusbio). Dead cells were identified using LIVE/DEAD Fixable Green Dead Cell Stain Kit (Thermo-Scientific) or 7-Aminoactinomycin D (7-AAD, Invitrogen). Flow-cytometry analysis was performed on a FACSCanto2 platform and FACSDiva Version 10.7.2 (BD). For cell sorting, dead cells were first removed using the Dead Cell Removal Kit (Miltenyi Biotec) and sorted with a FACSAria Fusion (BD).

### Statistical Analysis

The Chi-square test and Fisher's exact test were used to compare the distribution of the HLA-E and LMP-1 variants. Outliers of the flow cytometry data were first identified using the ROUT method and then compared between the groups with the RM one-way ANOVA (with the Geisser-Greenhouse correction), the Kruskal-Wallis Test and the Dunn's post-test, Wilcoxon signed-rank test or the Mann-Whitney test. A p-value<0.05 was considered statistically significant. Statistical differences were assessed with GraphPad Prism 9.

TABLE 1
Characteristics of the Study Cohort

| | Study Cohort | | | | | |
|---|---|---|---|---|---|---|
| | Primary EBV-Infection | | | EBV-Reactivation | | |
| | Asymptomatic | | | Immuno- | Immunocompromised | |
| | EBV- | Adolescent/ | Paediatric | competent | Transplant Recipients | |
| | Seropositive | Adult IM | IM | Elderly | Non-PTLD | PTLD |
| | N = 206 | N = 412 | N = 166 | N = 28 | N = 144 | N = 36 |
| Female (%) | N = 90 (43.6%) | N = 181 (43.9%) | N = 73 (43.9%) | N = 11 (39.2%) | N = 61 (42.4%) | N = 14 (38.9%) |
| Median Age | 22.5 (18-40) | 22.1 (15-35) | 5.8 (1-14) | 71.7 (60-92) | 37.8 (14-91) | 30.2 (14-74) |
| (min-max) | | | | | | |
| EBV-Status | | | | | | |
| EBV- RT qPCR | neg. | pos. | pos. | pos. | pos. | pos. |
| VCA IgM | neg. | pos. | pos. | pos. | pos. | pos. |
| VCA IgG | pos. | pos. | pos. | pos. | pos. | pos. |
| EBNA lgG | pos. | neg. | neg. | pos. | pos. | pos. |
| Clinical Diagnosis | none | IM * | IM * | Unspecific | Unspecific | PTLD |
| | | | | Symptoms ** | Symptoms ** | |
| Transplantation | | | | | | |
| Type | | | | | | |
| HSCT (%) | | | | | N = 15 (10.4%) | N = 16 (44.4%) |
| SOT (%) | | | | | N = 129 (89.6%) | N = 20 (55.6%) |
| First EBV-episode | | | | | 90 (29-312) | |
| (median days) | | | | | | |
| post-trans- | | | | | | |
| plantation (range) | | | | | | |
| PTLD-diagnosis | | | | | | 99 (35-302) |
| (median days) | | | | | | |
| post-trans- | | | | | | |
| plantation (range) | | | | | | |
| PTLD Type | | | | | | |
| Early Lesions (%) | | | | | | N = 4 (11.1%) |
| Monomorphic B | | | | | | N = 21 (58.3%) |
| cell PTLD (%) | | | | | | |
| Polymorphic | | | | | | N = 11 (30.6%) |
| PTLD (%) | | | | | | |

Abbreviations:
EBNA: Epstein-Barr virus nuclear antigen 1;
EBV: Epstein-Barr virus;
HSCT: hematopoietic stem cell transplantation;
IM: infectious mononucleosis;
neg: negative;
pos: positive;
PTLD: Post-transplant lymphoproliferative disorder;
RT qPCR: Real-time quantitative polymerase chain reaction;
SOT: solid organ transplantation;
VCA: viral-capsid antigen
* As defined by the triad of fever, lymphadenopathy and tonsillitis
** Unspecific symptoms include fever, weight loss, lymphadenopathy, night sweats or fatigue

### EXAMPLE 2

### Sequence Alterations of the SQAPLPCVL Peptide do not Elicit an EBV-Specific, HLA-E Restricted CD8+ T Cell Response

### Aim

This example was performed to address the question if sequence alterations of the SQAPLPCVL peptide elicit any EBV-specific, HLA-E restricted CD8⁺ T cell responses.

### Material and Methods

The marmoset B-lymphoblastoid cell line B95-8 was cultured in RPM1 1640 medium supplemented with 20 mM glutamine, 50 pg/ml gentamicin, 0.2 pg/ml amphotericin B and 10% FCS (Thermo-Scientific). Cells were then cultured in the presence of 12-O-tetradecanoyl-phorbol-12-acetate (Biomol) for two weeks. EBV was harvested from the supernatant and enriched by ultracentrifugation (30000 U/min, 90 min, 10° C.). The virus stocks were stored at −80° C. in RPMI 1640 medium+10% FCS+10% DMSO (all: Thermo-Scientific). The multiplicity of infection (MOI) of the viral stocks was determined on K562-CR2-HLA-E*0103/0103 cells by flow-cytometry.

Peripheral blood mononuclear cells (PBMCs) from 10 voluntary and healthy blood donors were isolated from buffy-coats by Ficoll-Paque PLUS density (Cytiva) gradient centrifugation according to the manufacture's instruction. CD8⁺ T cells were then enriched by magnetic labelling using the human CD8⁺ T Cell Isolation Kit according to the manufacturer's instruction (Miltenyi Biotec). Cells were stored frozen at −80° C. in 4×10⁶ viable cells per aliquots in 90% FCS+10% DMSO (Thermo-Fisher).

K562-CR2-HLA-E*0103/0103 cells were then inactivated using 20 μg/mL Mitomycin C at 37° C. for 30 minutes and then infected with the EBV B95-8 isolate (MOI=1) for 24 h. The cells were then pulsed with 300 μM of each of the respective peptides:
SQAPLPCVL (indicated as “1.AA S”), AQAPLPCVL (indicated as “1.AA X”), RQAPLPCVL (indicated as “1.AA X”), NQAPLPCVL (indicated as “1.AA X”), DQAPLPCVL (indicated as “1.AA X”), CQAPLPCVL (indicated as “1.AA X”), QQAPLPCVL (indicated as “1.AA X”), EQAPLPCVL (indicated as “1.AA X”), GQAPLPCVL (indicated as “1.AA X”), HQAPLPCVL (indicated as “1.AA X”), IQAPLPCVL (indicated as “1.AA X”), LQAPLPCVL (indicated as “1.AA X”), KQAPLPCVL (indicated as “1.AA X”), MQAPLPCVL (indicated as “1.AA X”), FQAPLPCVL (indicated as “1.AA X”), PQAPLPCVL (indicated as “1.AA X”), TQAPLPCVL (indicated as “1.AA X”), WQAPLPCVL (indicated as “1.AA X”), YQAPLPCVL (indicated as “1.AA X”), VQAPLPCVL (indicated as “1.AA X”).
SQAPLPCVL (indicated as “2.AA Q”), SAAPLPCVL (indicated as “2.AA X”), SRAPLPCVL (indicated as “2.AA X”), SNAPLPCVL (indicated as “2.AA X”), SDAPLPCVL (indicated as “2.AA X”), SCAPLPCVL (indicated as “2.AA X”), SEAPLPCVL (indicated as “2.AA X”), SGAPLPCVL (indicated as “2.AA X”), SHAPLPCVL (indicated as “2.AA X”), SIAPLPCVL (indicated as “2.AA X”), SLAPLPCVL (indicated as “2.AA X”), SKAPLPCVL (indicated as “2.AA X”), SMAPLPCVL (indicated as “2.AA X”), SFAPLPCVL (indicated as “2.AA X”), SPAPLPCVL (indicated as “2.AA X”), SSAPLPCVL (indicated as “2.AA X”), STAPLPCVL (indicated as “2.AA X”), SWAPLPCVL (indicated as “2.AA X”), SYAPLPCVL (indicated as “2.AA X”), SVAPLPCVL (indicated as “2.AA X”).
SQAPLPCVL (indicated as “3.AA A”), SQRPLPCVL (indicated as “3.AA X”), SQNPLPCVL (indicated as “2.AA X”), SQDPLPCVL (indicated as “2.AA X”), SQCPLPCVL (indicated as “2.AA X”), SQQPLPCVL (indicated as “2.AA X”), SQEPLPCVL (indicated as “2.AA X”), SQGPLPCVL (indicated as “2.AA X”), SQHPLPCVL (indicated as “2.AA X”), SQIPLPCVL (indicated as “2.AA X”), SQLPLPCVL (indicated as “2.AA X”), SQKPLPCVL (indicated as “2.AA X”), SQMPLPCVL (indicated as “2.AA X”), SQFPLPCVL (indicated as “2.AA X”), SQPPLPCVL (indicated as “2.AA X”), SQSPLPCVL (indicated as “2.AA X”), SQTPLPCVL (indicated as “2.AA X”), SQWPLPCVL (indicated as “2.AA X”), SQYPLPCVL (indicated as “2.AA X”), SQVPLPCVL (indicated as “2.AA X”).
SQAPLPCVL (indicated as “4.AA P”), SQAALPCVL (indicated as “4.AA X”), SQARLPCVL (indicated as “4.AA X”), SQANLPCVL (indicated as “4.AA X”), SQADLPCVL (indicated as “4.AA X”), SQACLPCVL (indicated as “4.AA X”), SQAQLPCVL (indicated as “4.AA X”), SQAELPCVL (indicated as “4.AA X”), SQAGLPCVL (indicated as “4.AA X”), SQAHLPCVL (indicated as “4.AA X”), SQAILPCVL (indicated as “4.AA X”), SQALLPCVL (indicated as “4.AA X”), SQAKLPCVL (indicated as “4.AA X”), SQAMLPCVL (indicated as “4.AA X”), SQAFLPCVL (indicated as “4.AA X”), SQASLPCVL (indicated as “4.AA X”), SQATLPCVL (indicated as “4.AA X”), SQAWLPCVL (indicated as “4.AA X”), SQAYLPCVL (indicated as “4.AA X”), SQAVLPCVL (indicated as “4.AA X”).
SQAPLPCVL (indicated as “5.AA L”), SQAPAPCVL (indicated as “5.AA X”), SQAPRPCVL (indicated as “5.AA X”), SQAPNPCVL (indicated as “5.AA X”), SQAPDPCVL (indicated as “5.AA X”), SQAPCPCVL (indicated as “5.AA X”), SQAPQPCVL (indicated as “5.AA X”), SQAPEPCVL (indicated as “5.AA X”), SQAPGPCVL (indicated as “5.AA X”), SQAPHPCVL (indicated as “5.AA X”), SQAPIPCVL (indicated as “5.AA X”), SQAPKPCVL (indicated as “5.AA X”), SQAPMPCVL (indicated as “5.AA X”), SQAPFPCVL (indicated as “5.AA X”), SQAPPPCVL (indicated as “5.AA X”), SQAPSPCVL (indicated as “5.AA X”), SQAPTPCVL (indicated as “5.AA X”), SQAPWPCVL (indicated as “5.AA X”), SQAPYPCVL (indicated as “5.AA X”), SQAPVPCVL (indicated as “5.AA X”).
SQAPLPCVL (indicated as “6.AA P”), SQAPLACVL (indicated as “6.AA X”), SQAPLRCVL (indicated as “6.AA X”), SQAPLNCVL (indicated as “6.AA X”), SQAPLDCVL (indicated as “6.AA X”), SQAPLCCVL (indicated as “6.AA X”), SQAPLQ CVL (indicated as “6.AA X”), SQAPLE CVL (indicated as “6.AA X”), SQAPLGCVL (indicated as “6.AA X”), SQAPLHCVL (indicated as “6.AA X”), SQAPLICVL (indicated as “6.AA X”), SQAPLLCVL (indicated as “6.AA X”), SQAPLKCVL (indicated as “6.AA X”), SQAPLMCVL (indicated as “6.AA X”), SQAPLFCVL (indicated as “6.AA X”), SQAPLSCVL (indicated as “6.AA X”), SQAPLTCVL (indicated as “6.AA X”), SQAPLWCVL (indicated as “6.AA X”), SQAPLYCVL (indicated as “6.AA X”), SQAPLVCVL (indicated as “6.AA X”).
SQAPLPCVL (indicated as “7.AA C”), SQAPLPAVL (indicated as “7.AA X”), SQAPLPRVL (indicated as “7.AA X”), SQAPLPNVL (indicated as “7.AA X”), SQAPLPDVL (indicated as “7.AA X”), SQAPLPQVL (indicated as “7.AA X”), SQAPLPEVL (indicated as “7.AA X”), SQAPLPGVL (indicated as “7.AA X”), SQAPLPHVL (indicated as “7.AA X”), SQAPLPIVL (indicated as “7.AA X”), SQAPLPLVL (indicated as “7.AA X”), SQAPLPKVL (indicated as “7.AA X”), SQAPLPMVL (indicated as “7.AA X”), SQAPLPFVL (indicated as “7.AA X”), SQAPLPPVL (indicated as “7.AA X”), SQAPLPSVL (indicated as “7.AA X”), SQAPLPTVL (indicated as “7.AA X”), SQAPLPWVL (indicated as “7.AA X”), SQAPLPYVL (indicated as “7.AA X”), SQAPLPVVL (indicated as “7.AA X”).
SQAPLPCVL (indicated as “8.AA V”), SQAPLPCAL (indicated as “8.AA X”), SQAPLPCRL (indicated as “8.AA X”), SQAPLPCNL (indicated as “8.AA X”), SQAPLPCDL (indicated as “8.AA X”), SQAPLPCCL (indicated as “8.AA X”), SQAPLPCQL (indicated as “8.AA X”), SQAPLPCEL (indicated as “8.AA X”), SQAPLPCGL (indicated as “8.AA X”), SQAPLPCHL (indicated as “8.AA X”), SQAPLPCTL (indicated as “8.AA X”), SQAPLPCLL (indicated as “8.AA X”), SQAPLPCKL (indicated as “8.AA X”), SQAPLPCML (indicated as “8.AA X”), SQAPLPCFL (indicated as “8.AA X”), SQAPLPCPL (indicated as “8.AA X”), SQAPLPCSL (indicated as “8.AA X”), SQAPLPCTL (indicated as “8.AA X”), SQAPLPCWL (indicated as “8.AA X”), SQAPLPCYL (indicated as “8.AA X”).
SQAPLPCVL (indicated as “9.AA L”), SQAPLPCVA (indicated as “9.AA X”), SQAPLPCVR (indicated as “9.AA X”), SQAPLPCVN (indicated as “9.AA X”), SQAPLPCVD (indicated as “9.AA X”), SQAPLPCVC (indicated as “9.AA X”), SQAPLPCVQ (indicated as “9.AA X”), SQAPLPCVE (indicated as “9.AA X”), SQAPLPCVG (indicated as “9.AA X”), SQAPLPCVH (indicated as “9.AA X”), SQAPLPCVI (indicated as “9.AA X”), SQAPLPCVK (indicated as “9.AA X”), SQAPLPCVM (indicated as “9.AA X”), SQAPLPCVF (indicated as “9.AA X”), SQAPLPCVP (indicated as “9.AA X”), SQAPLPCVS (indicated as “9.AA X”), SQAPLPCVT (indicated as “9.AA X”), SQAPLPCVW (indicated as “9.AA X”), SQAPLPCVY (indicated as “9.AA X”), SQAPLPCVV (indicated as “9.AA X”).
SQAPLPCKL (indicated as “8.AA X”), SQAPLPCVK (indicated as “9.AA X”) and SQAQLPCVL (indicated as “4.AA X”) correspond to the mutant peptides termed “BZLF-1_{39-47K46}”, “BZLF-1_{39.47K47}” and “BZLF-1_{39-47Q42}”, respectively in Tables I and II of Ulbrecht et al.³⁴. As noted, Ulbrecht et al.³⁴ only assessed binding/stabilization to HLA-E.

CD8⁺ MACS-enriched cells were then quickly thawed at 37° C., washed once, and rested overnight in RPMI, 10% FCS, 1% L-glutamine at 37° C. CD8⁺ T cells were then harvested by centrifugation (400×g, 5 min) and then washed once with Opti-MEM I Reduced Serum Medium. CD8⁺ T cells were then cultured together with EBV-infected and peptide pulsed K562-CR2-HLA-E*0103/0103 cells (Effector:Target, E:T, 1:2), 1× monensin and 1× brefeldin A (both: Biolegend) for 6 h. CD8⁺ T cells were then harvested, fixed with the FIX & PERM Cell Fixation & Cell Permeabilization Kit (Thermo-Scientific) and analysed by flow-cytometry for the expression of IFNγ.

### Results

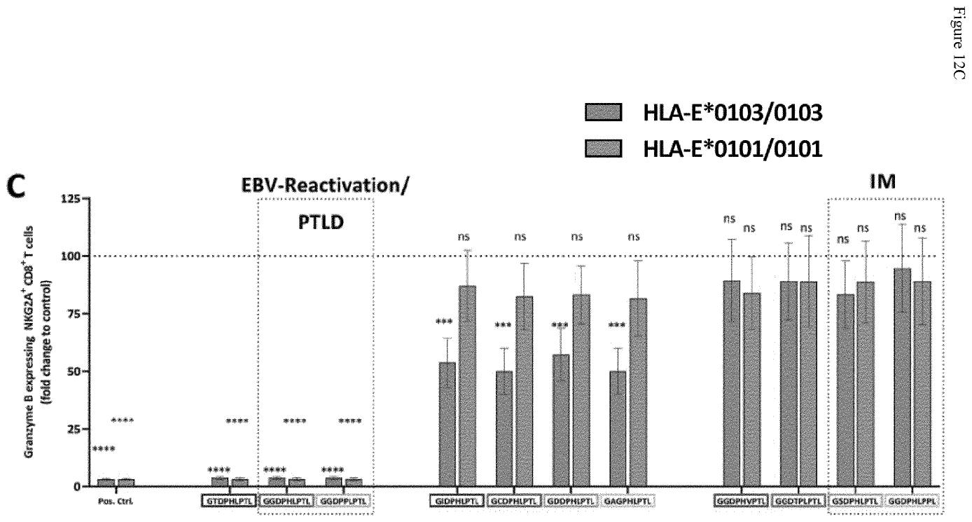

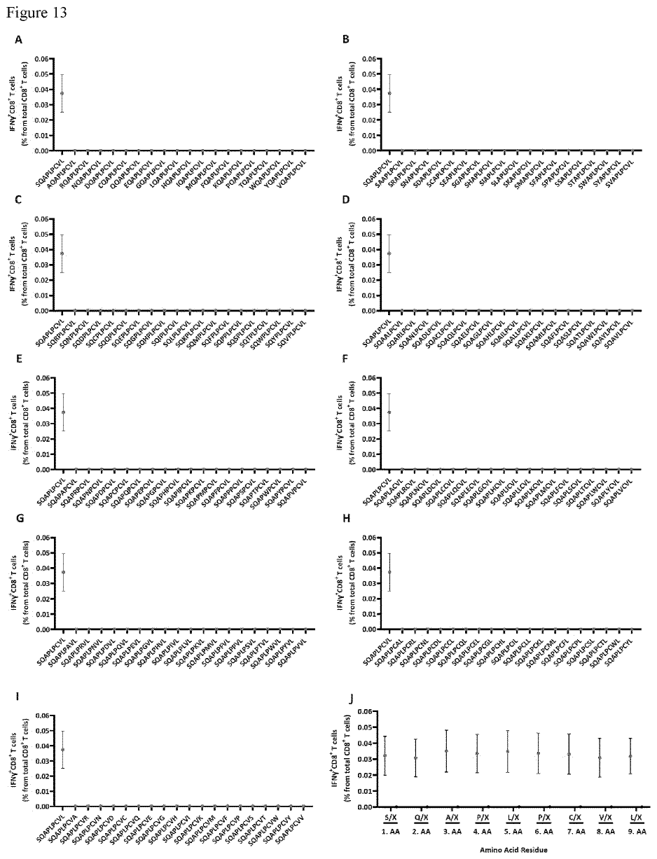

The results are shown in FIG. 13. FIG. 13 J shows the mean of the IFNγ. (i.e. activated cells) expressing CD8+ T cells (±SD), while panels A-I of FIG. 13 show the individual values of the alterations. For each amino acid, all peptides (expect for the SQAPLPCVL) were combined into one graph. Sequence alterations of the SQAPLPCVL peptide do not elicit an EBV-specific, HLA-E restricted CD8+ T cell response, as demonstrated by an undetectable number of IFNγ (i.e. not activated cells) expressing CD8⁺ T cells. Only the SQAPLPCVL peptide (i.e. peptide consisting of SEQ ID NO: 1) elicits an EBV-specific, HLA-E restricted CD8+ T cell response, as demonstrated by a detectable amount of IFNγ (i.e. activated cells) expressing CD8+ T cells.

### EXAMPLE 3

### Longer or Shorter Variants of the SQAPLPCVL Peptide do not Elicit any EBV-Specific, HLA-E Restricted CD8+ T Cell Response

### Aim

This example was performed to address the question if longer (+3AA, +2AA, +1AA) or shorter (−3AA, −2AA, −1AA) variants of the SQAPLPCVL peptide elicit any EBV-specific, HLA-E restricted CD8+ T cell responses.

### Material and Methods

The marmoset B-lymphoblastoid cell line B95-8 was cultured in RPM1 1640 medium supplemented with 20 mM glutamine, 50 pg/ml gentamicin, 0.2 pg/ml amphotericin B and 10% FCS (Thermo-Scientific). Cells were then cultured in the presence of 12-O-tetradecanoyl-phorbol-12-acetate (Biomol) for two weeks. EBV was harvested from the supernatant and enriched by ultracentrifugation (30000 U/min, 90 min, 10° C.). The virus stocks were stored at −80° C. in RPMI 1640 medium+10% FCS+10% DMSO (all: Thermo-Scientific). The multiplicity of infection (MOI) of the viral stocks was determined on K562-CR2-HLA-E*0103/0103 cells by flow-cytometry.

Peripheral blood mononuclear cells (PBMCs) from 10 voluntary and healthy blood donors were isolated from buffy-coats by Ficoll-Paque PLUS density (Cytiva) gradient centrifugation according to the manufacture's instruction. CD8⁺ T cells were then enriched by magnetic labelling using the human CD8⁺ T Cell Isolation Kit according to the manufacturer's instruction (Miltenyi Biotec). Cells were stored frozen at −80° C. in 4×10⁶ viable cells per aliquots in 90% FCS+10% DMSO (Thermo-Fisher).

K562-CR2-HLA-E*0103/0103 cells were then inactivated using 20 μg/mL Mitomycin C at 37° C. for 30 minutes and then infected with the EBV B95-8 isolate (MOI=1) for 24 h. The cells were then pulsed with 300 μM of each of the respective peptides:
QQPSQAPLPCVLWPV
QPSQAPLPCVLWPV
QQPSQAPLPCVLWP
QPSQAPLPCVLWP
QPSQAPLPCVLW
PSQAPLPCVLWP
PSQAPLPCVLW
SQAPLPCVLW
PSQAPLPCVL
SQAPLPCVL
QAPLPCVL
APLPCVL
PLPCVL
SQAPLPCV
SQAPLPC
SQAPLP

SQAPLPCVLW, SQAPLPCV, QAPLPCVL, SQAPLPC and SQAPLP correspond to the 10 mer BZLF-1₃₉₋₄₈, the 8 mer BZLF-1₃₉₋₄₆, the 8 mer BZLF-1₄₀₋₄₇, the 7 mer BZLF-1₃₉₋₄₅, the 6 mer BZLF-1₃₉₋₄₄ peptides, respectively, in Tables I and II of Ulbrecht et al.³⁴. As noted, Ulbrecht et al. 34 only assessed binding to/stabilization by HLA-E.

CD8⁺ MACS-enriched cells were then quickly thawed at 37° C., washed once, and rested overnight in RPMI, 10% FCS, 1% L-glutamine at 37° C. CD8⁺ T cells were then harvested by centrifugation (400×g, 5 min) and then washed once with Opti-MEM I Reduced Serum Medium. CD8⁺ T cells were then cultured together with EBV-infected and peptide pulsed K562-CR2-HLA-E*0103/0103 cells (Effector:Target, E:T, 1:2), 1× monensin and 1× brefeldin A (both: Biolegend) for 6 h. CD8⁺ T cells were then harvested, fixed with the FIX & PERM Cell Fixation & Cell Permeabilization Kit (Thermo-Scientific) and analysed by flow-cytometry for the expression of IFNγ.

### Results

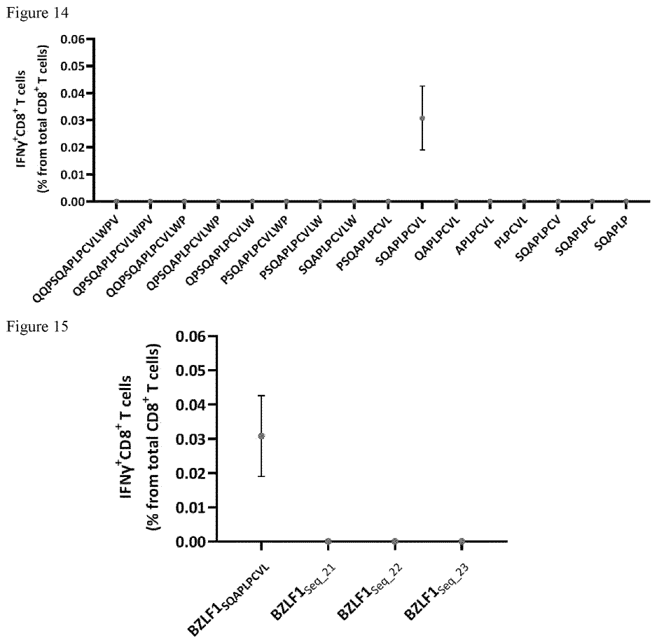

The results are shown in FIG. 14. FIG. 14 shows the mean of the IFNγ. (i.e. activated cells) expressing CD8+ T cells (±SD). Only the 9 amino acid long peptide sequence (SQAPLPCVL) elicits an EBV-specific, HLA-E restricted CD8+ T cell response, as demonstrated by a detectable amount of IFNγ (i.e. activated cells) expressing CD8+ T cells. None of the longer or shorter variants tested elicit an EBV-specific, HLA-E restricted CD8+ T cell response, as demonstrated by an undetectable amount of IFNγ (i.e. not activated cells) expressing CD8+ T cells.

### EXAMPLE 4

Larger Polypeptides, e.g. The Full Length BZLF1, Comprising the SQAPLPCVL Motif do not Elicit any EBV-Specific, HLA-E Restricted CD8+ T Cell Response

### Aim

This example was performed to address the question if the SQAPLPCVL motif in context of a larger polypeptide elicits any EBV-specific, HLA-E restricted CD8+ T cell responses.

### Material and Methods

The marmoset B-lymphoblastoid cell line B95-8 was cultured in RPM1 1640 medium supplemented with 20 mM glutamine, 50 pg/ml gentamicin, 0.2 pg/ml amphotericin B and 10% FCS (Thermo-Scientific). Cells were then cultured in the presence of 12-O-tetradecanoyl-phorbol-12-acetate (Biomol) for two weeks. EBV was harvested from the supernatant and enriched by ultracentrifugation (30000 U/min, 90 min, 10° C.). The virus stocks were stored at −80° C. in RPMI 1640 medium+10% FCS+10% DMSO (all: Thermo-Scientific). The multiplicity of infection (MOI) of the viral stocks was determined on K562-CR2-HLA-E*0103/0103 cells by flow-cytometry.

Peripheral blood mononuclear cells (PBMCs) from 10 voluntary and healthy blood donors were isolated from buffy-coats by Ficoll-Paque PLUS density (Cytiva) gradient centrifugation according to the manufacture's instruction. CD8⁺ T cells were then enriched by magnetic labelling using the human CD8⁺ T Cell Isolation Kit according to the manufacturer's instruction (Miltenyi Biotec). Cells were stored frozen at −80° C. in 4×10⁶ viable cells per aliquots in 90% FCS+10% DMSO (Thermo-Fisher).

K562-CR2-HLA-E*0103/0103 cells were then inactivated using 20 μg/mL Mitomycin C at 37° C. for 30 minutes and then infected with the EBV B95-8 isolate (MOI=1) for 24 h. The cells were then pulsed with 300 μM of each of the respective peptides:

(the SQAPLPCVL motif in context of the larger polypeptides is underlined)

BZLF1_{SQAPLPCVL}
(SEQ ID NO: 1)
Ser Gln Ala Pro Leu Pro Cys Val Leu
BZLF1_{Seq}_21
(SEQ ID NO: 201)
Met Met Asp Pro Asn Ser Thr Ser Glu Asp Val Lys
Phe Thr Pro Asp Pro Tyr Gln Val Pro Phe Val Gln
Ala Phe Asp Gln Ala Thr Arg Val Tyr Gln Asp Leu
Gly Gly Pro Ser Gln Ala Pro Leu Pro Cys Val Leu
Trp Pro Val Leu Pro Glu Pro Leu Pro Gln Gly Gln
Leu Thr Ala Tyr His Val Ser Thr Ala Pro Thr Gly
Ser Trp Phe Ser Ala Pro Gln Pro Ala Pro Glu Asn
Ala Tyr Gln Ala Tyr Ala Ala Pro Gln Leu Phe Pro
Val Ser Asp Ile Thr Gln Asn Gln Gln Thr Asn Gln
Ala Gly Gly Glu Ala Pro Gln Pro Gly Asp Asn Ser
Thr Val Gln Thr Ala Ala Ala Val Val Phe Ala Cys
Pro Gly Ala Asn Gln Gly Gln Gln Leu Ala Asp Ile
Gly Val Pro Gln Pro Ala Pro Val Ala Ala Pro Ala
Arg Arg Thr Arg Lys Pro Gln Gln Pro Glu Ser Leu
Glu Glu Cys Asp Ser Glu Leu Glu Ile Lys Arg Tyr
Lys Asn Arg Val Ala Ser Arg Lys Cys Arg Ala Lys
Phe Lys Gln Leu Leu Gln His Tyr Arg Glu Val Ala
Ala Ala Lys Ser Ser Glu Asn Asp Arg Leu Arg Leu
Leu Leu Lys Gln Met Cys Pro Ser Leu Asp Val Asp
Ser Ile Ile Pro Arg Thr Pro Asp Val Leu His Glu
Asp Leu Leu Asn Phe
BZLF1_{Seq}_22
(SEQ ID NO: 202)
Asp Leu Gly Gly Pro Ser Gln Ala Pro Leu Pro Cys
Val Leu Trp Pro Val Leu Pro Glu Pro Leu Pro Gln
Gly Gln Leu Thr Ala Tyr His Val Ser Thr Ala Pro
Thr Gly Ser Trp Phe Ser Ala Pro Gln Pro Ala Pro
Glu Asn Ala Tyr Gln Ala Tyr Ala Ala Pro Gln Leu
Phe Pro Val Ser Asp Ile
BZLF1_{Seq}_23
(SEQ ID NO: 203)
Arg Lys Pro Gln Gln Pro Glu Ser Leu Glu Glu Cys
Asp Ser Glu Leu Glu Ile Lys Arg Tyr Lys Asn Arg
Val Ala Ser Arg Lys Cys Arg Ala Lys Phe Lys Gln
Leu Leu Gln His Tyr Arg Glu Val Ala Ala Ala Lys
Ser Ser Glu

SEQ ID NOs: 201, 202 and 203 correspond to SEQ ID NOs: 21, 22 and 23, respectively, disclosed in WO 2019/123169 A1.

CD8⁺ MACS-enriched cells were then quickly thawed at 37° C., washed once, and rested overnight in RPMI, 10% FCS, 1% L-glutamine at 37° C. CD8⁺ T cells were then harvested by centrifugation (400×g, 5 min) and then washed once with Opti-MEM I Reduced Serum Medium. CD8⁺ T cells were then cultured together with EBV-infected and peptide pulsed K562-CR2-HLA-E*0103/0103 cells (Effector:Target, E:T, 1:2), 1× monensin and 1× brefeldin A (both: Biolegend) for 6 h. CD8⁺ T cells were then harvested, fixed with the FIX & PERM Cell Fixation & Cell Permeabilization Kit (Thermo-Scientific) and analysed by flow-cytometry for the expression of IFNγ.

### Results

The results are shown in FIG. 15. FIG. 15 shows the mean of the IFNγ. (i.e. activated cells) expressing CD8+ T cells (±SD). Only the 9 amino acid long peptide sequence (SQAPLPCVL) elicits an EBV-specific, HLA-E restricted CD8⁺ T cell response, as demonstrated by a detectable amount of IFNγ (i.e. activated cells) expressing CD8⁺ T cells. None of the other peptides comprising the SQAPLPCVL are capable of eliciting an EBV-specific, HLA-E restricted CD8⁺ T cell response, as demonstrated by an undetectable amount of IFNγ (i.e. not activated cells) expressing CD8⁺ T cells.

### EXAMPLE 5

### SQAPLPCVL-Specific, HLA-E-Restricted CD8+ T Cells do not Express NKG2C and CD94

### Aim

This example was performed to address the question if EBV-specific, HLA-E restricted CD8+ T cells express CD94/NKG2C?

### Material and Methods

Peripheral blood mononuclear cells (PBMCs) from 10 voluntary and healthy blood donors were isolated from buffy-coats by Ficoll-Paque PLUS density (Cytiva) gradient centrifugation according to the manufacture's instruction. CD8⁺ T cells were then enriched by magnetic labelling using the human CD8⁺ T Cell Isolation Kit according to the manufacturer's instruction (Miltenyi Biotec). Cells were stored frozen at −80° C. in 4×10⁶ viable cells per aliquots in 90% FCS+10% DMSO (Thermo-Fisher). CD8⁺ MACS-enriched cells were quickly thawed at 37° C., washed once, and rested overnight in RPMI, 10% FCS, 1% L-glutamine at 37° C. CD8⁺ T cells were then harvested by centrifugation (400×g, 5 min) and then washed once with Opti-MEM I Reduced Serum Medium. CD8⁺ T cells were then cultured together with peptide pulsed K562-HLA-E*0103/0103 (Effector:Target, E:T, 1:2), 1× monensin and 1× brefeldin A (both: Biolegend) for 6 h. CD8⁺ T cells were then harvested, fixed with the FIX & PERM Cell Fixation & Cell Permeabilization Kit (Thermo-Scientific) and analysed by flow-cytometry, as described below. SQAPLPCVL-specific, HLA-E-restricted CD8+ T cells of each donor were identified as IFNγ positive cells and in comparison to cells, stimulated with K562-HLA-E*0103/0103 or K562 cells, but without peptides, respectively. SQAPLPCVL-specific, HLA-E-restricted CD8+ T cells and all remaining SQAPLPCVL-specific, HLA-E-restricted CD8+ T cells were sorted and analyzed for the expression of and NKG2C/CD94 by flow-cytometry.

### Results

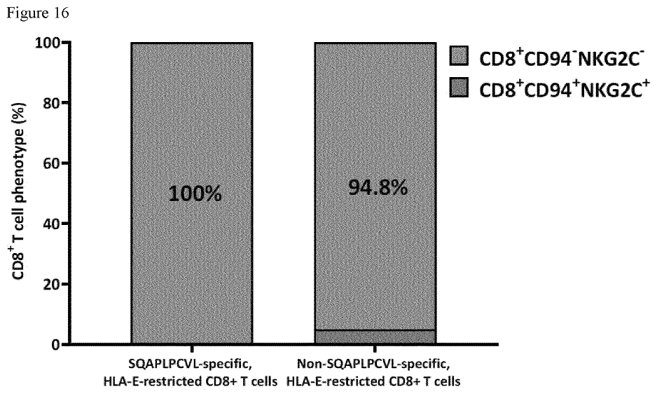

The results are shown in FIG. 16. FIG. 16 shows the mean (±SD) of the NKG2C/CD94-expressing and non-expressing SQAPLPCVL-specific, HLA-E-restricted CD8+ T cells as well all remaining SQAPLPCVL-specific, HLA-E-restricted CD8+ T cells, respectively. SQAPLPCVL-specific, HLA-E-restricted CD8+ T cells expressed no NKG2C/CD94. Thus, the SQAPLPCVL peptide elicits a SQAPLPCVL-specific HLA-E-restricted CD8+ T cell response that can be distinguished from a non SQAPLPCVL-specific HLA-E-restricted CD8+ T cell response.

### EXAMPLE 6

### Ineffective Immune Control of Epstein-Barr Virus-Induced Autoreactive Responses is an Important Cause of Multiple Sclerosis

### Summary

Multiple sclerosis (MS) is a demyelinating disease of the CNS. Epstein-Barr virus (EBV) contributes to the MS pathogenesis, as high-levels of EBV EBNA₃₈₆₋₄₀₅-specific antibodies cross-react with the CNS-derived GlialCAM₃₇₀₋₃₈₉. However, it is unclear why only some individuals with such high autoreactive antibody titers develop MS. Here we show that autoreactive cells are eliminated by distinct immune responses, which are determined by genetic variations of the host as well as of the infecting EBV- and Human Cytomegalovirus (HCMV). We demonstrate that potent cytotoxic NKG2C⁺ and NKG2D⁺ NK cells and distinct EBV-specific T-cell responses kill autoreactive GlialCAM₃₇₀₋₃₈₉-specific cells. Furthermore, immune evasion of these autoreactive cells was induced by EBV-variant-specific upregulation of the immunomodulatory HLA-E. These defined virus and host genetic pre-dispositions are associated with an up to 260-fold increased risk of MS. Our findings thus allow the early identification of patients at risk for MS and suggest new therapeutic options against MS.

### Introduction

Multiple sclerosis (MS) is a chronic autoinflammatory demyelinating disease of the central nervous system (CNS). Demyelination is the result of a pathological autoimmune-mediated inflammatory process, in which Epstein-Barr virus (EBV) may be critically involved 37. EBV is a ubiquitous herpesvirus that persistently infects more than 90% of the adult human population worldwide 38. Primary EBV infection causes infectious mononucleosis (IM) in about 13-22% of the patients, which is hallmarked by fever, lymphadenopathy, and tonsillitis². EBV establishes a lifelong persistent infection in memory B-cells, from which sporadic reactivations occur. Virus replication is controlled by classical and HLA-E-restricted CD8⁺ T-cells as well as by EBV-specific antibodies, which elicit antibody-dependent cellular phagocytosis (ADCP) and antibody-dependent cellular cytotoxicity (ADCC) responses against EBV-infected cells^{39,40} for example, an EBV specific HLA-E-restricted CD8⁺ T-cell response can be elicited by immunization via the SQAPLPCVL peptide inducing an SQAPLPCVL-specific HLA-E-restricted CD8+ T cell response; see Examples 1-5.

EBV is increasingly considered an important factor in MS pathogenesis. There is a substantially higher risk to develop MS after primary EBV infection³⁷, and this is especially linked to the development of high-level EBV EBNA-1-specific antibody titers in individual hosts⁴¹. Mechanistically, this is probably due to the molecular mimicry between an EBNA-1-derived antigen (EBNA₃₈₆₋₄₀₅) and the CNS glial cell adhesion molecule (GlialCAM₃₇₀₋₃₈₉) that results in cross-reactive EBNA₃₈₆₋₄₀₅-specific immune responses against the GlialCAM₃₇₀₋₃₈₉ peptide⁴⁰. Mouse studies revealed that auto-reactive EBNA₃₈₆₋₄₀₅-specific antibody responses lead to the development of MS-like demyelinating diseases and thus confirmed a link between the presence of EBNA-1-specific immune responses and MS^{40,41}.

However, since MS occurs only in a small fraction of EBV-infected individuals and EBNA₃₈₆₋₄₀₅-specific immune responses are also observed in healthy individuals, there must be additional factors that protect from the development of MS⁴². Observational studies indicated that infections with human Cytomegalovirus (HCMV)⁴³ may moderately protect against MS, although the underlying mechanisms are yet unclear^{37,44,45}.

The aim of this study was to investigate, which factors determine whether individuals, who develop high-level EBNA₃₈₆₋₄₀₅-specific autoimmune responses against the GlialCAM₃₇₀₋₃₈₉, further evolve into MS.

### Results

EBNA₃₈₆₋₄₀₅-specific IgG titers in MS patients and healthy controls Given that immune responses against EBNA₃₈₆₋₄₀₅ are cross-reactive against the human CNS-derived GlialCAM₃₇₀₋₃₈₉⁴⁰, we first investigated whether the presence of high EBNA₃₈₆₋₄₀₅-specific IgG titers in an individual are associated with MS. We recruited a cohort of 270 EBNA-1 seropositive MS patients. We then assessed the time since primary EBV infection in the patients and therefore collected follow-up plasma samples of all patients back to the time of their EBV seroconversion in a median interval of 4.2 months. The observed median time span between EBV seroconversion and MS diagnosis was 8.2 years. Seroconversion was accompanied by clinically evident IM in 202/270 (74.8%) of the patients. To compare the EBNA-1-specific antibody levels of MS patients with those of healthy persons, we included one EBV seropositive healthy individual for each MS patient, matched according to sex, age, time since EBV seroconversion, and occurrence of IM (Table 2). Furthermore, from all 270 control subjects, a follow-up was available for >5 years after the matching time point, and it was confirmed by follow-up records that none of them developed MS or other autoimmune diseases or neuro-inflammatory disorders within this follow-up period.

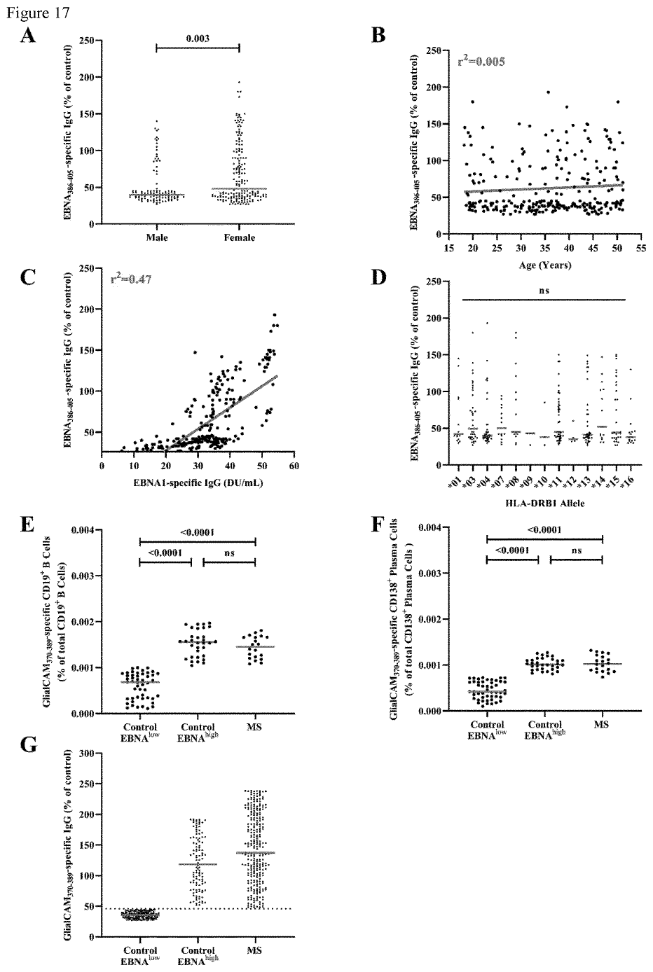

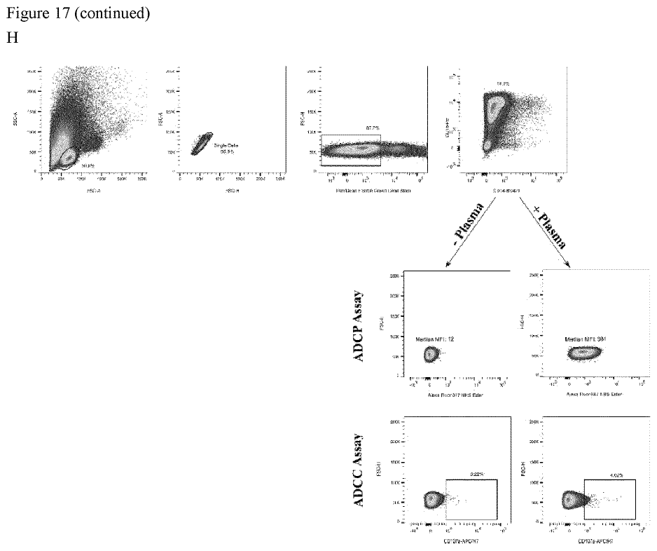

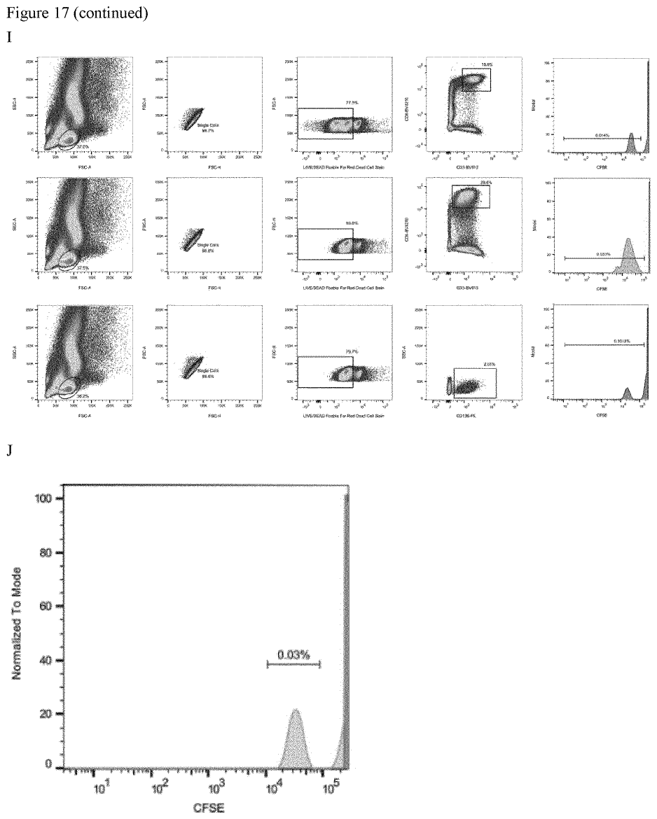

We then determined the EBNA₃₈₆₋₄₀₅-specific IgG antibody titers in healthy controls. As shown in FIG. 17A-B, females and individuals with high total EBNA-1-specific IgG levels had moderately elevated EBNA₃₈₆₋₄₀₅-specific IgG antibody titers. These findings are consistent with previously published studies that demonstrated that a female sex and high EBNA-1-specific IgG levels are independent risk factors for MS⁴⁶⁻⁴⁸. The age and the HLA-DRB1*15:01 allele, which is a known genetic risk factor for MS 49 were, however, not significantly associated with the EBNA₃₈₆₋₄₀₅-specific IgG antibody titers (FIG. 17C-D).

We then compared EBNA₃₈₆₋₄₀₅-specific IgG antibody titers between patients and matched control persons. All MS patients showed EBNA₃₈₆₋₄₀₅-specific IgG antibody levels above a threshold of 46% normalized to a reference plasma sample. EBNA₃₈₆₋₄₀₅-specific IgG titers were significantly higher in MS patients than in the overall control cohort (median 95.5% and 42%, respectively, p<0.0001, Mann-Whitney-Test). However, EBNA₃₈₆₋₄₀₅-specific IgG antibody levels above the 46% cut-off were also observed in 108/270 (40%) of control individuals.

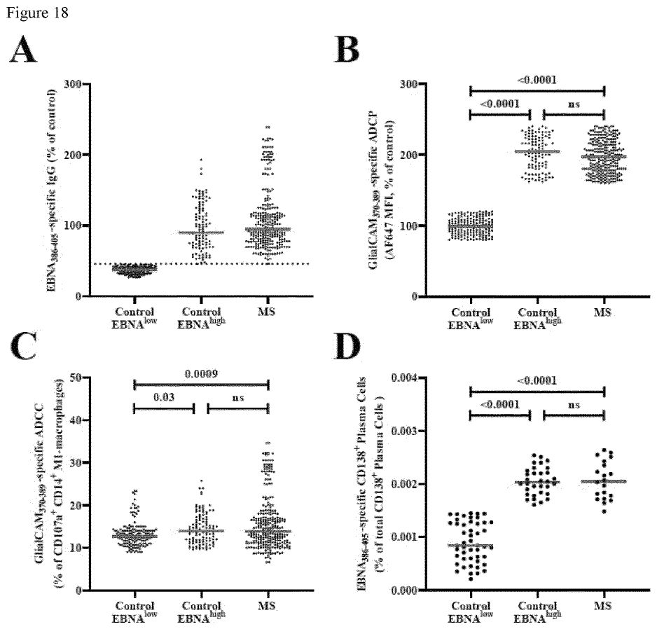

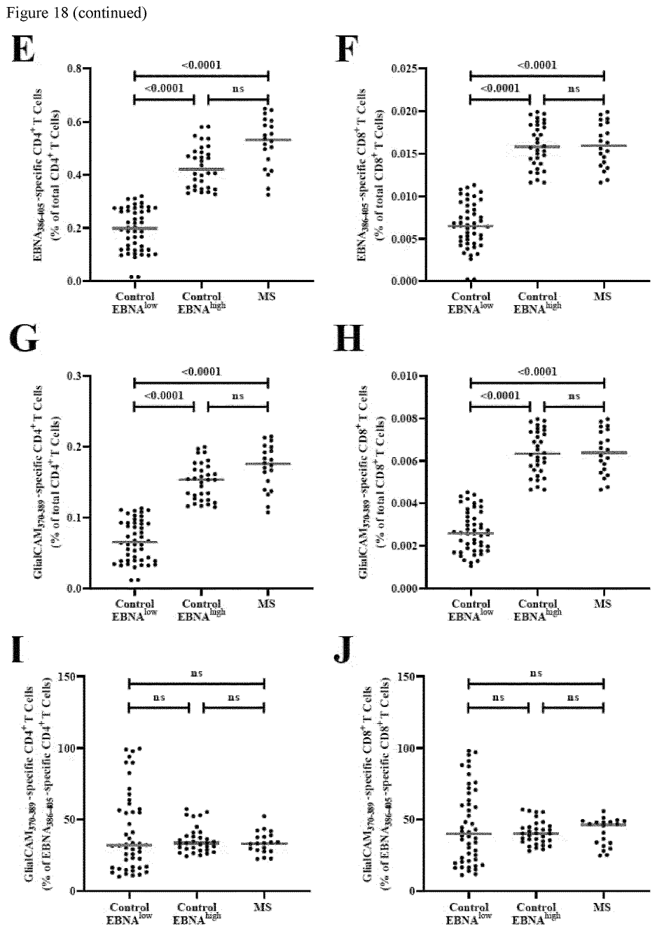

This raises the question of what protects healthy individuals with high EBNA₃₈₆₋₄₀₅-specific IgG antibody levels from the development of MS. For further analyses, we thus stratified the control cohort into individuals with low (EBNA^{low}) and high (EBNA^{high}) EBNA₃₈₆₋₄₀₅-specific IgG antibody levels (FIG. 18A, Table 2, Table 3).

EBNA₃₈₆₋₄₀₅- and GlialCAM₃₇₀₋₃₈₉-Specific Immune Responses in MS Patients and Healthy Controls

We then analyzed whether there are differences in the cross-reactive immune responses elicited by EBNA₃₈₆₋₄₀₅-specific IgG antibodies against the human CNS-derived GlialCAM₃₇₀₋₃₈₉ peptide between MS patients, EBNA^{high}, and EBNA^{low} controls. Therefore, we first evaluated the GlialCAM₃₇₀₋₃₈₉-specific B-cell, plasma cell and IgG antibody levels in MS patients, EBNA^{high}, and EBNA^{low} controls. As shown in FIG. 17E-G, MS patients and EBNA^{high} controls had significantly higher GlialCAM₃₇₀₋₃₈₉-specific B-cell, plasma cell and IgG antibody levels, compared to EBNA^{low} controls. We then also investigated whether and to which extent EBNA₃₈₆₋₄₀₅-specific IgG antibodies elicit ADCP and ADCC responses against the GlialCAM₃₇₀₋₃₈₉ peptide. As M1-macrophages are highly prevalent in MS lesions and contribute to the pathogenesis of MS⁵⁰, we used M1-macrophages, derived from 12 healthy EBV-seropositive blood donors, as effector cells for the analyses. We found that EBNA₃₈₆₋₄₀₅-specific IgG antibodies led to GlialCAM₃₇₀₋₃₈₉-specific ADCP (FIG. 18B, FIG. 17H) and ADCC (FIG. 18C, FIG. 17H). Both responses were significantly higher in MS patients and EBNA^{high} controls compared to EBNA^{low} controls.

We further assessed to which extent EBV infections elicit EBNA₃₈₆₋₄₀₅-specific plasma cell, CD4⁺ T-cell and CD8⁺ T-cell responses in patients and controls. PBMCs were obtained from N=20 MS patients (7.4%), and for each MS patient, PBMCs from N=2-3 EBNA^{low} (N=48) and N=1-2 EBNA^{high} (N=32) matched controls were available, respectively. MS patients and EBNA^{high} controls showed significantly higher EBNA₃₈₆₋₄₀₅-specific plasma cell (FIG. 18D, FIG. 17I), CD4⁺ T-cell (FIG. 18E, FIG. 17I-J), and CD8⁺ T-cell (FIG. 18F, FIG. 17I) levels compared to the EBNA^{low} group.

We further analyzed whether EBNA₃₈₆₋₄₀₅-specific T-cells are also functional against the GlialCAM₃₇₀₋₃₈₉ peptide. CD4+(FIG. 18G) and CD8⁺ T-cells (FIG. 18H) were found to be reactive against GlialCAM₃₇₀₋₃₈₉, but significantly more cells were reactive in MS patients and EBNA^{high} controls than in the EBNA^{low} controls. To evaluate whether the same frequency of EBNA₃₈₆₋₄₀₅-specific T-cells cross-reacted with GlialCAM₃₇₀₋₃₈₉, we stimulated sorted EBNA₃₈₆₋₄₀₅-specific CD4⁺ (FIG. 18I) and CD8⁺ (FIG. 18J) T-cells with GlialCAM₃₇₀₋₃₈₉ peptide. EBNA₃₈₆₋₄₀₅-specific CD4⁺ and CD8⁺ T-cells from MS patients, EBNA^{high}, and EBNA^{low} controls showed similar reactivities against GlialCAM₃₇₀₋₃₈₉.

Thus, both MS patients and EBNA^{high} healthy controls exhibit similarly high-level EBNA₃₈₆₋₄₀₅-specific immune responses with potent effector functions against GlialCAM₃₇₀₋₃₈₉ compared to EBNA^{low} controls.

### Cellular Cytotoxicity Against GlialCAM₃₇₀₋₃₈₉-Specific Cells

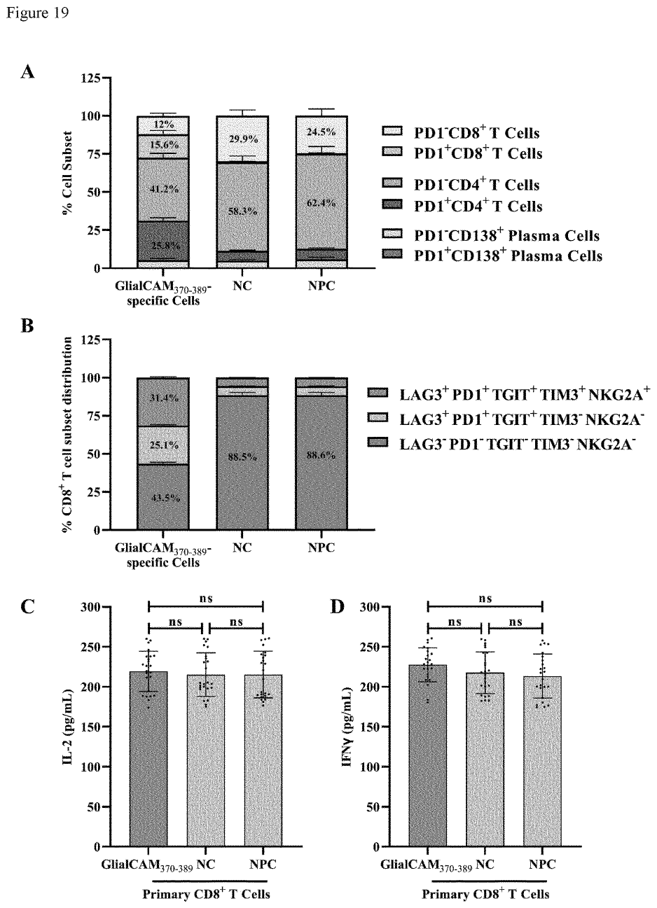

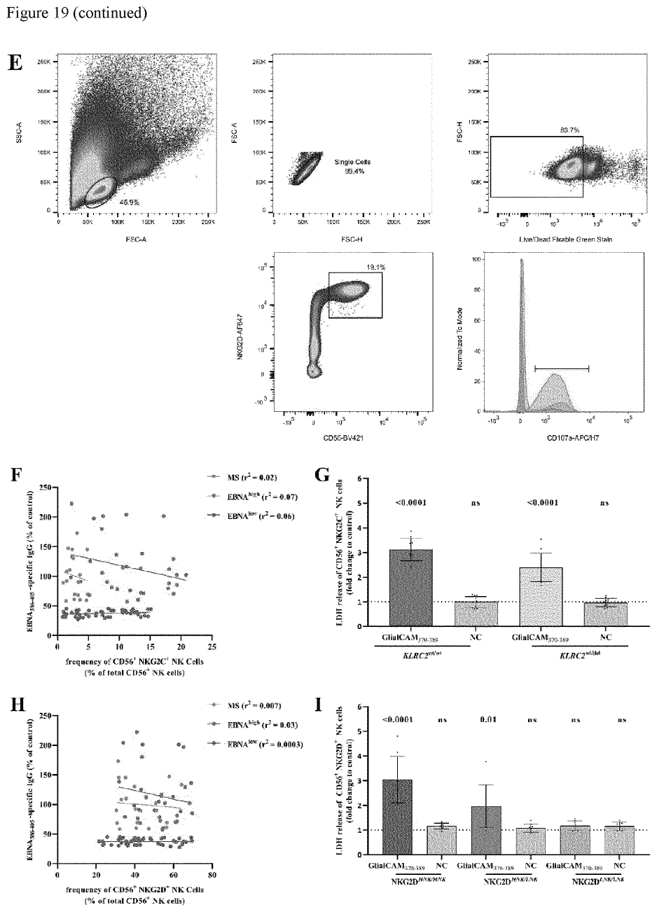

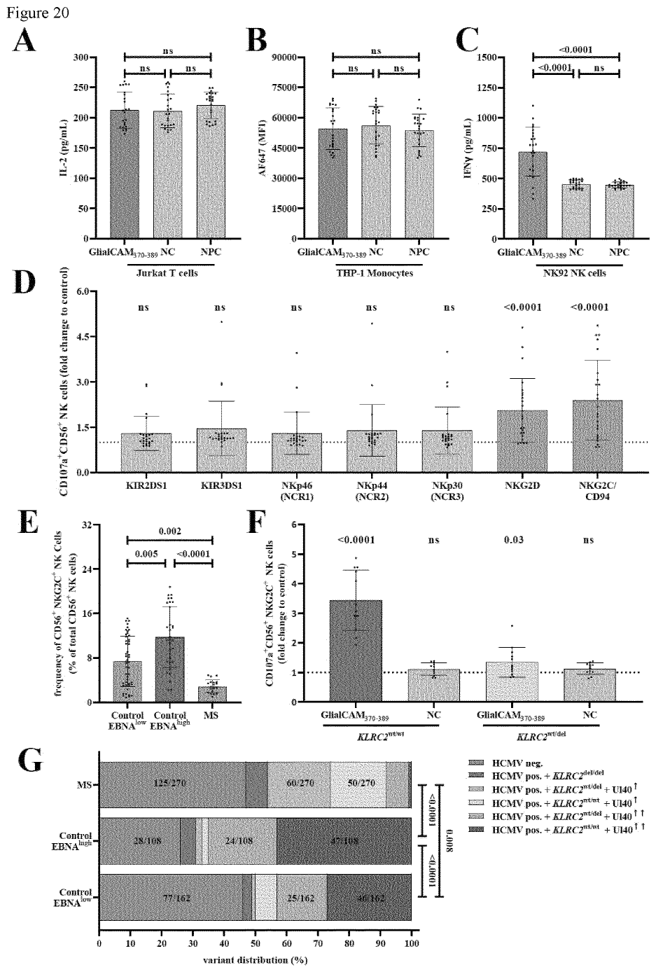

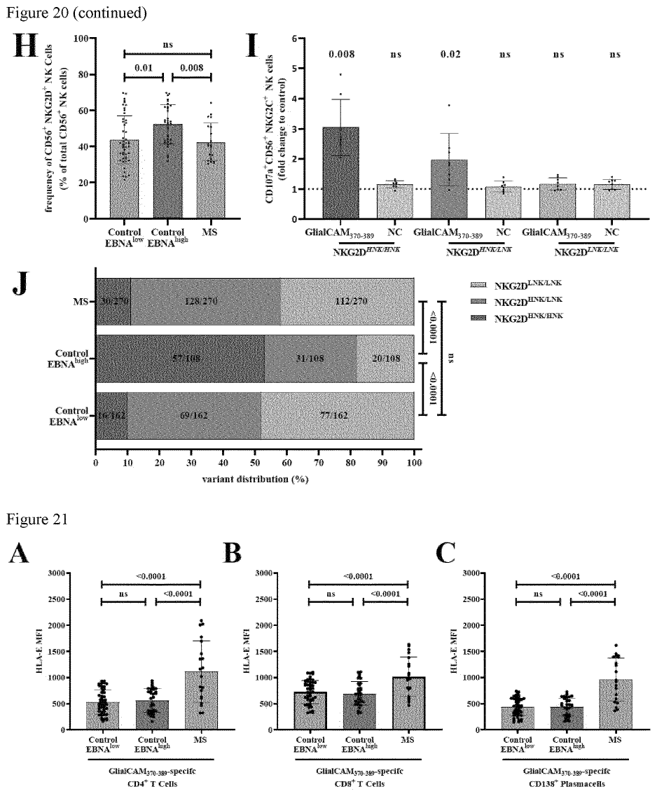

As we found no differences in the cross-reactive immune responses between MS patients and healthy individuals with high EBNA₃₈₆₋₄₀₅-specific IgG antibody levels, we hypothesized that the EBNA^{high} controls, in contrast to the MS patients, may be able to sufficiently suppress these GlialCAM₃₇₀₋₃₈₉-specific immune responses, and thereby evade the development of MS. To test this hypothesis, we extensively stimulated PBMCs from 24 healthy individuals with the GlialCAM₃₇₀₋₃₈₉ peptide until the majority of GlialCAM₃₇₀₋₃₈₉-specific CD8⁺ T-cells expressed the exhaustion marker PD-1 and >30% of GlialCAM₃₇₀₋₃₈₉-specific CD8⁺ T-cells showed a terminally exhausted⁵¹ LAG3⁺PD1⁺TGIT⁺TIM⁺NKG2A⁺ phenotype (FIG. 19A-B). We then sorted GlialCAM₃₇₀₋₃₈₉-specific T and B-cells and co-cultured these stressed autoimmune GlialCAM₃₇₀₋₃₈₉-specific with isolated CD8⁺ T-cells from the same donor or Jurkat T-cell, THP-1 monocyte, or NK92 NK cell lines. We then analyzed the phagocytosis and the T- and NK cell-mediated activation against these cells (FIG. 20A-C, FIG. 19C-D). Only NK92 cells responded to the cells, stressed by GlialCAM₃₇₀₋₃₈₉, and developed activation marker levels, i.e. secreted IFNγ levels, which were significantly above that of non-stressed control cells. We further analyzed the activation of distinct primary NK cell subsets and therefore co-cultured stressed GlialCAM₃₇₀₋₃₈₉-specific T and B-cells from 24 healthy EBV- and HCMV-seropositive individuals with sorted KIR2DS1⁺, KIR3DS1⁺, NKp46⁺, NKp44⁺, NKp30⁺, NKG2D⁺, or NKG2C⁺ NK cells from the same donor. Only NKG2C⁺ (FIG. 20D) and NKG2D+(FIG. 20D, FIG. 19E) NK cells showed significantly increased activation levels elicited by stressed GlialCAM₃₇₀₋₃₈₉-specific cells.

NKG2C⁺ NK cell-mediated cellular cytotoxicity against GlialCAM₃₇₀₋₃₈₉-specific cells We thus evaluated the role of NKG2C⁺ NK cells in the development of MS and first determined peripheral blood-derived NKG2C⁺ NK cell levels in patients and controls. MS patients showed significantly lower NKG2C⁺ NK cell levels than EBNA^{high} and EBNA^{low} controls, respectively (FIG. 20E, FIG. 19F). As a high level of cytotoxic NKG2C⁺ NK cells is caused by prior HCMV infection, we tested MS patients and controls for the presence of HCMV-specific IgG antibodies, which indicates a prior HCMV infection. Significantly more EBNA^{high} controls were HCMV seropositive compared to EBNA^{low} controls and MS patients (Table 3).

Functionally, the expansion of NKG2C⁺ NK cells in response to HCMV infections depends on a highly polymorphic HCMV UL40-encoded peptide in the respective infecting HCMV variant, which upregulates the NKG2C ligand HLA-E on the surface of HCMV-infected cells⁵². As the individual UL40 variants have different effects on HLA-E upregulation, we further determined the UL40 variants in the infecting HCMV variants of MS patients and controls. HCMV variants were obtained from all 145 HCMV-seropositive MS patients during an episode of HCMV reactivation between EBV seroconversion and MS diagnosis, as well as from all HCMV seropositive EBNA^{high} (N=80) and EBNA^{low} (N=85) controls in any of the serum samples collected from the individuals over time. We identified 18 different UL40 variants in the infecting HCMV variants (Table 4) and then tested the ability of all variants to stabilize HLA-E on the cell surface. Therefore, we incubated HLA-E-expressing RMA-S cells with each of the 18 UL40 peptides and measured HLA-E expression on the cell surface. 95% and 85.4% of EBNA^{high} and EBNA^{low} controls, respectively, carried HCMV variants that encoded UL40 variants leading to a high-level of HLA-E stabilization, as defined by an HLA-E upregulation of >99.5%. However, only 20.7% of MS patients were infected with such HCMV variants (Table 4).

The activation and proliferation of NKG2C⁺ NK cells also depend on a specific deletion in the human NKG2C-encoding KLRC2 gene. Individuals with the KLRC2^{wt/del} variant express lower NKG2C NK levels, while individuals with the KLRC2^{del/del} genotype lack NKG2C⁺ NK cells⁵³. To assess the impact of these variants on the development of MS, we tested the occurrence of the KLRC2 variants in MS patients and controls. KLRC2^{wt/del} and KLRC2^{del/del} variants were significantly over-represented in MS patients (Table 5). To analyze further the impact of the KLRC2 variants on the NKG2C⁺ NK cell-mediated cytotoxicity, we co-cultured stressed GlialCAM₃₇₀₋₃₈₉-specific T and B-cells from 24 healthy individuals with NKG2C⁺ NK cells encoding either the KLRC2^{wt/wt} or KLRC2^{wt/del} variant from the same healthy blood donors. KLRC2^{wt/wt} elicited increased cytotoxic responses against stressed GlialCAM₃₇₀₋₃₈₉ specific cells, compared to KLRC2^{wt/del} variants (FIG. 20F, FIG. 19G).

Finally, we assessed the distribution of the combination of the following parameters: HCMV infection, UL40 variants of the infecting HCMV variant, and KLRC2_{wt/del} genotype, in patients and control individuals. MS patients showed extremely rarely the KLRC2^{wt/wt} genotype and a high binding UL40 peptide. This is in significant contrast to the EBNA^{high} and EBNA^{low} controls, in whom these specific combinations reflecting a high-level NKG2C⁺ NK cell response dominated (FIG. 20G). Thus, our data demonstrate that potent NKG2C⁺ NK cell responses can efficiently control GlialCAM₃₇₀₋₃₈₉-specific stressed cells, but that these responses are overall absent or weak in MS patients.

NKG2D⁺ NK Cell-Mediated Cellular Cytotoxicity Against GlialCAM₃₇₀₋₃₈₉-Specific Cells

To further evaluate whether the NKG2D⁺ NK cell-mediated control of GlialCAM₃₇₀₋₃₈₉-specific cells can prevent the development of MS, we first determined the peripheral blood-derived NKG2D⁺ NK levels in MS patients and controls. EBNA^{high} controls showed significantly higher NKG2D⁺ NK levels than MS patients and EBNA^{low} controls (FIG. 20H, FIG. 19H). As the proliferation and activation of NKG2D⁺ NK cells depend on distinct genetic variants, which result in an either low (LNK) or high NK cytotoxicity (HNK) phenotype⁵⁴, we further tested whether these NKG2D^{HNK/LNK} variants have an impact on the development of MS. We co-cultured stressed GlialCAM₃₇₀₋₃₈₉-specific T and B-cells from 24 healthy individuals with NKG2D⁺ NK cells, expressing either the NKG2D^{HNK/HNK}, NKG2D^{HNK/LNK} or NKG2D^{LNK/LNK} genotype, respectively, from the same healthy blood donor. The NKG2D^{HNK/HNK} showed a significantly increased cytotoxic response against the stressed GlialCAM₃₇₀₋₃₈₉ cells (FIG. 20I, FIG. 19I). We then compared the distribution of the NKG2D^{HNK/LNK} variants between patients and controls. The highly cytotoxic homozygous NKG2D^{HNK/LNK} variant was significantly over-represented in EBNA^{high} controls, whereas the NKG2D^{HNK/LNK} and NKG2D^{LNK/LNK} genotypes (FIG. 20J) occurred more frequently in MS patients and EBNA^{low} controls. Thus, potent NKG2D⁺ NK cell responses can efficiently kill GlialCAM₃₇₀₋₃₈₉-specific cells and are highly prevalent in EBNA^{high} controls.

### Stressed GlialCAM₃₇₀₋₃₈₉-Specific Cells Up-Regulate HLA-E

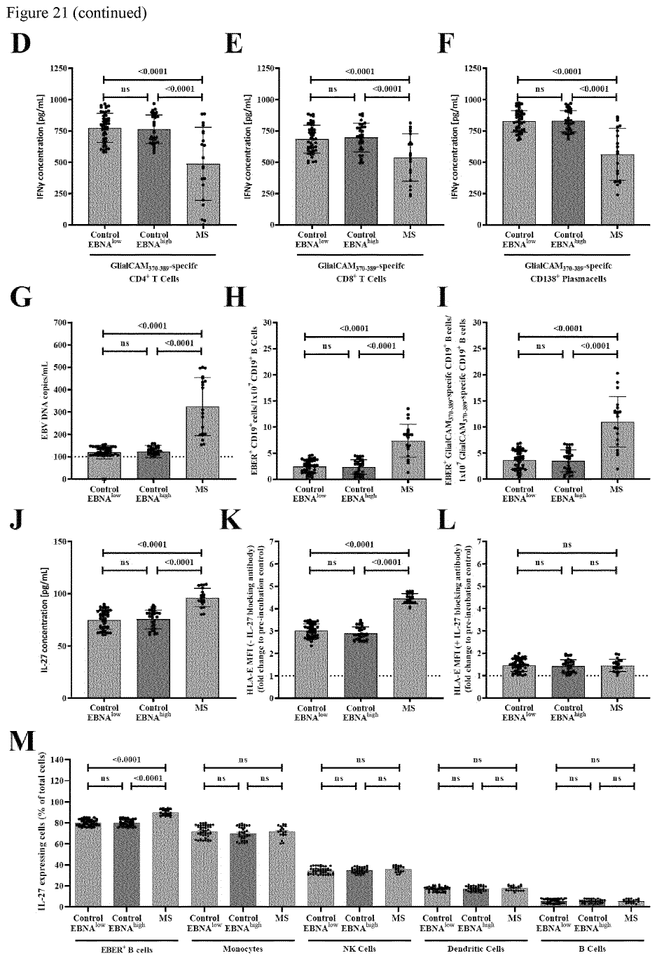

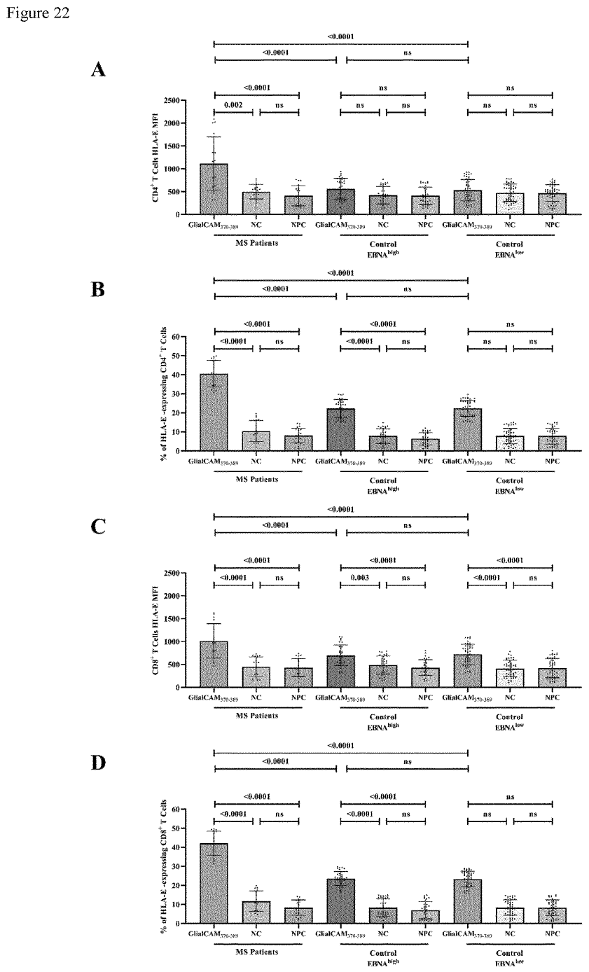

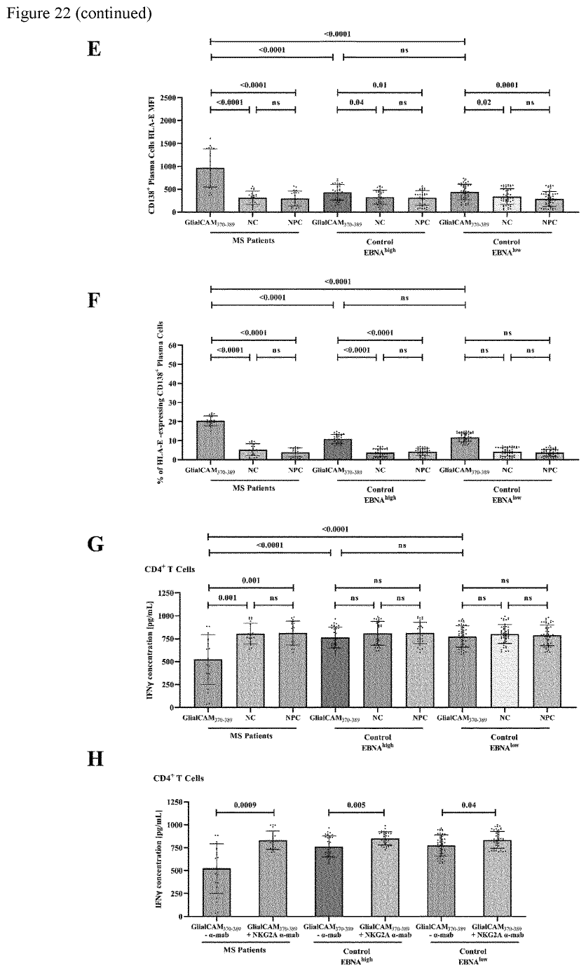

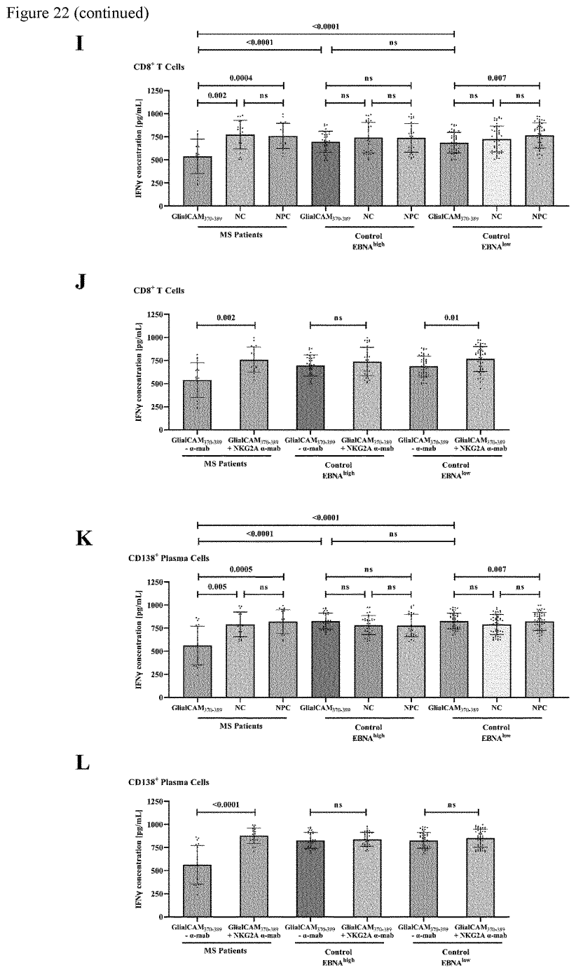

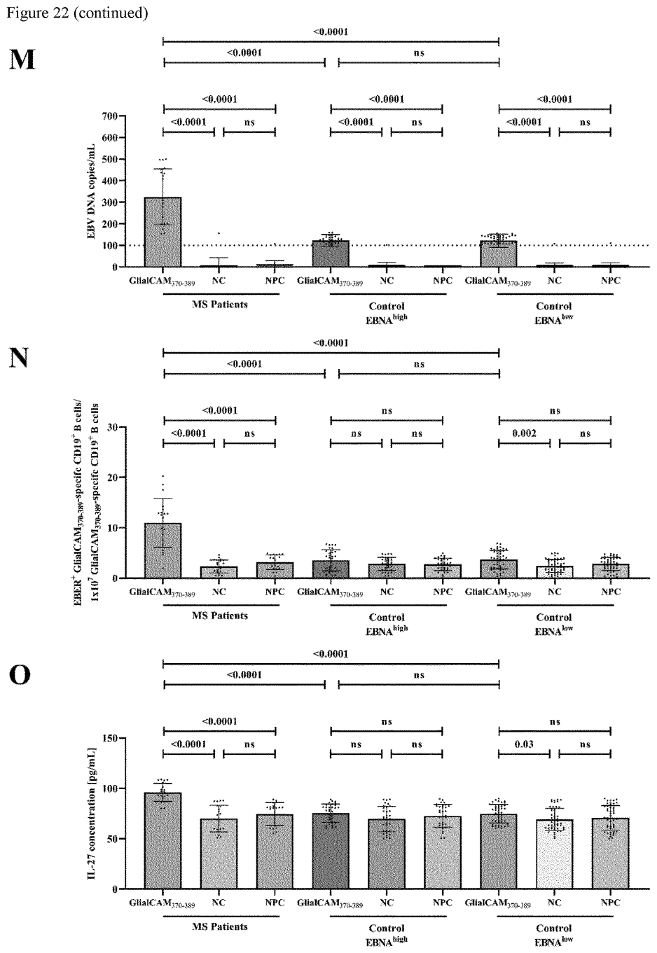

As the killing of stressed GlialCAM₃₇₀₋₃₈₉-specific cells by distinct cytotoxic NK cell responses seems to be a key factor to avoid development of MS, we further hypothesized that also cellular resistance mechanisms to cytotoxicity may contribute to MS pathogenesis. Cellular resistance may be caused by the upregulation of HLA-E on the cell surface. This upregulation of HLA-E prevents stressed cells from being killed via the engagement of the inhibitory NKG2A receptor⁵⁵. We therefore first determined the frequency of HLA-E expressing cells as well as the level of HLA-E expression on PBMCs stimulated with GlialCAM₃₇₀₋₃₈₉. GlialCAM₃₇₀₋₃₈₉-specific CD4⁺ T-cells (FIG. 21A, FIG. 22A-B), CD8⁺ T-cells (FIG. 21B, FIG. 22C-D), and plasma cells (FIG. 21C, FIG. 22E-F) from MS patients showed significantly more HLA-E-expressing cells as well as higher HLA-E expression levels than cells derived from EBNA^{high} or EBNA^{low} controls.

To further evaluate whether the high HLA-E expression in MS patients translates into potent inhibition of NKG2A⁺ NK cells, we co-cultured the sorted GlialCAM₃₇₀₋₃₈₉-specific cells with NKG2A⁺ NKG2D⁻NKG2C⁻ NK cells, derived from N=24 healthy blood donors. GlialCAM₃₇₀₋₃₈₉-specific cells from patients with MS showed a significant inhibition of NKG2A⁺ cells (FIG. 21D-F, FIGS. 22G,I,K). This effect, however, was reversible, when monoclonal NKG2A-blocking antibodies (Monalizumab) were added to the co-culture (FIGS. 22H,J,L). Thus, GlialCAM₃₇₀₋₃₈₉-specific cells from patients with MS can efficiently evade regulatory and cytotoxic immune responses via the upregulation of HLA-E and the resulting inhibition of NKG2A⁺ cells.

### EBV-Infected Cell-Derived IL-27-Mediated Immune Evasion of GlialCAM₃₇₀₋₃₈₉-Specific Cells

HLA-E expression can be triggered by EBV-reactivation in infected B-cells and the EBV-encoded LMP-1 protein thereby induces the secretion of IL-27, which may further upregulate HLA-E in adjacent non-infected cells⁵⁶. We thus investigated whether EBV-reactivation is higher in MS patients compared to control individuals. We stimulated PBMCs from MS patients, EBNA^{high}, and EBNA^{low} controls, with the GlialCAM₃₇₀₋₃₈₉-peptide and then measured the EBV DNA load and the frequency of EBV-infected B-cells in the supernatant. A significantly higher EBV DNA load (FIG. 21G, FIG. 22M) and significantly more EBV-infected total (FIG. 21H) as well as GlialCAM₃₇₀₋₃₈₉-specific (FIG. 21I, FIG. 22N) B-cells were identified in MS patients-derived samples than in those of both control groups.

To assess the role of EBV-mediated IL-27 secretion in the pathogenesis of MS, we also measured the IL-27 concentration in the supernatant of the GlialCAM₃₇₀₋₃₈₉ stimulated PBMCs from patients and controls. PBMCs, derived from MS patients produced significantly more IL-27 than those obtained from EBNA^{high} and EBNA^{low} controls (FIG. 21J. FIG. 22O). When we then transferred the supernatant onto fresh PBMCs from the same donor, the IL-27-containing supernatant increased HLA-E expression, especially in cells obtained from MS patients (FIG. 21K). After addition of an IL-27 blocking antibody, this effect was, however, no longer observed (FIG. 21L).

To finally assess which cells secrete high IL-27 levels in response to the GlialCAM₃₇₀₋₃₈₉-stimulation, we measured the percentage of distinct IL-27-expressing cell subsets after stimulation. MS-patient-derived EBV-infected B-cells showed somewhat more IL-27-expressing cells compared to EBNA^{high} and EBNA^{low} control individuals (FIG. 21M). Our data thus implicate that the stimulation of B-cells derived from MS patients results in more EBV-infected as well as more IL-27-expressing cells than from healthy control persons. The increased EBV-reactivation and IL-27 expression are subsequently correlated with increased HLA-E expression and inhibition of NKG2A⁺ effector cells in MS patients.

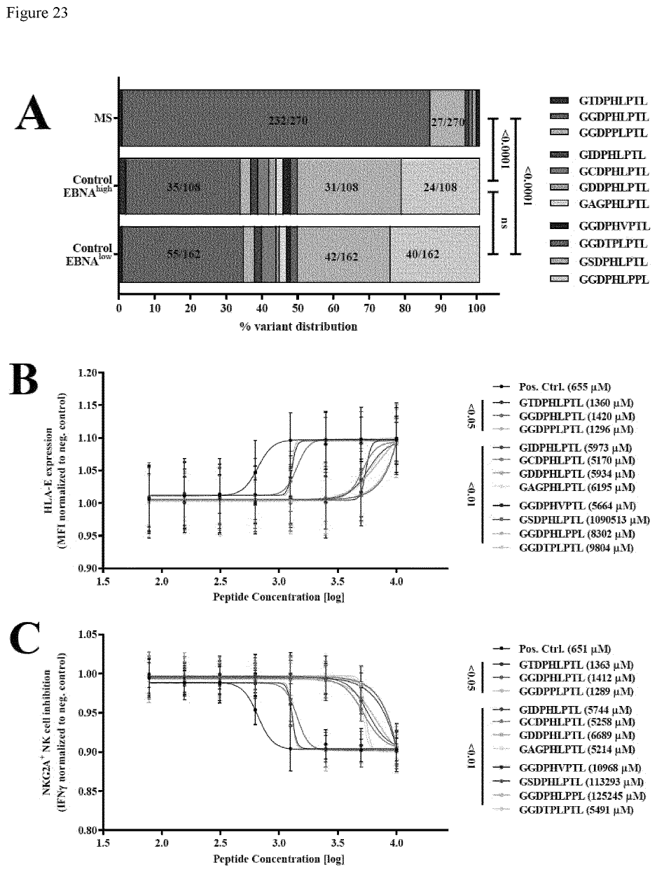

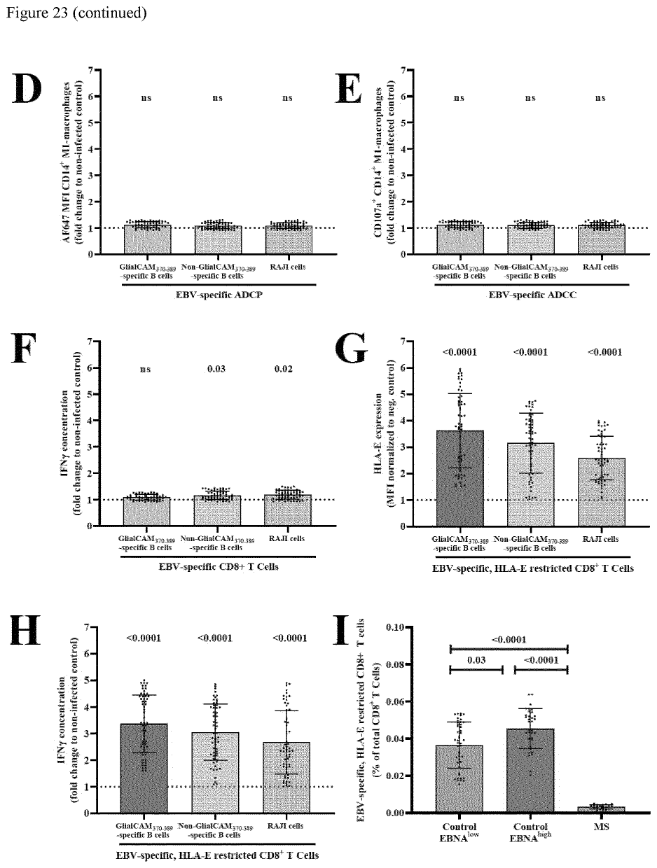

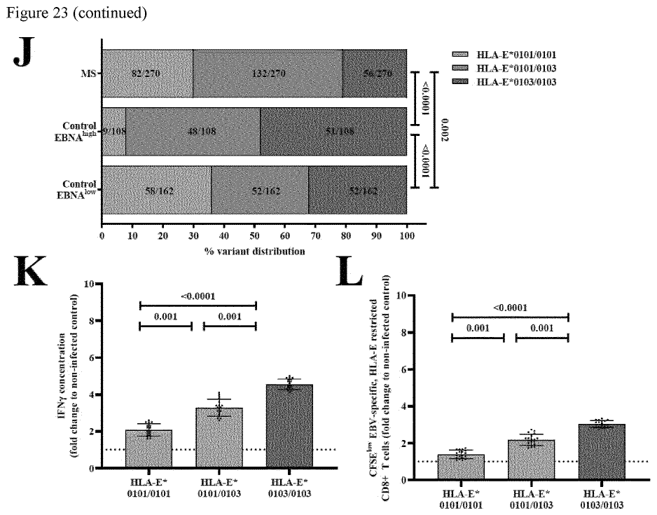

EBV LMP-1 peptides mediate immune evasion in MS patients Independent of IL-27, HLA-E is also stabilized by an EBV LMP-1-derived peptide, which is expressed in latently EBV-infected cells and mediates immune evasion from NKG2A-expressing NK cells⁵⁷. As this peptide is highly polymorphic and its variants differ in affinity to HLA-E⁵⁷, we hypothesized that distinct EBV variants, encoding for highly affine LMP-1 peptides, may increase the expression of HLA-E on the surface of GlialCAM₃₇₀₋₃₈₉-specific cells, thereby facilitating development of MS. To clarify this, we sequenced all EBV-isolates identified in the individual MS patients and controls, obtained either during primary infection in patients with IM or during an episode of EBV reactivation in asymptomatic individuals. EBNA^{high} and EBNA^{low} controls showed a high diversity that included 11 different LMP-1-peptide variants. In contrast, a limited peptide diversity was found in patients with MS, hallmarked by the high frequency of the GGDPHLPTL and GGDPPLPTL variants (FIG. 23A). Based on these results, we further investigated whether EBV isolates carrying the GGDPHLPTL and GGDPPLPTL LMP-1-peptide variants were associated with an especially efficient HLA-E-upregulation and inhibition of NKG2A⁺ NK cells, and thereby with a potent immune evasion. Therefore, we transfected GlialCAM₃₇₀₋₃₈₉-specific B-cells from 24 healthy blood donors with varying concentrations of each of the 11 individual LMP-1 peptides identified in patients and control individuals.

Transfection with GGDPHLPTL, GGDPPLPTL, and the rarely occurring GTDPHLPTL peptide variant led to a stable upregulation of HLA-E on the surface of GlialCAM₃₇₀₋₃₈₉-specific B-cells (FIG. 23B). To confirm, whether this LMP-1-triggered HLA-E upregulation is also associated with potent inhibition of NKG2A⁺ cells, we co-cultured the LMP-1 peptide-pulsed GlialCAM₃₇₀₋₃₈₉-specific B-cells with NKG2A⁺ NKG2D⁻NKG2C⁻ NK cells and subsequently measured the IFNγ concentration in the supernatant. LMP-1 GGDPHLPTL and GGDPPLPTL peptides potently inhibited NKG2A⁺ cells (FIG. 23C).

In summary, we found that nearly all MS patients carried EBV variants with two LMP-1 variants, which caused potent HLA-E upregulation and inhibition of NKG2A⁺ cells. This further contributes to the immune evasion of EBV-infected GlialCAM₃₇₀₋₃₈₉-specific B-cells, and thereby potentially to development of MS.

Killing of EBV-Infected GlialCAM₃₇₀₋₃₈₉-Specific Cells by HLA-E-Restricted CD8⁺ T-Cells

As our data revealed that EBV-infected GlialCAM₃₇₀₋₃₈₉-specific B-cells may drive the development of MS, we next hypothesized that the efficient control of EBV-infected GlialCAM₃₇₀₋₃₈₉-specific B-cells may prevent the development of MS. To analyze, which EBV-specific immune responses can specifically control these cells, we next evaluated the total EBV-specific ADCP, ADCC, and overall CD8⁺ T-cell responses against EBV-infected GlialCAM₃₇₀₋₃₈₉-specific B-cells. GlialCAM₃₇₀₋₃₈₉-specific B-cells or non-GlialCAM₃₇₀₋₃₈₉-specific B-cells were isolated from 60 healthy blood donors and then in vitro infected with the EBV B95-8 strain. As an additional control, the RAJI B-cell line was also infected with EBV B95-8 strain.

Neither EBV-specific ADCP, ADCC nor EBV-specific CD8⁺ T-cell responses were elicited, as demonstrated by a similar activation in response to EBV-infected and non-infected GlialCAM₃₇₀₋₃₈₉-specific B-cells (FIG. 23D-F). However, as EBV-infected GlialCAM₃₇₀₋₃₈₉-specific B-cells highly express HLA-E (FIG. 23G), we further specifically analyzed the ability of EBV-specific, HLA-E-restricted CD8⁺ T-cells to kill EBV-infected GlialCAM₃₇₀₋₃₈₉-specific B-cells. We co-cultured EBV-infected GlialCAM₃₇₀₋₃₈₉-specific B-cells from 60 healthy blood donors with EBV-specific, HLA-E-restricted CD8⁺ T-cells from the same donor. As shown in FIG. 23H, these cells showed a robust response against EBV-infected GlialCAM₃₇₀₋₃₈₉-specific B-cells. When we further analyzed the frequency of EBV-specific, HLA-E-restricted CD8⁺ T-cells in patients and control individuals, we observed that MS patients showed significantly lower EBV-specific, HLA-E-restricted CD8⁺ T-cell levels than EBNA^{high} and EBNA^{low} controls (FIG. 23I).

EBV-specific, HLA-E-restricted CD8⁺ T-cell levels are correlated with host genetic HLA-E variants encoding HLA-E*0101 or HLA-E*0103 alleles and are higher in individuals encoding the high-expressing HLA-E*0103/0103 variant 39. Therefore, we determined HLA-E*0101/0103 variants in MS patients and controls. The HLA-E*0103/0103 variant was significantly overrepresented in EBNA^{high} controls, whereas the HLA-E*0101/0103 and HLA-E*0101/0101 dominated in MS patients and EBNA^{low} control individuals (FIG. 23J).

To further evaluate the impact of the HLA-E variants, we co-cultured EBV-infected GlialCAM₃₇₀₋₃₈₉-specific B-cells, encoding for HLA-E*0101/0101 (N=20), HLA-E*0101/0103 (N=20), or HLA-E*0103/0103 (N=20), respectively, with EBV-specific, HLA-E-restricted CD8⁺ T-cells. The HLA-E*0103/0103 variant was associated with significantly increased activation (FIG. 23K) and proliferation (FIG. 23L) of HLA-E-restricted CD8⁺ T-cells in response to EBV-infected GlialCAM₃₇₀₋₃₈₉-specific B-cells.

### Individual and Combined Risk Factors for MS

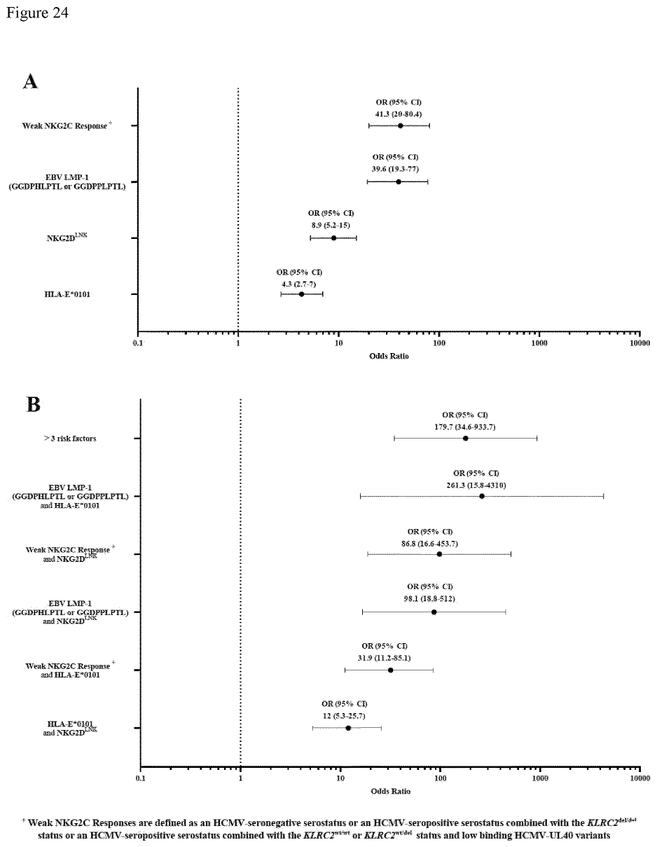

Based on our findings, we finally analyzed to which extent a single marker is associated with protection from MS in the healthy EBNA^{high} control individuals, despite their high EBNA^{high} IgG antibody titers. Especially the weak or absent NKG2C⁺ NK cell response, including the HCMV-serostatus, the UL40 peptide variant of the infecting HCMV variant, and the KLRC2 status (OR: 41.3) as well as the LMP-1 peptide variants encoded by the infecting EBV variant (OR: 39.6) were significantly overrepresented in healthy EBNA^{high} controls and associated with protection from MS (FIG. 24A).

Finally, we investigated whether there was a specific combination of factors that could predict the individual likelihood of protection from MS in individuals with high EBNA-1 antibody levels. The combination of risk LMP-1 variants and the HLA-E*0103/0103 variant in the host as well as the occurrence of ≥3 risk factors were associated with a 261.3- and 179.7-fold increased risk to develop MS in addition to high EBNA₃₈₆₋₄₀₅ IgG antibody titers, respectively (FIG. 24B).

### Discussion

Here, we report that MS pathogenesis is based on an ineffective control of EBNA-1-mediated autoimmune response. The development of MS was earlier associated with high cross-reactive EBNA-1 specific antibody levels in humans⁴⁰, albeit the majority of EBNA^{high} individuals do not develop MS. Our data now provide evidence that the main protective factors against MS are distinct cytotoxic NK cell responses. So far, the role of NK cells in the development of MS remained inconclusive and while some studies reported high NK cell numbers in the cerebrospinal fluid of MS patients and a contribution to the MS immunopathology, other studies highlighted the immunoregulatory role of NK cells⁵⁸. Here, we demonstrate that only specific cytotoxic NK cell responses, in particular the well-characterized NKG2C⁺ and NKG2D⁺ NK cell subsets 59-61 are correlated with the control of GlialCAM₃₇₀₋₃₈₉-specific autoreactive cells. We found that the efficacy of the NKG2C⁺ NK cell responses was significantly associated with previous infection with HCMV, which lead to high NKG2C⁺ NK cell levels, as the previously reported result of an HCMV-specific imprint in the NK cell repertoire⁶². So far, the role of HCMV in the MS pathogenesis is not clarified. However, earlier observational studies demonstrated a somewhat lower MS prevalence in HCMV seropositive individuals and suggested that infections with HCMV may moderately contribute to protection against MS^{37,44,45,63,64}. Other studies reported a better clinical outcome in HCMV seropositive MS patients and authors suggested that HCMV-mediated anti-inflammatory and immunomodulatory effects may have a protective effect^{64,65} We now revealed that the protection against MS was not only associated with previous HCMV infections but was highly dependent on the UL40 peptide variant encoded by the respective infecting HCMV isolates. VMAPRTLVL, VMAPRTLIL, VMAPRTLLL, and VMAPRTLFL UL40 peptides elicited a more potent NKG2C⁺ NK cell response, which was further associated with protection from MS in our study cohort. Protection was, however, only achieved in persons who did not display a homozygous deletion of the KLRC2 receptor and were thus able to confer an NKG2C⁺ NK cell response. These findings explain the earlier identified moderate association between both KLRC2 deletion variants and MS⁶⁶. Our data revealed that HCMV-seropositive individuals with high level of EBNA-1-specific antibodies who carry a highly potent UL40 peptide variant as well as the wild-type KLRC2 genotype displayed a 41.3-fold lower likelihood of developing MS. Our data are of special interest, as recently published studies correlated high NKG2C⁺ NK cell levels in MS patients with a low disability score and a decreased risk of disability progression^{45,66} It is thus likely that the NKG2C⁺ NK cell response can also positively influence MS course. Furthermore, the data also suggest that therapeutic vaccination with the specific highly potent UL40 peptides could be a new option to limit the progression of MS, by leading to high-level NKG2C+NK cell responses.

We also showed that an efficient NKG2D⁺ NK cell response was protective against MS and could efficiently kill stressed GlialCAM₃₇₀₋₃₈₉-specific autoreactive cells. MS patients displayed overall low NKG2D⁺ NK cell levels and encoded significantly more often for the low cytotoxicity NKG2D^{LNK} genetic variants. NKG2D⁺ NK cells recently gained interest as ex vivo studies identified a subset of regulatory NKG2D-expressing NK cells in the cerebrospinal fluid of MS patients, which efficiently killed resting microglia and activated macrophages, i.e. cell subsets which are essential for the MS pathogenesis 58. While the overall role of NK cells in MS is still unclear, our data are supported by animal studies demonstrating that NK cells have an immunoregulatory role and can prevent MS-like disease by efficient suppression of autoreactive T-cells⁵⁸.

MS patients in our study were hallmarked by increased EBV reactivation from EBV-infected GlialCAM₃₇₀₋₃₈₉-specific B-cells. The B-cell receptor antigen stimulation and the subsequent induction of the PKC, MAPK, and PI3-K signalling pathways are known factors for the initiation of the EBV lytic cycle and the reactivation from EBV latency⁶⁷ and we speculate that these pathways may be associated with an increased viral replication in MS patients. Additional studies are, however, required to further elucidate the mechanism of EBV reactivation in GlialCAM₃₇₀₋₃₈₉-specific cells, derived from MS patients.

In our study, EBV-infected GlialCAM₃₇₀₋₃₈₉-specific B-cells from MS patients also showed a somewhat increased IL-27 expression. Our results are in line with previously published studies that demonstrated that the EBV-encoded LMP-1 protein leads to expression of the EBI3 subunit of IL-27⁶⁸. Further studies are, however, required to assess whether the increased IL-27 expression in EBV-infected GlialCAM₃₇₀₋₃₈₉-specific B-cells from MS patients may be associated with an increased expression of LMP-1. Our findings are of special interest, as increased IL-27 expression levels were recently identified in the serum and CNS of MS patients^{56,69,70}. The pro-inflammatory effects of IL-27 in MS patients are diverse and include the activation of CD4⁺ and CD8⁺ T lymphocytes⁷¹. Other studies in murine MS models highlighted, however, the anti-inflammatory and neuroprotective roles of IL-27, which is also associated with the inhibition of Th17 and Th9 cell responses as well as the anti-inflammatory cytokine IL-10⁷²⁻⁷⁵. On a functional level, IL-27 also has an immunoregulatory function, as IL-27 leads to increased HLA-E expression levels in in vitro studies⁵⁶. The role of HLA-E in MS pathogenesis recently gained interest, as T-cells in MS patients showed higher HLA-E expression levels⁷⁶ and the disruption of the HLA-E/NKG2A axis was associated with reduced cellular infiltrates and diminished MS-like disease in mouse models⁷⁷. It was, however, unknown whether these results translate to human MS. We now could demonstrate that the HLA-E/NKG2A axis efficiently prevents the killing of autoreactive T-cells in humans and that the IL-27-mediated upregulation of HLA-E and further inhibition of NKG2A-expressing NK cells contributes to the development of MS.

In addition, HLA-E plays an important role in presenting EBV-derived peptides on infected cells⁵⁷. While earlier studies assessed an overall relation between EBV and MS 37, we now demonstrate that development of MS is significantly associated with specific variants of the LMP-1 peptide, which is encoded by the infecting EBV strain and are presented via HLA-E to mediate a potent immune evasion from NKG2A⁺ cells⁵⁷. Nearly all MS patients carried the GGDPHLPTL and GGDPPLPTL LMP-1 peptides, which both lead to a particularly strong inhibition of NKG2A⁺ cells and allowed thereby the evasion from the host immune response and better survival of EBV-infected cells. These data demonstrate that infection with distinct EBV-isolates, encoding for specific LMP-1 peptide variants, are associated with a higher risk for MS.

Interestingly, some individuals infected with EBV variant carrying both LMP-1 variants did not develop MS. According to our findings this could be explained by their individually different abilities to limit EBV replication by potent EBV-specific, HLA-E-restricted CD8⁺ T-cell responses. While these cells were observed earlier, potentially in close proximity to HLA-E upregulating MS lesions^{5,78}, we could now clarify that EBV-specific, HLA-E-restricted CD8⁺ T-cells efficiently control EBV-infected GlialCAM₃₇₀₋₃₈₉-specific B-cells and thus contribute to protection against MS. These data suggest that an immunization with the SQAPLPCVL peptide inducing an SQAPLPCVL-specific HLA-E-restricted CD8⁺ T cell response can contribute to the protection against MS. We found that these responses were especially potent in individuals carrying the highly expressed HLA-E*0103/0103 genotype. In our study, HLA-E*0103/0103 was overrepresented in the high EBNA-1 antibody control population and to some degree prevented development of MS by a higher proliferation and activation of EBV-specific, HLA-E-restricted CD8⁺ T-cells. So far, the HLA-E*0101 allele was not identified as a potential high-risk factor for MS in genome-wide association studies 79. However, in our study, the HLA-E alleles were especially associated with the risk for MS when, together with the LMP-1-encoding EBV variants, a combined risk factor was analysed.

In summary, we demonstrate in our study that different specific immune mechanisms protect individuals from MS, despite of the presence of cross-reactive EBNA-1-specific immune responses, and provide thus new insights in the pathogenesis of MS. Our data further suggest that there may be new therapeutic options for the prevention and therapy of MS.

### Ethics Declaration

The study was approved by the Institutional Review Board of the Medical University of Vienna (IRB number: 1339/2022).

### Experimental Model and Study Participant Details

### Human Study Cohort

For this single-center study a total of 12,708 EBV-seropositive MS patients and healthy controls were initially recruited. From all these individuals, N=270 MS patients fulfilled the following inclusion criteria:
1. MS diagnosis according to the McDonald criteria⁸⁰.
2. Retrospective follow-up serum samples (with maximal intervals of 12 months) until EBV seroconversion of the patients serologically proven by EBV-specific IgG antibodies.
3. EBV-EBNA-1-specific antibodies detectable at the time of MS diagnosis.
4. Clinical information about IM diagnosis available.
5. EBV-DNA available for sequence analysis, obtained at the time of IM or in asymptomatic patients during a reactivation episode at another time point before MS diagnosis.
6. Infecting HCMV-DNA available from HCMV seropositive patients obtained between EBV seroconversion and MS diagnosis.

All MS patients were part of the Vienna Multiple Sclerosis Database (VMSD). Within this database, plasma, serum and cerebrospinal fluid samples are collected upon consent at diagnosis and regularly during the course of the disease. The majority of MS patients were of European (N=252), followed by Middle Eastern (N=17) and Asian (N=1) nationality. None of the patients enrolled were treated with MS-specific treatment before diagnosis. 38 MS patients (14.1%) were treated with corticosteroids within two months prior to MS diagnosis. At the time of diagnosis, 24.4%, 23.7%, and 51.9% MS patients had ≤3, 4-9, and ≥10 brain lesions, respectively.

From all MS patients retrospective follow-up serum samples in a 1-11 (median: 4.2) month interval until EBV seroconversion were available. 202 (74.8%) patients with MS had clinically evident IM during EBV seroconversion. IM was clinically confirmed in all patients by the triad of fever, lymphadenopathy, and tonsillitis, and serologically by EBV-VCA-specific IgM, but non-detectable EBV-EBNA-specific IgG antibodies. The time of seroconversion in IM patients was defined as clinical evidence of IM together with the plasma sample with the first detectable EBV-VCA-specific IgM antibodies.

68 (25.2%) patients with MS had an asymptomatic primary EBV infection, hallmarked by the absence of any clinical symptoms during seroconversion. From all MS patients with asymptomatic primary EBV infection retrospective follow-up serum samples in a 1-9 (median: 4.2) month interval until EBV seroconversion were available. The time of seroconversion in these patients was defined as the plasma sample with the first EBV-VCA-specific IgM and/or EBV-EBNA-specific IgG and/or total EBV-EBV-specific IgG antibodies, following an EBV seronegative sample. The seroconversion occurred after 1.4-4.5 (median: 3.1) months after the last EBV-seronegative sample.

For all MS patients, the time between seroconversion and MS was defined as the time between the first time EBV-seropositive sample and the time of the first clinically proven MS diagnosis. For each patient with MS, one healthy control individual from the initial 12,708 individuals were included in the study. Control individuals were healthy, voluntary blood donors that were matched according to sex, age, time of EBV seroconversion, and the occurrence of IM in the past to the MS cohort using case-control matching (SPSS 27). All controls fulfilled the following inclusion criteria:
1. No diagnosis of MS or other autoimmune disease or neuro-inflammatory disorder.
2. Retrospective follow-up serum samples (with maximal intervals of 12 months) until EBV seroconversion of the patients serologically proven by EBV-specific IgG antibodies at a matched time point for MS diagnosis.
3. EBV-EBNA-1-specific antibodies detectable at the time of MS diagnosis.
4. Clinical information about IM diagnosis available.
5. EBV-DNA available for sequence analysis, obtained at the time of IM or in asymptomatic patients during a reactivation episode at another time point before a matched time point for MS diagnosis.
6. Infecting HCMV-DNA available from HCMV seropositive patients obtained between EBV seroconversion to a matched time point for MS diagnosis.

Thus, at least two plasma samples from each MS patient and healthy control were included in the study cohort. One plasma sample was collected from patients immediately (0-6 days) after MS diagnosis or at a matched time point after EBV seroconversion for the controls. In one additional plasma sample EBV-DNA (>200 copies/mL) was detected by EBV-qPCR. This sample was collected from IM patients for IM diagnosis and from patients with asymptomatic primary EBV infection during an episode of EBV reactivation in the blood. In all HCMV seropositive study participants (MS:125/270 and controls:105/270) one additional HCMV-DNA positive (>100 copies/mL) plasma sample was available.

From 20 MS patients (7.4%) and 80 controls (29.6%), additional peripheral blood mononuclear cells (PBMC) were available, which were collected from MS patients immediately (0-6 days) after MS diagnosis, or at a matched time point after EBV seroconversion for the controls.

For the functional assays, we also included peripheral blood mononuclear cells (PBMCs) from 60 healthy, HCMV-, and EBV-seropositive voluntary blood donors.

### Primary Cells

PBMCs from MS patients, EBNA^{high} controls, EBNA^{low} controls or healthy, voluntary blood donors were isolated from whole blood by Ficoll-Paque PLUS density (Cytiva) gradient centrifugation according to the manufacturer's instructions and subsequently stored frozen at −80° C. in 5×10⁶ viable cells per aliquot in 90% FCS+10% DMSO (both Thermo-Fisher). CD14⁺CD16⁺ monocytes from 12 healthy blood donors were isolated from the buffy coat using CD16+ Monocyte Isolation Kit (Miltenyi Biotec) and differentiated into CD14⁺CD16⁺ M1 macrophages using M1-Macrophage Generation Medium XF (ProCell), according to the manufacturer's instructions.

CD56⁺ NK cells were enriched by magnetic labeling using a human CD56⁺ NK Cell Isolation Kit according to the manufacturer's instructions (Miltenyi Biotec). For some experiments, CD56⁺KIR2DS1⁺, CD56⁺KIR3DS1⁺, CD56⁺NKp46⁺, CD56⁺NKp44⁺, CD56⁺NKp30⁺, CD56⁺NKG2A⁺NKG2D⁻NKG2C⁻, CD56⁺NKG2D⁺, CD56⁺NKG2A⁻NKG2C⁺ NK cells were then sorted using FACSAria Fusion (BD Bioscience).

CD8⁺ T-cells were enriched by magnetic labeling using a human CD8⁺ T-Cell Isolation Kit, according to the manufacturer's instructions (Miltenyi Biotec). For some experiments, EBV-specific CD8⁺ T-cells were identified and sorted using an EBV-peptide mix (PepMix Collection EBV, JPT) coupled to HLA-A*02:01, HLA-A*03:01, HLA-A*11:01, and HLA-A*24:02 APC-conjugated tetramers, according to the manufacturer's instructions (Tetramer Shop). EBV-specific, HLA-E-restricted CD8⁺ T-cells were identified and sorted using RMA-S/HLA-E/LFA-3 cells as described in detail before³⁹.

All cells were stored frozen at −80° C. in 1×10⁶ viable cells per aliquot in 90% FCS+10% DMSO (both Thermo Fisher).

### Cell Lines

The human T-cell leukemia cell line Jurkat (German Collection of Microorganisms and Cell Cultures, ACC 282, RRID: CVCL_0065, Male), the human acute monocytic leukemia cell line THP-1 (German Collection of Microorganisms and Cell Cultures, ACC 16, RRID: CVCL_0006, Male) and the human lymphoblast-like RAJI cell line (German Collection of Microorganisms and Cell Cultures, ACC 319, RRID: CVCL_0511, Male) were maintained in in 90% RPMI 1640+10% heat-inactivated FBS+2 mM L-glutamine (all Thermo Fisher). The natural killer lymphoma cell line NK92 (German Collection of Microorganisms and Cell Cultures, ACC 488, RRID: CVCL_2142, Male) was maintained as described in detail before⁸¹. The mouse lymphoma cell line RMA-S/HLA-E/LFA-3 (kindly provided by Chiara Romagnani, DRFZ, Berlin) was maintained in RPMI-1640+20 mM glutamine+10% FCS+20 μM β-mercaptoethanol+100 U/ml penicillin-streptomycin (all Thermo Fisher)+400 μg/ml hygromycin B+1 mg/ml G418 (both Invitrogen).

The marmoset B-lymphoblastoid cell line B95-8 (German Collection of Microorganisms and Cell Cultures, ACC 100, RRID: CVCL_1953, female) was cultured in RPM1 1640 medium supplemented with 20 mM glutamine, 50 pg/ml gentamicin, 0.2 pg/ml amphotericin B and 10% FCS (Thermo-Scientific).

All cells were maintained at 37° C. and in a 5% CO2 atmosphere. All the cell lines were regularly determined negative for Mycoplasma using the MycoAlert Mycoplasma Detection Kit (Biozym). Cells were used for experiments within 10 passages from thawing.

### Virus

B95-8 cells were cultured in the presence of 12-O-tetradecanoyl-phorbol-12-acetate (Biomol) for two weeks. EBV was harvested from the supernatant and enriched by ultracentrifugation (30000 U/min, 90 min, 10° C.). The virus stocks were stored at −80° C. in RPMI 1640 medium+10% FCS+10% DMSO (all: Thermo-Scientific). The multiplicity of infection (MOI) of the viral stocks was determined on RAJI cells by flow-cytometry.

### Method Details

### EBV- and HCMV-Detection and Serology

Viral DNA was isolated from plasma samples using the TanBead (Taiwan Advanced Nanotech Inc). Nucleic acids were eluted in 50 μl nuclease-free H₂O. EBV-DNA and HCMV-DNA were detected and quantified by TaqMan assays, using recently published protocols⁴⁶. HCMV-specific IgG, EBNA-1-specific IgG (DRG International), and total EBV-specific IgG (Euroimmune) antibodies were detected and quantified in plasma by ELISA.

### Identification of EBNA₃₈₆₋₄₀₅-Specific Cells

5×10⁶ viable CFSE (CellTrace, CFSE Cell Proliferation Kit, Thermo Fisher) stained PBMCs were cultured in RPMI-1640+10% FCS (both Thermo Fisher) in the presence or absence of 10 μg EBNA₃₈₆₋₄₀₅ (SQSSSSGSPPRRPPPGRRPF, Peptides&Elephants) and 10 ng/mL IL-2 (Peprotech) at 37° C. Every three days, fresh 10 μg EBNA₃₈₆₋₄₀₅ and 10 ng/mL IL-2 were added. After 7 days of co-culture, EBNA₃₈₆₋₄₀₅-specific CD4⁺ T-cells, CD8⁺ T-cells, and CD138⁺ plasma cells were either fixed and analyzed by flow cytometry or sorted by FACS, both for CFSE and in comparison with the non-peptide control.

Identification of GlialCAM₃₇₀₋₃₈₉-Specific CD4⁺ T-Cells or CD8⁺ T-Cells

5×10⁶ viable CFSE stained PBMCs or 2×10⁵ viable EBNA₃₈₆₋₄₀₅-specific CD4⁺ T-cells, or CD8⁺ T-cells were cultured in the presence or absence of 10 μg GlialCAM₃₇₀₋₃₈₉ (ATGRTHSSPPRAPSSPGRSR, Peptides&Elephants) for 12 h at 37° C. in RPMI-1640+10% FCS (both Thermo Fisher), and 1× Monensin and 1× Brefeldin A (BioLegend). GlialCAM₃₇₀₋₃₈₉-specific CD4⁺ T-cells or CD8⁺ T-cells were fixed and analyzed and identified as IFNβ⁺ cells in comparison with the non-peptide control by flow cytometry.

### Identification of GlialCAM₃₇₀₋₃₈₉-Specific B- and Plasma-Cells

5×10⁶ viable CFSE stained PBMCs were cultured in the presence or absence of 10 μg GlialCAM₃₇₀₋₃₈₉ (ATGRTHSSPPRAPSSPGRSR, Peptides&Elephants) in RPMI-1640+10% FCS (both Thermo Fisher) and 10 ng/mL IL-2 (Peprotech) at 37° C. Every three days, fresh 10 μg GlialCAM₃₇₀₋₃₈₉ and 10 ng/mL IL-2 were added. After 7 days of co-culture, GlialCAM₃₇₀₋₃₈₉-specific CD19+B-cells and CD138+ plasma cells were fixed and analyzed, in comparison with the non-peptide control, for CFSE by flow cytometry.

Detection of EBNA₃₈₆₋₄₀₅- or GlialCAM₃₇₀₋₃₈₉-Specific IgG Antibodies

EBNA₃₈₆₋₄₀₅-specific or GlialCAM₃₇₀₋₃₈₉-specific IgG antibodies were quantified by ELISA as described before⁴⁰. Briefly, 96-well plates (Thermo Fisher Scientific) were coated with 1 μg/mL EBNA₃₈₆₋₄₀₅ or GlialCAM₃₇₀₋₃₈₉ and then incubated with 1:100 diluted patient plasma. EBNA₃₈₆₋₄₀₅- or GlialCAM₃₇₀₋₃₈₉-specific IgG antibodies were detected using H1RP-conjugated goat anti-human IgG (1:12500) and TMB substrate (both Thermo Fisher).

### GlialCAM₃₇₀₋₃₈₉-Specific IgG Effector Functions

GlialCAM₃₇₀₋₃₈₉ (ATGRTHSSPPRAPSSPGRSR) was labeled with AF647 NHS Ester (Thermo Fisher), according to the manufacturer's instructions, and cultured together with CD14+CD16+M1 macrophages in Opti-MEM I Reduced Serum Medium (Gibco) and 5 μL mouse-anti human APC-H7 CD107 mAB for 8 h at 37° C. GlialCAM₃₇₀₋₃₈₉-specific ADCP responses were assessed as the mean AF647 MFI of the CD14⁺CD16⁺ cells as described before by flow cytometry⁸². A higher AF647 MFI demonstrates in this assay a more efficient uptake of fluorescently labelled GlialCAM370-389 peptides. GlialCAM₃₇₀₋₃₈₉-specific ADCC responses were assessed in parallel with CD107a⁺CD14+CD16+M1 macrophages by flow-cytometry.

### NKG2D, KLRC2, HLA-E, HCMV UL40 and EBV LMP-1 Genotyping

Genomic and viral DNA was isolated from 200 μL of plasma using the TanBead extractor. Nucleic acids were eluted in 50 μl nuclease-free H₂O. Low-resolution genotyping of the HLA-DRB1 locus was performed by nested PCR as described before 83. NKG2D^{HNK/LNK} was determined based on the rs1049174 SNP by TaqMan assay using a commercial TaqMan SNP Genotyping Assay (C_9345347_10) according to the manufacturer's recommendations.

HLA-E genotyping was performed using a recently published TaqMan assay and HLA-E*0101- and HLA-E*0103-specific probes^{84,29} KLRC2^{wt/del} variants were determined by touchdown PCR as recently described⁸⁵. HCMV UL40 and EBV LMP-1 variants were determined using nested PCR followed by Sanger sequencing, as reported before^{44,12} DNA sequences were translated into protein sequences using the Expasy tool developed by the Swiss-Prot group and supported by the Swiss Institute of Bioinformatics (https://web.expasy.org/translate/).

### Killing Assay

5×10⁶ viable CFSE stained PBMCs were cultured in RPMI-1640+10% FCS and the presence of 10 μg GlialCAM₃₇₀₋₃₈₉ (ATGRTHSSPPRAPSSPGRSR), 10 μg of random 20 amino acid peptides (negative control, GenScript) or without peptides (non-peptide control) and 10 ng/mL IL-2 (Peprotech) at 37° C. Every three days, fresh 10 μg GlialCAM₃₇₀₋₃₈₉ and 10 ng/mL IL-2 were added. After 14 days of co-culture, GlialCAM₃₇₀₋₃₈₉-specific CD4⁺ T-cells, CD8⁺ T-cells, and CD138⁺ plasma cells all sorted for CFSE by FACS. For the negative or non-peptide control all CD4⁺ T-cells, CD8⁺ T-cells, and CD138⁺ plasma cells were sorted. The GlialCAM₃₇₀₋₃₈₉-specific CD4⁺ T-cells, CD8⁺ T-cells, and CD138+ plasma cells from each donor were then pooled, labeled with AF647 NHS Ester, and co-cultured for 6 h in a target: to effector (E:T) ratio of 1:1 with Jurkat T-cells, or THP-1 monocytes, or enriched primary CD8⁺ T-cells from 24 healthy blood donors, or sorted KIR2DS1⁺CD56⁺ NK cells, or sorted KIR3DS1⁺CD56⁺ NK cells, or sorted NKp46⁺CD56⁺ NK cells, or sorted NKp44⁺CD56⁺ NK cells, or sorted NKp30⁺CD56⁺ NK cells, or sorted NKG2D⁺ CD56⁺, or sorted NKG2C⁺CD56⁺ NK cells from 24 healthy blood donors, respectively, in Opti-MEM I Reduced Serum Medium (Gibco). The activation of isolated primary CD8⁺ T-cells, Jurkat T-cells and NK92 NK cells was assessed using IL-2 and IFNγ ELISA (Thermo Fisher) from the centrifugation cleared (1000×g, 5 min) supernatant, according to the manufacturer's instructions. The activation of THP-1 cells was assessed by determining the mean AF647 MFI via flow cytometry. For the activation of the NK cell subsets, 5 μL of mouse-anti-human APC-H7 CD107 mAB was added to the co-culture, and the percentage of CD107a⁺CD56⁺KIR2DS1⁺ NK cells, or CD107a⁺CD56⁺KIR3DS1⁺ NK cells, or CD107a⁺CD56⁺NKp46⁺ NK cells, or CD107a⁺CD56⁺NKp44⁺ NK cells, or CD107a⁺CD56⁺NKp30⁺ NK cells, or CD107a⁺CD56⁺NKG2D⁺, or CD107a⁺CD56⁺NKG2C⁺ NK cells was assessed via flow cytometry. For some experiments, the lysis of the CD4⁺ T-cells, CD8⁺ T-cells, and CD138⁺ plasma cells by NK cell subsets was also quantified from the centrifugation cleared (1000×g, 5 min) supernatant after co-culture by an bioluminescent LDH release assay (LDH-Glo Cytotoxicity Assay, Promega), according to the manufacturer's instruction.

Detection of EBV-Infected Cells EBV-infected cells were detected by FISH cytometry assay as described before⁸⁶.

### HLA-E Upregulation and NK Cell Inhibition Assay

5×10⁶ viable CFSE stained PBMCs were cultured in RPMI-1640+10% FCS in the presence of 10 μg GlialCAM₃₇₀₋₃₈₉ (ATGRTHSSPPRAPSSPGRSR), 10 μg of random 20 amino acid peptides (negative control, GenScript) or without peptides (non-peptide control) and 10 ng/mL IL-2 (Peprotech) at 37° C. Every three days, fresh 10 μg GlialCAM₃₇₀₋₃₈₉ and 10 ng/mL IL-2 were added. After 14 days of co-culture, GlialCAM₃₇₀₋₃₈₉-specific (CFSE^{low}) CD4⁺ T-cells, CD8⁺ T-cells, and CD138⁺ plasma cells were analyzed for HLA-E expression via flow cytometry. For some experiments, the supernatant was removed, cleared by centrifugation (1000×g, 5 min), and used for IL-27 ELISA (Thermo Fisher) according to the manufacturer's instructions. For supernatant transfer experiments, the supernatant was removed, cleared by centrifugation (1000×g, 5 min), and transferred to 5×10⁶ viable PBMCs from the same donor. In some experiments, 120 ng/mL IL-27 blocking antibody (MM27-7B, Thermo Fisher) was also added. After 16 h of incubation at 37° C., PBMCs were fixed and analysed for the HLA-E expression by flow-cytometry.

For the IL-27 expression analysis, 5×10⁶ viable CFSE stained PBMCs were cultured in RPMI-1640+10% FCS in the presence of 10 μg GlialCAM₃₇₀₋₃₈₉ (ATGRTHSSPPRAPSSPGRSR), 10 μg of random 20 amino acid peptides (negative control, GenScript) or without peptides (non-peptide control) and 10 ng/mL IL-2 (Peprotech) at 37° C. Every three days, fresh 10 μg GlialCAM₃₇₀₋₃₈₉ and 10 ng/mL IL-2 were added. After 14 days of co-culture, GlialCAM₃₇₀₋₃₈₉-specific CD19⁺ B-cells, CD14 monocytes, CD56⁺ NK cells, CD1⁺ DC, and CD19⁺ B-cells were fixed analyzed for the intracellular IL-27 expression via flow cytometry.

For the NK cell inhibition assays, GlialCAM₃₇₀₋₃₈₉-specific (CFSE^{low}) CD4⁺ T-cells, CD8⁺ T-cells, and CD138⁺ plasma cells were sorted and co-cultured together in an E:T ratio of 1:1 with sorted CD56⁺NKG2A⁺ NKG2D⁻NKG2C⁻ NK cells from 24 healthy blood donors in OptiMem (Thermo Fisher) for 12 h at 37° C. For some experiments α-NKG2A blocking monoclonal antibodies (Monalizumab, 10 μg/mL, Innate Pharma) was added to the co-culture. The supernatant was removed, cleared by centrifugation (1000×g, 5 min), and then used for IFNγ (both Thermo Fisher) according to the manufacturer's instructions.

### LMP-1 Dependent HLA-E Up-Regulation Assay

For the HLA-E stabilization experiments, 5×10⁶ viable CFSE stained PBMCs were cultured in RPMI-1640+10% FCS in the presence of 10 μg GlialCAM₃₇₀₋₃₈₉ (ATGRTHSSPPRAPSSPGRSR), 10 μg of random 20 amino acid peptides (negative control, GenScript) or without peptides (non-peptide control) and 10 ng/mL IL-2 (Peprotech) at 37° C. Every three days, fresh 10 μg GlialCAM₃₇₀₋₃₈₉ and 10 ng/mL IL-2 were added. After 7 days of co-culture, GlialCAM₃₇₀₋₃₈₉-specific B-cells were sorted. 1×10⁵ GlialCAM₃₇₀₋₃₈₉-specific B-cells were then individually transfected with the indicated concentrations of the LMP-1 peptides (Peptides&Elephants) using the Pierce Protein Transfection Reagent according to the manufacturer's instructions (Thermo Fisher). After 16 h, the cells were either fixed and analyzed for HLA-E expression by flow cytometry, as described below, or washed once with Opti-MEM I Reduced Serum Medium (Gibco) and subsequently used in NKG2A⁺ NK cell inhibition experiments. For the NKG2A⁺ NK cell inhibition experiments, sorted NKG2A⁺NKG2C⁻NKG2D⁻ NK cells from 24 healthy blood donors were cultured together with peptide-pulsed GlialCAM₃₇₀₋₃₈₉ B-cells (E:T, 1:1) for 6 h at 37° C. After co-cultivation, the supernatant was removed, cleared by centrifugation (1000×g, 5 min), and analyzed using IFNγ ELISA according to the manufacturer's recommendations (Thermo Fisher).

### EBV-Infected Cell Cytotoxicity Assay

CFSE stained PBMCs were cultured in the presence of 10 μg GlialCAM₃₇₀₋₃₈₉ (ATGRTHSSPPRAPSSPGRSR), 10 μg of random 20 amino acid peptides (negative control, GenScript) or without peptides (non-peptide control) and 10 ng/mL IL-2 (Peprotech) at 37° C. Every three days, fresh 10 μg GlialCAM_{370-389 and} 10 ng/mL IL-2 were added. After 7 days, GlialCAM₃₇₀₋₃₈₉-specific B-cells (GlialCAM₃₇₀₋₃₈₉) or all remaining non-GliaCAM₃₇₀₋₃₈₉-specific B-cells were sorted by FACS. 5×10⁶ viable GlialCAM₃₇₀₋₃₈₉-specific B-cells or non-GlialCAM₃₇₀₋₃₈₉-specific B-cells or RAJI cells were then infected with the marmoset B-lymphoblastoid cell line B95-8 derived EBV strain (MOI=1)³⁹ for 3 days at 37° C. The cells were then either fixed and analyzed for HLA-E expression by flow cytometry, as described below, or washed once with Opti-MEM I Reduced Serum Medium (Gibco) and subsequently used in killing assays. For the ADCP and ADCC assays EBV-infected B-cells were labeled with AF647 NHS Ester (Thermo Fisher), according to the manufacturer's instructions, and cultured together with CD14⁺CD16⁺ monocyte-derived CD14⁺CD16⁺ M1 macrophages and 5 μL mouse-anti human APC-H7 CD107 mAB. GlialCAM₃₇₀₋₃₈₉-specific ADCP responses were assessed as the mean AF647 MFI as described before⁸². GlialCAM₃₇₀₋₃₈₉-specific ADCC responses were assessed in parallel with CD107a⁺CD14⁺CD16⁺ M1 macrophages.

For CD8⁺ T-cell-mediated killing assays, enriched total EBV-specific CD8⁺ T-cells or EBV-specific, HLA-E-restricted CD8⁺ T-cells were cultured together with EBV-infected GlialCAM₃₇₀₋₃₈₉s-specific B-cells for 8 h at 37° C. The supernatant was then used for IFNγ ELISA (Thermo Fisher) according to the manufacturer's instructions.

For the CFSE stained EBV-specific, HLA-E-restricted CD8⁺ T-cell activation and proliferation experiments, GlialCAM₃₇₀₋₃₈₉-specific B-cells were infected with the marmoset B-lymphoblastoid cell line B95-8 derived EBV strain (MOI=1)³⁹ for 3 days at 37° C. The EBV-infected GlialCAM₃₇₀₋₃₈₉-specific B-cells were then co-cultured together with EBV-infected GlialCAM₃₇₀₋₃₈₉s-specific B-cells in 90% RPMI 1640+10% heat-inactivated FCS (Thermo-Fisher) at 37° C. After 3 days the medium was replaced. The supernatant was removed, cleared by centrifugation (1000×g, 5 min) and analysed by IFNγ ELISA according to the manufacturer's recommendations (Thermo Fisher). After additional 4 days, the cells were harvested, fixed and analyzed by flow-cytometry.

### Flow Cytometry

The following conjugated mouse anti-human mAB were used for flow cytometry: PerCPCy5.5-PD-1 (EH12.1), PE-LAG-3 (T47-530), BV421-CD56 (NCAM 16.2), BV510-NKG2A (131411), PE-NKG2D (1D11), BV421-CD8 (RPA-T8), BV510-CD3 (UCHT1), BV421-CD14 (MφP9), PE-CD16 (B73.1), PE/Cyanine7-CD19 (SJ25C1), PE-CD14 (M5E2), BV421-CD4 (RPA-T4), BV510-CD8 (SK1), PE-CD138 (MI15), APC/H7-CD107a (H4A3) (all: BD), AF647-KIR2DS1 (1127B), AF647-NKG2C (134591, R&D Systems), APC/Cy7-IFNγ (B27), PE/Cyanine7-HLA-E (3D12), APC-CD1d (R3G12), PE/Cy7-TIGIT (A15153G), APC/Cy7-TIM-3 (F38-2E2) AF647-NKG2D (1D11), AF647-NKp46 (9E2), AF647-NKp44 (P44-8), AF647-NKp30 (P30-15), AF647-NKG2D (1D11), (all from Biolegend). Polyclonal KIR3DS1 (Novos Biologicals) antibodies were coupled to the Alexa Fluor647 Antibody Labeling Kit Thermo Fisher), according to the manufacturer's instruction. Dead cells were identified using LIVE/DEAD Fixable Green Dead Cell Stain Kit, LIVE/DEAD Fixable Far Red Dead Cell Stain Kit, LIVE/DEAD Fixable Aqua Dead Cell Stain Kit (all from Thermo-Scientific) or 7-Aminoactinomycin D (7-AAD, Invitrogen). Flow-cytometry analysis was performed on a FACSCanto2 platform and FACSDiva Version 10.7.2 (BD). For cell sorting, dead cells were first removed using the Dead Cell Removal Kit (Miltenyi Biotec) and sorted with a FACSAria Fusion (BD).

### Statistical Analysis

The Chi-square test and Fisher's exact test were used to compare the distribution of genetic variants. Outliers of the flow cytometry data were first identified using the ROUT method and then compared between the groups using RM one-way ANOVA (with the Geisser-Greenhouse correction) or the Mann-Whitney-Test or Wilcoxon matched T-test. Statistical significance was set at p<0.05. Statistical differences were assessed using GraphPad Prism 9.

| | MS | Healthy Control | | |
|---|---|---|---|---|
| Characteristic | N = 270 | N = 270 | | |
| Sex | | | | |
| Female (%) | N = 168 (62.2%) | N = 168 (62.2%) | | |
| Male (%) | N = 102 (37,8%) | N = 102 (37.8%) | | |
| Median Age (min-max) | 37.2 (16.3-59.7) | 34.7 (18.2-51.3) | | |
| EBV-seroconversion (median years) | 8.2 (3.1-11.8) | 7.7 (2.9-12.4) | | |
| pre-MS diagnosis or matched time | | | | |
| point for Healthy Controls (range) | | | | |
| Primary EBV-Infection | | | | |
| Infectious Mononucleosis (%) | N = 202 (74.8%) | N = 202 (74.8%) | | |
| Asymptomatic (%) | N = 68 (25.1%) | N = 68 (25.1%) | | |
| | | EBNA^{high} | EBNA^{low} | |
| | | N = 108 | N = 162 | P-value |
| Sex | | | | |
| Female (%) | | N = 92 (85.2%) | N = 76 (46.9%) | <0.0001 |
| Male | | N = 16 (14.8%) | N = 86 (53.1%) | (OR: 6.5; 95% |
| | | | | CI: 3.52-12.03) |
| Median Age (min-max) | | 33.9 (18.2-49.4) | 35.6 (18.7-51.3) | 0.93 |
| EBV-seroconversion (median years) | | 7.5 (2.9-10.1) | 7.9 (3.4-12.4) | 0.84 |
| pre-MS diagnosis or matched time | | | | |
| point for Healthy Controls (range) | | | | |
| Primary EBV-Infection | | | | |
| Infectious Mononucleosis (%) | | N = 89 (82.4%) | N = 113 (69.8%) | 0.02 |
| Asymptomatic (%) | | N = 19 (17.6%) | N = 49 (30.2%) | (OR: 2.03; 95% |
| | | | | CI: 1.12-3.7) |

TABLE 3
EBV EBNA-1-specific IgG Antibody Titers and HCMV-seroprevalence in the Study Cohort

| Serostatus | MS | EBNA^{High} | EBNA^{Low} | P Value |
|---|---|---|---|---|
| EBV EBNA-1 IgG Titer | | | | |
| Mean EBNA-1 IgG | 37.91 | 39.31 | 29.41 | MS vs. EBNA^{High}: p = 0.3 |
| [DRG Units/mL] (±SD) | (±7.28) | (±9.9) | (±7.46) | MS vs. EBN^{low}: p = 0.0009 |
| | | | | EBNA^{High} vs. EBNA^{low}: p = 0.001 |
| HCMV Serostatus | | | | |
| HCMV Seronegative | N = 125 (46.3%) | N = 28 (25.9%) | N = 77 (47.5%) | MS vs. EBNA^{High}: p = 0.003 |
| | | | | (OR: 2.4, 1.5-4) |
| HCMV Seropositive | N = 145 (53.7%) | N = 80 (74.1%) | N = 85 (52.5%) | MS vs. EBNA^{low}: p = 0.8 |
| | | | | (OR: 1.1, 0.7-1.6) |
| | | | | EBNA^{High} vs. EBNA^{low}: p = 0.0004 |
| | | | | (OR: 2.6, 1.5-4.4) |

TABLE 4
Distribution of UL40 Variants in the Study Cohort

| | | | | | | | Mean | HLA-E | |
|---|---|---|---|---|---|---|---|---|---|
| UL40 Variant | MS (%) | | EBNA^{High} (%) | | EBNA^{Low} (%) | | MFI | Upregulation | Interpretation |
| VMAPRTLFL | N = 1 | (0.7%) | N = 1 | (1.3%) | N = 0 | (0%) | 3797 | 100.0% | High Binding (↑↑) |
| VMAPRTLVL | N = 1 | (0.7%) | N = 17 | (21.3%) | N = 15 | (16.9%) | 3782 | 99.6% | High Binding (↑↑) |
| VMAPRTLIL | N = 27 | (18.6%) | N = 48 | (60%) | N = 53 | (53.6%) | 3780 | 99.6% | High Binding (↑↑) |
| VMAPRTLLL | N = 1 | (0.7%) | N = 10 | (12.5%) | N = 8 | (9%) | 3776 | 99.5% | High Binding (↑↑) |
| VMTPRTLIL | N = 21 | (14.5%) | N = 1 | (1.3%) | N = 2 | (2.2%) | 2633 | 69.3% | Low Binding (↑) |
| VMTPRTLLL | N = 19 | (13.1%) | N = 1 | (1.3%) | N = 2 | (2.2%) | 2519 | 66.3% | Low Binding (↑) |
| VMTPRTLVL | N = 20 | (13.8%) | N = 1 | (1.3%) | N = 2 | (2.2%) | 2488 | 65.5% | Low Binding (↑) |
| VMAPRSLLL | N = 5 | (3.4%) | N = 0 | (0%) | N = 1 | (1.1%) | 2351 | 61.9% | Low Binding (↑) |
| VMAPRILVL | N = 7 | (4.8%) | N = 1 | (1.3%) | N = 0 | (0%) | 2299 | 60.5% | Low Binding (↑) |
| VMAPRSLIL | N = 3 | (2.1%) | N = 0 | (0%) | N = 0 | (0%) | 2074 | 54.6% | Low Binding (↑) |
| VLAPRTLLL | N = 5 | (3.4%) | N = 0 | (0%) | N = 0 | (0%) | 2059 | 54.2% | Low Binding (↑) |
| VMAPRILIL | N = 6 | (4.1%) | N = 0 | (0%) | N = 1 | (1.1%) | 2056 | 54.2% | Low Binding (↑) |
| VMGPRTLIL | N = 5 | (3.4%) | N = 0 | (0%) | N = 1 | (1.1%) | 1858 | 48.9% | Low Binding (↑) |
| VVAPRTLVL | N = 5 | (3.4%) | N = 0 | (0%) | N = 1 | (1.1%) | 1840 | 48.5% | Low Binding (↑) |
| GMAPRTLLL | N = 5 | (3.4%) | N = 0 | (0%) | N = 1 | (1.1%) | 1575 | 41.5% | Low Binding (↑) |
| VIAPRTLIL | N = 4 | (2.8%) | N = 0 | (0%) | N = 1 | (1.1%) | 1541 | 40.6% | Low Binding (↑) |
| VMAPWTLVL | N = 4 | (2.8%) | N = 0 | (0%) | N = 1 | (1.1%) | 1503 | 39.6% | Low Binding (↑) |
| VMAPWTLIL | N = 6 | (4.1%) | N = 0 | (0%) | N = 0 | (0%) | 1210 | 31.9% | Low Binding (↑) |

TABLE 5
Distribution of KLRC2 Genotypes in the Study Cohort

| NKG2C Status | MS | | EBNA^{High} | | EBNA^{Low} | | P Value (OR, 95% CI) |
|---|---|---|---|---|---|---|---|
| KLRC2^{wt/wt} | N = 133 | (49.3%) | N = 70 | (64.8%) | N = 99 | (61.1%) | MS vs. EBNA^{High}: p = 0.03 |
| KLRC2^{wt/del} | N = 113 | (41.9%) | N = 32 | (29.6%) | N = 60 | (37%) | MS vs. EBNA^{low}: p = 0.03 |
| KLRC2^{del/del} | N = 24 | (8.9%) | N = 6 | (5.5%) | N = 6 | (3.7%) | EBNA^{High} vs. EBNA^{low}: p = 0.4 |

### EXAMPLE 7

SQAPLPCVL-Specific HLA-E-Restricted CD8⁺ T Cells Prevent the Proliferation of EBV⁺ Lymphoma Cells

### Introduction

### Epstein-Barr Virus-Associated Lymphoproliferative Diseases

Epstein-Barr virus (EBV) is a ubiquitous herpesvirus that infects over 90% of the adult human population worldwide 3. Primary EBV infection may result in infectious mononucleosis (IM), which is hallmarked by fever, lymphadenopathy, and tonsillitis. After primary infection, EBV establishes a life-long persistent infection in memory B cells, from which sporadic reactivations may occur. EBV reactivations in most immunocompetent individuals are either asymptomatic or result in mild diseases, characterized by unspecific symptoms such as fever or fatigue. EBV reactivations are, however, also associated with the development of EBV-associated malignant and lymphoproliferative diseases in some individuals.

EBV is globally associated with the development of EBV⁺ lymphoproliferative diseases (EBV⁺LDs), such as Hodgkin lymphomas (HL) as well as EBV⁺ B cell and EBV⁺ NK/T cell non-Hodgkin lymphomas (nHL). Among all HL cases, about 40%, mainly of the classical-nodular sclerosis (cHL-NSHD) subtype are associated with EBV. EBV⁺HL are hallmarked by the presence of clonal EBV genomes and EBV-encoded proteins in the HL-defining Hodgkin Reed-Sternberg (HRS) tumor cells. EBV⁺HRS cells originate from germinal center B cells, which are surrounded by inflammatory infiltrates. Diffuse large B cell lymphoma (DLBCL) and peripheral T cell lymphoma (PTCL) are frequently occurring types of high-grade nHL. About 10% of all DLBCL and 21% of all PTCL, both mainly of the not otherwise specified subtype, are EBV-DNA-positive and express EBV-encoded proteins 87. EBV is also associated with mainly geographically restricted EBV⁺LDs such as the endemic Burkitt lymphoma (EBV⁺BL). EBV⁺BL, a B cell nHL, frequently occurs in children in malaria-endemic regions, such as Equatorial Africa⁸⁸.

In solid-organ (SOT) and hematopoietic stem cell transplant (HSCT) recipients, EBV may cause post-transplant lymphoproliferative disorders (EBV⁺PTLD). In Europe and the United States, over 80% of PTLD cases are EBV-associated, and the risk of developing EBV⁺PTLD is generally higher in the first year post-transplantation due to primary infection of EBV-naive transplant recipients by an EBV-positive transplanted organ⁸⁹⁻⁹¹. Based on a histopathologic analysis, four main types of PTLD are described: i) Monomorphic PTLD, ii) Polymorphic PTLD, iii) Early lesions, and iv) cHL. The monomorphic PTLD is the most common form of EBV⁺PTLD. The lymphoma cells are usually B cell-derived and resemble the EBV⁺nHL, EBV⁺ DLBCL, and EBV⁺BL⁹¹. EBV⁺PTLD is the second most common malignant complication following transplantation after skin cancers. Depending on the type of transplantation and immunosuppression, EBV⁺PTLD occurs with an incidence of 2,000/100,0000 to 20,000/100,000 transplanted individuals⁹².

EBV-associated malignant diseases result worldwide in more than 239,700 new cases annually³. The global incidence of EBV-associated malignancies is estimated to be 5.8/100,000 individuals and ˜1% of all occurring tumors are estimated to be associated with EBV⁹³. The therapeutic options for EBV⁺LDs are generally limited and may include immunotherapy, chemotherapy, radiotherapy, and HSCT 94. Nonetheless, studies estimated that EBV-associated malignancies are leading to more than 137,900 annual deaths worldwide³. So far, no EBV vaccines are available that could prevent the development of EBV⁺LDs in non-transplanted individuals and EBV⁺PTLDs high-risk transplant recipients.

### Epstein-Barr Virus Gene Expression

As a distinguishing feature, the life cycle of EBV consists of two phases: lytic replication and latency. During reactivation, the gene expression profile of EBV is hallmarked by the expression of the BZLF1 or BRLF immediate early (IE) genes that induce the lytic EBV life cycle. This IE gene expression results in the expression of over 80 subsequent lytic early genes that are further required for the lytic viral DNA replication, virus assembly, and release of progeny virions²¹.

During latency, EBV DNA is present in the nucleus of infected cells, and distinct EBV-derived latent proteins are expressed. These latency-associated proteins prevent apoptosis and induce B cell proliferation, thereby enabling the malignant transformation of B cells. Importantly, different EBV⁺LDs express distinct sets of EBV-derived latent proteins, and while EBV⁺BL shows type I latency (EBNA-1), EBV⁺HL and EBV⁺PTCL nHL display type II latency (EBNA1, LMP1, LMP2). In contrast, most of the EBV⁺DLBCLs show a type II or type III latency (all EBNAs, LMP1, and LMP2) protein expression²¹. The most commonly described latency program in EBV⁺PTLD is latency III⁹⁵. While the EBV expression profile in EBV⁺LDs is hallmarked by latency-associated proteins, some studies have recently reported that some proteins of the lytic cycle are also expressed in a subset of tumor cells. A recent publication demonstrated that e.g. BZLF1 is expressed in the majority of EBV⁺PTLD and EBV⁺DLBCL lymphomas⁹⁶.

### EBV-Specific Immune Responses

Without early and intensive interventions, EBV⁺LDs are generally associated with poor overall survival, especially at late stages of the disease³. As the majority of the adult population carries EBV and undergoes sporadic EBV reactivations, and these malignant EBV-associated diseases only occur in a fraction of EBV-seropositive patients, the question remains why do some individuals with reactivating EBV progress towards EBV⁺LDs, while in most others, the reactivation episodes are efficiently controlled. It was subsequently hypothesized that there are distinct, individually determining factors in the human EBV-specific immune responses that control EBV replication, eliminate EBV-infected as well as transformed cells, and thus provide the continuous immune surveillance that is critical for virus-host homeostasis.

So far, most studies evaluated, whether cellular EBV-specific immune responses can prevent the development of EBV⁺LDs. Studies demonstrated that innate natural killer (NK) cells can, to some extent, reduce the EBV-mediated malignant transformation of B cells via cytolysis of lytically EBV replicating B cells^{11,97,98}. Based on these studies, the question arose, whether NK cell lines can be used for the treatment of EBV⁺LDs. NK92 is a highly cytotoxic NK cell line that has been isolated from a patient with a highly aggressive EBV-NK cell nHL. These NK92 are expressing the full spectrum of activating, but only a limited set of inhibitory receptors. It was reported that NK92 cells show a high cytotoxic activity selectively against EBV-infected B cells¹⁰⁰. Importantly, earlier studies already demonstrated the safety of irradiated NK92 cell infusions into patients with advanced malignancies^{101,102} NK-92 is so far the only FDA-approved cell line to be used in clinical trials and more than 100 patients with various malignancies received infusions with unmodified, NK92-CD16a or chimeric antigen receptor targeted NK92 so far¹⁰⁰. These studies demonstrated that NK92 infusion led in some cases to disease remission in patients with renal cell cancer, melanomas, and hematological malignancies, such as myeloid leukemia, myelomas, and HL¹⁰⁰.

Other studies in individuals with broad T cell defects highlighted mainly the role of EBV-specific CD8⁺ T cells for anti-EBV immunity in general and the prevention of EBV⁺LDs in particular^{21,103} The majority of EBV-specific CD8⁺ T cell responses are directed against lytic EBV-derived antigens, while latent EBV antigen-specific CD8⁺ T cells are lower in frequency²¹. In some IM patients, nearly half of the total CD8⁺ T cell pool binds to EBV-derived antigens²⁰. Studies subsequently evaluated, whether these pronounced naturally occurring EBV-specific CD8⁺ T cell responses can protect against the development of EBV⁺LDs, but could not find a correlation between large expansions of classical, highly activated EBV-specific CD8⁺ T cells and the protection of EBV-associated malignant diseases¹⁰⁴.

Studies thus evaluated whether adoptive EBV-specific CD8⁺ T cell therapies could prevent the development or improve the clinical prognosis of EBV⁺LDs. Studies in HSCT EBV⁺PTLD patients using donor-derived EBV-specific CD8⁺ T cells, or SOT EBV⁺PTLD patients using autologous EBV-specific CD8⁺ T cells, respectively, demonstrated e.g. that adoptive cellular therapy using total ex vivo expanded EBV-specific CD8⁺ T cells could achieve complete remission¹⁰⁵. When administered prophylactically, transferred EBV-specific CD8⁺ T cells could efficiently prevent the development of EBV⁺PTLD¹⁰⁵. Autologous EBV-specific CD8⁺ T cells were also recently used to treat patients with EBV⁺LDs that express the Type I-II latency program. These studies demonstrated, however, that these EBV⁺LDs show only low and modest clinical responses to adoptively transferred EBV-specific CD8⁺ T cells that were expanded with EBV infected B cells^{106,107} These EBV-specific CD8⁺ T cells are often manufactured using donor-derived lymphoblastoid B cell lines (LCLs) that are first generated using the B95-8 strain and subsequently used as antigen-presenting cells for the expansion of donor-derived or autologous CD8⁺ T cells¹⁰⁶. In order to focus the transferred T cells towards EBV latency II recognition, other recently published studies transferred enriched LMP-specific antigen-specific CD8⁺ T cells. In these studies, up to 56% of patients infused with EBV-specific CD8⁺ T cells were in remission up to four years after treatment, and a further 26% showed clinical responses for relapsed or refractory disease^{106,108} In general, these studies demonstrate that adoptive EBV-specific CD8⁺ T cell therapies are a therapeutic option for the prevention and treatment of EBV⁺LDs and EBV⁺PTLDs but should be tailored towards EBV antigen expression in the respective tumors.

### EBV-Specific and HLA-Restricted T Cell Responses

Among the broad CD8⁺ T cell responses, a small subset of EBV-specific CD8⁺ T cells recognize with their ap T cell receptor not the classical MHC class I molecules, but the non-classical HLA-E. HLA-E is highly conserved in European populations, and only two allelic variants, the high-expressing HLA-E*0103 and the low-expressing HLA-E*0101 are prevalent⁹. This limited polymorphism results in a restricted set of EBV-derived peptides that can be presented via HLA-E on the surface of EBV-infected cells. Although EBV may encode for >40 proteins that stabilize HLA-E on the surface of EBV-infected cells, only the highly-conserved EBV-encoded peptide (SQAPLPCVL), derived from the EBV BZLF1 protein, is able to also induce highly potent HLA-E-restricted CD8⁺ T cell responses^{10,24}; see Examples 1-5.

Recently, it was shown that individuals after IM had significantly lower SQAPLPCVL-specific HLA-E-restricted CD8⁺ T cell levels, compared to individuals that had asymptomatic primary infections, demonstrating that these cells may mediate protection from IM during primary infection³⁹. These data suggest that SQAPLPCVL-specific HLA-E-restricted CD8⁺ T cells could also prevent the development of EBV⁺ LDs. Importantly, recently published studies demonstrated that HRS cells in EBV⁺HL are characterized by high frequencies of HLA-E-expressing cells. An increased HLA-E expression was further associated with an advanced clinical stage of cHL^{109,110} These studies may suggest that EBV⁺LDs are highly susceptible to EBV-specific HLA-E-restricted CD8⁺ T cell responses.

Interestingly, a recently published study demonstrated that BZLF1 is also expressed in the majority of EBV⁺PTLDs and EBV⁺DLBCLs⁹⁶. The same study demonstrated that the vaccination of a humanized mouse model with BZLF1-transduced dendritic cells did not only induce BZLF-specific CD8⁺ T cells but also significantly prolonged survival from fatal EBV⁺LDs⁹⁶. These data are of special interest, as BZLF1 is highly immunogenic in humans, and raise the question, whether BZLF1-specific immune responses can, to some extent prevent the development of EBV-associated malignancies. Thus, BZLF1-specific CD8⁺ T cells might prevent the development or improve the outcome of EBV-associated lymphomas¹⁰⁶

### Results

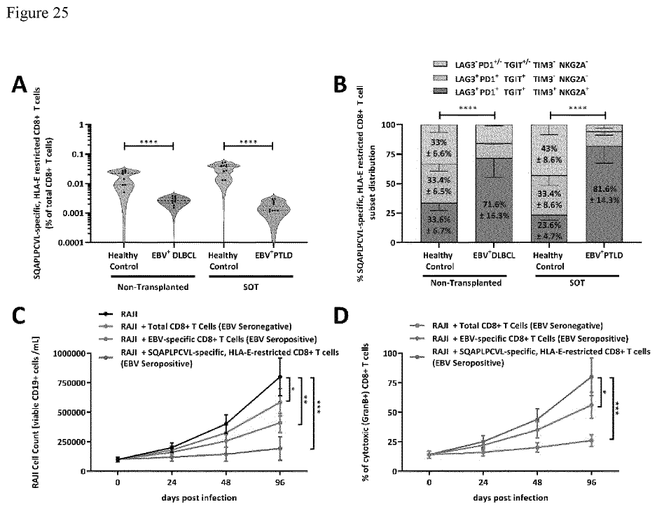

We therefore hypothesized that patients with EBV⁺LDs may have impaired SQAPLPCVL-specific HLA-E-restricted CD8⁺ T cell responses. To test our hypothesis, we recruited a study cohort of non-transplanted EBV⁺DLBCL and transplanted EBV⁺PTLD patients. We then compared the SQAPLPCVL-specific and HLA-E-restricted CD8⁺ T cell levels to non-transplanted and transplanted controls, who did not develop any EBV-associated malignancies (“healthy controls”). As shown in FIG. 25A, non-transplanted and transplanted controls without EBV-associated malignancies showed significantly higher SQAPLPCVL-specific and HLA-E-restricted CD8⁺ T cell levels compared to the EBV⁺DLBCL and EBV⁺PTLD patients, respectively.

We further analyzed the phenotype of SQAPLPCVL-specific HLA-E-restricted CD8⁺ T cells derived from the EBV⁺DLBCL and EBV⁺PTLD patients as well as the respective non-transplanted and transplanted controls. We therefore tested the SQAPLPCVL-specific HLA-E-restricted CD8⁺ T cells for the expression of LAG3, PD1, and TGIT, which reflect a repeated cellular stimulation, as well as the expression of TIM3 and NKG2A, which represent a terminally exhausted and non-functional CD8⁺ T cell phenotype 5. While the majority of SQAPLPCVL-specific HLA-E-restricted CD8⁺ T cells in the non-diseased controls showed a recently activated LAG3⁻PD1^{+/-}TGIT^{+/-}TIM⁻NKG2A⁻ or LAG3⁺PD1⁺TGIT⁺TIM⁻NKG2A⁻ phenotype, the terminally exhausted LAG3⁺PD1⁺TGIT⁺TIM⁺NKG2A⁺ phenotype significantly dominated in EBV⁺DLBCL and EBV⁺PTLD patients (FIG. 25B). Thus, the SQAPLPCVL-specific HLA-E-restricted CD8⁺ T cell responses presents with a highly exhausted phenotype in EBV⁺LD patients.

To test, whether the SQAPLPCVL-specific HLA-E-restricted CD8⁺ T cells can also prevent the proliferation of EBV⁺ lymphoma cells, we co-cultured the EBV-infected lymphoblast-like HLA-E-expressing RAJI cell line¹¹¹, originally derived from an EBV+BL patient, with sorted SQAPLPCVL-specific HLA-E-restricted CD8⁺ T cells from healthy EBV-seropositive blood donors. As controls, we also co-cultured RAJI cells with sorted total EBV-specific CD8⁺ T cells from the same healthy EBV-seropositive blood donors or total CD8⁺ T cells from healthy EBV-negative blood donors, respectively. As shown in FIG. 25C, SQAPLPCVL-specific HLA-E-restricted CD8⁺ T cells were the most efficient in the limitation of the proliferation of RAJI cells. We then also tested and compared the cytotoxic activation levels of the SQAPLPCVL-specific HLA-E-restricted CD8⁺ T cells, total EBV-specific CD8⁺ T cells, or total CD8⁺ T cells from EBV-seronegative blood donors. As shown in FIG. 25D, SQAPLPCVL-specific HLA-E-restricted CD8⁺ T cells showed significantly higher cytotoxic responses compared to total EBV-specific CD8⁺ T cells or total CD8⁺ T cell from EBV-seronegatives. These data combined show that the SQAPLPCVL-specific HLA-E-restricted CD8⁺ T cell response, which can be induced for example by an immunization via the SQAPLPCVL peptide, prevents the proliferation of EBV⁺ lymphoma cells and can thus be used to treat or prevent EBV-associated lymphoproliferative diseases.

### List of Abbreviations

| | |
|---|---|
| BAC | bacterial artificial chromosome |
| BL | Burkitt lymphoma |
| cHL-NSHD | classical-nodular sclerosis Hodgkin lymphoma |
| DLBCL | diffuse large B cell lymphoma |
| EBV | Epstein-Barr virus |
| HL | Hodgkin lymphoma |
| HRS | Hodgkin Reed-Sternberg |
| HSCT | hematopoietic stem cell transplantation |
| IE | immediate early |
| IFNγ | interferon γ |
| IM | infectious mononucleosis |
| LCL | lymphoblastoid B cell line |
| LDs | lymphoproliferative disease |
| MOI | multiplicity of infection |
| nHL | non-Hodgkin lymphoma |
| NK cell | natural killer cell |
| NPC | nasopharyngeal carcinoma |
| PTCL | peripheral T cell lymphoma |
| PTLD | post-transplant lymphoproliferative disorder |
| SOT | solid-organ transplantation |
| TCR | T cell receptor |
| TSchG | Tierschutzgesetz (Animal Welfare Act) |
| WT | wild-type |

### EXAMPLE 8

### SQAPLPCVL-Specific HLA-E-Restricted CD8⁺ T Cell Immune Responses Contribute to the Prevention of the Development of EBV-Associated Gastric Carcinoma

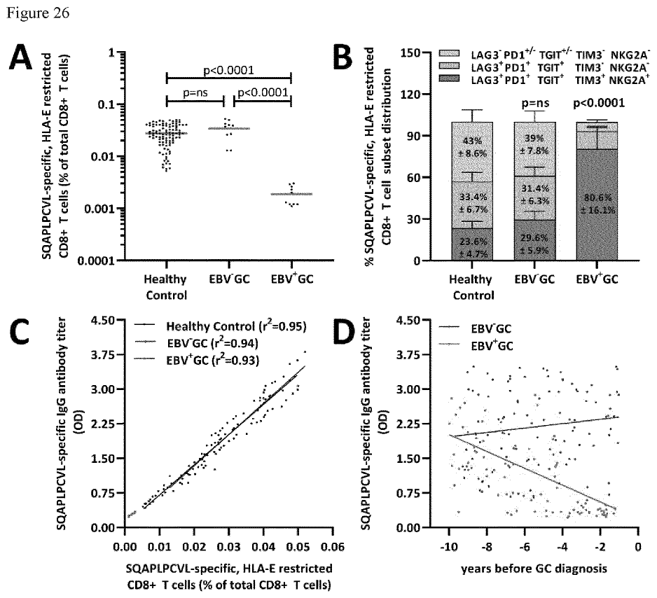

Epstein-Barr virus-associated gastric cancers (EBV⁺GCs or EBV-associated gastric carcinoma) contribute to >16% of all GCs in the US¹¹². EBV⁺GCs are usually adenocarcinomas that derive from a single EBV-infected epithelial cell¹¹³. Although most humans are EBV-infected, EBV⁺GCs occur only in a fraction of all individuals 3. Distinct EBV-specific immune responses may thus control EBV replication, eliminate transformed cells and prevent the development of EBV⁺GCs. Recently, “non-classical” EBV-specific CD8⁺ T cell responses were described that depend on the presentation of the EBV BZLF1-derived SQAPLPCVL peptide (Gene ID: 3783744) on highly conserved human HLA-E molecules¹⁰; see Examples 1-5. As demonstrated herein, these SQAPLPCVL-specific HLA-E-restricted CD8⁺ T cells are highly potent EBV-specific effector cells³⁹; see Examples 1-5. It is expected that these cells can also prevent the development or improve the clinical course of EBV⁺GCs. We thus performed a study to determine the frequency and phenotype of SQAPLPCVL-specific HLA-E-restricted CD8⁺ T cells in EBV⁺GC patients. We found that SQAPLPCVL-specific HLA-E-restricted CD8⁺ T cells occurred not only rarely in EBV⁺GC patients (FIG. 26A), but displayed also a terminally exhausted phenotype, hallmarked by the inhibitory NKG2A-receptor (Gene ID: 3821, FIG. 26B). To further evaluate, whether these immune responses may be a novel correlate of protection for EBV⁺GCs, we quantified the corresponding peptide-specific antibodies and did not only find a strong correlation to SQAPLPCVL-specific HLA-E-restricted CD8⁺ T cell frequencies, but also descending antibody concentrations before the EBV⁺GC diagnosis (FIG. 26C-D). These data demonstrate that potent SQAPLPCVL-specific HLA-E-restricted CD8⁺ T cell immune responses contribute to the prevention of the development of EBV⁺GCs. These data further suggest that the SQAPLPCVL-specific HLA-E-restricted CD8⁺ T cell response, which can be induced for example by an immunization via the SQAPLPCVL peptide, can be used to treat or prevent EBV-associated gastric carcinoma.

### LIST OF REFERENCES

1. Hocqueloux L, Causse X, Valery A, et al. The high burden of hospitalizations for primary EBV infection: a 6-year prospective survey in a French hospital. Clin Microbiol Infect. 2015; 21(11):1041.e1041-1047.
2. Rostgaard K, Balfour H H, Jr., Jarrett R, et al. Primary Epstein-Barr virus infection with and without infectious mononucleosis. PloS one. 2019; 14 (12):e0226436-e0226436.
3. Wong Y, Meehan M T, Burrows S R, Doolan D L, Miles J J. Estimating the global burden of Epstein-Barr virus-related cancers. Journal of Cancer Research and Clinical Oncology. 2022; 148(1):31-46.
4. Pearse W B, Vakkalagadda C V, Helenowski I, et al. Prognosis and Outcomes of Patients with Post-Transplant Lymphoproliferative Disorder: A Single Center Retrospective Review. Blood. 2020; 136 (Supplement 1):9-10.
5. Jorgensen P B, Livbjerg A H, Hansen H J, Petersen T, Höllsberg P. Epstein-Barr virus peptide presented by HLA-E is predominantly recognized by CD8(bright) cells in multiple sclerosis patients. PLoS One. 2012; 7(9):e46120.
6. Hatton O, Strauss-Albee D M, Zhao N Q, et al. NKG2A-Expressing Natural Killer Cells Dominate the Response to Autologous Lymphoblastoid Cells Infected with Epstein-Barr Virus. Frontiers in immunology. 2016; 7:607-607.
7. Martinez O M, Krams S M. The Immune Response to Epstein Barr Virus and Implications for Posttransplant Lymphoproliferative Disorder. Transplantation. 2017; 101(9):2009-2016.
8. Pietra G, Romagnani C, Manzini C, Moretta L, Mingari M C. The emerging role of HLA-E-restricted CD8+ T lymphocytes in the adaptive immune response to pathogens and tumors. J Biomed Biotechnol. 2010; 2010:907092.
9. Rolle A, Jager D, Momburg F. HLA-E Peptide Repertoire and Dimorphism-Centerpieces in the Adaptive N K Cell Puzzle? Frontiers in Immunology. 2018; 9(2410).
10. Mbiribindi B, Pena J K, Arvedson M P, et al. Epstein-Barr virus peptides derived from latent cycle proteins alter NKG2A+ NK cell effector function. Scientific Reports. 2020; 10(1):19973.
11. Azzi T, Lünemann A, Murer A, et al. Role for early-differentiated natural killer cells in infectious mononucleosis. Blood. 2014; 124(16):2533-2543.
12. Carel J C, Frazier B, Ley T J, Holers V M. Analysis of epitope expression and the functional repertoire of recombinant complement receptor 2 (CR2/CD21) in mouse and human cells. J Immunol. 1989; 143(3):923-930.
13. van Esser J W, van der Holt B, Meijer E, et al. Epstein-Barr virus (EBV) reactivation is a frequent event after allogeneic stem cell transplantation (SCT) and quantitatively predicts EBV-lymphoproliferative disease following T-cell-depleted SCT. Blood. 2001; 98(4):972-978.
14. Knowles D M, Cesarman E, Chadburn A, et al. Correlative morphologic and molecular genetic analysis demonstrates three distinct categories of posttransplantation lymphoproliferative disorders. Blood. 1995; 85(2):552-565.
15. Shah N, Eyre T A, Tucker D, et al. Front-line management of post-transplantation lymphoproliferative disorder in adult solid organ recipient patients—A British Society for Haematology Guideline. British Journal of Haematology. 2021; 193(4):727-740.
16. Hwang A E, Hamilton A S, Cockburn M G, et al. Evidence of genetic susceptibility to infectious mononucleosis: a twin study. Epidemiology and infection. 2012; 140(11):2089-2095.
17. Rostgaard K, Wohlfahrt J, Hjalgrim H. A genetic basis for infectious mononucleosis: evidence from a family study of hospitalized cases in Denmark. Clin Infect Dis. 2014; 58(12):1684-1689.
18. McAulay K A, Higgins C D, Macsween K F, et al. HLA class I polymorphisms are associated with development of infectious mononucleosis upon primary EBV infection. The Journal of Clinical Investigation. 2007; 117(10):3042-3048.
19. Houldcroft C J, Kellam P. Host genetics of Epstein-Barr virus infection, latency and disease. Reviews in medical virology. 2015; 25(2):71-84.
20. Long H M, Meckiff B J, Taylor G S. The T-cell Response to Epstein-Barr Virus-New Tricks From an Old Dog. Frontiers in Immunology. 2019; 10.
21. Tangye S G, Palendira U, Edwards E S J. Human immunity against EBV—lessons from the clinic. Journal of Experimental Medicine. 2017; 214(2):269-283.
22. Dunmire S K, Grimm J M, Schmeling D O, Balfour H H, Jr., Hogquist K A. The Incubation Period of Primary Epstein-Barr Virus Infection: Viral Dynamics and Immunologic Events. PLOS Pathogens. 2015; 11(12):e1005286.
23. Callan M F, Tan L, Annels N, et al. Direct visualization of antigen-specific CD8+ T cells during the primary immune response to Epstein-Barr virus In vivo. J Exp Med. 1998; 187(9):1395-1402.
24. Romagnani C, Pietra G, Falco M, et al. Identification of HLA-E-specific alloreactive T lymphocytes: A cell subset that undergoes preferential expansion in mixed lymphocyte culture and displays a broad cytolytic activity against allogeneic cells. Proceedings of the National Academy of Sciences. 2002; 99(17):11328-11333.
25. Al-Mansour Z, Nelson B P, Evens A M. Post-transplant lymphoproliferative disease (PTLD): risk factors, diagnosis, and current treatment strategies. Curr Hematol Malig Rep. 2013; 8(3):173-183.
26. Lünemann A, Vanoaica L D, Azzi T, Nadal D, Münz C. A distinct subpopulation of human NK cells restricts B cell transformation by EBV. JImmunol. 2013; 191(10):4989-4995.
27. Martin P, Krsnik I, Navarro B, et al. HLA Allele E*01:01 Is Associated with a Reduced Risk of EBV-Related Classical Hodgkin Lymphoma Independently of HLA-A*01/*02. PLOS ONE. 2015; 10(8):e0135512.
28. Vietzen H, Pollak K, Honsig C, Jaksch P, Puchhammer-Stockl E. NKG2C Deletion Is a Risk Factor for Human Cytomegalovirus Viremia and Disease After Lung Transplantation. J Infect Dis. 2018; 217(5):802-806.
29. Paquay M M, Schellekens J, Tilanus M G. A high-throughput Taqman approach for the discrimination of HLA-E alleles. Tissue Antigens. 2009; 74(6):514-519.
30. Lorenzetti M A, Gantuz M, Altcheh J, De Matteo E, Chabay P A, Preciado M V. Epstein-Barr virus BZLF1 gene polymorphisms: malignancy related or geographically distributed variants? Clinical Microbiology and Infection. 2014; 20(11):0861-0869.
31. Aberle S W, Puchhammer-Stöckl E. Diagnosis of herpesvirus infections of the central nervous system. J Clin Virol. 2002; 25 Suppl 1:S79-85.
32. Heatley S L, Pietra G, Lin J, et al. Polymorphism in human cytomegalovirus UL40 impacts on recognition of human leukocyte antigen-E (HLA-E) by natural killer cells. The Journal of biological chemistry. 2013; 288(12):8679-8690.
33. Escalante, Gabriela M., et al. “Four Decades of Prophylactic EBV Vaccine Research: A Systematic Review and Historical Perspective.” Frontiers in immunology 13 (2022): 867918-867918.
34. Ulbrecht, Matthias, et al. “Interaction of HLA-E with peptides and the peptide transporter in vitro: implications for its function in antigen presentation.” The Journal of Immunology 160.9 (1998): 4375-4385.
35. Sharpe, H. R., et al. “HLA-E: exploiting pathogen-host interactions for vaccine development.” Clinical & Experimental Immunology 196.2 (2019): 167-177.
36. Abels, Wiebke C., et al. “Peptide Presentation Is the Key to Immunotherapeutical Success.” Polypeptide-New Insight into Drug Discovery and Development (2018).
37. Bjornevik, K., Cortese, M., Healy Brian, C., Kuhle, J., Mina Michael, J., Leng, Y., Elledge Stephen, J., Niebuhr David, W., Scher Ann, I., Munger Kassandra, L., and Ascherio, A. (2022). Longitudinal analysis reveals high prevalence of Epstein-Barr virus associated with multiple sclerosis. Science (New York, N. Y.) 375, 296-301. 10.1126/science.abj8222.
38. Dunmire, S. K., Verghese, P. S., and Balfour, H. H., Jr. (2018). Primary Epstein-Barr virus infection. Journal of clinical virology: the official publication of the Pan American Society for Clinical Virology 102, 84-92. 10.1016/j.jcv.2018.03.001.
39. Vietzen, H., Furlano, P. L., Cornelissen, J. J., Böhmig, G. A., Jaksch, P., and Puchhammer-Stöckl, E. (2022). HLA-E-Restricted Immune Responses Are Crucial for the Control of EBV Infections and the Prevention of PTLD. Blood. 10.1182/blood.2022017650.
40. Lanz, T. V., Brewer, R. C., Ho, P. P., Moon, J. S., Jude, K. M., Fernandez, D., Fernandes, R. A., Gomez, A. M., Nadj, G. S., Bartley, C. M., et al. (2022). Clonally expanded B cells in multiple sclerosis bind EBV EBNA1 and GlialCAM. Nature 603, 321-327. 10.1038/s41586-022-04432-7.
41. Levin, L. I., Munger, K. L., Rubertone, M. V., Peck, C. A., Lennette, E. T., Spiegelman, D., and Ascherio, A. (2005). Temporal Relationship Between Elevation of Epstein-Barr Virus Antibody Titers and Initial Onset of Neurological Symptoms in Multiple Sclerosis. JAMA 293, 2496-2500. 10.1001/jama.293.20.2496.
42. Läderach, F., and Münz, C. (2022). Altered Immune Response to the Epstein-Barr Virus as a Prerequisite for Multiple Sclerosis. Cells 11. 10.3390/cells11172757.
43. Fowler, K., Mucha, J., Neumann, M., Lewandowski, W., Kaczanowska, M., Grys, M., Schmidt, E., Natenshon, A., Talarico, C., Buck, P. O., and Diaz-Decaro, J. (2022). A systematic literature review of the global seroprevalence of cytomegalovirus: possible implications for treatment, screening, and vaccine development. BMC Public Health 22, 1659. 10.1186/s12889-022-13971-7.
44. Grut, V., Biström, M., Salzer, J., Stridh, P., Jons, D., Gustafsson, R., Fogdell-Hahn, A., Huang, J., Brenner, N., Butt, J., et al. (2021). Cytomegalovirus seropositivity is associated with reduced risk of multiple sclerosis—a presymptomatic case-control study. European Journal of Neurology 28, 3072-3079. https://doi.org/10.1111/ene.14961.
45. Alari-Pahissa, E., Moreira, A., Zabalza, A., Alvarez-Lafuente, R., Munteis, E., Vera, A., Arroyo, R., Alvarez-Cermeno, J. C., Villar, L. M., López-Botet, M., and Martinez-Rodriguez, J. E. (2018). Low cytomegalovirus seroprevalence in early multiple sclerosis: a case for the ‘hygiene hypothesis’? Eur J Neurol 25, 925-933. 10.1111/ene.13622.
46. Lünemann, J. D., Tintoré, M., Messmer, B., Strowig, T., Rovira, A., Perkal, H., Caballero, E., Münz, C., Montalban, X., and Comabella, M. (2010). Elevated Epstein-Barr virus-encoded nuclear antigen-1 immune responses predict conversion to multiple sclerosis. Annals of Neurology 67, 159-169. https://doi.org/10.1002/ana.21886.
47. Rommer, P. S., Ellenberger, D., Hellwig, K., Haas, J., Pöhlau, D., Stahmann, A., and Zettl, U. K. (2020). Relapsing and progressive M S: the sex-specific perspective. Ther Adv Neurol Disord 13, 1756286420956495. 10.1177/1756286420956495.
48. Orton, S. M., Herrera, B. M., Yee, I. M., Valdar, W., Ramagopalan, S. V., Sadovnick, A. D., and Ebers, G. C. (2006). Sex ratio of multiple sclerosis in Canada: a longitudinal study. Lancet Neurol 5, 932-936. 10.1016/si474-4422(06)70581-6.
49. Martin, R., Sospedra, M., Eiermann, T., and Olsson, T. (2021). Multiple sclerosis: doubling down on MHC. Trends Genet 37, 784-797. 10.1016/j.tig.2021.04.012.
50. Vogel, D. Y. S., Vereyken, E. J. F., Glim, J. E., Heijnen, P. D. A. M., Moeton, M., van der Valk, P., Amor, S., Teunissen, C. E., van Horssen, J., and Dijkstra, C. D. (2013). Macrophages in inflammatory multiple sclerosis lesions have an intermediate activation status. Journal of Neuroinflammation 10, 809. 10.1186/1742-2094-10-35.
51. Borst, L., Sluijter, M., Sturm, G., Charoentong, P., Santegoets, S. J., van Gulijk, M., van Elsas, M. J., Groeneveldt, C., van Montfoort, N., Finotello, F., et al. (2022). NKG2A is a late immune checkpoint on CD8 T cells and marks repeated stimulation and cell division. International journal of cancer 150, 688-704. https://doi.org/10.1002/ijc.33859.
52. Hammer, Q., Ruckert, T., Borst, E. M., Dunst, J., Haubner, A., Durek, P., Heinrich, F., Gasparoni, G., Babic, M., Tomic, A., et al. (2018). Peptide-specific recognition of human cytomegalovirus strains controls adaptive natural killer cells. Nat Immunol 19, 453-463. 10.1038/s41590-018-0082-6.
53. Goodier, M. R., White, M. J., Darboe, A., Nielsen, C. M., Goncalves, A., Bottomley, C., Moore, S. E., and Riley, E. M. (2014). Rapid NK cell differentiation in a population with near-universal human cytomegalovirus infection is attenuated by NKG2C deletions. Blood 124, 2213-2222. 10.1182/blood-2014-05-576124.
54. Espinoza, J. L., Nguyen, V. H., Ichimura, H., Pham, T. T. T., Nguyen, C. H., Pham, T. V., Elbadry, M. I., Yoshioka, K., Tanaka, J., Trung, L. Q., et al. (2016). A functional polymorphism in the NKG2D gene modulates N K-cell cytotoxicity and is associated with susceptibility to Human Papilloma Virus-related cancers. Scientific reports 6, 39231. 10.1038/srep39231.
55. Wang, X., Xiong, H., and Ning, Z. (2022). Implications of NKG2A in immunity and immune-mediated diseases. Front Immunol 13, 960852. 10.3389/fimmu.2022.960852.
56. Morandi, F., Airoldi, I., and Pistoia, V. (2014). IL-27 driven upregulation of surface HLA-E expression on monocytes inhibits IFN-7 release by autologous NK cells. J Immunol Res 2014, 938561. 10.1155/2014/938561.
57. Mbiribindi, B., Pena, J. K., Arvedson, M. P., Moreno Romero, C., McCarthy, S. R., Hatton, O. L., Esquivel, C. O., Martinez, O. M., and Krams, S. M. (2020). Epstein-Barr virus peptides derived from latent cycle proteins alter NKG2A+ NK cell effector function. Scientific reports 10, 19973. 10.1038/s41598-020-76344-3.
58. Beli8n, J., Goris, A., and Matthys, P. (2022). Natural Killer Cells in Multiple Sclerosis: Entering the Stage. Front Immunol 13, 869447. 10.3389/fimmu.2022.869447.
59. Kim, K. H., Yu, H. T., Hwang, I., Park, S., Park, S.-H., Kim, S., and Shin, E.-C. (2019). Phenotypic and Functional Analysis of Human N K Cell Subpopulations According to the Expression of FcFRIT and NKG2C. Frontiers in Immunology 10, 2865.
60. Rückert, T., Lareau, C. A., Mashreghi, M. F., Ludwig, L. S., and Romagnani, C. (2022). Clonal expansion and epigenetic inheritance of long-lasting NK cell memory. Nat Immunol 23, 1551-1563. 10.1038/s41590-022-01327-7.
61. Wensveen, F. M., Jelenčić, V., and Polić, B. (2018). NKG2D: A Master Regulator of Immune Cell Responsiveness. Front Immunol 9, 441. 10.3389/fimmu.2018.00441.
62. Lopez-Verges, S., Milush, J. M., Schwartz, B. S., Pando, M. J., Jarjoura, J., York, V. A., Houchins, J. P., Miller, S., Kang, S. M., Norris, P. J., et al. (2011). Expansion of a unique CD57(+)NKG2Chi natural killer cell subset during acute human cytomegalovirus infection. Proceedings of the National Academy of Sciences of the United States of America 108, 14725-14732. 10.1073/pnas.1110900108.
63. Waubant, E., Mowry, E. M., Krupp, L., Chitnis, T., Yeh, E. A., Kuntz, N., Ness, J., Chabas, D., Strober, J., McDonald, J., et al. (2011). Common viruses associated with lower pediatric multiple sclerosis risk. Neurology 76, 1989-1995. 10.1212/WNL.0b013e31821e552a.
64. Vanheusden, M., Stinissen, P., t Hart, B. A., and Hellings, N. (2015). Cytomegalovirus: a culprit or protector in multiple sclerosis? Trends Mol Med 21, 16-23. 10.1016/j.molmed.2014.11.002.
65. Zivadinov, R., Nasuelli, D., Tommasi, M. A., Serafin, M., Bratina, A., Ukmar, M., Pirko, I., Johnson, A. J., Furlan, C., Pozzi-Mucelli, R. S., et al. (2006). Positivity of cytomegalovirus antibodies predicts a better clinical and radiological outcome in multiple sclerosis patients. Neurological Research 28, 262-269. 10.1179/016164106X98134.
66. Martínez-Rodríguez, J. E., Cobo-Calvo, A., Villar, L. M., Munteis, E., Blanco, Y., Rasal, R., Vera, A., Muntasell, A., Alvarez-Lafuente, R., Saiz, A., et al. (2016). Adaptive natural killer cell response to cytomegalovirus and disability progression in multiple sclerosis. Mult Scler 22, 741-752. 10.1177/1352458515601215.
67. Li, H., Liu, S., Hu, J., Luo, X., Li, N., A, M. B., and Cao, Y. (2016). Epstein-Barr virus lytic reactivation regulation and its pathogenic role in carcinogenesis. Int J Biol Sci 12, 1309-1318. 10.7150/ijbs.16564.
68. Larousserie, F., Bardel, E., Pflanz, S., Arnulf, B., Lome-Maldonado, C., Hermine, O., Brégeaud, L., Perennec, M., Brousse, N., Kastelein, R., and Devergne, O. (2005). Analysis of interleukin-27 (EBI3/p28) expression in Epstein-Barr virus- and human T-cell leukemia virus type 1-associated lymphomas: heterogeneous expression of EBI3 subunit by tumoral cells. Am J Pathol 166, 1217-1228. 10.1016/s0002-9440(10)62340-1.
69. Lalive, P. H., Kreutzfeldt, M., Devergne, O., Metz, I., Bruck, W., Merkler, D., and Pot, C. (2017). Increased interleukin-27 cytokine expression in the central nervous system of multiple sclerosis patients. Journal of Neuroinflammation 14, 144. 10.1186/s12974-017-0919-1.
70. Senecal, V., Deblois, G., Beauseigle, D., Schneider, R., Brandenburg, J., Newcombe, J., Moore, C. S., Prat, A., Antel, J., and Arbour, N. (2016). Production of IL-27 in multiple sclerosis lesions by astrocytes and myeloid cells: Modulation of local immune responses. Glia 64, 553-569. 10.1002/glia.22948.
71. Clenet, M. L., Laurent, C., Lemaitre, F., Farzam-Kia, N., Tastet, O., Devergne, O., Lahav, B., Girard, M., Duquette, P., Prat, A., et al. (2021). The IL-27/IL-27R axis is altered in CD4(+) and CD8(+) T lymphocytes from multiple sclerosis patients. Clin Transl Immunology 10, e1262. 10.1002/cti2.1262.
72. Yoshida, H., and Hunter, C. A. (2015). The Immunobiology of Interleukin-27. Annual review of immunology 33, 417-443. 10.1146/annurev-immunol-032414-112134.
73. Fitzgerald, D. C., Zhang, G. X., El-Behi, M., Fonseca-Kelly, Z., Li, H., Yu, S., Saris, C. J., Gran, B., Ciric, B., and Rostami, A. (2007). Suppression of autoimmune inflammation of the central nervous system by interleukin 10 secreted by interleukin 27-stimulated T cells. Nat Immunol 8, 1372-1379. 10.1038/ni1540.
74. Batten, M., Li, J., Yi, S., Kljavin, N. M., Danilenko, D. M., Lucas, S., Lee, J., de Sauvage, F. J., and Ghilardi, N. (2006). Interleukin 27 limits autoimmune encephalomyelitis by suppressing the development of interleukin 17-producing T cells. Nature Immunology 7, 929-936. 10.1038/ni1375.
75. Murugaiyan, G., Beynon, V., Pires Da Cunha, A., Joller, N., and Weiner, H. L. (2012). IFN-γ limits Th9-mediated autoimmune inflammation through dendritic cell modulation of IL-27. Journal of immunology (Baltimore, Md.: 1950) 189, 5277-5283. 10.4049/jimmunol.1200808.
76. Laroni, A., Armentani, E., Kerlero de Rosbo, N., Ivaldi, F., Marcenaro, E., Sivori, S., Gandhi, R., Weiner, H. L., Moretta, A., Mancardi, G. L., and Uccelli, A. (2016). Dysregulation of regulatory CD56bright NK cells/T cells interactions in multiple sclerosis. Journal of Autoimmunity 72, 8-18. https://doi.org/10.1016/j.jaut.2016.04.003.
77. Kaur, G., Trowsdale, J., and Fugger, L. (2013). Natural killer cells and their receptors in multiple sclerosis. Brain 136, 2657-2676. 10.1093/brain/awsl59.
78. Durrenberger, P. F., Webb, L. V., Sim, M. J., Nicholas, R. S., Altmann, D. M., and Boyton, R. J. (2012). Increased HLA-E expression in white matter lesions in multiple sclerosis. Immunology 137, 317-325. 10.1111/imm.12012.
79. Consortium*†, I. M. S. G., Patsopoulos, N. A., Baranzini, S. E., Santaniello, A., Shoostari, P., Cotsapas, C., Wong, G., Beecham, A. H., James, T., Replogle, J., et al. (2019). Multiple sclerosis genomic map implicates peripheral immune cells and microglia in susceptibility. Science (New York, N. Y.) 365, eaav7188. doi:10.1126/science.aav7188.
80. Thompson, A. J., Banwell, B. L., Barkhof, F., Carroll, W. M., Coetzee, T., Comi, G., Correale, J., Fazekas, F., Filippi, M., Freedman, M. S., et al. (2018). Diagnosis of multiple sclerosis: 2017 revisions of the McDonald criteria. Lancet Neurol 17, 162-173. 10.1016/s1474-4422(17)30470-2.
81. Vietzen, H., Gorzer, I., Honsig, C., Jaksch, P., and Puchhammer-Stöckl, E. (2020). HCMV-Specific Antibody Response And Development Of ADCC Against HCMV After Lung Transplantation. The Journal of infectious diseases. 10.1093/infdis/jiaa097.
82. Eberhard, S., Vietzen, H., Gorzer, I., Jaksch, P., and Puchhammer-Stöckl, E. (2021). Analysis and Fine Specificity of the HCMV-Specific Cell-Free and Cell-Associated Antibody-Dependent Cellular Phagocytosis (ADCP) Responses in Lung Transplant Recipients. Int J Mol Sci 22. 10.3390/ijms22158206.
83. Bein, G., Glaser, R., and Kirchner, H. (1992). Rapid HLA-DRB1 genotyping by nested PCR amplification. Tissue antigens 39, 68-73. https://doi.org/10.1111/j.1399-0039.1992.tb01909.x.
84. Vietzen, H., Zoufaly, A., Traugott, M., Aberle, J., Aberle, S. W., and Puchhammer-Stöckl, E. (2021). Deletion of the NKG2C receptor encoding KLRC2 gene and HLA-E variants are risk factors for severe COVID-19. Genet Med, 1-5. 10.1038/s41436-020-01077-7.
85. Vietzen, H., Rückert, T., Hartenberger, S., Honsig, C., Jaksch, P., Geleff, S., Hammer, Q., Romagnani, C., Segura-Wang, M., and Puchhammer-Stöckl, E. (2021). Extent of Cytomegalovirus Replication in the Human Host Depends on Variations of the HLA-E/UL40 Axis. mBio 12. 10.1128/mBio.02996-20.
86. Kimura, H., Miyake, K., Yamauchi, Y., Nishiyama, K., Iwata, S., Iwatsuki, K., Gotoh, K., Kojima, S., Ito, Y., and Nishiyama, Y. (2009). Identification of Epstein-Barr Virus (EBV)-Infected Lymphocyte Subtypes by Flow Cytometric In Situ Hybridization in EBV-Associated Lymphoproliferative Diseases. The Journal of infectious diseases 200, 1078-1087. 10.1086/605610.
87. Castillo, J. J., Beltran, B. E., Miranda, R. N., Paydas, S., Winer, E. S., and Butera, J. N. (2011). Epstein-Barr Virus-Positive Diffuse Large B-Cell Lymphoma of the Elderly: What We Know So Far. The Oncologist 16, 87-96. 10.1634/theoncologist.2010-0213.
88. Leoncini, L. (2022). Epstein-Barr virus positivity as a defining pathogenetic feature of Burkitt lymphoma subtypes. British Journal of Haematology 196, 468-470. https://doi.org/10.1111/bjh.17922.
89. Parker, A., Bowles, K., Bradley, J. A., Emery, V., Featherstone, C., Gupte, G., Marcus, R., Parameshwar, J., Ramsay, A., and Newstead, C. (2010). Diagnosis of post-transplant lymphoproliferative disorder in solid organ transplant recipients—BCSH and BTS Guidelines. Br J Haematol 149, 675-692. 10.1111/j.1365-2141.2010.08161.x.
90. Opelz, G., and Dohler, B. (2004). Lymphomas after solid organ transplantation: a collaborative transplant study report. American journal of transplantation: official journal of the American Society of Transplantation and the American Society of Transplant Surgeons 4, 222-230. 10.1046/j.1600-6143.2003.00325.x.
91. Nijland, M. L., Kersten, M. J., Pals, S T., Bemelman, F. J., and Ten Berge, I. J. (2016). Epstein-Barr Virus-Positive Posttransplant Lymphoproliferative Disease After Solid Organ Transplantation: Pathogenesis, Clinical Manifestations, Diagnosis, and Management. Transplant Direct 2, e48. 10.1097/txd.0000000000000557.
92. Thirumalai, D., Watson, C., Xun, P., Sadetsky, N., Schaible, K., and Barlev, A. (2021). Incidence of Post-Transplant Lymphoproliferative Disease: A Systematic Literature Review. Blood 138, 4564. https://doi.org/10.1182/blood-2021-147715.
93. Shen, J., Luo, M., Liu, L., Chen, Q.-Y., Tang, L.-Q., and Mai, H.-Q. (2021). Incidence and trend of Epstein-Barr virus-related cancer: A surveillance, epidemiology, and end results program based study. Journal of Clinical Oncology 39, 10575-10575. 10.1200/JCO.2021.39.15_suppl.10575.
94. Lv, K., Yin, T., Yu, M., Chen, Z., Zhou, Y., and Li, F. (2022). Treatment Advances in EBV Related Lymphoproliferative Diseases. Frontiers in Oncology 12. 10.3389/fonc.2022.838817.
95. Tse, E., and Kwong, Y. L. (2015). Epstein Barr virus-associated lymphoproliferative diseases: the virus as a therapeutic target. Exp Mol Med 47, e136. 10.1038/emm.2014.102.
96. Hartlage, A. S., Liu, T., Patton, J. T., Garman, S. L., Zhang, X., Kurt, H., Lozanski, G., Lustberg, M. E., Caligiuri, M. A., and Baiocchi, R. A. (2015). The Epstein-Barr Virus Lytic Protein BZLF1 as a Candidate Target Antigen for Vaccine Development. Cancer Immunology Research 3, 787-794. 10.1158/2326-6066.CIR-14-0242.
97. Chijioke, O., Landtwing, V., and Münz, C. (2016). N K Cell Influence on the Outcome of Primary Epstein-Barr Virus Infection. Front Immunol 7, 323. 10.3389/fimmu.2016.00323.
98. Chung, B. K., Tsai, K., Allan, L. L., Zheng, D. J., Nie, J. C., Biggs, C. M., Hasan, M. R., Kozak, F. K., van den Elzen, P., Priatel, J. J., and Tan, R. (2013). Innate immune control of EBV-infected B cells by invariant natural killer T cells. Blood 122, 2600-2608. 10.1182/blood-2013-01-480665.
100. Klingemann, H., Boissel, L., and Toneguzzo, F. (2016). Natural Killer Cells for Immunotherapy—Advantages of the NK-92 Cell Line over Blood N K Cells. Frontiers in Immunology 7. 10.3389/fimmu.2016.00091.
101. Arai, S., Meagher, R., Swearingen, M., Myint, H., Rich, E., Martinson, J., and Klingemann, H. (2008). Infusion of the allogeneic cell line NK-92 in patients with advanced renal cell cancer or melanoma: a phase I trial. Cytotherapy 10, 625-632. 10.1080/14653240802301872.
102. Tonn, T., Schwabe, D., Klingemann, H. G., Becker, S., Esser, R., Koehl, U., Suttorp, M., Seifried, E., Ottmann, O. G., and Bug, G. (2013). Treatment of patients with advanced cancer with the natural killer cell line NK-92. Cytotherapy 15, 1563-1570. 10.1016/j.jcyt.2013.06.017.
103. Palendira, U., and Rickinson, A. B. (2015). Primary immunodeficiencies and the control of Epstein-Barr virus infection. Ann N Y Acad Sci 1356, 22-44. 10.1111/nyas.12937.
104. Hislop, A. D., Taylor, G. S., Sauce, D., and Rickinson, A. B. (2007). Cellular Responses to Viral Infection in Humans: Lessons from Epstein-Barr Virus. Annual review of immunology 25, 587-617. 10.1146/annurev.immunol.25.022106.141553.
105. Heslop, H. E., Slobod, K. S., Pule, M. A., Hale, G. A., Rousseau, A., Smith, C. A., Bollard, C. M., Liu, H., Wu, M.-F., Rochester, R. J., et al. (2010). Long-term outcome of EBV-specific T-cell infusions to prevent or treat EBV-related lymphoproliferative disease in transplant recipients. Blood 115, 925-935. 10.1182/blood-2009-08-239186.
106. Heslop, H. E., Sharma, S., and Rooney, C. M. (2021). Adoptive T-Cell Therapy for Epstein-Barr Virus-Related Lymphomas. Journal of Clinical Oncology 39, 514-524. 74 10.1200/JCO.20.01709.
107. Wu, J., Cao, Y., Zhang, Q., Liu, W., Zhou, X., Ming, X., Meng, F., Zhang, Y., Li, C., Huang, L., et al. (2022). Chimeric Antigen Receptor-Modified T Cell Immunotherapy for Relapsed and Refractory Adult Burkitt Lymphoma. Front Immunol 13, 879983. 10.3389/fimmu.2022.879983.
108. Bollard, C. M., Gottschalk, S., Torrano, V., Diouf, O., Ku, S., Hazrat, Y., Carrum, G., Ramos, C., Fayad, L., Shpall, E. J., et al. (2014). Sustained complete responses in patients with lymphoma receiving autologous cytotoxic T lymphocytes targeting Epstein-Barr virus latent membrane proteins. Journal of clinical oncology: official journal of the American Society of Clinical Oncology 32, 798-808. 10.1200/jco.2013.51.5304.
109. Chiu, J., Ernst, D. M., and Keating, A. (2018). Acquired Natural Killer Cell Dysfunction in the Tumor Microenvironment of Classic Hodgkin Lymphoma. Front Immunol 9, 267. 10.3389/fimmu.2018.00267.
110. Kren, L., Fabian, P., Slaby, O., Janikova, A., Soucek, O., Sterba, J., Krenova, Z., Michalek, J., and Kral, Z. (2012). Multifunctional immune-modulatory protein HLA-E identified in classical Hodgkin lymphoma: possible implications. Pathol Res Pract 208, 45-49. 10.1016/j.prp.2011.11.004.
111. Lo Monaco, E., Tremante, E., Cerboni, C., Melucci, E., Sibilio, L., Zingoni, A., Nicotra, M. R., Natali, P. G., and Giacomini, P. (2011). Human leukocyte antigen E contributes to protect tumor cells from lysis by natural killer cells. Neoplasia 13, 822-830. 10.1593/neo.101684.
112. Takada K. Epstein-Barr virus and gastric carcinoma. Mol Pathol. 2000; 53(5):255-61.
113. Yang J, Liu Z, Zeng B, Hu G, Gan R. Epstein-Barr virus-associated gastric cancer: A distinct subtype. Cancer Lett. 2020; 495:191-9.
114. Swerdlow, S. H., Campo, E., Pileri, S. A., Harris, N. L., Stein, H., Siebert, R., Advani, R., Ghielmini, M., Salles, G. A., Zelenetz, A. D., and Jaffe, E. S. (2016). The 2016 revision of the World Health Organization classification of lymphoid neoplasms. Blood 127, 2375-2390. 10.1182/blood-2016-01-643569.

1. An Epstein-Barr-Virus (EBV) vaccine, comprising a SQAPLPCVL peptide or a nucleic acid encoding a SQAPLPCVL peptide and, optionally, a pharmaceutically acceptable carrier, for use in the treatment of EBV or an EBV-induced disease.

2. The EBV vaccine for use of claim 1, wherein the EBV-induced disease is selected from the group of infectious mononucleosis (IM), lymphoproliferative diseases and/or malignant diseases.

3. The EBV vaccine for use of claim 1 or 2, wherein the EBV-induced disease is infectious mononucleosis (IM) caused by a primary infection with EBV (which may include postinfectious chronic fatigue syndrome), post-transplant lymphoproliferative disorder (PTLD), malignant post-transplant lymphoproliferative disorder (PTLD), Burkitt lymphoma, hemophagocytic lymphohistiocytosis, EBV-associated Hodgkin lymphoma, EBV-associated gastric carcinoma, nasopharyngeal carcinoma, B-cell lymphoma, lymphomatoid granulomatosis, multiple sclerosis, long COVID, myalgic encephalomyelitis or chronic fatigue syndrome.

4. The EBV vaccine for use of any one of claims 1-3, wherein the treatment of EBV is the treatment of EBV infection, preferably wherein EBV infection is EBV primary infection or the treatment of EBV infection following EBV reactivation or EBV reinfections, preferably wherein the treatment is a prophylactic treatment (prevention).

5. The EBV vaccine for use of any one of claims 1-4, wherein the treatment comprises administering the EBV vaccine to a subject in an effective amount.

6. The EBV vaccine for use of claim 5, wherein an effective amount is a total dose of 30-3000 μg.

7. The EBV vaccine for use of claim 5 or 6, wherein the EBV vaccine is administered preferably by intradermal injection, intramuscular injection or subcutaneous injection.

8. The EBV vaccine for use of any one of claims 1-7, wherein the treatment is a prophylactic treatment (prevention) and/or a therapeutic/acute treatment.

9. The EBV vaccine for use of any one of claims 1-8, wherein the subject has been exposed to EBV, is infected with EBV or is at risk of EBV infection or an EBV-induced disease.

10. The EBV vaccine for use of any one of claims 1-9, wherein the subject has received or will receive a transplant, such as an organ or stem cell transplant and/or, wherein the subject has a congenital or acquired immunodeficiency.

11. The EBV vaccine for use of claim 10, wherein a subject has received or will receive a solid-organ or hematopoietic stem cell transplant.

12. The EBV vaccine for use of any one of claims 1-11, wherein the subject is not infected with EBV.

13. The EBV vaccine for use of any one of claims 1-12, wherein the subject is a human.

14. The EBV vaccine for use of claim 13, wherein the human has an HLA-E*0101/0101, HLA-E*0101/0103 or HLA-E*0103/0103 genotype.

15. The EBV vaccine for use of claim 13 or 14, wherein the subject is a human in an EBV endemic region or population.

16. The EBV vaccine for use of any one of claims 13-15, wherein the subject is a young child, an adolescent/teenager, an adult, or an elderly person.

17. The EBV vaccine for use of any one of claims 1-16, wherein a single dose of the EBV vaccine is administered.

18. The EBV vaccine for use of claim 17, wherein the treatment further comprises administering one or more booster doses of the EBV vaccine.

19. The EBV vaccine for use of any one of claims 1-18, wherein the EBV vaccine produces or is capable of producing an EBV-specific immune response (in the subject to be treated/to whom the vaccine is to be administered).

20. The EBV vaccine for use of claim 19, wherein the immune response is a T cell response.

21. The EBV vaccine for use of claim 19 or 20, wherein the immune response is a HLA-E-restricted CD8⁺ T cell response.

22. The EBV vaccine for use of claim 21, wherein the HLA-E-restricted CD8⁺ T cell response comprises a HLA-E-restricted NKG2A⁺ CD8⁺ T cell response and/or a NKG2A⁻ CD8⁺ T cell response.

23. The EBV vaccine for use of claim 21 or 22, wherein the HLA-E-restricted CD8⁺ T cell response is a SQAPLPCVL-specific HLA-E-restricted CD8⁺ T cell response, optionally comprising a SQAPLPCVL-specific HLA-E-restricted NKG2A⁺ CD8⁺ T cell response and/or a SQAPLPCVL-specific HLA-E-restricted NKG2A⁻ CD8⁺ T cell response.

24. The EBV vaccine for use of any one of claims 1 to 23, wherein the EBV vaccine and/or the immune response prevent EBV spread and/or EBV replication or is capable of preventing EBV spread and/or EBV replication (in the subject to be treated/to whom the vaccine is to be administered).

25. The EBV vaccine for use of any one of claims 1-24, wherein the EBV vaccine results in long-term immunity to EBV or an EBV-induced disease.

26. The EBV vaccine for use of any one of claims 1-25, wherein the EBV vaccine results in EBV-specific memory B cells or memory T cells.

27. The EBV vaccine for use of any one of claims 1-26, wherein the nucleic acid encoding a SQAPLPCVL peptide is a non-self-replicating desoxyribonucleic acid (DNA), such as a vector.

28. The EBV vaccine for use of any one of claims 1-27, wherein the nucleic acid encoding a SQAPLPCVL peptide is a non-self-replicating ribonucleic acid (RNA), such as a mRNA, optionally wherein the RNA is a modified RNA, such as a modified mRNA.

29. A method of treating EBV or an EBV-induced disease comprising administering an effective amount of an EBV vaccine to a subject, wherein the EBV vaccine comprises a SQAPLPCVL peptide or a nucleic acid encoding a SQAPLPCVL peptide and, optionally a pharmaceutically acceptable carrier.

30. The method of claim 29, wherein the EBV-induced disease is selected from the group of infectious mononucleosis (IM), lymphoproliferative diseases or malignant diseases.

31. The method of claim 29 or 30 wherein the EBV-induced disease is infectious mononucleosis (IM) caused by a primary infection with EBV (which may include postinfectious chronic fatigue syndrome), post-transplant lymphoproliferative disorder (PTLD), malignant post-transplant lymphoproliferative disorder (PTLD), Burkitt lymphoma, hemophagocytic lymphohistiocytosis, EBV-associated Hodgkin lymphoma, EBV-associated gastric carcinoma, nasopharyngeal carcinoma, B-cell lymphoma, lymphomatoid granulomatosis, multiple sclerosis, long COVID, myalgic encephalomyelitis or chronic fatigue syndrome.

32. The method of any one of claims 29-31, wherein the treatment of EBV is the treatment of EBV infection, preferably wherein EBV infection is EBV primary infection or the treatment of EBV infection following EBV reactivation or EBV reinfections, preferably wherein the treatment is a prophylactic treatment (prevention).

33. The method of any one of claims 29-32, wherein an effective amount is a total dose of 30-3000 μg.

34. The method of any one of claims 29-33, wherein the EBV vaccine is administered by intradermal injection, intramuscular injection, or subcutaneous injection.

35. The method of any one of claims 29-34, wherein the treatment is a prophylactic treatment (prevention) and/or a therapeutic/acute treatment.

36. The method of any one of claims 29-35, wherein the subject has been exposed to EBV, is infected with EBV or is at risk of EBV infection.

37. The method of any one of claims 29-36, wherein the subject has received or will receive a transplant such as an organ or stem cell transplant and/or, wherein the subject has a congenital or acquired immunodeficiency.

38. The method of claim 37, wherein a subject has received or will receive a solid-organ or hematopoietic stem cell transplant.

39. The method of any one of claims 29-38, wherein the subject is not infected with EBV.

40. The method of any one of claims 29-39, wherein the subject is a human.

41. The method of claim 40, wherein the human has an HLA-E*0101/0101, HLA-E*0101/0103 or HLA-E*0103/0103 genotype.

42. The method of claim 40 or 41, wherein the subject is a human in an EBV endemic region or population.

43. The method of any one of claims 40-42, wherein the subject is a young child, an adolescent/teenager, an adult or an elderly person.

44. The method of any one of claims 29-43, wherein a single dose of the EBV vaccine is administered.

45. The method of claim 44, wherein the method further comprises administering one or more booster doses of the EBV vaccine.

46. The method of any one of claims 29-45, wherein the EBV vaccine produces an EBV-specific immune response.

47. The method of claim 46, wherein the immune response is a T cell response.

48. The method of claim 46 or 47, wherein the immune response is a HLA-E-restricted CD8⁺ T cell response.

49. The method of claim 48, wherein the HLA-E-restricted CD8⁺ T cell response comprises a HLA-E-restricted NKG2A⁺ CD8⁺ T cell response and/or a NKG2A⁺ CD8⁺ T cell response.

50. The method of claim 48 or 49, wherein the HLA-E-restricted CD8⁺ T cell response is a SQAPLPCVL-specific HLA-E-restricted CD8⁺ T cell response, optionally comprising a SQAPLPCVL-specific HLA-E-restricted NKG2A⁺ CD8⁺ T cell response and/or a SQAPLPCVL-specific HLA-E-restricted NKG2A⁻ CD8⁺ T cell response.

51. The method of any one of claims 29-50, wherein the administration of the EBV vaccine and/or the immune response prevent EBV spread and/or EBV replication or is capable of preventing EBV spread and/or EBV replication (in the subject to be treated/to whom the vaccine is to be administered).

52. The method of any one of claims 29-51, wherein the EBV vaccine results in long-term immunity to EBV or an EBV-induced disease.

53. The method of any one of claims 29-52, wherein the EBV vaccine results in EBV-specific memory B cells or memory T cells.

54. The method of any one of claims 29-53, wherein the nucleic acid encoding a SQAPLPCVL peptide is a non-self-replicating desoxyribonucleic acid (DNA), such as a vector.

55. The method of any one of claims 29-53, wherein the nucleic acid encoding a SQAPLPCVL peptide is a non-self-replicating ribonucleic acid (RNA), such as a mRNA, optionally wherein the RNA is a modified RNA, such as a modified mRNA.

56. A method of inducing an EBV-specific immune response in a subject, comprising administering an EBV vaccine to a subject, wherein the EBV vaccine comprises a SQAPLPCVL peptide or a nucleic acid encoding a SQAPLPCVL peptide and optionally a pharmaceutically acceptable carrier.

57. A method to produce HLA-E-restricted CD8⁺ T cells optionally comprising HLA-E-restricted NKG2A⁺ CD8⁺ T cells and/or HLA-E-restricted NKG2A⁺ CD8⁺ T cells in a subject,
or to elicit a HLA-E-restricted CD8⁺ T cell response optionally comprising a NKG2A⁺ CD8⁺ T cell response and/or a NKG2A⁻ CD8⁺ T cell response in a subject, comprising administering an EBV vaccine to the subject, wherein the EBV vaccine comprises a SQAPLPCVL peptide or a nucleic acid encoding a SQAPLPCVL peptide and, optionally, a pharmaceutically acceptable carrier.

58. An in vitro or ex vivo method to produce HLA-E-restricted CD8⁺ T cells optionally comprising HLA-E-restricted NKG2A⁺ CD8⁺ T cells and/or HLA-E-restricted NKG2A-CD8⁺ T cells, or to elicit a HLA-E-restricted CD8⁺ T cell response optionally comprising a NKG2A⁺ CD8⁺ T cell response and/or a NKG2A⁻ CD8⁺ T cell response in a cell, comprising contacting a cell with an EBV vaccine, wherein the EBV vaccine comprises a SQAPLPCVL peptide or a nucleic acid encoding a SQAPLPCVL peptide and, optionally, a pharmaceutically acceptable carrier.

59. An Epstein-Barr-Virus (EBV) vaccine, comprising a SQAPLPCVL peptide or a nucleic acid encoding a SQAPLPCVL peptide and, optionally, a pharmaceutically acceptable carrier.

60. A SQAPLPCVL peptide or a nucleic acid encoding a SQAPLPCVL peptide for use as a vaccine.

61. A SQAPLPCVL peptide or a nucleic acid encoding a SQAPLPCVL peptide for use as a vaccine against EBV.

62. A SQAPLPCVL peptide or a nucleic acid encoding a SQAPLPCVL peptide for use as a medicament.

63. A SQAPLPCVL peptide or a nucleic acid encoding a SQAPLPCVL peptide for use in the treatment of EBV or an EBV-induced disease.

64. The SQAPLPCVL peptide or a nucleic acid encoding a SQAPLPCVL peptide for use of claim 63, wherein the treatment of EBV is the treatment of EBV infection, preferably wherein EBV infection is EBV primary infection or the treatment of EBV infection following EBV reactivation or EBV reinfections, preferably wherein the treatment is a prophylactic treatment (prevention).


SEQUENCE LISTING (223 sequences)

SEQ ID NO: 1 - protein, 9 aa, synthetic construct
SQAPLPCVL

SEQ ID NO: 2 - protein, 9 aa, synthetic construct
GGDPHLPTL

SEQ ID NO: 3 - protein, 9 aa, synthetic construct
GSDPHLPTL

SEQ ID NO: 4 - protein, 9 aa, synthetic construct
GGDPHLPPL

SEQ ID NO: 5 - protein, 9 aa, synthetic construct
GGDPPLPTL

SEQ ID NO: 6 - protein, 9 aa, synthetic construct
GCDPHLPTL

SEQ ID NO: 7 - protein, 9 aa, synthetic construct
GIDPHLPTL

SEQ ID NO: 8 - protein, 9 aa, synthetic construct
GAGPHLPTL

SEQ ID NO: 9 - protein, 9 aa, synthetic construct
GGDTPLPTL

SEQ ID NO: 10 - protein, 9 aa, synthetic construct
GDDPHLPTL

SEQ ID NO: 11 - protein, 9 aa, synthetic construct
GGDPHVPTL

SEQ ID NO: 12 - protein, 9 aa, synthetic construct
GTDPHLPTL

SEQ ID NO: 13 - protein, 9 aa, synthetic construct
GSDPHLPPL

SEQ ID NO: 14 - protein, 9 aa, synthetic construct
VMAPRTLIL

SEQ ID NO: 15 - protein, 9 aa, synthetic construct
AQAPLPCVL

SEQ ID NO: 16 - protein, 9 aa, synthetic construct
RQAPLPCVL

SEQ ID NO: 17 - protein, 9 aa, synthetic construct
NQAPLPCVL

SEQ ID NO: 18 - protein, 9 aa, synthetic construct
DQAPLPCVL

SEQ ID NO: 19 - protein, 9 aa, synthetic construct
CQAPLPCVL

SEQ ID NO: 20 - protein, 9 aa, synthetic construct
QQAPLPCVL

SEQ ID NO: 21 - protein, 9 aa, synthetic construct
EQAPLPCVL

SEQ ID NO: 22 - protein, 9 aa, synthetic construct
GQAPLPCVL

SEQ ID NO: 23 - protein, 9 aa, synthetic construct
LQAPLPCVL

SEQ ID NO: 24 - protein, 9 aa, synthetic construct
HQAPLPCVL

SEQ ID NO: 25 - protein, 9 aa, synthetic construct
IQAPLPCVL

SEQ ID NO: 26 - protein, 9 aa, synthetic construct
MQAPLPCVL

SEQ ID NO: 27 - protein, 9 aa, synthetic construct
FQAPLPCVL

SEQ ID NO: 28 - protein, 9 aa, synthetic construct
KQAPLPCVL

SEQ ID NO: 29 - protein, 9 aa, synthetic construct
PQAPLPCVL

SEQ ID NO: 30 - protein, 9 aa, synthetic construct
TQAPLPCVL

SEQ ID NO: 31 - protein, 9 aa, synthetic construct
WQAPLPCVL

SEQ ID NO: 32 - protein, 9 aa, synthetic construct
YQAPLPCVL

SEQ ID NO: 33 - protein, 9 aa, synthetic construct
VQAPLPCVL

SEQ ID NO: 34 - protein, 9 aa, synthetic construct
SAAPLPCVL

SEQ ID NO: 35 - protein, 9 aa, synthetic construct
SRAPLPCVL

SEQ ID NO: 36 - protein, 9 aa, synthetic construct
SNAPLPCVL

SEQ ID NO: 37 - protein, 9 aa, synthetic construct
SDAPLPCVL

SEQ ID NO: 38 - protein, 9 aa, synthetic construct
SCAPLPCVL

SEQ ID NO: 39 - protein, 9 aa, synthetic construct
SEAPLPCVL

SEQ ID NO: 40 - protein, 9 aa, synthetic construct
SGAPLPCVL

SEQ ID NO: 41 - protein, 9 aa, synthetic construct
SHAPLPCVL

SEQ ID NO: 42 - protein, 9 aa, synthetic construct
SIAPLPCVL

SEQ ID NO: 43 - protein, 9 aa, synthetic construct
SLAPLPCVL

SEQ ID NO: 44 - protein, 9 aa, synthetic construct
SKAPLPCVL

SEQ ID NO: 45 - protein, 9 aa, synthetic construct
SMAPLPCVL

SEQ ID NO: 46 - protein, 9 aa, synthetic construct
SFAPLPCVL

SEQ ID NO: 47 - protein, 9 aa, synthetic construct
SPAPLPCVL

SEQ ID NO: 48 - protein, 9 aa, synthetic construct
SSAPLPCVL

SEQ ID NO: 49 - protein, 9 aa, synthetic construct
STAPLPCVL

SEQ ID NO: 50 - protein, 9 aa, synthetic construct
SWAPLPCVL

SEQ ID NO: 51 - protein, 9 aa, synthetic construct
SYAPLPCVL

SEQ ID NO: 52 - protein, 9 aa, synthetic construct
SVAPLPCVL

SEQ ID NO: 53 - protein, 9 aa, synthetic construct
SQRPLPCVL

SEQ ID NO: 54 - protein, 9 aa, synthetic construct
SQNPLPCVL

SEQ ID NO: 55 - protein, 9 aa, synthetic construct
SQDPLPCVL

SEQ ID NO: 56 - protein, 9 aa, synthetic construct
SQCPLPCVL

SEQ ID NO: 57 - protein, 9 aa, synthetic construct
SQQPLPCVL

SEQ ID NO: 58 - protein, 9 aa, synthetic construct
SQEPLPCVL

SEQ ID NO: 59 - protein, 9 aa, synthetic construct
SQGPLPCVL

SEQ ID NO: 60 - protein, 9 aa, synthetic construct
SQHPLPCVL

SEQ ID NO: 61 - protein, 9 aa, synthetic construct
SQIPLPCVL

SEQ ID NO: 62 - protein, 9 aa, synthetic construct
SQLPLPCVL

SEQ ID NO: 63 - protein, 9 aa, synthetic construct
SQKPLPCVL

SEQ ID NO: 64 - protein, 9 aa, synthetic construct
SQMPLPCVL

SEQ ID NO: 65 - protein, 9 aa, synthetic construct
SQFPLPCVL

SEQ ID NO: 66 - protein, 9 aa, synthetic construct
SQPPLPCVL

SEQ ID NO: 67 - protein, 9 aa, synthetic construct
SQSPLPCVL

SEQ ID NO: 68 - protein, 9 aa, synthetic construct
SQTPLPCVL

SEQ ID NO: 69 - protein, 9 aa, synthetic construct
SQWPLPCVL

SEQ ID NO: 70 - protein, 9 aa, synthetic construct
SQYPLPCVL

SEQ ID NO: 71 - protein, 9 aa, synthetic construct
SQVPLPCVL

SEQ ID NO: 72 - protein, 9 aa, synthetic construct
SQAALPCVL

SEQ ID NO: 73 - protein, 9 aa, synthetic construct
SQARLPCVL

SEQ ID NO: 74 - protein, 9 aa, synthetic construct
SQANLPCVL

SEQ ID NO: 75 - protein, 9 aa, synthetic construct
SQADLPCVL

SEQ ID NO: 76 - protein, 9 aa, synthetic construct
SQACLPCVL

SEQ ID NO: 77 - protein, 9 aa, synthetic construct
SQAQLPCVL

SEQ ID NO: 78 - protein, 9 aa, synthetic construct
SQAELPCVL

SEQ ID NO: 79 - protein, 9 aa, synthetic construct
SQAGLPCVL

SEQ ID NO: 80 - protein, 9 aa, synthetic construct
SQAHLPCVL

SEQ ID NO: 81 - protein, 9 aa, synthetic construct
SQAILPCVL

SEQ ID NO: 82 - protein, 9 aa, synthetic construct
SQALLPCVL

SEQ ID NO: 83 - protein, 9 aa, synthetic construct
SQAKLPCVL

SEQ ID NO: 84 - protein, 9 aa, synthetic construct
SQAMLPCVL

SEQ ID NO: 85 - protein, 9 aa, synthetic construct
SQAFLPCVL

SEQ ID NO: 86 - protein, 9 aa, synthetic construct
SQASLPCVL

SEQ ID NO: 87 - protein, 9 aa, synthetic construct
SQATLPCVL

SEQ ID NO: 88 - protein, 9 aa, synthetic construct
SQAWLPCVL

SEQ ID NO: 89 - protein, 9 aa, synthetic construct
SQAYLPCVL

SEQ ID NO: 90 - protein, 9 aa, synthetic construct
SQAVLPCVL

SEQ ID NO: 91 - protein, 9 aa, synthetic construct
SQAPAPCVL

SEQ ID NO: 92 - protein, 9 aa, synthetic construct
SQAPRPCVL

SEQ ID NO: 93 - protein, 9 aa, synthetic construct
SQAPNPCVL

SEQ ID NO: 94 - protein, 9 aa, synthetic construct
SQAPDPCVL

SEQ ID NO: 95 - protein, 9 aa, synthetic construct
SQAPCPCVL

SEQ ID NO: 96 - protein, 9 aa, synthetic construct
SQAPQPCVL

SEQ ID NO: 97 - protein, 9 aa, synthetic construct
SQAPEPCVL

SEQ ID NO: 98 - protein, 9 aa, synthetic construct
SQAPGPCVL

SEQ ID NO: 99 - protein, 9 aa, synthetic construct
SQAPHPCVL

SEQ ID NO: 100 - protein, 9 aa, synthetic construct
SQAPIPCVL

SEQ ID NO: 101 - protein, 9 aa, synthetic construct
SQAPKPCVL

SEQ ID NO: 102 - protein, 9 aa, synthetic construct
SQAPMPCVL

SEQ ID NO: 103 - protein, 9 aa, synthetic construct
SQAPFPCVL

SEQ ID NO: 104 - protein, 9 aa, synthetic construct
SQAPPPCVL

SEQ ID NO: 105 - protein, 9 aa, synthetic construct
SQAPSPCVL

SEQ ID NO: 106 - protein, 9 aa, synthetic construct
SQAPTPCVL

SEQ ID NO: 107 - protein, 9 aa, synthetic construct
SQAPWPCVL

SEQ ID NO: 108 - protein, 9 aa, synthetic construct
SQAPYPCVL

SEQ ID NO: 109 - protein, 9 aa, synthetic construct
SQAPVPCVL

SEQ ID NO: 110 - protein, 9 aa, synthetic construct
SQAPLACVL

SEQ ID NO: 111 - protein, 9 aa, synthetic construct
SQAPLRCVL

SEQ ID NO: 112 - protein, 9 aa, synthetic construct
SQAPLNCVL

SEQ ID NO: 113 - protein, 9 aa, synthetic construct
SQAPLDCVL

SEQ ID NO: 114 - protein, 9 aa, synthetic construct
SQAPLCCVL

SEQ ID NO: 115 - protein, 9 aa, synthetic construct
SQAPLQCVL

SEQ ID NO: 116 - protein, 9 aa, synthetic construct
SQAPLECVL

SEQ ID NO: 117 - protein, 9 aa, synthetic construct
SQAPLGCVL

SEQ ID NO: 118 - protein, 9 aa, synthetic construct
SQAPLHCVL

SEQ ID NO: 119 - protein, 9 aa, synthetic construct
SQAPLICVL

SEQ ID NO: 120 - protein, 9 aa, synthetic construct
SQAPLLCVL

SEQ ID NO: 121 - protein, 9 aa, synthetic construct
SQAPLKCVL

SEQ ID NO: 122 - protein, 9 aa, synthetic construct
SQAPLMCVL

SEQ ID NO: 123 - protein, 9 aa, synthetic construct
SQAPLFCVL

SEQ ID NO: 124 - protein, 9 aa, synthetic construct
SQAPLSCVL

SEQ ID NO: 125 - protein, 9 aa, synthetic construct
SQAPLTCVL

SEQ ID NO: 126 - protein, 9 aa, synthetic construct
SQAPLWCVL

SEQ ID NO: 127 - protein, 9 aa, synthetic construct
SQAPLYCVL

SEQ ID NO: 128 - protein, 9 aa, synthetic construct
SQAPLVCVL

SEQ ID NO: 129 - protein, 9 aa, synthetic construct
SQAPLPAVL

SEQ ID NO: 130 - protein, 9 aa, synthetic construct
SQAPLPRVL

SEQ ID NO: 131 - protein, 9 aa, synthetic construct
SQAPLPNVL

SEQ ID NO: 132 - protein, 9 aa, synthetic construct
SQAPLPDVL

SEQ ID NO: 133 - protein, 9 aa, synthetic construct
SQAPLPQVL

SEQ ID NO: 134 - protein, 9 aa, synthetic construct
SQAPLPEVL

SEQ ID NO: 135 - protein, 9 aa, synthetic construct
SQAPLPGVL

SEQ ID NO: 136 - protein, 9 aa, synthetic construct
SQAPLPHVL

SEQ ID NO: 137 - protein, 9 aa, synthetic construct
SQAPLPIVL

SEQ ID NO: 138 - protein, 9 aa, synthetic construct
SQAPLPLVL

SEQ ID NO: 139 - protein, 9 aa, synthetic construct
SQAPLPKVL

SEQ ID NO: 140 - protein, 9 aa, synthetic construct
SQAPLPMVL

SEQ ID NO: 141 - protein, 9 aa, synthetic construct
SQAPLPFVL

SEQ ID NO: 142 - protein, 9 aa, synthetic construct
SQAPLPPVL

SEQ ID NO: 143 - protein, 9 aa, synthetic construct
SQAPLPSVL

SEQ ID NO: 144 - protein, 9 aa, synthetic construct
SQAPLPTVL

SEQ ID NO: 145 - protein, 9 aa, synthetic construct
SQAPLPWVL

SEQ ID NO: 146 - protein, 9 aa, synthetic construct
SQAPLPYVL

SEQ ID NO: 147 - protein, 9 aa, synthetic construct
SQAPLPVVL

SEQ ID NO: 148 - protein, 9 aa, synthetic construct
SQAPLPCAL

SEQ ID NO: 149 - protein, 9 aa, synthetic construct
SQAPLPCRL

SEQ ID NO: 150 - protein, 9 aa, synthetic construct
SQAPLPCNL

SEQ ID NO: 151 - protein, 9 aa, synthetic construct
SQAPLPCDL

SEQ ID NO: 152 - protein, 9 aa, synthetic construct
SQAPLPCCL

SEQ ID NO: 153 - protein, 9 aa, synthetic construct
SQAPLPCQL

SEQ ID NO: 154 - protein, 9 aa, synthetic construct
SQAPLPCEL

SEQ ID NO: 155 - protein, 9 aa, synthetic construct
SQAPLPCGL

SEQ ID NO: 156 - protein, 9 aa, synthetic construct
SQAPLPCHL

SEQ ID NO: 157 - protein, 9 aa, synthetic construct
SQAPLPCIL

SEQ ID NO: 158 - protein, 9 aa, synthetic construct
SQAPLPCLL

SEQ ID NO: 159 - protein, 9 aa, synthetic construct
SQAPLPCKL

SEQ ID NO: 160 - protein, 9 aa, synthetic construct
SQAPLPCML

SEQ ID NO: 161 - protein, 9 aa, synthetic construct
SQAPLPCFL

SEQ ID NO: 162 - protein, 9 aa, synthetic construct
SQAPLPCPL

SEQ ID NO: 163 - protein, 9 aa, synthetic construct
SQAPLPCSL

SEQ ID NO: 164 - protein, 9 aa, synthetic construct
SQAPLPCTL

SEQ ID NO: 165 - protein, 9 aa, synthetic construct
SQAPLPCWL

SEQ ID NO: 166 - protein, 9 aa, synthetic construct
SQAPLPCYL

SEQ ID NO: 167 - protein, 9 aa, synthetic construct
SQAPLPCVA

SEQ ID NO: 168 - protein, 9 aa, synthetic construct
SQAPLPCVR

SEQ ID NO: 169 - protein, 9 aa, synthetic construct
SQAPLPCVN

SEQ ID NO: 170 - protein, 9 aa, synthetic construct
SQAPLPCVD

SEQ ID NO: 171 - protein, 9 aa, synthetic construct
SQAPLPCVC

SEQ ID NO: 172 - protein, 9 aa, synthetic construct
SQAPLPCVQ

SEQ ID NO: 173 - protein, 9 aa, synthetic construct
SQAPLPCVE

SEQ ID NO: 174 - protein, 9 aa, synthetic construct
SQAPLPCVG

SEQ ID NO: 175 - protein, 9 aa, synthetic construct
SQAPLPCVH

SEQ ID NO: 176 - protein, 9 aa, synthetic construct
SQAPLPCVI

SEQ ID NO: 177 - protein, 9 aa, synthetic construct
SQAPLPCVK

SEQ ID NO: 178 - protein, 9 aa, synthetic construct
SQAPLPCVM

SEQ ID NO: 179 - protein, 9 aa, synthetic construct
SQAPLPCVF

SEQ ID NO: 180 - protein, 9 aa, synthetic construct
SQAPLPCVP

SEQ ID NO: 181 - protein, 9 aa, synthetic construct
SQAPLPCVS

SEQ ID NO: 182 - protein, 9 aa, synthetic construct
SQAPLPCVT

SEQ ID NO: 183 - protein, 9 aa, synthetic construct
SQAPLPCVW

SEQ ID NO: 184 - protein, 9 aa, synthetic construct
SQAPLPCVY

SEQ ID NO: 185 - protein, 9 aa, synthetic construct
SQAPLPCVV

SEQ ID NO: 186 - protein, 15 aa, synthetic construct
QQPSQAPLPCVLWPV

SEQ ID NO: 187 - protein, 14 aa, synthetic construct
QPSQAPLPCVLWPV

SEQ ID NO: 188 - protein, 14 aa, synthetic construct
QQPSQAPLPCVLWP

SEQ ID NO: 189 - protein, 13 aa, synthetic construct
QPSQAPLPCVLWP

SEQ ID NO: 190 - protein, 12 aa, synthetic construct
QPSQAPLPCVLW

SEQ ID NO: 191 - protein, 12 aa, synthetic construct
PSQAPLPCVLWP

SEQ ID NO: 192 - protein, 11 aa, synthetic construct
PSQAPLPCVLW

SEQ ID NO: 193 - protein, 10 aa, synthetic construct
SQAPLPCVLW

SEQ ID NO: 194 - protein, 10 aa, synthetic construct
PSQAPLPCVL

SEQ ID NO: 195 - protein, 8 aa, synthetic construct
QAPLPCVL

SEQ ID NO: 196 - protein, 7 aa, synthetic construct
APLPCVL

SEQ ID NO: 197 - protein, 6 aa, synthetic construct
PLPCVL

SEQ ID NO: 198 - protein, 8 aa, synthetic construct
SQAPLPCV

SEQ ID NO: 199 - protein, 7 aa, synthetic construct
SQAPLPC

SEQ ID NO: 200 - protein, 6 aa, synthetic construct
SQAPLP

SEQ ID NO: 201 - protein, 245 aa, synthetic construct
MMDPNSTSEDVKFTPDPYQVPFVQAFDQATRVYQDLGGPSQAPLPCVLWPVLPEPLPQGQLTAYHVSTAPTGSWFSAPQPAPENAYQAYAAPQLFPVSDITQNQQTNQAGGEAPQPGDNSTVQTAAAVVFACPGANQGQQLADIGVPQPAPVAAPARRTRKPQQPESLEECDSELEIKRYKNRVASRKCRAKFKQLLQHYREVAAAKSSENDRLRLLLKQMCPSLDVDSIIPRTPDVLHEDLLNF

SEQ ID NO: 202 - protein, 66 aa, synthetic construct
DLGGPSQAPLPCVLWPVLPEPLPQGQLTAYHVSTAPTGSWFSAPQPAPENAYQAYAAPQLFPVSDI

SEQ ID NO: 203 - protein, 51 aa, synthetic construct
RKPQQPESLEECDSELEIKRYKNRVASRKCRAKFKQLLQHYREVAAAKSSE

SEQ ID NO: 204 - protein, 9 aa, synthetic construct
VMAPRTLVL

SEQ ID NO: 205 - protein, 9 aa, synthetic construct
VMAPRTLLL

SEQ ID NO: 206 - protein, 9 aa, synthetic construct
VMAPRTLFL

SEQ ID NO: 207 - protein, 20 aa, synthetic construct
SQSSSSGSPPRRPPPGRRPF

SEQ ID NO: 208 - protein, 20 aa, synthetic construct
ATGRTHSSPPRAPSSPGRSR

SEQ ID NO: 209 - protein, 9 aa, synthetic construct
VMTPRTLIL

SEQ ID NO: 210 - protein, 9 aa, synthetic construct
VMTPRTLLL

SEQ ID NO: 211 - protein, 9 aa, synthetic construct
VMTPRTLVL

SEQ ID NO: 212 - protein, 9 aa, synthetic construct
VMAPRSLLL

SEQ ID NO: 213 - protein, 9 aa, synthetic construct
VMAPRILVL

SEQ ID NO: 214 - protein, 9 aa, synthetic construct
VMAPRSLIL

SEQ ID NO: 215 - protein, 9 aa, synthetic construct
VLAPRTLLL

SEQ ID NO: 216 - protein, 9 aa, synthetic construct
VMAPRILIL

SEQ ID NO: 217 - protein, 9 aa, synthetic construct
VMGPRTLIL

SEQ ID NO: 218 - protein, 9 aa, synthetic construct
VVAPRTLVL

SEQ ID NO: 219 - protein, 9 aa, synthetic construct
GMAPRTLLL

SEQ ID NO: 220 - protein, 9 aa, synthetic construct
VIAPRTLIL

SEQ ID NO: 221 - protein, 9 aa, synthetic construct
VMAPWTLVL

SEQ ID NO: 222 - protein, 9 aa, synthetic construct
VMAPWTLIL

SEQ ID NO: 223 - protein, 9 aa, synthetic construct
GDPKHPKSF